(19) United States
(12) Patent Application Publication
(10) Pub. No.: US 2024/0425502 A1
(43) Pub. Date: Dec. 26, 2024

(54) BICYCLIC HETEROAROMATIC COMPOUNDS AND THEIR USE AS PEST CONTROL AGENTS

(71) Applicant: PI INDUSTRIES LTD., Udaipur -Rajasthan (IN)

(72) Inventors: Narasimha Murthy CHEEMALA, Andhra Pradesh (IN); Sudipta BHATTACHARYYA, West Bengal (IN); Jeevan Singh VERMA, Madhya Pradesh (IN); Gagan KUKREJA, Pitampura Delhi (IN); Ankur Aniruddh UPADHYAY, Gujarat (IN); Komal NAGDA, Udaipur, Rajasthan (IN); Ajay YADAV, Udaipur, Rajasthan (IN); Ravikumar Suryanarayana SARAGUR, Bengaluru, Karnataka (IN); Alexander G.M. KLAUSENER, Pulheim (DE)

(21) Appl. No.: 18/709,849

(22) PCT Filed: Nov. 14, 2022
(86) PCT No.: PCT/IB2022/060907

§ 371 Date: May 14, 2024

(30) Foreign Application Priority Data
Nov. 15, 2021 (IN) 202111052372
Aug. 6, 2022 (IN) 202211045008

(51) Int. Cl. C07D 471/04; A01N 43/90; A01P 3/00; A01P 5/00; A01P 7/02; A01P 7/04; A01P 13/00; A01P 21/00; C05G 3/60

(52) U.S. Cl. CPC C07D 471/04; A01N 43/90; A01P 3/00; A01P 5/00; A01P 7/02; A01P 7/04; A01P 13/00; A01P 21/00; C05G 3/60

(57) ABSTRACT

The present invention discloses an N-substituted bicyclic heteroaromatic compound of formula (I),

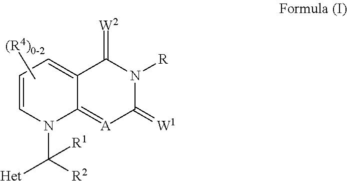

wherein, R, R¹, R², R⁴, W¹, W², A and Het are as defined in the detailed description. The present invention further discloses methods for preparation of compounds of formula (I) and use of the compounds of formula (I) as a pest control agent.

### FIELD OF THE INVENTION

The present invention relates to compounds of formula (I). More particularly, the present invention relates to N-substituted bicyclic heteroaromatic compounds of formula (I) and a process for the preparation thereof. The present invention further relates to compositions comprising those compounds, and to their use as pest control agents.

### BACKGROUND OF THE INVENTION

Invertebrate pests, in particular arthropods and nematodes, destroy growing and harvested crops thereby causing severe economic loss to the food supply. Just as well, insects also attack wooden dwellings and commercial structures causing large economic losses to properties as well. Therefore, there is an ongoing need for new agents for combating invertebrate pests such as insects, arachnids and nematodes. The currently available modern insecticides and acaricides have to satisfy many requirements, for example regarding the level of activity, long lasting efficacy, broadness of insecticidal spectrum as well as environmental and toxicological safeness and additional beneficial effects, and the possible use thereof. Efforts have been made during the past decades to develop selective insecticides that are acting specifically on biochemical modes of action being present only in insects or mites but additionally showing properties that differ from known insecticides in an advantageous way.

Fused bicyclic heteroaromatic compounds with pesticidal activity are known in the prior art, for example, from WO2007115647, WO2012136751, WO2014076272, WO2020/025658 and WO2020/011808.

However, there still is a continuous need for new compounds which are even more effective in combating invertebrate pests, less toxic, environmentally safer and/or have different modes of action.

Surprisingly, it has now been found that the N-substituted bicyclic heteroaromatic compounds of formula (I) have advantages over the prior art, for example, by showing improved insecticidal activity.

In view of the above, the present invention envisages such compounds that satisfy or overcome drawbacks associated with the prior art. It has now surprisingly been found that certain novel, pesticidally active N-substituted bicyclic heteroaromatic compounds being subject of this invention, have favorable properties as pesticides and are environmentally safer, as desired.

### SUMMARY OF THE INVENTION

Accordingly, the present invention provides N-substituted bicyclic heteroaromatic compounds of formula (I) or salts, stereo-isomers, metal complexes, polymorphs, or N-oxides thereof,

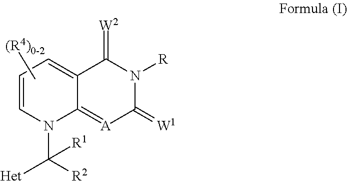

wherein, R, R¹, R², R⁴, W¹, W², A and Het are as defined in the detailed description.

In one embodiment, the present invention provides a process for preparing the compounds of formula (I) or salts thereof.

In another embodiment, the present invention provides a composition for controlling or preventing invertebrate pests comprising a biologically effective amount of a compound of formula (I) or salts, stereo-isomers, metal complexes, polymorphs, or N-oxides thereof and at least one additional component selected from the group consisting of surfactants and auxiliaries.

In yet another embodiment, the composition further comprises at least one additional biologically active and compatible compound selected from fungicides, insecticides, nematicides, acaricides, biopesticides, herbicides, plant growth regulators, antibiotics, fertilizers or nutrients.

In still another embodiment, the present invention provides the use of the compounds of formula (I) or salts, stereo-isomers, metal complexes, polymorphs, or N-oxides, compositions or combinations thereof, for combating invertebrate pests in agricultural and horticultural crops and parasites on animals or wooden dwellings and commercial structures.

In yet still another embodiment, the present invention provides a method of combating invertebrate pests comprising contacting the invertebrate pests, their habitat, breeding ground, food supply, plant, seed, soil, area, material or environment in which the invertebrate pests are growing or may grow, or the materials, plants, seeds, soils, surfaces or spaces to be protected from pest attack or infestation with a biologically effective amount of the compounds of formula (I) or salts, stereo-isomers, metal complexes, polymorphs, or N-oxides thereof as well as compositions or combinations thereof.

### DETAILED DESCRIPTION OF THE INVENTION

### Definitions

The definitions provided herein for the terminologies used in the present disclosure are for illustrative purposes only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Tematoda), acanthocephala and tapeworms (Cestoda).

The term “agronomic” refers to the production of field crops such as for food, feed and fiber and includes the growth of corn, soybeans and other legumes, rice, cereals (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruits (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

The compounds of the present disclosure may be present either in pure form or as mixtures of different possible isomeric forms such as stereoisomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated.

The term “enantiomerically enriched” refers to a chiral substance whose enantiomeric ratio is greater than 50:50 but less than 100:0. The term “aliphatic compound/s” or “aliphatic group/s” used herein is an organic compound/s whose carbon atoms are linked in straight chains, branched chains, or non-aromatic rings.

The term “alkyl”, used either alone or in compound words such as “alkylthio” or “haloalkyl” or —N(alkyl) or alkylcarbonylalkyl or alkylsuphonylamino includes straight-chain or branched C₁ to C₁₀ alkyl, preferably C₁ to C₈ alkyl, more preferably C₁ to C₆ alkyl, most preferably C₁ to C₄ alkyl. Representative examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “cyanoalkyl” as used herein refers to straight-chain or branched alkyl groups having 1 to 10 carbon atoms (“C₁-C₁₀-cyanoalkyl”), preferably 1 to 6 (“C₁-C₆-cyanoalkyl”) carbon atoms (as mentioned above), where 1 or 2, preferably 1, of the hydrogen atoms in these groups are replaced by a cyano (CN) group. Non limiting examples are cyanomethyl, 1-cyanoethyl, 2-cyanoethyl, 1-cyanopropyl, 2-cyanopropyl, 3-cyanopropyl, 1-cyanobutyl, 2-cyanobutyl, 3-cyanobutyl, 4-cyanobutyl and the like.

The term “alkenyl”, used either alone or in compound words includes straight-chain or branched C₂ to C₁₀ alkenes, preferably C₂ to C₈ alkenes, more preferably C₂ to C₆ alkenes, most preferably C₂ to C₄ alkenes. Representative examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

The term “alkynyl”, used either alone or in compound words includes straight-chain or branched C₂ to C₁₀ alkynes, preferably C₂ to C₈ alkynes, more preferably C₂ to C₆ alkynes, most preferably C₂ to C₄ alkynes. Non-limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “alkynyl” can also include moieties comprised of multiple triple bonds such as 2,5-hexadiynyl.

The term “cycloalkyl” as used herein refers to alkyl closed to form a ring groups having 3 to 10 (“C₃-C₁₀-cycloalkyl”) carbon atoms; preferably C₃-C₈-cycloalkyl, more preferably C₃-C₆-cycloalkyl. Non-limiting examples include but are not limited to cyclopropyl, cyclobutyl, cyclopentyl and cyclohexyl. This definition also applies to cycloalkyl as a part of a composite substituent, for example cycloalkylalkyl etc., unless specifically defined elsewhere.

“Bicycloalkyl” or “bicyclic alkyl” means a saturated or partially unsaturated fused, spiro or bridged bicyclic ring assembly. In particular embodiments, “bicycloalkyl,” either alone or represented along with another radical, can be a (C₄-C₁₅)bicycloalkyl, a (C₄-C₁₀)bicycloalkyl, a (C₆-C₁₀)bicycloalkyl or a (C₈-C₁₀)bicycloalkyl. Alternatively, “bicycloalkyl,” either alone or represented along with another radical, can be a (C₇)bicycloalkyl, a (C₈)bicycloalkyl or a (C₁₀)bicycloalkyl.

The term “cycloalkenyl” means alkenyl closed to form a ring including monocyclic, partially unsaturated hydrocarbyl groups. Non-limiting examples include but are not limited to cyclopropenyl, cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. Non-limiting examples include but are not limited to cyclopropynyl, cyclopentynyl and cyclohexynyl. This definition also applies to cycloalkynyl as a part of a composite substituent, for example cycloalkynylalkyl etc., unless specifically defined elsewhere.

The term “cycloalkoxy”, “cycloalkenyloxy” and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “cyanocycloalkyl” as used herein refers to alkyl closed to form a ring groups having 3 to 10 (“C₃-C₁₀-cyanoalkyl”) carbon atoms (as mentioned above), where 1 or 2, preferably 1, of the hydrogen atoms in these groups are replaced by a cyano (CN) group. Non limiting examples are 1-cyanocyclopropyl, 2-cyanocyclopropyl, 1-cyanocyclobutyl, 2-cyanocyclobutyl, 3-cyanocyclobutyl, 1-cyanocyclopentyl, 2-cyanocyclopentyl, 3-cyclopentyl, 4-cyclopentyl, 1-cyanohexyl, 2-cyanohexyl, 3-cyanocyclohexyl, 4-cyanocyclohexyl and the like.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl”, “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2- fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non-limiting examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂.

The term “hydroxy” means —OH, amino means —NRR, wherein R can be H or any possible substituent such as alkyl. Carbonyl means —C(O)—, carbonyloxy means —OC(O)—, sulfinyl means SO, sulfonyl means S(O)₂, nitro means —NO₂, cyano means —CN.

The term “alkoxy” used either alone or in compound words includes C₁ to C₁₀ alkoxy, preferably C₁ to C₈ alkoxy, more preferably C₁ to C₆ alkoxy, most preferably C₁ to C₄ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “Alkoxyalkyl” denotes alkoxy substitution on alkyl. Non-limiting examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkoxyalkoxy” denotes alkoxy substitution on alkoxy.

The term “alkylthio” or “(alkylsulfanyl: S-alkyl)” as used herein refers to a straight-chain or branched saturated alkyl group having 1 to 10 carbon atoms, preferably 1 to 6 carbon atoms, more preferably 1 to 4 carbon atoms which is attached via a sulfur atom. The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, halocycloalkylalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

The term “alkylthioalkyl” denotes alkylthio substitution on alkyl. Representative examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. The term “cycloalkylalkylamino” denotes cycloalkyl substitution on alkyl amino.

The terms alkoxyalkoxyalkyl, alkylaminoalkyl, dialkylaminoalkyl, cycloalkylaminoalkyl, cycloalkylaminocarbonyl and the like, are defined analogously to “alkylthioalkyl” or cycloalkylalkylamino.

The term “alkoxycarbonyl” is an alkoxy group bonded to a skeleton via a carbonyl group (—CO—).

This definition also applies to alkoxycarbonyl as a part of a composite substituent, for example cycloalkylalkoxycarbonyl and the like, unless specifically defined elsewhere.

The term “alkylsulfinyl” (alkylsulfoxyl: S(═O)-alkyl), as used herein, refers to a straight-chain or branched saturated alkyl group (as mentioned above) having 1 to 10 carbon atoms, preferably 1 to 6 carbon atoms (C₁-C₆-alkylsulfinyl), more preferably 1 to 4 carbon atoms bonded through the sulfur atom of the sulfinyl group at any position in the alkyl group.Non-limiting examples of “alkylsulfinyl” include but are not limited to methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, hexylsulfinyl, 1,1-dimethylpropylsulfinyl, 1,2-dimethylpropylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl and the different isomers. The term “arylsulfinyl” includes Ar—S(O), wherein Ar can be any carbocyle or heterocylcle. This definition also applies to alkylsulfinyl as a part of a composite substituent, for example haloalkylsulfinyl etc., unless specifically defined elsewhere.

The term “alkylsulfonyl” (S(═O)₂-alkyl) as used herein refers to a straight-chain or branched saturated alkyl group having 1 to 10 carbon atoms, preferably 1 to 6 carbon atoms (C₁-C₆-alkylsulfonyl), preferably 1 to 4 carbon atoms, which is bonded via the sulfur atom of the sulfonyl group at any position in the alkyl group. Non-limiting examples of “alkylsulfonyl” include but are not limited to methylsulfonyl, ethylsulfonyl, propylsulfonyl, 1-methylethylsulfonyl, butylsulfonyl, 1-methylpropylsulfonyl, 2-methylpropylsulfonylsulfonyl, 1,1-dimethylethylsulfonyl, pentylsulfonyl, 1-methylbutylsulfonyl, 2-methylbutylsulfonyl, 3-methylbutylsulfonyl, 2,2-dimethylpropylsulfonyl, 1-ethylpropylsulfonyl, hexylsulfonyl, 1,1-dimethylpropylsulfonyl, 1,2-dimethylpropylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl and the different isomers. The term “arylsulfonyl” includes Ar—S(O)₂, wherein Ar can be any carbocyle or heterocylcle. This definition also applies to alkylsulfonyl as a part of a composite substituent, for example alkylsulfonylalkyl etc., unless defined elsewhere.

“Alkylamino”, “dialkylamino”, and the like, are defined analogously to the above examples.

The term “aryl” as used herein is a group that contains any carbon-based aromatic group including, but not limited to phenyl, naphthalene, biphenyl, anthracene, and the like. The aryl group can be substituted or unsubstituted. In addition, the aryl group can be a single ring structure or comprise multiple ring structures that are either fused ring structures or attached via one or more bridging groups such as a carbon-carbon bond.

The term “aryl” also comprises “aralkyl” refers to aryl hydrocarbon radicals including an alkyl portion as defined above. Examples include benzyl, phenylethyl, and 6-naphthylhexyl. As used herein, the term “aralkenyl” refers to aryl hydrocarbon radicals including an alkenyl portion, as defined above, and an aryl portion, as defined above. Examples include styryl, 3-(benzyl) prop-2-enyl, and 6-naphthylhex-2-enyl.

The term “hetero” in connection with rings refers to a ring in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs.

The term “aromatic” indicates that the Huckel rule is satisfied and the term “non-aromatic” indicates that the Huckel rule is not satisfied.

The term “heterocycle” or “heterocyclic” or “heterocyclic ring system” includes “aromatic heterocycle” or “heteroaryl bicyclic ring system” and “nonaromatic heterocycle ring system” or polycyclic or bicyclic (spiro, fused, bridged, non-fused) ring compounds in which ring may be aromatic or non-aromatic, wherein the heterocycle ring contains at least one heteroatom selected from N, O, S(O)₀₋₂, and or C ring member of the heterocycle may be replaced by C(═O), C(═S), C(═CR*R*) and C═NR*, * indicates integers.

The term “non-aromatic heterocycle” or “non-aromatic heterocyclic” means three- to fifteen-membered, preferably three- to twelve-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxiranyl, oxetanyl, aziridinyl, azetidinyl, thietanyl, thietanyl 1-oxide, thietanyl 1,1-dioxide, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines, 2,3,4,5-tetrahydro[1H]azepin-1- or -2- or -3- or -4- or -5- or -6- or -7- yl, 3,4,5,6-tetra-hydro[2H]azepin-2- or -3- or -4- or -5- or -6- or- 7-yl, 2,3,4,7-tetrahydro[1H]azepin-1- or -2- or -3- or -4- or -5- or -6- or- 7- yl, 2,3,6,7-tetrahydro[1H]azepin-1- or -2- or -3- or -4- or -5- or -6- or -7- yl, hexahydroazepin-1- or -2- or -3- or -4- yl, tetra- and hexahydrooxepinyl such as 2,3,4,5-tetrahydro[1 H]oxepin-2- or -3- or -4- or -5- or -6- or -7- yl, 2,3,4,7-tetrahydro[1H]oxepin-2- or -3- or -4- or -5- or -6- or -7- yl, 2,3,6,7-tetrahydro[1H]oxepin-2- or -3- or -4- or -5- or -6- or -7- yl, hexahydroazepin-1- or -2- or -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl. This definition also applies to heterocyclyl as a part of a composite substituent, for example heterocyclylalkyl etc., unless specifically defined elsewhere.

The term “heteroaryl” or “aromatic heterocyclic” means a 5 or 6-membered, fully unsaturated monocyclic ring system containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,2,3-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazol-l-yl, 1-imidazolyl, 1,2,3-triazol-1-yl and 1,3,4-triazol-1-yl.

6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) pyridinyl, pyridazinyl, pyrimidinyl, pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: for example (but not limited to) indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: for example (but not limited to) quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl. This definition also applies to heteroaryl as a part of a composite substituent, for example heteroarylalkyl etc., unless specifically defined elsewhere.

The term “Trialkylsilyl” includes three branched and/or straight-chain alkyl radicals attached to and linked through a silicon atom such as trimethylsilyl, triethylsilyl and t-butyl-dimethylsilyl. “Halotrialkylsilyl” denotes at least one of the three alkyl radicals is partially or fully substituted with halogen atoms which may be the same or different. The term “alkoxytrialkylsilyl” denotes at least one of the three alkyl radicals is substituted with one or more alkoxy radicals which may be the same or different. The term “trialkylsilyloxy” denotes a trialkylsilyl moiety attached through oxygen.

Non-limiting examples of “alkylcarbonyl” include C(O)CH₃, C(O)CH₂CH₂CH₃ and C(O)CH(CH₃)₂. Non-limiting examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Non-limiting examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Non-limiting examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH₃)C(═O). Non-limiting examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O). Non-limiting examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylalkyl amino and the like are defined analogously.

Non-limiting examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O).

The term “amide” means A-R′C═ONR″—B, wherein R′ and R″ indicates substituents and A and B indicate any group.

The term “thioamide” means A-R′C═SNR″—B, wherein R′ and R″ indicates substituents and A and B indicate any group.

The term “(halo)Cᵢ-Cⱼ-cycloalkyl-Cᵢ-Cⱼ-alkyl” indicates Cᵢ-Cⱼ-cycloalkyl-Cᵢ-Cⱼ-alkyl or Cᵢ-Cⱼ-cycloalkyl-Cᵢ-Cⱼ-alkyl may optionally be substituted with one or more halogen such as Cᵢ-Cⱼ-halocycloalkyl-Cᵢ-Cⱼ-alkyl, Cᵢ-Cⱼ-cycloalkyl-Cᵢ-Cⱼ-haloalkyl, Cᵢ-Cⱼ-halocycloalkyl-Cᵢ-Cⱼ-haloalkyl. Examples for illustrating “(halo)Cᵢ-Cⱼ-cycloalkyl-Cᵢ-Cⱼ-alkyl” includes but not limited to cyclopropyl-methyl, cyclopropyl-ethyl, cyclobutyl-methyl, cyclobutyl- ethyl, cyclopentyl-methyl, cyclopentyl-ethyl, chlorocyclopropyl-methyl, cyclopropyl-chloromethyl, chlorocyclopropyl-chloromethyl, chlorocyclopropyl-ethyl and cyclopropyl-chloroethyl.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of formula (I) is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

When a group contains a substituent which can be hydrogen, then, when this substituent is taken as hydrogen, it is recognized that said group is being unsubstituted.

The term “may optionally be substituted” is used herein interchangeably with the phrase “substituted or unsubstituted”. Unless otherwise indicated, an optionally substituted group may have a substituent at each substitutable position of the group, and each substitution is independent of the other. An optionally substituted group also may have no substituents. Therefore the phrase “may optionally be substituted with one or more substituents” means that the number of substituents may vary from zero up to the number of available positions for substitution.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form apart of the prior art base or were common general knowledge in the field relevant to the disclosure as it existed anywhere before the priority date of this application.

The numerical values mentioned in the description and the description/claims though might form a critical part of the present invention, any deviation from such numerical values shall still fall within the scope of the present invention if that deviation follows the same scientific principle as that of the present invention disclosed in the present invention. The inventive compound of the present invention may, if appropriate, be present as mixtures of different possible isomeric forms, especially of stereoisomers, for example E and Z, threo and erythro, and also optical isomers, but if appropriate also of tautomers. Both the E and the Z isomers, and also the threo and erythro isomers, and the optical isomers, any desired mixtures of these isomers and the possible tautomeric forms are disclosed and claimed.

The term “pest” for the purpose of the present disclosure includes but is not limited to fungi, stramenopiles (oomycetes), bacteria, nematodes, mites, ticks, insects and rodents. Also pest is an animal or plant detrimental to humans or human concerns including crops, livestock and forestry.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present disclosure the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts; coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plant for the purpose of the present invention include but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for use of humans and animals.

The term “plant parts” is understood to mean all parts and organs of plants above and below the ground. For the purpose of the present disclosure the term plant parts includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxillary buds, meristems, nodes and internodes.

The term “locus thereof” includes soil, surroundings of plant or plant parts and equipment or tools used before, during or after sowing/planting a plant or a plant part.

Application of the compounds of the present disclosure or the compound of the present disclosure in a composition, optionally comprising other compatible compounds, to a plant or a plant material or locus thereof include application by a technique known to a person skilled in the art which includes but is not limited to spraying, coating, dipping, fumigating, impregnating, injecting and dusting.

The term “applied” means adhered to a plant or plant part either physically or chemically including impregnation.

Accordingly, the present invention provides N-substituted bicyclic heteroaromatic compounds of formula (I),

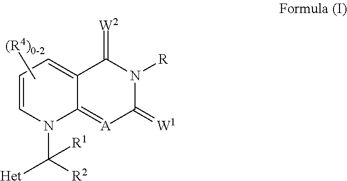

wherein,
R is selected from the group consisting of hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₁₀-alkyl-OR⁵, —C(═O)—R″, —C₁-C₁₀-alkyl-C(═O)—R″, —C₁-C₆-alkyl-NR′R″, —C(═O)—NR′R″, —C₁-C₁₀-alkyl-C(═O)—NR′R″, —C₂-C₆-alkenyl-C(═O)—N(R′R″), —C₁-C₁₀-alkyl-S(O)ₙR⁶, —S(O)ₙR⁶, —S(O)₂-phenyl, —S(O)₂-C₃-C₁₀-heterocyclyl, C₃-C₁₀-cycloalkyl, C₃-C₁₀-halocycloalkyl, C₃-C₁₀-cyanocycloalkyl, (halo)C₃-C₁₀-cycloalkyl-C₁-C₁₀-alkyl, C₃-C₁₀-cyanocycloalkyl-C₁-C₁₀-alkyl, C₄-C₁₀-cycloalkenyl, C₄-C₁₀-cycloalkynyl, bicyclic C₅-C₁₂-alkyl, bicyclic C₇-C₁₂-alkenyl, C₆-C₁₀-aryl, C₆-C₁₀-aryl-C₁-C₆-alkyl, C₃-C₁₀-heterocyclyl and C₃-C₁₀-heterocyclyl-C₁-C₆-alkyl; wherein each group may optionally be substituted with one or more R^{b};
   R^{b} is selected from the group consisting of halogen, CN, C₁-C₆-alkyl, C₁-C₆-cyanoalkyl, C₁-C₆-haloalkyl, —OR⁵, —C(═O)—R″, —NR′R″, —S(O)ₙR″ and C₃-C₆-cycloalkyl;
or
R is selected from the group consisting of phenyl, naphthyl and C₃-C₁₀-heterocyclyl; wherein each group may optionally be substituted with one or more R^{X};
   R^{x} is independently selected from the group consisting of halogen, hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₆-alkyl-OR⁵, —C(═O)—R″, —NR′R″, —C₁-C₆-alkyl-NR′R″, —NR′C(═O)—R″, —C(═O)—NR′R″, —C₁-C₁₀-alkyl-C(═O)—NR′R″, —C₁-C₆-alkyl-S(O)ₙR⁶, —S(O)ₙR⁶, C₃-C₁₀-cycloalkyl, C₃-C₁₀-halocycloalkyl, C₃-C₁₀-cyanocycloalkyl, —SF₅, —SCN, —S(═O)₀₋₁(R⁸)(═NR⁷), —N═S(═O)₀₋₁(R⁹)(R¹⁰), (halo)C₃-C₁₀-cycloalkyl-C₁-C₆-alkyl, C₃-C₁₀-cycloalkyl-C₁-C₆-alkenyl, C₃-C₁₀-cycloalkyl-C₁-C₆-alkynyl, C₃-C₁₀-cyanocycloalkyl-C₁-C₆-alkyl, C₄-C₁₀-cycloalkenyl, bicyclic C₅-C₁₂-alkyl, bicyclic C₇-C₁₂-alkenyl, phenyl, benzyl, phenylethyl, C₃-C₆-heterocyclyl and C₃-C₆-heterocyclyl-C₁-C₆-alkyl; wherein each group may optionally be substituted with one or more R′;
   R^{c} is selected from the group consisting of halogen, CN, C₁-C₆-alkyl and C₁-C₆-alkoxy;

A represents CR^{a} or N;
R^{a} is selected from the group consisting of hydrogen, cyano, C₁-C₄ alkyl, C₁-C₄ haloalkyl, C₁-C₄ cycloalkyl and —(C═O)—R″;

W¹ and W² are independently selected from O or S;

“n” is an integer ranging from 0 to 2;

R¹ and R² are independently selected from the group consisting of hydrogen, halogen, cyano, C₁-C₄ alkyl and C₁-C₄ haloalkyl;

Het is selected from the 5-6 membered heterocyclyl ring; which may optionally be substituted with one or more R³;

R³ is independently selected from the group consisting of hydrogen, halogen, cyano, C₁-C₆ alkyl and C₁-C₆ haloalkyl;

R⁴ is selected from the group consisting of hydrogen, halogen, cyano, C₁-C₆ alkyl, C₂-C₆ alkenyl, C₂-C₆ alkynyl, C₂-C₆ haloalkenyl, C₂-C₆ haloalkynyl, C₁-C₆ haloalkyl, C₁-C₆ alkoxy and C₁-C₆ haloalkoxy;

R⁵ is selected from the group consisting of hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₂-C₆ haloalkynyl-C₁-C₄-alkyl, C₃-C₁₀-cycloalkyl, C₃-C₁₀-halocycloalkyl and (halo)C₃-C₁₀-cycloalkyl-C₁-C₆-alkyl;

R⁶ is selected from the group consisting of hydrogen, —NR′R″, C₁-C₆ alkyl, C₁-C₆ haloalkyl, C₃-C₁₀-cycloalkyl, C₃-C₁₀-halocycloalkyl and (halo)C₃-C₁₀-cycloalkyl-C₁-C₆-alkyl;

R⁷ is selected from the group consisting of hydrogen, cyano, C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₆ alkylcarbonyl and C₁-C₄ haloalkylcarbonyl;

R⁸ is selected from the group consisting of C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₆ haloalkyl and C₃-C₆ halocycloalkyl;

R⁹ and R¹⁰ are independently selected from the group consisting of C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₄ haloalkyl and C₃-C₆ halocycloalkyl; or

R⁹ and R¹⁰ together with the S atom to which they are attached may form a 4- to 6-membered heterocyclic ring, wherein the C atoms of the heterocyclic ring may be optionally replaced by C(═O) or C(═S); wherein said ring may optionally be substituted with one or more R′;

R″ is selected from the group consisting of hydrogen and C₁-C₆-alkyl; wherein said alkyl may optionally be substituted with one or more halogen or C₃-C₆-cycloalkyl;

R″ is selected from the group consisting of hydrogen, C₁-C₆-alkyl, C₁-C₆-haloalkyl, C₃-C₁₀-cycloalkyl, —N(R′)₂ and —OR′; wherein each group may optionally be substituted with one or more halogen; or

R″ represent phenyl; wherein said phenyl may optionally be substituted with the group selected from halogen, cyano, C₁-C₄ alkyl, C₁-C₄ alkoxy or C₁-C₄ haloalkyl;

or

R′ and R″ together with the N atom to which they are attached may form a 4- to 6-membered heterocyclic ring, wherein the C atoms of the heterocyclic ring may be optionally replaced by C(═O) or C(═S); wherein said ring may optionally be substituted with one or more R^{c};

and/or salts, stereo-isomers, metal complexes, polymorphs, or N-oxides thereof.

In one embodiment, the compound of formula (I) is represented by the compound of formula (IA);

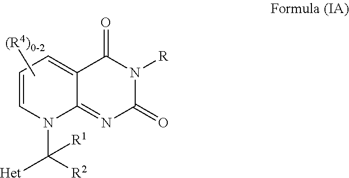

wherein, R¹, R², R⁴, R and Het are as defined above.

In another embodiment, the compound of formula (I) is represented by the compound of formula (IB);

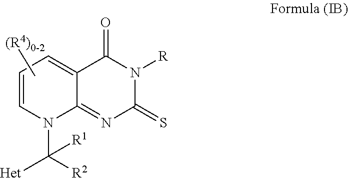

wherein, R¹, R², R⁴, R and Het are as defined above.

In yet another embodiment, the compound of formula (I) is represented by the compound of formula (IC);

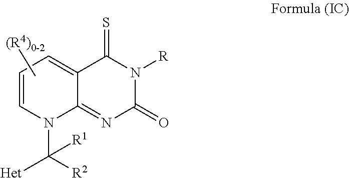

wherein, R¹, R², R⁴, R and Het are as defined above.

In yet another embodiment, the compound of formula (I) is represented by the compound of formula (ID);

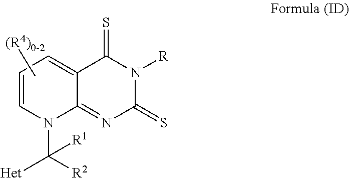

wherein, R¹, R², R⁴, R and Het are as defined above.

The following list provides definitions, including preferred definitions, for substituents W¹, W², R, R^{x}, A, R^{a}, R^{b}, R^{c}, n, R¹, R², Het, R³, R⁴, R⁵, R⁶, R⁷, R⁸, R⁹, R¹⁰, R′ and R″ with reference to the compounds of formula (I) of the present invention. For any one of these substituents, any of the definitions given below may be combined with any definition of any other substituent given below or elsewhere in this document.

In one embodiment of the present invention, W¹ and W² are independently selected from 0 or S.

In another embodiment of the present invention, W¹ and W² are O.

In yet another embodiment of the present invention, W¹ and W² are S.

In yet another embodiment of the present invention, W¹ is S and W² is O.

In a preferred embodiment of the present invention, W¹ and W² are O.

In one embodiment of the present invention, R is selected from the group consisting of hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₁₀-alkyl-OR⁵, —C(═O)—R″, —C₁-C₁₀-alkyl-C(═O)—R″, —C₁-C₆-alkyl-NR′R″, —C(═O)—NR′R″, —C₁-C₁₀-alkyl-C(═O)—NR′R″, —C₂-C₆-alkenyl-C(═O)—N(R′R″), —C₁-C₁₀-alkyl-S(O)ₙR⁶, —S(O)ₙR⁶, —S(O)₂-phenyl, —S(O)₂-C₅-C₁₀-heterocyclyl, C₃-C₁₀-cycloalkyl, C₃-C₁₀-cyanocycloalkyl, (halo)C₃-C₁₀-cycloalkyl-C₁-C₁₀-alkyl, C₃-C₁₀-cyanocycloalkyl-C₁-C₁₀-alkyl, phenyl-C₁-C₆-alkyl and C₃-C₁₀-heterocyclyl-C₁-C₆-alkyl; wherein each group may optionally be substituted with one or more R^{b}.

In another embodiment of the present invention, R is selected from hydrogen, cyano, C₁-C₁₀-alkyl, C₁-C₁₀-cyanoalkyl, C₂-C₁₀-alkenyl, C₂-C₁₀-alkynyl, C₁-C₁₀-haloalkyl, —C(═O)—R″, —OR⁵, —C₁-C₁₀-alkyl-C(═O)—R″, —C₁-C₁₀-alkyl-OR⁵, S(O)₂-phenyl, —S(O)₂-C₃-C₁₀-heterocyclyl, C₃-C₁₀-cycloalkyl, C₃-C₁₀-cyanocycloalkyl, (halo)C₃-C₁₀-cycloalkyl-C₁-C₆-alkyl, phenyl-C₁-C₄-alkyl or C₃-C₁₀-heterocyclyl-C₁-C₄-alkyl; wherein each group may optionally be substituted with one or more R^{b}.

In a preferred embodiment of the present invention, R is selected from hydrogen, cyano, C₁-C₆-alkyl, C₁-C₆-cyanoalkyl, C₂-C₆-alkenyl, C₂-C₆-alkynyl, C₁-C₆-haloalkyl, —C(═O)—R″, —OR⁵, —C₁-C₆-alkyl-C(═O)—R″, —C₁-C₆-alkyl-OR⁵, S(O)₂-phenyl, —S(O)₂—C₃-C₁₀-heterocyclyl, C₃-C₆-cycloalkyl, C₃-C₆-cyanocycloalkyl, (halo)C₃-C₆-cycloalkyl-C₁-C₃-alkyl, phenyl-C₁-C₃-alkyl or C₃-C₆-heterocyclyl-C₁-C₃-alkyl; wherein each group may optionally be substituted with one or more R^{b}.

In one embodiment of the present invention, R^{b} is independently selected from halogen, CN, C₁-C₆-alkyl, C₁-C₆-cyanoalkyl, C₁-C₆-haloalkyl, —OR⁵, —C(═O)—R″, —NR′R″, —S(O)ₙR″ or C₃-C₆-cycloalkyl.

In a preferred embodiment of the present invention, R^{b} is independently selected from CN, halogen, methyl, ethyl, cyclopropyl.

In another embodiment of the present invention, R is selected from phenyl or naphthyl; wherein each group may optionally be substituted with one or more R^{x}.

In yet another embodiment of the present invention, R is C₃-C₁₀-heterocyclyl; wherein said C₃-C₁₀-heterocyclyl group may optionally be substituted with one or more R^{x}.

In yet another embodiment of the present invention, R is C₃-C₁₀-heterocyclyl; wherein said C₃-C₁₀-heterocyclyl is selected from oxetanyl, azetidinyl, thietanyl, thietanyl 1-oxide, thietanyl 1,1-dioxide, tetrahydro-2H-pyran-4-yl, thienyl, furanyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,2,3-triazolyl, 1,3,4-oxadiazolyl, 1,3,4-thiadiazolyl, 1,3,4-triazolyl, tetrazolyl, pyridinyl, pyridazinyl, pyrimidinyl, pyrazinyl, indolyl, benzimidazolyl, indazolyl, benzofuranyl, benzothiazolyl, benzoxazolyl, 2,2-difluorobenzo[d][1,3]dioxol-5-yl, quinolinyl or isoquinolinyl; wherein said heterocyclyl rings are attached via any of the carbon or nitrogen ring atom and said heterocyclyl rings may optionally be substituted with one or more R^{x}.

In yet another embodiment of the present invention, R is selected from hydrogen, cyano, C₁-C₆-alkyl, C₁-C₆-cyanoalkyl, C₂-C₆-alkenyl, C₂-C₆-alkynyl, C₁-C₆-haloalkyl, —C(═O)—R″, —OR⁵, —C₁-C₆-alkyl-C(═O)—R″, —C₁-C₆-alkyl-OR⁵, —S(O)₂-phenyl, —S(O)₂—C₃-C₁₀-heterocyclyl, C₃-C₆-cycloalkyl, C₃-C₆-cyanocycloalkyl, (halo)C₃-C₆-cycloalkyl-C₁-C₃-alkyl, phenyl-C₁-C₃-alkyl or C₃-C₆-heterocyclyl-C₁-C₃-alkyl; wherein each group may optionally be substituted with one or more R^{b}; or R is selected from phenyl, naphthyl, oxetanyl, azetidinyl, thietanyl, thietanyl 1-oxide, thietanyl 1,1-dioxide, tetrahydro-2H-pyran-4-yl, thienyl, furanyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,2,3-triazolyl, 1,3,4-oxadiazolyl, 1,3,4-thiadiazolyl, 1,3,4-triazolyl, tetrazolyl, pyridinyl, pyridazinyl, pyrimidinyl, pyrazinyl, indolyl, benzimidazolyl, indazolyl, benzofuranyl, benzothiazolyl, benzoxazolyl, 2,2-difluorobenzo[d][1,3]dioxol-5-yl, quinolinyl or isoquinolinyl; wherein each group may optionally be substituted with one or more R^{x}.

In a preferred embodiment of present invention, R is selected from phenyl, naphthyl, pyridinyl, pyrazolyl, thiazolyl, pyrrolyl, imidazolyl, thiophen-3-yl, tetrahydro-2H-pyran-4-yl or 2,2-difluorobenzo[d][1,3]dioxol-5-yl; wherein each group may optionally be substituted with one or more R^{x}.

In a more preferred embodiment of the present invention, R is phenyl; wherein said phenyl may optionally be substituted with one or more R^{x}.

In a most preferred embodiment of the present invention, R is selected from:

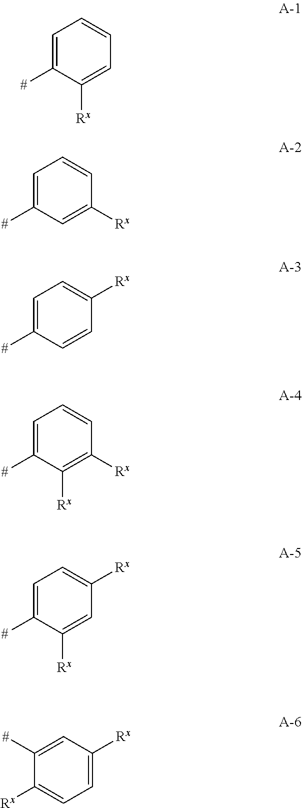

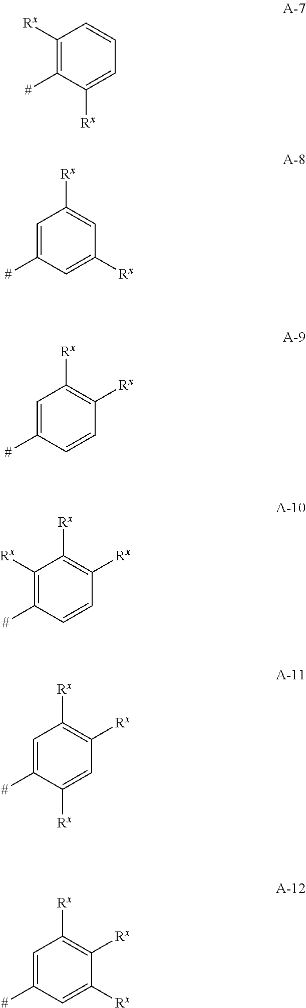

In one embodiment of the present invention, R^{x} is independently selected from halogen, hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₆-alkyl-OR⁵, C(═O)—R″, —NR′R″, —S(O)ₙR⁶, SF₅, —SCN, —S(═O)₀₋₁(R⁸)(═NR⁷)—N═S(═O)₀₋₁(R⁹)(R¹⁰), C₃-C₁₀-cycloalkyl, C₃-C₁₀-halocycloalkyl, C₃-C₁₀-cyanocycloalkyl, (halo)C₃-C₁₀-cycloalkyl-C₁-C₆-alkyl, C₃-C₁₀-cyanocycloalkyl-C₁-C₆-alkyl, phenyl or C₃-C₆-heterocyclyl; wherein each group may optionally be substituted with one or more R^{c}.

In another embodiment of the present invention, R^{x} is independently selected from halogen, hydrogen, cyano, NO₂, C₁-C₆-alkyl, C₁-C₆-cyanoalkyl, C₂-C₆-alkenyl, C₂-C₆-alkynyl, C₁-C₆-haloalkyl, C₂-C₆-haloalkenyl, C₂-C₆-haloalkynyl, C₃-C₆-cycloalkyl, —OR⁵, —C(═O)—R″, —NR′R″, —C(═O)—NR′R″, —S(═O)₀₋₁(R⁸)(═NR⁷), —N═S(═O)₀₋₁(R⁹)(R¹⁰) or —S(O)ₙR⁶; wherein each group may optionally be substituted with one or more R^{c}.

In another embodiment of the present invention, R^{c} is selected from halogen, CN, C₁-C₄-alkyl, C₁-C₄-haloalkyl, C₁-C₄-alkoxy, C₁-C₄-haloalkoxy, —C(═O)—R″, —NR′R″, —S(O)ₙR″ or C₃-C₆-cycloalkyl.

In yet another embodiment of the present invention, R^{c} is selected from halogen, CN, C₁-C₄-alkyl, C₁-C₄-haloalkyl, C₁-C₄-alkoxy or C₁-C₄-haloalkoxy.

In a preferred embodiment of the present invention, R^{x} is independently selected from halogen, hydrogen, cyano, methoxy, ethoxy, isopropoxy, cyclopropoxy, cyclobutoxy, allyloxy, propargyloxy, cyclopropylmethyloxy, cyclopropyl, difluoromethyl, trifluoroethyl, trifluoromethyl, methyl, ethyl, propyl, —SMe, —SCF₃,—SO₂Me, —NO₂, dimethylamino, diethylamino, fluoromethoxy, difluoromethoxy, fluoroethoxy, trifluoromethoxy or trifluoroethoxy; wherein each group may optionally be substituted with one or more R^{c}.

In one embodiment of the present invention, R⁵ is selected from hydrogen, C₁-C₄ alkyl, C₂-C₄ alkenyl, C₂-C₄ alkynyl, C₂-C₄ haloalkenyl, C₂-C₄ haloalkynyl, C₂-C₄ haloalkynyl-C₁-C₂-alkyl, C₁-C₄ haloalkyl, C₃-C₆-cycloalkyl, C₃-C₆-halocycloalkyl, C₃-C₆-cycloalkyl-C₁-C₄-alkyl or (halo)C₃-C₆-cycloalkyl-C₁-C₄-alkyl.

In another embodiment of the present invention, R⁵ is selected from hydrogen, C₁-C₃ alkyl, C₁-C₃ haloalkyl, C₃-C₆-cycloalkyl, C₃-C₆-halocycloalkyl or C₃-C₆-cycloalkyl-C₁-C₃-alkyl.

In a preferred embodiment of the present invention, R⁵ is selected from hydrogen, methyl, ethyl, propyl, butyl, allyl, propargyl, difluoromethyl, trifluoromethyl, trifluoroethyl cyclopropyl, cyclobutyl, cyclopentyl, cyclopropyl methyl, cyclopropyl ethyl, cyclobutyl methyl or cyclobutyl ethyl.

In one embodiment of the present invention, OR⁵ is selected from hydroxy, methoxy, ethoxy, propoxy, butyloxy, allyloxy, propargyloxy, cyclopropyloxy, cyclobutyloxy, cyclopentyloxy, cyclopropylmethoxy, cyclopropylethoxy, cyclobutylmethoxy, fluoromethoxy, difluoromethoxy, fluoroethoxy, trifluoromethoxy or trifluoroethoxy.

In one embodiment of the present invention, OR′ is selected from hydroxy, methoxy, ethoxy, propoxy, butyloxy, cyclopropyloxy, cyclobutyloxy, cyclopentyloxy, fluoromethoxy, difluoromethoxy, fluoroethoxy, trifluoromethoxy or trifluoroethoxy.

In one embodiment, C₁-C₆-alkyl-OR⁵ is selected from hydroxymethyl, methoxymethyl, ethoxymethyl, propoxymethyl, hydroxyethyl, methoxyethyl, ethoxyethyl, propoxyethyl, cyclopropylmethoxymethyl, cyclobutylmethoxymethyl or cyclopentylmethoxymethyl.

In one embodiment of the present invention, R⁶ is selected from hydrogen, —NR′R″, C₁-C₄ alkyl, C₁-C₄ haloalkyl, C₃-C₆-cycloalkyl, C₃-C₆-halocycloalkyl or C₃-C₆-cycloalkyl-C₁-C₄-alkyl.

In another embodiment of the present invention, R⁶ is selected from hydrogen, —NR′R″, C₁-C₃ alkyl, C₁-C₃ haloalkyl, C₃-C₆-cycloalkyl, C₃-C₆-halocycloalkyl or C₃-C₆-cycloalkyl-C₁-C₆-alkyl.

In a preferred embodiment of the present invention, R⁶ is selected from methyl, ethyl, propyl, difluoromethyl, trifluoromethyl, cyclopropyl, cyclobutyl, cyclopentyl, cyclopropyl methyl, cyclopropyl ethyl, cyclobutyl methyl or cyclobutyl ethyl.

In one embodiment of the present invention, R⁷ is selected from hydrogen, cyano, C₁-C₄ alkyl, C₂-C₄ alkenyl, C₂-C₄alkynyl, C₃-C₆ cycloalkyl, (halo)C₃-C₆ cycloalkyl-C₁-C₃ alkyl, C₁-C₃ alkyl carbonyl or C₁-C₃ haloalkyl carbonyl.

In another embodiment of the present invention, R⁸ is selected from C₁-C₄ alkyl, C₂-C₄ alkenyl, C₂-C₄ alkynyl, C₃-C₆ cycloalkyl, C₃-C₆ cycloalkyl-C₁-C₃ alkyl, C₁-C₄ haloalkyl or C₃-C₆ halocycloalkyl.

In yet another embodiment of the present invention, R⁹ and R¹⁰ are independently selected from C₁-C₄ alkyl, C₂-C₄ alkenyl, C₂-C₄ alkynyl, C₃-C₆ cycloalkyl, C₃-C₆ cycloalkyl-C₁-C₃ alkyl, C₁-C₄ haloalkyl or C₃-C₆ halocycloalkyl.

In one embodiment of the present invention, R′ is selected from hydrogen or C₁-C₃-alkyl; wherein said alkyl may optionally be substituted with one or more halogen or C₃-C₆-cycloalkyl.

In another embodiment of the present invention, R″ is selected from hydrogen, C₁-C₃-alkyl, C₁-C₃-haloalkyl, C₃-C₆-cycloalkyl, —N(R′)₂ or —OR′.

In another embodiment of the present invention, R′ and R″ together with the N atom to which they are attached may form a 4- to 6-membered heterocyclic ring, wherein the C atoms of the heterocyclic ring may be optionally replaced by C(═O) or C(═S); wherein said ring may optionally be substituted with one or more R^{c}.

In one embodiment of the present invention, R¹ and R² are independently selected from hydrogen, halogen, cyano, C₁-C₄ alkyl or C₁-C₄ haloalkyl.

In another embodiment of the present invention, R¹ and R² are independently selected from hydrogen or halogen.

In a preferred embodiment of the present invention, R¹ and R² are hydrogen.

In one embodiment of the present invention, R³ is independently selected from hydrogen, halogen, cyano, C₁-C₄ alkyl or C₁-C₄ haloalkyl.

In another embodiment of the present invention, R³ is independently selected from halogen or C₁-C₄ haloalkyl.

In yet another embodiment of the present invention, R³ is independently selected from

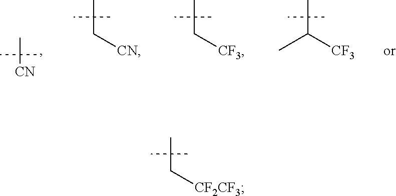

wherein

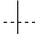

is attachment bond.

In a preferred embodiment of the present invention, R³ is independently selected from halogen, CN, difluoromethyl or trifluoromethyl.

In a more preferred embodiment of the present invention, R³ is chloro.

In one embodiment of the present invention, R⁴ is selected from hydrogen, halogen, cyano, C₁-C₄ alkyl, C₂-C₄ alkenyl, C₂-C₄ alkynyl, C₂-C₄ haloalkenyl, C₂-C₄ haloalkynyl, C₁-C₄ haloalkyl, C₁-C₄ alkoxy or C₁-C₄ haloalkoxy.

In another embodiment of the present invention, R⁴ is selected from hydrogen, halogen, methoxy or cyano.

In a preferred embodiment of the present invention, R⁴ is hydrogen.

In one embodiment of the present invention, A represents CR^{a} or N; wherein R^{a} is selected from the group consisting of hydrogen, cyano, C₁-C₄ alkyl, C₁-C₄ haloalkyl, C₁-C₄ cycloalkyl and —(C═O)—R″;

In a preferred embodiment of the present invention, A is nitrogen (N).

In one embodiment of the present invention, Het is selected from a 5-6 membered heterocyclyl ring; which may optionally be substituted with R³.

In another embodiment of the present invention, Het is selected from one of Het-1 to Het-6:

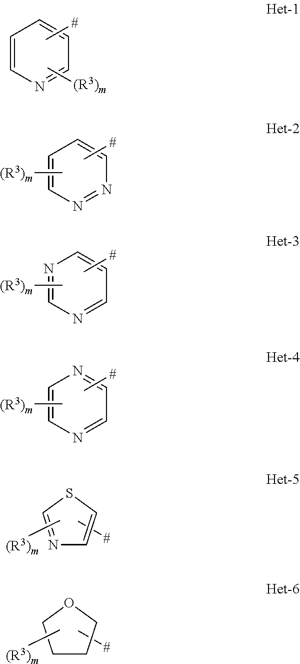

wherein,
#denote the attachment point to the remainder of the molecule;
m is 0, 1, or 2; and R³ is the same as defined herein above.

In yet another embodiment of the present invention, Het is selected from one of Het-1-a to Het-6-b:

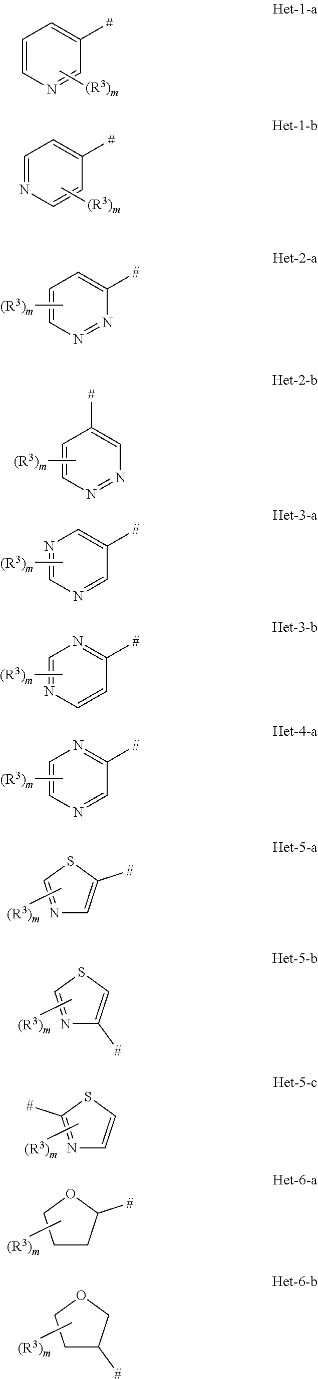

wherein,
#denote the attachment point to the remainder of the molecule;
m is 0, 1, or 2; and R³ is the same as defined herein above.

In a preferred embodiment of the present invention, Het is selected from one of:

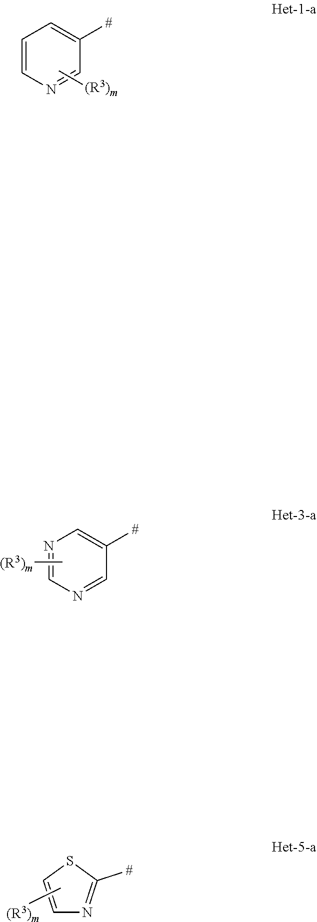

wherein,
#denote the attachment point to the remainder of the molecule;
m is 0, 1, or 2; and R³ is the same as defined herein above.

In a more preferred embodiment of the present invention, Het is selected from one of:

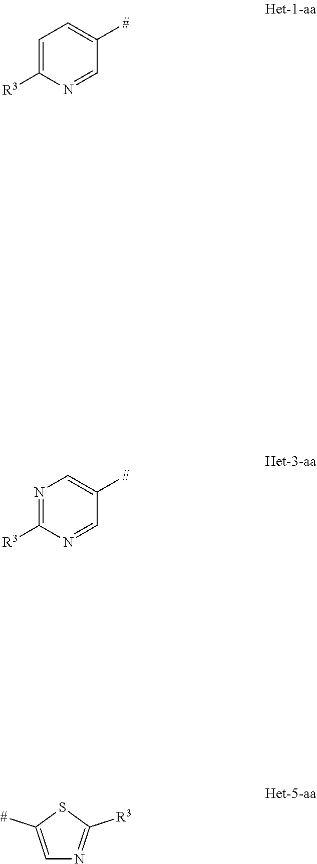

wherein,
#denote the attachment point to the remainder of the molecule;
R³ is independently selected from halogen or C₁-C₄ haloalkyl. Preferably halogen, difluoromethyl or trifluoromethyl. More preferably halogen is chloro.

In another preferred embodiment, the present invention provides compounds of formula (I),

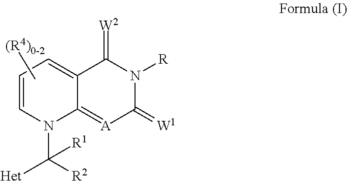

wherein,
R is selected from the group consisting of phenyl, naphthyl and C₃-C₁₀-heterocyclyl; wherein each group may optionally be substituted with one or more R^{x};
R^{x} is independently selected from the group consisting of halogen, hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₆-alkyl-OR⁵, —C(═O)—R″, —NR′R″, —C₁-C₆-alkyl-NR′R″, —NR′C(═O)—R″, —C(═O)—NR′R″, —C₁-C₁₀-alkyl-C(═O)—NR′R″, —C₁-C₆-alkyl-S(O)ₙR⁶, —S(O)ₙR⁶, C₃-C₁₀-cycloalkyl, C₃-C₁₀-halocycloalkyl, C₃-C₁₀-cyanocycloalkyl, —SF₅, —SCN, —S(═O)₀₋₁(R⁸)(═NR⁷), —N═S(═O)₀₋₁(R⁹)(R¹⁰), (halo)C₃-C₁₀-cycloalkyl-C₁-C₆-alkyl, C₃-C₁₀-cycloalkyl-C₁-C₆-alkenyl, C₃-C₁₀-cycloalkyl-C₁-C₆-alkynyl, C₃-C₁₀-cyanocycloalkyl-C₁-C₆-alkyl, C₄-C₁₀-cycloalkenyl, bicyclic C₅-C₁₂-alkyl, bicyclic C₇-C₁₂-alkenyl, phenyl, benzyl, phenylethyl, C₃-C₆-heterocyclyl and C₃-C₆-heterocyclyl-C₁-C₆-alkyl; wherein each group may optionally be substituted with one or more R^{c}; R^{c} is selected from the group consisting of halogen, CN, C₁-C₆-alkyl and C₁-C₆-alkoxy; A represents CR^{a} or N;
R^{a} is selected from the group consisting of hydrogen, cyano, C₁-C₄ alkyl, C₁-C₄ haloalkyl, C₁-C₄ cycloalkyl and —(C═O)—R″;
W¹and W² are independently selected from O or S;
“n” is an integer ranging from 0 to 2;
R¹ and R² are independently selected from the group consisting of hydrogen, halogen, cyano, C₁-C₄ alkyl and C₁-C₄ haloalkyl; Het is selected from the 5-6 membered heterocyclyl ring; which may optionally be substituted with one or more R³;
R³ is independently selected from the group consisting of hydrogen, halogen, cyano, C₁-C₄ alkyl and C₁-C₄ haloalkyl;
R⁴ is selected from the group consisting of hydrogen, halogen, cyano, C₁-C₄ alkyl, C₂-C₄ alkenyl, C₂-C₄ alkynyl, C₂-C₄ haloalkenyl, C₂-C₄ haloalkynyl, C₁-C₄ haloalkyl, C₁-C₄ alkoxy and C₁-C₄ haloalkoxy;
R⁵ is selected from the group consisting of hydrogen, C₁-C₄ alkyl, C₂-C₄ alkenyl, C₂-C₄ alkynyl, C₂-C₄ haloalkenyl, C₂-C₄ haloalkynyl, C₂-C₄ haloalkynyl-C₁-C₂-alkyl, C₁-C₄ haloalkyl, C₃-C₆-cycloalkyl, C₃-C₆-halocycloalkyl, C₃-C₆-cycloalkyl-C₁-C₄-alkyl and (halo)C₃-C₆-cycloalkyl-C₁-C₄-alkyl;
R⁶ is selected from the group consisting of hydrogen, —NR′R″, C₁-C₄ alkyl, C₁-C₄ haloalkyl, C₃-C₁₀-cycloalkyl, C₃-C₁₀-halocycloalkyl and (halo)C₃-C₁₀-cycloalkyl-C₁-C₆-alkyl;
R⁷ is selected from the group consisting of hydrogen, cyano, C₁-C₄ alkyl, C₂-C₄alkenyl, C₂-C₄alkynyl, C₃-C₆ cycloalkyl, (halo)C₃-C₆ cycloalkyl-C₁-C₃ alkyl, C₁-C₃ alkylcarbonyl and C₁-C₃ haloalkylcarbonyl;
R⁸ is independently selected from the group consisting of C₁-C₄ alkyl, C₂-C₄alkenyl, C₂-C₄ alkynyl, C₃-C₆ cycloalkyl, (halo)C₃-C₆ cycloalkyl-C₁-C₃ alkyl, C₁-C₄ haloalkyl and C₃-C₆ halocycloalkyl;
R⁹ and R¹⁰ are independently selected from the group consisting of C₁-C₄ alkyl, C₂-C₄ alkenyl, C₂-C₄ alkynyl, C₃-C₆ cycloalkyl, (halo)C₃-C₆ cycloalkyl-C₁-C₃ alkyl, C₁-C₄ haloalkyl and C₃-C₆ halocycloalkyl; or
R⁹ and R¹⁰ together with the S atom to which they are attached may form a 4- to 6-membered heterocyclic ring, wherein the C atoms of the heterocyclic ring may be optionally replaced by C(═O) or C(═S); wherein said ring may optionally be substituted with one or more R′;
R′ is selected from the group consisting of hydrogen and C₁-C₆-alkyl; wherein said alkyl may optionally be substituted with one or more halogen or C₃-C₆-cycloalkyl;
R″ is selected from the group consisting of hydrogen, C₁-C₆-alkyl, C₁-C₆-haloalkyl, C₃-C₁₀-cycloalkyl, —N(R′)₂ and —OR′; wherein each group may optionally be substituted with one or more halogen; or
R″ represents phenyl; wherein said phenyl may optionally be substituted with the group selected from halogen, cyano, C₁-C₄ alkyl, C₁-C₄ alkoxy or C₁-C₄ haloalkyl; wherein said alkyl may optionally be substituted with one or more halogen;
or
R′ and R″ together with the N atom to which they are attached may form a 4- to 6-membered heterocyclic ring, wherein the C atoms of the heterocyclic ring may be optionally replaced by C(═O) or C(═S); wherein said ring may optionally be substituted with one or more R′;
and/or salts, stereo-isomers, metal complexes, polymorphs or N-oxides thereof.

In yet another preferred embodiment, the present invention provides compounds of formula (I),

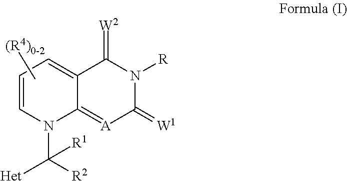

wherein,
R is selected from the group consisting of phenyl and C₃-C₁₀-heterocyclyl; wherein phenyl and C₃-C₁₀-heterocyclyl are may optionally be substituted with one or more R^{x}.
R^{x} is independently selected from the group consisting of halogen, hydrogen, cyano, NO₂, C₁-C₆-alkyl, C₁-C₆-cyanoalkyl, C₂-C₆-alkenyl, C₂-C₆-alkynyl, C₁-C₆-haloalkyl, C₂-C₆-haloalkenyl, C₂-C₆-haloalkynyl, C₃-C₆-cycloalkyl, —OR⁵, —C₁-C₆-alkyl-OR⁵, —C(═O)—R″, —NR′R″, —C₁-C₆-alkyl-NR′R″, —NR′C(═O)—R″, —C(═O)—NR′R″, —C₁-C₆-alkyl, —C(═O)—NR′R″, —C₁-C₆-alkyl-S(O)ₙR⁶, —S(O)ₙR⁶, —S(═O)₀₋₁(R⁸)(═NR⁷) and —N═S(═O)₀₋₁(R⁹)(R¹⁰); wherein each group may optionally be substituted with one or more R′;
R^{c} is selected from the group consisting of halogen, CN, C₁-C₄-alkyl and C₁-C₄-alkoxy;
A represent nitrogen (N);
W¹ and W² are 0;
“n” is an integer ranging from 0 to 2;
R¹ and R² are hydrogen;
Het is selected from one of Het-1-a, Het-3-a or Het-5-a:

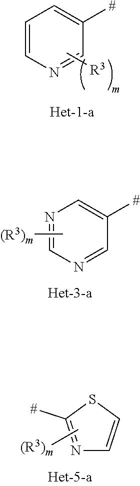

“m” is an integer ranging from 0 to 2;
R³ is selected from halogen or C₁-C₄ haloalkyl;
R⁴ is selected from the group consisting of hydrogen, halogen, cyano, C₁-C₄ alkyl and C₁-C₄ haloalkyl;
R⁵ is selected from the group consisting of hydrogen, C₁-C₃ alkyl, C₁-C₃ haloalkyl, C₃-C₆-cycloalkyl, C₃-C₆-halocycloalkyl and C₃-C₆-cycloalkyl-C₁-C₃-alkyl;
R⁶ is selected from the group consisting of hydrogen, —NR′R″, C₁-C₃ alkyl, C₁-C₃ haloalkyl, C₃-C₆-cycloalkyl, C₃-C₆-halocycloalkyl and C₃-C₆-cycloalkyl-C₁-C₆-alkyl;
R⁷ is selected from the group consisting of hydrogen, cyano, C₁-C₄ alkyl, C₂-C₄alkenyl, C₂-C₄alkynyl, C₃-C₆ cycloalkyl, (halo)C₃-C₆ cycloalkyl-C₁-C₃ alkyl, C₁-C₃ alkyl carbonyl and C₁-C₃ haloalkyl carbonyl;
R⁸ is independently selected from the group consisting of C₁-C₄ alkyl, C₂-C₄alkenyl, C₂-C₄ alkynyl, C₃-C₆ cycloalkyl, C₃-C₆ cycloalkyl-C₁-C₃ alkyl, C₁-C₄ haloalkyl and C₃-C₆ halocycloalkyl;
R⁹ and R¹⁰ are independently selected from the group consisting of C₁-C₄ alkyl, C₂-C₄ alkenyl, C₂-C₄ alkynyl, C₃-C₆ cycloalkyl, C₃-C₆ cycloalkyl-C₁-C₃ alkyl, C₁-C₄ haloalkyl or C₃-C₆ halocycloalkyl; or
R⁹ and R¹⁰ together with the S atom to which they are attached may form a 4- to 6-membered heterocyclic ring, wherein the C atoms of the heterocyclic ring may be optionally replaced by C(═O) or C(═S); wherein said ring may optionally be substituted with one or more R^{c};
R′ is selected from the group consisting of hydrogen and C₁-C₃-alkyl; wherein said alkyl may optionally be substituted with one or more halogen or C₃-C₆-cycloalkyl;
R″ is selected from the group consisting of hydrogen, C₁-C₃-alkyl, C₁-C₃-haloalkyl, C₃-C₆-cycloalkyl, —N(R′)₂ and —OR′; wherein each group may optionally be substituted with one or more halogen; or
R″ represents phenyl; wherein said phenyl may optionally be substituted with the group selected from halogen, cyano, C₁-C₄ alkyl, C₁-C₄ alkoxy or C₁-C₄ haloalkyl;
or
R′ and R″ together with the N atom to which they are attached may form a 4- to 6-membered heterocyclic ring, wherein the C atoms of the heterocyclic ring may be optionally replaced by C(═O) or C(═S); wherein said ring may optionally be substituted with one or more R^{c}; and/or salts, stereo-isomers, metal complexes, polymorphs or N-oxides thereof.

In a more preferred embodiment, the compounds of formula (I) are selected from 3-allyl-8-((6-chloropyridin-3-yl)methyl)pyrido[2,3-d]pyrimidine-2,4(3H,8H)-dione; 8-((6-chloropyridin-3-yl)methyl)-3-ethylpyrido[2,3-d]pyrimidine-2,4(3H,8H)-dione; 8-((6-chloropyridin-3-yl)methyl)-3-methylpyrido[2,3-d]pyrimidine-2,4(3H,8H)-dione; 8-((6-chloropyridin-3-yl)methyl)-3-(cyclopropylmethyl)pyrido[2,3-d]pyrimidine-2,4(3H,8H)-dione; 8-((6-chloropyridin-3-yl)methyl)-3-(2-methylallyl)pyrido[2,3-d]pyrimidine-2,4(3H,8H)-dione; 8-((6-chloropyridin-3-yl)methyl)-3-((2-chlorothiazol-5-yl)methyl)pyrido[2,3-d]pyrimidine-2,4(3H,8H)-dione; 3,8-bis((6-chloropyridin-3-yl)methyl)pyrido[2,3-d]pyrimidine-2,4(3H,8H)-dione; ethyl 2-(8-((6-chloropyridin-3-yl)methyl)-2,4-dioxo-4,8-dihydropyrido[2,3-d]pyrimidin-3(2H)-yl)acetate; 3-(3-chloropropyl)-8-((6-chloropyridin-3-yl)methyl)pyrido[2,3-d]pyrimidine-2,4(3H,8H)-dione; 8-((6-chloropyridin-3-yl)methyl)pyrido[2,3-d]pyrimidine-2,4(3H,8H)-dione acetate; 8-((6-chloropyridin-3-yl)methyl)-3-(3-fluoropropyl)pyrido[2,3-d]pyrimidine-2,4(3H,8H)-dione; 4-(8-((6-chloropyridin-3-yl)methyl)-2,4-dioxo-4,8-dihydropyrido[2,3-d]pyrimidin-3(2H)-yl)butanenitrile; 3-butyl-8-((6-chloropyridin-3-yl)methyl)pyrido[2,3-d]pyrimidine-2,4(3H,8H)-dione; 8-((6-chloropyridin-3-yl)methyl)-3-propylpyrido[2,3-d]pyrimidine-2,4(3H,8H)-dione; 8-((6-chloropyridin-3-yl)methyl)-3-(prop-2-yn-1-yl)pyrido[2,3-d]pyrimidine-2,4(3H,8H)-dione; 8-((6-chloropyridin-3-yl)methyl)-3-isopropylpyrido[2,3-d]pyrimidine-2,4(3H,8H)-dione; 2-(8-((6-chloropyridin-3-yl)methyl)-2,4-dioxo-4,8-dihydropyrido[2,3-d]pyrimidin-3(2H)-yl)acetonitrile; 8-((6-chloropyridin-3-yl)methyl)-3-(pent-4-en-1-yl)pyrido[2,3-d]pyrimidine-2,4(3H,8H)-dione; 8-((6-chloropyridin-3-yl)methyl)-3-(cyclobutylmethyl)pyrido[2,3-d]pyrimidine-2,4(3H,8H)-dione; 3-(but-3-en-1-yl)-8-((6-chloropyridin-3-yl)methyl)pyrido[2,3-d]pyrimidine-2,4(3H,8H)-dione; 8-((6-chloropyridin-3-yl)methyl)-3-(4-methylpent-3-en-1-yl)pyrido[2,3-d]pyrimidine-2,4(3H,8H)-dione; 8-((6-chloropyridin-3-yl)methyl)-3-(4,4,4- trifluorobutyl)pyrido[2,3-d]pyrimidine-2,4(3H,8H)-dione; 8-((6-chloropyridin-3-yl)methyl)-3-(2-fluoroethyl)pyrido[2,3-d]pyrimidine-2,4(3H,8H)-dione; 8-((6-chloropyridin-3-yl)methyl)-3-(5,6,6-trifluorohex-5-en-1-yl)pyrido[2,3-d]pyrimidine-2,4(3H,8H)-dione; 8-((6-chloropyridin-3-yl)methyl)-3-(2-ethoxyethyl)pyrido[2,3-d]pyrimidine-2,4(3H,8H)-dione; 8-((6-chloropyridin-3-yl)methyl)-3-((2,2-difluorocyclopropyl)methyl)pyrido[2,3-d]pyrimidine-2,4(3H,8H)-dione; 8-((6-chloropyridin-3-yl)methyl)-3-(4,4-difluorobut-3-en-1-yl)pyrido[2,3-d]pyrimidine-2,4(3H,8H)-dione; 8-((6-chloropyridin-3-yl)methyl)-3-(2,2,2-trifluoroethyl)pyrido[2,3-d]pyrimidine-2,4(3H,8H)-dione; 8-((6-chloropyridin-3-yl)methyl)-3-(2,2-difluoroethyl)pyrido[2,3-d]pyrimidine-2,4(3H,8H)-dione; 8-((6-chloropyridin-3-yl)methyl)-2,4-dioxo-4,8-dihydropyrido[2,3-d]pyrimidine-3(2H)-carbonitrile; 8-((6-chloropyridin-3-yl)methyl)-3-(2,2,3,3,3-pentafluoropropyl)pyrido[2,3-d]pyrimidine-2,4(3H,8H)-dione; 8-((6-chloropyridin-3-yl)methyl)-3-(methoxymethyl)pyrido[2,3-d]pyrimidine-2,4(3H,8H)-dione; 8-((2-chlorothiazol-5-yl)methyl)pyrido[2,3-d]pyrimidine-2,4(3H,8H)-dione; 8-((2-chlorothiazol-5-yl)methyl)-3-ethylpyrido[2,3-d]pyrimidine-2,4(3H,8H)-dione; 8-((2-chlorothiazol-5-yl)methyl)-3-methylpyrido[2,3-d]pyrimidine-2,4(3H,8H)-dione; 2-(8-((2-chlorothiazol-5-yl)methyl)-2,4-dioxo-4,8-dihydropyrido[2,3-d]pyrimidin-3(2H)-yl)acetonitrile; 8-((2-chlorothiazol-5-yl)methyl)-3-isopropylpyrido[2,3-d]pyrimidine-2,4(3H,8H)-dione; 8-((2-chlorothiazol-5-yl)methyl)-2,4-dioxo-4,8-dihydropyrido[2,3-d]pyrimidine-3(2H)-carbonitrile; 8-((2-chlorothiazol-5-yl)methyl)-3-(2-fluoroethyl)pyrido[2,3-d]pyrimidine-2,4(3H,8H)-dione; 8-((2-chlorothiazol-5-yl)methyl)-3-(2,2,2-trifluoroethyl)pyrido[2,3-d]pyrimidine-2,4(3H,8H)-dione; 8-((2-chlorothiazol-5-yl)methyl)-3-(2,2-difluoroethyl)pyrido[2,3-d]pyrimidine-2,4(3H,8H)-dione; 8-((2-chlorothiazol-5-yl)methyl)-3-(2,2,3,3,3-pentafluoropropyl)pyrido[2,3-d]pyrimidine-2,4(3H,8H)-dione; 3-allyl-8-((2-chlorothiazol-5-yl)methyl)pyrido[2,3-d]pyrimidine-2,4(3H,8H)-dione; 8-((2-chlorothiazol-5-yl)methyl)-3-(prop-2-yn-1-yl)pyrido[2,3-d]pyrimidine-2,4(3H,8H)-dione; 3-acetyl-8-((2-chlorothiazol-5-yl)methyl)pyrido[2,3-d]pyrimidine-2,4(3H,8H)-dione; 8-((2-chlorothiazol-5-yl)methyl)-3-(4,4,4-trifluorobutyl)pyrido[2,3-d]pyrimidine-2,4(3H,8H)-dione; 8-((2-chlorothiazol-5-yl)methyl)-3-(cyclopropylmethyl)pyrido[2,3-d]pyrimidine-2,4(3H,8H)-dione; 8-((2-chlorothiazol-5-yl)methyl)-3-(cyclopropanecarbonyl)pyrido[2,3-d]pyrimidine-2,4(3H,8H)-dione; 3-acetyl-8-((6-chloropyridin-3-yl)methyl)pyrido[2,3-d]pyrimidine-2,4(3H,8H)-dione; 8-((2-chlorothiazol-5-yl)methyl)-3-(4,4-difluorobut-3-en-1-yl)pyrido[2,3-d]pyrimidine-2,4(3H,8H)-dione; 8-((6-chloropyridin-3-yl)methyl)-3-(cyclopropanecarbonyl)pyrido[2,3-d]pyrimidine-2,4(3H,8H)-dione; 3-(3-chloropropyl)-8-((2-chlorothiazol-5-yl)methyl)pyrido[2,3-d]pyrimidine-2,4(3H,8H)-dione; 8-((6-chloropyridin-3-yl)methyl)-3-phenylpyrido[2,3-d]pyrimidine-2,4(3H,8H)-dione; 8-((6-chloropyridin-3-yl)methyl)-3-isopentylpyrido[2,3-d]pyrimidine-2,4(3H,8H)-dione; 8-((6-chloropyridin-3-yl)methyl)-3-(3-cyclopropylprop-2-yn-1-yl)pyrido[2,3-d]pyrimidine-2,4(3H,8H)-dione; 8-((6-chloropyridin-3-yl)methyl)-3-(4-methoxypyridin-2-yl)pyrido[2,3-d]pyrimidine-2,4(3H,8H)-dione; 8-((6-chloropyridin-3-yl)methyl)-3-(3-methoxyphenyl)pyrido[2,3-d]pyrimidine-2,4(3H,8H)-dione; 8-((6-chloropyridin-3-yl)methyl)-3-(2-methoxyphenyl)pyrido[2,3-d]pyrimidine-2,4(3H,8H)-dione; 8-((6-chloropyridin-3-yl)methyl)-3-(4-methoxyphenyl)pyrido[2,3-d]pyrimidine-2,4(3H,8H)-dione; 3-(tert-butyl)-8-((6-chloropyridin-3-yl)methyl)pyrido[2,3-d]pyrimidine-2,4(3H,8H)-dione; 8-((6-chloropyridin-3-yl)methyl)-3-(3-methylbutan-2-yl)pyrido[2,3-d]pyrimidine-2,4(3H,8H)-dione; 8-((2-chlorothiazol-5-yl)methyl)-3-phenylpyrido[2,3-d]pyrimidine-2,4(3H,8H)-dione; 3-(sec-butyl)-8-((6-chloropyridin-3-yl)methyl)pyrido[2,3-d]pyrimidine-2,4(3H,8H)-dione; 8-((6-chloropyridin-3-yl)methyl)-3-cyclopropylpyrido[2,3-d]pyrimidine-2,4(3H,8H)-dione; 8-((2-chlorothiazol-5-yl)methyl)-3-(3-methylbutan-2-yl)pyrido[2,3-d]pyrimidine-2,4(3H,8H)-dione; 3-(sec-butyl)-8-((2-chlorothiazol-5-yl)methyl)pyrido[2,3-d]pyrimidine-2,4(3H,8H)-dione; 8-((2-chlorothiazol-5-yl)methyl)-3-cyclopropylpyrido[2,3-d]pyrimidine-2,4(3H,8H)-dione; 1-(8-((6-chloropyridin-3-yl)methyl)-2,4-dioxo-4,8-dihydropyrido[2,3-d]pyrimidin-3(2H)-yl)cyclopropane-1-carbonitrile; 1-(8-((2-chloropyrimidin-5-yl)methyl)-2,4-dioxo-4,8-dihydropyrido[2,3-d]pyrimidin-3(2H)-yl)cyclopropane-1-carbonitrile; 1-(8-((2-chlorothiazol-5-yl)methyl)-2,4-dioxo-4,8-dihydropyrido[2,3-d]pyrimidin-3(2H)-yl)cyclopropane-1-carbonitrile; 8-((2-chlorothiazol-5-yl)methyl)-3-(4-methoxyphenyl)pyrido[2,3-d]pyrimidine-2,4(3H,8H)-dione; 8-((2-chlorothiazol-5-yl)methyl)-3-(3-methoxyphenyl)pyrido[2,3-d]pyrimidine-2,4(3H,8H)-dione; 8-((2-chlorothiazol-5-yl)methyl)-3-(2-methoxyphenyl)pyrido[2,3-d]pyrimidine-2,4(3H,8H)-dione; 8-((6-chloropyridin-3-yl)methyl)-3-(4-methylpentan-2-yl)pyrido[2,3-d]pyrimidine-2,4(3H,8H)-dione; 8-((6-chloropyridin-3-yl)methyl)-3-(pentan-2-yl)pyrido[2,3-d]pyrimidine-2,4(3H,8H)-dione; 8-((6-chloropyridin-3-yl)methyl)-3-(1-methoxypropan-2-yl)pyrido[2,3-d]pyrimidine-2,4(3H,8H)-dione; 8-((6-chloropyridin-3-yl)methyl)-3-(1-methoxybutan-2-yl)pyrido[2,3-d]pyrimidine-2,4(3H,8H)-dione; 8-((2-chlorothiazol-5-yl)methyl)-3-(4-methylpentan-2-yl)pyrido[2,3-d]pyrimidine-2,4(3H,8H)-dione; 3-(4-chlorophenyl)-8-((6-chloropyridin-3-yl)methyl)pyrido[2,3-d]pyrimidine-2,4(3H,8H)-dione; 3-(4-chlorophenyl)-8-((2-chlorothiazol-5-yl)methyl)pyrido[2,3-d]pyrimidine-2,4(3H,8H)-dione; 8-((6-chloropyridin-3-yl)methyl)-3-(1-methyl-1H-pyrazol-4-yl)pyrido[2,3-d]pyrimidine-2,4(3H,8H)-dione; 3-(4-chlorophenyl)-8-((2-chloropyrimidin-5-yl)methyl)pyrido[2,3-d]pyrimidine-2,4(3H,8H)-dione; 8-((2-chloropyrimidin-5-yl)methyl)-3-ethylpyrido[2,3-d]pyrimidine-2,4(3H,8H)-dione; 8-((2-chloropyrimidin-5-yl)methyl)-3-isopropylpyrido[2,3-d]pyrimidine-2,4(3H,8H)-dione; 8-((6-chloropyridin-3-yl)methyl)-3-(tert-pentyl)pyrido[2,3-d]pyrimidine-2,4(3H,8H)-dione; 8-((2-chlorothiazol-5-yl)methyl)-3-(pentan-2-yl)pyrido[2,3-d]pyrimidine-2,4(3H,8H)-dione; 8-((6-chloropyridin-3-yl)methyl)-3-(3-(trifluoromethyl)phenyl)pyrido[2,3-d]pyrimidine-2,4(3H,8H)-dione; 8-((2-chlorothiazol-5-yl)methyl)-3-(3-(trifluoromethyl)phenyl)pyrido[2,3-d]pyrimidine-2,4(3H,8H)-dione; 8-((2-chlorothiazol-5-yl)methyl)-3-(1-methyl-1H-pyrazol-4-yl)pyrido[2,3-d]pyrimidine-2,4(3H,8H)-dione; 8-((2-chlorothiazol-5-yl)methyl)-3-(1-methoxypropan-2-yl)pyrido[2,3-d]pyrimidine-2,4(3H,8H)-dione; 8-((2-chlorothiazol-5-yl)methyl)-3-(1-methoxybutan-2-yl)pyrido[2,3-d]pyrimidine-2,4(3H,8H)-dione; 8-((6-chloropyridin-3-yl)methyl)-3-(1-methylcyclopropyl)pyrido[2,3-d]pyrimidine-2,4(3H,8H)-dione; 8-((6-chloropyridin-3-yl)methyl)-3-(2-methoxyethyl)pyrido[2,3-d]pyrimidine-2,4(3H,8H)-dione; 3-(3-chlorophenyl)-8-((6-chloropyridin-3-yl)methyl)pyrido[2,3-d]pyrimidine-2,4(3H,8H)-dione; 3-(8-((6-chloropyridin-3-yl)methyl)-2,4-dioxo-4,8-dihydropyrido[2,3-d]pyrimidin-3(2H)-yl)benzonitrile; 3-(3-chlorophenyl)-8-((2-chloropyrimidin-5-yl)methyl)pyrido[2,3-d]pyrimidine-2,4(3H,8H)-dione; 3-(8-((2-chloropyrimidin-5-yl)methyl)-2,4-dioxo-4,8-dihydropyrido[2,3-d]pyrimidin-3(2H)-yl)benzonitrile; 8-((6-chloropyridin-3-yl)methyl)-3-(1,1,1-trifluoropropan-2-yl)pyrido[2,3-d]pyrimidine-2,4(3H,8H)-dione; 8-((2-chloropyrimidin-5-yl)methyl)-3-(1-methylcyclopropyl)pyrido[2,3-d]pyrimidine-2,4(3H,8H)-dione; 8-((2-chloropyrimidin-5-yl)methyl)-3-(2-methoxyethyl)pyrido[2,3-d]pyrimidine-2,4(3H,8H)-dione; 8-((6-chloropyridin-3-yl)methyl)-3-(3,5-dichlorophenyl)pyrido[2,3-d]pyrimidine-2,4(3H,8H)-dione; 8-((6-chloropyridin-3-yl)methyl)-3-(4-(trifluoromethyl)phenyl)pyrido[2,3-d]pyrimidine-2,4(3H,8H)-dione; 4-(8-((6-chloropyridin-3-yl)methyl)-2,4-dioxo-4,8-dihydropyrido[2,3-d]pyrimidin-3(2H)-yl)benzonitrile; 8-((6-chloropyridin-3-yl)methyl)-3-(3,4-dichlorophenyl)pyrido[2,3-d]pyrimidine-2,4(3H,8H)-dione; 8-((6-chloropyridin-3-yl)methyl)-3-(4-fluorophenyl)pyrido[2,3-d]pyrimidine-2,4(3H,8H)-dione; 3-(4-bromophenyl)-8-((6-chloropyridin-3-yl)methyl)pyrido[2,3-d]pyrimidine-2,4(3H,8H)-dione; 3-(3,5-bis(trifluoromethyl)phenyl)-8-((6-chloropyridin-3-yl)methyl)pyrido[2,3-d]pyrimidine-2,4(3H,8H)-dione; 3-(3-bromo-5-chlorophenyl)-8-((6-chloropyridin-3-yl)methyl)pyrido[2,3-d]pyrimidine-2,4(3H,8H)-dione; 3-(3-chloro-5-(trifluoromethyl)phenyl)-8-((6-chloropyridin-3-yl)methyl)pyrido[2,3-d]pyrimidine-2,4(3H,8H)-dione; 8-((6-chloropyridin-3-yl)methyl)-3-(1-ethyl-1H-pyrazol-4-yl)pyrido[2,3-d]pyrimidine-2,4(3H,8H)-dione; 8-((6-chloropyridin-3-yl)methyl)-3-(3-ethoxyphenyl)pyrido[2,3-d]pyrimidine-2,4(3H,8H)-dione; 8-((6-chloropyridin-3-yl)methyl)-3-(1-methyl-1H-pyrazol-5-yl)pyrido[2,3-d]pyrimidine-2,4(3H,8H)-dione; 8-((6-chloropyridin-3-yl)methyl)-3-(2-fluorophenyl)pyrido[2,3-d]pyrimidine-2,4(3H,8H)-dione; 8-((6-chloropyridin-3-yl)methyl)-3-(3-fluorophenyl)pyrido[2,3-d]pyrimidine-2,4(3H,8H)-dione; 8-((6-chloropyridin-3-yl)methyl)-3-(3,4-difluorophenyl)pyrido[2,3-d]pyrimidine-2,4(3H,8H)-dione; 8-((6-chloropyridin-3-yl)methyl)-3-(3,5-difluorophenyl)pyrido[2,3-d]pyrimidine-2,4(3H,8H)-dione; 8-((2-chloropyrimidin-5-yl)methyl)-3-(4-fluorophenyl)pyrido[2,3-d]pyrimidine-2,4(3H,8H)-dione; 8-((2-chloropyrimidin-5-yl)methyl)-3-(3-fluorophenyl)pyrido[2,3-d]pyrimidine-2,4(3H,8H)-dione; 8-((6-chloropyridin-3-yl)methyl)-3-(2,4-dichlorophenyl)pyrido[2,3-d]pyrimidine-2,4(3H,8H)-dione; 8-((2-chloropyrimidin-5-yl)methyl)-3-(3,4-difluorophenyl)pyrido[2,3-d]pyrimidine-2,4(3H,8H)-dione; 8-((6-chloropyridin-3-yl)methyl)-3-(m-tolyl)pyrido[2,3-d]pyrimidine-2,4(3H,8H)-dione; 8-((6-chloropyridin-3-yl)methyl)-3-(4-(methylthio)phenyl)pyrido[2,3-d]pyrimidine-2,4(3H,8H)-dione; 8-((6-chloropyridin-3-yl)methyl)-3-(3-(trifluoromethoxy)phenyl)pyrido[2,3-d]pyrimidine-2,4(3H,8H)-dione; 8-((6-chloropyridin-3-yl)methyl)-3-(3-(difluoromethoxy)phenyl)pyrido[2,3-d]pyrimidine-2,4(3H,8H)-dione; 8-((6-chloropyridin-3-yl)methyl)-3-(p-tolyl)pyrido[2,3-d]pyrimidine-2,4(3H,8H)-dione; 8-((6-chloropyridin-3-yl)methyl)-3-(4-(trifluoromethoxy)phenyl)pyrido[2,3-d]pyrimidine-2,4(3H,8H)-dione; 8-((6-chloropyridin-3-yl)methyl)-3-(4-(difluoromethoxy)phenyl)pyrido[2,3-d]pyrimidine-2,4(3H,8H)-dione; 8-((2-chloropyrimidin-5-yl)methyl)-3-(m-tolyl)pyrido[2,3-d]pyrimidine-2,4(3H,8H)-dione; 8-((2-chloropyrimidin-5-yl)methyl)-3-(4-(methylthio)phenyl)pyrido[2,3-d]pyrimidine-2,4(3H,8H)-dione; 8-((2-chloropyrimidin-5-yl)methyl)-3-(3-(trifluoromethoxy)phenyl)pyrido[2,3-d]pyrimidine-2,4(3H,8H)-dione; 8-((2-chloropyrimidin-5-yl)methyl)-3-(3-(difluoromethoxy)phenyl)pyrido[2,3-d]pyrimidine-2,4(3H,8H)-dione; 8-((6-chloropyridin-3-yl)methyl)-3-cyclohexylpyrido[2,3-d]pyrimidine-2,4(3H,8H)-dione; 8-((2-chlorothiazol-5-yl)methyl)-3-cyclohexylpyrido[2,3-d]pyrimidine-2,4(3H,8H)-dione; 2-chloro-5-(8-((6-chloropyridin-3-yl)methyl)-2,4-dioxo-4,8-dihydropyrido[2,3-d]pyrimidin-3(2H)-yl)benzonitrile; 3-(4-chloro-2-fluorophenyl)-8-((6-chloropyridin-3-yl)methyl)pyrido[2,3-d]pyrimidine-2,4(3H,8H)-dione; 8-((6-chloropyridin-3-yl)methyl)-3-cyclopentylpyrido[2,3-d]pyrimidine-2,4(3H,8H)-dione; 8-((6-chloropyridin-3-yl)methyl)-3-(4-methylcyclohexyl)pyrido[2,3-d]pyrimidine-2,4(3H,8H)-dione; 3-(4-chloro-3-fluorophenyl)-8-((6-chloropyridin-3-yl)methyl)pyrido[2,3-d]pyrimidine-2,4(3H,8H)-dione; 8-((6-chloropyridin-3-yl)methyl)-3-(2,4-difluorophenyl)pyrido[2,3-d]pyrimidine-2,4(3H,8H)-dione; 8-((2-chloropyrimidin-5-yl)methyl)-3-(3,5-difluorophenyl)pyrido[2,3-d]pyrimidine-2,4(3H,8H)-dione; 8-((2-chlorothiazol-5-yl)methyl)-3-(3,5-difluorophenyl)pyrido[2,3-d]pyrimidine-2,4(3H,8H)-dione; 8-((2-chlorothiazol-5-yl)methyl)-3-(p-tolyl)pyrido[2,3-d]pyrimidine-2,4(3H,8H)-dione; 8-((2-chlorothiazol-5-yl)methyl)-3-(3-fluorophenyl)pyrido[2,3-d]pyrimidine-2,4(3H,8H)-dione; 8-((2-chloropyrimidin-5-yl)methyl)-3-(p-tolyl)pyrido[2,3-d]pyrimidine-2,4(3H,8H)-dione; 3-(4-chlorophenyl)-8-((6-(trifluoromethyl)pyridin-3-yl)methyl)pyrido[2,3-d]pyrimidine-2,4(3H,8H)-dione; 2-chloro-5-(8-((2-chloropyrimidin-5-yl)methyl)-2,4-dioxo-4,8-dihydropyrido[2,3-d]pyrimidin-3(2H)-yl)benzonitrile; 3-(4-chloro-2-fluorophenyl)-8-((2-chloropyrimidin-5-yl)methyl)pyrido[2,3-d]pyrimidine-2,4(3H,8H)-dione; 8-((2-chloropyrimidin-5-yl)methyl)-3-cyclohexylpyrido[2,3-d]pyrimidine-2,4(3H,8H)-dione; 8-((2-chlorothiazol-5-yl)methyl)-3-(4-(methylthio)phenyl)pyrido[2,3-d]pyrimidine-2,4(3H,8H)-dione; 8-((2-chlorothiazol-5-yl)methyl)-3-(3-(trifluoromethoxy)phenyl)pyrido[2,3-d]pyrimidine-2,4(3H,8H)-dione; 8-((2-chlorothiazol-5-yl)methyl)-3-(3-(difluoromethoxy)phenyl)pyrido[2,3-d]pyrimidine-2,4(3H,8H)-dione; 8-((6-chloropyridin-3-yl)methyl)-3-(4-(methylsulfonyl)phenyl)pyrido[2,3-d]pyrimidine-2,4(3H,8H)-dione; 8-((2-chlorothiazol-5-yl)methyl)-3-(4-fluorophenyl)pyrido[2,3-d]pyrimidine-2,4(3H,8H)-dione; 3-(3-chloro-4-fluorophenyl)-8-((2-chloropyrimidin-5-yl)methyl)pyrido[2,3-d]pyrimidine-2,4(3H,8H)-dione; 8-((6-chloropyridin-3-yl)methyl)-3-(4-(dimethylamino)phenyl)pyrido[2,3-d]pyrimidine-2,4(3H,8H)-dione; 3-(4-fluorophenyl)-8-((6-(trifluoromethyl)pyridin-3-yl)methyl)pyrido[2,3-d]pyrimidine-2,4(3H,8H)-dione; 3-(3-chlorophenyl)-8-((6-(trifluoromethyl)pyridin-3-yl)methyl)pyrido[2,3-d]pyrimidine-2,4(3H,8H)-dione; 8-((2-chloropyrimidin-5-yl)methyl)-3-(3,4-dichlorophenyl)pyrido[2,3-d]pyrimidine-2,4(3H,8H)-dione; 3-(4-bromophenyl)-8-((2-chloropyrimidin-5-yl)methyl)pyrido[2,3-d]pyrimidine-2,4(3H,8H)-dione; 8-((2-chloropyrimidin-5-yl)methyl)-3-(4-(difluoromethoxy)phenyl)pyrido[2,3-d]pyrimidine-2,4(3H,8H)-dione; 8-((6-chloropyridin-3-yl)methyl)-3-(tetrahydro-2H-pyran-4-yl)pyrido[2,3-d]pyrimidine-2,4(3H,8H)-dione; 3-(2-fluorophenyl)-8-((6-(trifluoromethyl)pyridin-3-yl)methyl)pyrido[2,3-d]pyrimidine-2,4(3H,8H)-dione; 8-((2-chlorothiazol-5-yl)methyl)-3-(tetrahydro-2H-pyran-4-yl)pyrido[2,3-d]pyrimidine-2,4(3H,8H)-dione; 3-(3-chloro-4-fluorophenyl)-8-((6-chloropyridin-3-yl)methyl)pyrido[2,3-d]pyrimidine-2,4(3H,8H)-dione; 3-(3-chloro-4-fluorophenyl)-8-((2-chlorothiazol-5-yl)methyl)pyrido[2,3-d]pyrimidine-2,4(3H,8H)-dione; 2-chloro-5-(8-((2-chlorothiazol-5-yl)methyl)-2,4-dioxo-4,8-dihydropyrido[2,3-d]pyrimidin-3(2H)-yl)benzonitrile; 3-(4-chloro-3-methoxyphenyl)-8-((2-chlorothiazol-5-yl)methyl)pyrido[2,3-d]pyrimidine-2,4(3H,8H)-dione; 3-(2-chloro-4-fluorophenyl)-8-((6-chloropyridin-3-yl)methyl)pyrido[2,3-d]pyrimidine-2,4(3H,8H)-dione; 3-(3-chloro-2-fluorophenyl)-8-((6-chloropyridin-3-yl)methyl)pyrido[2,3-d]pyrimidine-2,4(3H,8H)-dione; 3-(4-chloro-3-methoxyphenyl)-8-((6-chloropyridin-3-yl)methyl)pyrido[2,3-d]pyrimidine-2,4(3H,8H)-dione; 3-(4-chloro-3-methoxyphenyl)-8-((2-chloropyrimidin-5-yl)methyl)pyrido[2,3-d]pyrimidine-2,4(3H,8H)-dione; 3-(3-(trifluoromethyl)phenyl)-8-((6-(trifluoromethyl)pyridin-3-yl)methyl)pyrido[2,3-d]pyrimidine-2,4(3H,8H)-dione; 8-((6-chloropyridin-3-yl)methyl)-3-(2,3-difluorophenyl)pyrido[2,3-d]pyrimidine-2,4(3H,8H)-dione; 8-((6-chloropyridin-3-yl)methyl)-3-(2,6-difluorophenyl)pyrido[2,3-d]pyrimidine-2,4(3H,8H)-dione; 3-benzyl-8-((6-chloropyridin-3-yl)methyl)pyrido[2,3-d]pyrimidine-2,4(3H,8H)-dione; 8-((2-chloropyrimidin-5-yl)methyl)-3-cyclopentylpyrido[2,3-d]pyrimidine-2,4(3H,8H)-dione; 8-((2-chlorothiazol-5-yl)methyl)-3-cyclopentylpyrido[2,3-d]pyrimidine-2,4(3H,8H)-dione; 8-((6-chloropyridin-3-yl)methyl)-3-(thiophen-3-yl)pyrido[2,3-d]pyrimidine-2,4(3H,8H)-dione; 8-((2-chloropyrimidin-5-yl)methyl)-3-(thiophen-3-yl)pyrido[2,3-d]pyrimidine-2,4(3H,8H)-dione; 8-((2-chlorothiazol-5-yl)methyl)-3-(thiophen-3-yl)pyrido[2,3-d]pyrimidine-2,4(3H,8H)-dione; 3-ethyl-8-((6-(trifluoromethyl)pyridin-3-yl)methyl)pyrido[2,3-d]pyrimidine-2,4(3H,8H)-dione; 8-((2-chlorothiazol-5-yl)methyl)-3-(3,4-dichlorophenyl)pyrido[2,3-d]pyrimidine-2,4(3H,8H)-dione; 8-((2-chlorothiazol-5-yl)methyl)-3-(4-(difluoromethoxy)phenyl)pyrido[2,3-d]pyrimidine-2,4(3H,8H)-dione; 8-((2-chloropyrimidin-5-yl)methyl)-3-(4-methylcyclohexyl)pyrido[2,3-d]pyrimidine-2,4(3H,8H)-dione; 8-((2-chlorothiazol-5-yl)methyl)-3-(4-methylcyclohexyl)pyrido[2,3-d]pyrimidine-2,4(3H,8H)-dione; 8-((6-chloropyridin-3-yl)methyl)-3-(3-(difluoromethyl)phenyl)pyrido[2,3-d]pyrimidine-2,4(3H,8H)-dione; 8-((2-chlorothiazol-5-yl)methyl)-3-(3-(difluoromethyl)phenyl)pyrido[2,3-d]pyrimidine-2,4(3H,8H)-dione; 3-(4-chlorophenyl)-8-((6-chloropyridin-3-yl)methyl)-2-thioxo-2,8-dihydropyrido[2,3-d]pyrimidin-4(3H)-one; 8-((6-chloropyridin-3-yl)methyl)-3-(4-fluorophenyl)-2-thioxo-2,8-dihydropyrido[2,3-d]pyrimidin-4(3H)-one; 8-((6-chloropyridin-3-yl)methyl)-2-thioxo-3-(3-(trifluoromethyl)phenyl)-2,8-dihydropyrido[2,3-d]pyrimidin-4(3H)-one; 3-(4-bromophenyl)-8-((6-chloropyridin-3-yl)methyl)-2-thioxo-2,8-dihydropyrido[2,3-d]pyrimidin-4(3H)-one; 3-(4-chlorophenyl)-8-((tetrahydrofuran-3-yl)methyl)pyrido[2,3-d]pyrimidine-2,4(3H,8H)-dione; 3-(4-chloro-2-fluorophenyl)-8-((2-chlorothiazol-5-yl)methyl)pyrido[2,3-d]pyrimidine-2,4(3H,8H)-dione; 8-((6-chloropyridin-3-yl)methyl)-3-(4-methoxy-3-(trifluoromethoxy)phenyl)pyrido[2,3-d]pyrimidine-2,4(3H,8H)-dione; 3-(3-chloro-4-(trifluoromethoxy)phenyl)-8-((6-chloropyridin-3-yl)methyl)pyrido[2,3-d]pyrimidine-2,4(3H,8H)-dione; 8-((6-chloropyridin-3-yl)methyl)-3-(o-tolyl)pyrido[2,3-d]pyrimidine-2,4(3H,8H)-dione; 3-(2-chlorophenyl)-8-((6-chloropyridin-3-yl)methyl)pyrido[2,3-d]pyrimidine-2,4(3H,8H)-dione; 8-((2-chloropyrimidin-5-yl)methyl)-3-(2,2-difluorobenzo[d][1,3]dioxol-5-yl)pyrido[2,3-d]pyrimidine-2,4(3H,8H)-dione; 8-((6-chloropyridin-3-yl)methyl)-3-(2,2-difluorobenzo[d][1,3]dioxol-5-yl)pyrido[2,3-d]pyrimidine-2,4(3H,8H)-dione; 8-((2-chloropyrimidin-5-yl)methyl)-3-(2-fluorophenyl)pyrido[2,3-d]pyrimidine-2,4(3H,8H)-dione; 8-((2-chloropyrimidin-5-yl)methyl)-3-(3,5-dichlorophenyl)pyrido[2,3-d]pyrimidine-2,4(3H,8H)-dione; 3-(3-bromophenyl)-8-((6-chloropyridin-3-yl)methyl)pyrido[2,3-d]pyrimidine-2,4(3H,8H)-dione; 8-((2-chloropyrimidin-5-yl)methyl)-3-(3-(difluoromethyl)phenyl)pyrido[2,3-d]pyrimidine-2,4(3H,8H)-dione; 8-((6-chloropyridin-3-yl)methyl)-3-(3-(difluoromethyl)phenyl)-2-thioxo-2,8-dihydropyrido[2,3-d]pyrimidin-4(3H)-one; 8-((2-chloropyrimidin-5-yl)methyl)-3-(2,3-difluorophenyl)pyrido[2,3-d]pyrimidine-2,4(3H,8H)-dione; 8-((2-chloropyrimidin-5-yl)methyl)-3-(2,6-difluorophenyl)pyrido[2,3-d]pyrimidine-2,4(3H,8H)-dione; 8-((2-chloropyrimidin-5-yl)methyl)-3-(2,4-difluorophenyl)pyrido[2,3-d]pyrimidine-2,4(3H,8H)-dione; 3-(4-chloro-3-fluorophenyl)-8-((2-chloropyrimidin-5-yl)methyl)pyrido[2,3-d]pyrimidine-2,4(3H,8H)-dione; 8-((6-chloropyridin-3-yl)methyl)-2-thioxo-3-(4-(trifluoromethoxy)phenyl)-2,8-dihydropyrido[2,3-d]pyrimidin-4(3H)-one; 3-(4-chlorophenyl)-8-((2-chloropyrimidin-5-yl)methyl)-2-thioxo-2,8-dihydropyrido[2,3-d]pyrimidin-4(3H)-one; 8-((6-chloropyridin-3-yl)methyl)-3-(3-(2,2,2-trifluoroethoxy)phenyl)pyrido[2,3-d]pyrimidine-2,4(3H,8H)-dione; 3-(3-chloro-4-(trifluoromethoxy)phenyl)-8-((2-chlorothiazol-5-yl)methyl)pyrido[2,3-d]pyrimidine-2,4(3H,8H)-dione; 8-((2-chloropyrimidin-5-yl)methyl)-3-(4-fluoro-3-(trifluoromethyl)phenyl)pyrido[2,3-d]pyrimidine-2,4(3H,8H)-dione; 8-((2-chlorothiazol-5-yl)methyl)-3-(2,3-difluorophenyl)pyrido[2,3-d]pyrimidine-2,4(3H,8H)-dione; 8-((2-chlorothiazol-5-yl)methyl)-3-(2,6-difluorophenyl)pyrido[2,3-d]pyrimidine-2,4(3H,8H)-dione; 8-((2-chloropyrimidin-5-yl)methyl)-3-(4-(2,2,2-trifluoroethoxy)phenyl)pyrido[2,3-d]pyrimidine-2,4(3H,8H)-dione; 3-(3-chloro-2-fluorophenyl)-8-((2-chlorothiazol-5-yl)methyl)pyrido[2,3-d]pyrimidine-2,4(3H,8H)-dione; 3-(3-chloro-4-fluorophenyl)-8-((6-chloropyridin-3-yl)methyl)-2-thioxo-2,8-dihydropyrido[2,3-d]pyrimidin-4(3H)-one; 3-(4-chloro-3-(trifluoromethyl)phenyl)-8-((6-chloropyridin-3-yl)methyl)pyrido[2,3-d]pyrimidine-2,4(3H,8H)-dione; 3-(4-chloro-3-methoxyphenyl)-8-((6-chloropyridin-3-yl)methyl)-2-thioxo-2,8-dihydropyrido[2,3-d]pyrimidin-4(3H)-one; 8-((6-chloropyridin-3-yl)methyl)-3-(thiophen-3-yl)-2-thioxo-2,8-dihydropyrido[2,3-d]pyrimidin-4(3H)-one; 8-((6-chloropyridin-3-yl)methyl)-3-(4-fluoro-3-(trifluoromethyl)phenyl)pyrido[2,3-d]pyrimidine-2,4(3H,8H)-dione; 8-((6-chloropyridin-3-yl)methyl)-3-(4-(2,2,2-trifluoroethoxy)phenyl)pyrido[2,3-d]pyrimidine-2,4(3H,8H)-dione; 3-(4-chloro-3-(trifluoromethyl)phenyl)-8-((2-chloropyrimidin-5-yl)methyl)pyrido[2,3-d]pyrimidine-2,4(3H,8H)-dione; 3-(2-chloro-4-fluorophenyl)-8-((2-chloropyrimidin-5-yl)methyl)pyrido[2,3-d]pyrimidine-2,4(3H,8H)-dione; 3-(3-chloro-2-fluorophenyl)-8-((2-chloropyrimidin-5-yl)methyl)pyrido[2,3-d]pyrimidine-2,4(3H,8H)-dione; 3-(3-chloro-4-(trifluoromethoxy)phenyl)-8-((2-chloropyrimidin-5-yl)methyl)pyrido[2,3-d]pyrimidine-2,4(3H,8H)-dione; 8-((6-chloropyridin-3-yl)methyl)-3-(2,4-difluorophenyl)-2-thioxo-2,8-dihydropyrido[2,3-d]pyrimidin-4(3H)-one; 8-((6-chloropyridin-3-yl)methyl)-3-(3-fluoro-4-methoxyphenyl)pyrido[2,3-d]pyrimidine-2,4(3H,8H)-dione; 3-(3-bromo-4-methoxyphenyl)-8-((6-chloropyridin-3-yl)methyl)pyrido[2,3-d]pyrimidine-2,4(3H,8H)-dione; 3-(3-bromophenyl)-8-((2-chloropyrimidin-5-yl)methyl)pyrido[2,3-d]pyrimidine-2,4(3H,8H)-dione; 3-(3-bromo-4-fluorophenyl)-8-((6-chloropyridin-3-yl)methyl)pyrido[2,3-d]pyrimidine-2,4(3H,8H)-dione; 3-(4-bromo-3-fluorophenyl)-8-((6-chloropyridin-3-yl)methyl)pyrido[2,3-d]pyrimidine-2,4(3H,8H)-dione; 3-(3-bromo-4-fluorophenyl)-8-((2-chloropyrimidin-5-yl)methyl)pyrido[2,3-d]pyrimidine-2,4(3H,8H)-dione; 3-(4-bromo-3-fluorophenyl)-8-((2-chloropyrimidin-5-yl)methyl)pyrido[2,3-d]pyrimidine-2,4(3H,8H)-dione; 3-(2-chloro-4-fluorophenyl)-8-((6-chloropyridin-3-yl)methyl)-2-thioxo-2,8-dihydropyrido[2,3-d]pyrimidin-4(3H)-one; 3-(2-chloro-4-fluorophenyl)-8-((2-chlorothiazol-5-yl)methyl)pyrido[2,3-d]pyrimidine-2,4(3H,8H)-dione; 8-((6-chloropyridin-3-yl)methyl)-3-(1-methyl-1H-pyrazol-3-yl)pyrido[2,3-d]pyrimidine-2,4(3H,8H)-dione; 8-((6-chloropyridin-3-yl)methyl)-3-(1-phenylethyl)pyrido[2,3-d]pyrimidine-2,4(3H,8H)-dione; 8-((6-chloropyridin-3-yl)methyl)-3-(3,4-dichlorophenyl)-2-thioxo-2,8-dihydropyrido[2,3-d]pyrimidin-4(3H)-one; 8-((6-chloropyridin-3-yl)methyl)-3-(1-phenylethyl)-2-thioxo-2,8-dihydropyrido[2,3-d]pyrimidin-4(3H)-one; 8-((2-chlorothiazol-5-yl)methyl)-3-(4-fluoro-3-(trifluoromethyl)phenyl)pyrido[2,3-d]pyrimidine-2,4(3H,8H)-dione; 8-((2-chlorothiazol-5-yl)methyl)-3-(4-(2,2,2-trifluoroethoxy)phenyl)pyrido[2,3-d]pyrimidine-2,4(3H,8H)-dione; 8-((2-chloropyrimidin-5-yl)methyl)-3-(3-fluoro-5-(trifluoromethyl)phenyl)pyrido[2,3-d]pyrimidine-2,4(3H,8H)-dione; 8-((2-chloropyrimidin-5-yl)methyl)-3-(4-cyclopropylphenyl)pyrido[2,3-d]pyrimidine-2,4(3H,8H)-dione; 8-((6-chloropyridin-3-yl)methyl)-3-(3-((trifluoromethyl)thio)phenyl)pyrido[2,3-d]pyrimidine-2,4(3H,8H)-dione; 8-((2-chlorothiazol-5-yl)methyl)-3-(2,2-difluorobenzo[d][1,3]dioxol-5-yl)pyrido[2,3-d]pyrimidine-2,4(3H,8H)-dione; 3-(4-bromo-3-fluorophenyl)-8-((2-chlorothiazol-5-yl)methyl)pyrido[2,3-d]pyrimidine-2,4(3H,8H)-dione and 3-(3-bromophenyl)-8-((2-chlorothiazol-5-yl)methyl)pyrido[2,3-d]pyrimidine-2,4(3H,8H)-dione.

The compounds of the present invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the present invention may be present as a mixture of stereoisomers, individual stereoisomers or as an optically active form.

An anion part of the salt, in case the compound of formula (I) is cationic or capable of forming a cation, can be inorganic or organic. Alternatively, a cation part of the salt, in case the compound of formula (I) is anionic or capable of forming an anion, can be inorganic or organic. Examples of an inorganic anion part of the salt include but are not limited to chloride, bromide, iodide, fluoride, sulfate, phosphate, nitrate, nitrite, hydrogen carbonates and hydrogen sulfate. Examples of an organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkylphosphonates, arylphosphonates, aryldiphosphonates, p-toluenesulphonate, and salicylate. Examples of an inorganic cation part of the salt include but are not limited to alkali and alkaline earth metals. Examples of an organic cation part of the salt include but are not limited to pyridine, methyl amine, imidazole, benzimidazole, hitidine, phosphazene, tetramethyl ammonium, tetrabutylammonium, choline and trimethylamine.

Metal ions in metal complexes of the compound of formula (I) are especially ions of the elements of the second main group, e.g., calcium and magnesium, of the third and fourth main group, e.g., aluminium, tin and lead, and also of the first to eighth transition groups, e.g., chromium, manganese, iron, cobalt, nickel, copper, zinc and others. Particular preference is given to the metal ions of the elements of the fourth period and the first to eighth transition groups. Here, the metals can be present in the various valencies that they can assume.

In one embodiment, the present invention provides a compound of formula (I) in the form of salts, metal complexes, stereo-isomers, polymorphs or N-oxides thereof and its composition with the excipient, inert carrier or any other essential ingredient such as surfactants, additives, solid diluents and liquid diluents. Salts of the compounds of the formula (I) are preferably veterinary and/or agriculturally acceptable salts, preferably agriculturally acceptable salts. They can be formed in a customary manner, e.g. by reacting the compound with an acid of the anion in question if the compound of formula (I) has a basic functionality.

The term “N-oxide” includes any compound of formula (I) which has at least one tertiary nitrogen atom that is oxidized to an N-oxide moiety.

The compounds of formula (I), (including all stereoisomers, N-oxides, and salts thereof), typically exist in more than one form, and formula (I) thus includes all crystalline and non-crystalline forms of the compounds that formula (I) represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound which can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in chemical, physical and biological properties such as crystal shape, density, hardness, color, chemical stability, melting point, hygroscopicity, suspensibility, dissolution rate and biological availability. One skilled in the art will appreciate that a polymorph of a compound represented by formula (I) can exhibit beneficial effects (e.g., suitability for preparation of useful formulations, improved biological performance) relative to another polymorph or a mixture of polymorphs of the same compound represented by formula (I). Preparation and isolation of a particular polymorph of a compound represented by formula (I) can be achieved by methods known to those skilled in the art including, for example, crystallization using selected solvents and temperatures.

In one embodiment, the present invention provides a process for preparing the compound of formula (I) wherein A is N; or salts thereof,

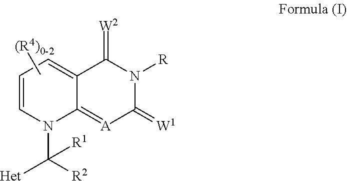

The compounds of the present invention as defined by formula (I) and/or in table (I) may be prepared, in known manner, in a variety of ways as described in the below schemes. The compounds of the present invention can be made as shown in the following schemes, in which, unless otherwise stated, the definition of each variable is as defined above for a compound of formula (I).

### General Synthetic Schemes

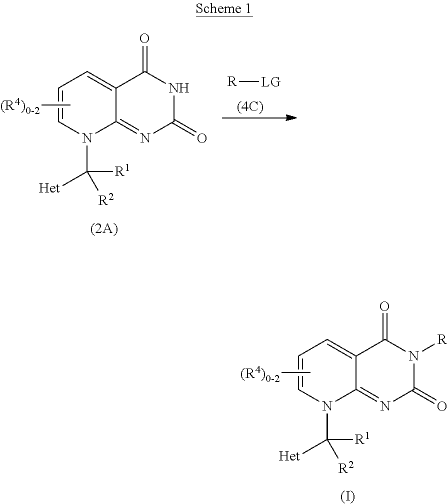

wherein, R, R¹, R², R⁴, LG and Het have the meanings as described above.

A compound of formula (I) can be prepared from a compound of formula (2A) via N-alkylation by reacting it with a compound of formula (4C). LG is preferably a halogen (preferably iodide or bromide) or tosylate or nosylate, or mesylate, whereas the reaction is performed in the presence of a suitable base, such as sodium or potassium or cesium carbonate or sodium hydride, optionally in the presence of a suitable catalyst such as sodium iodide or tert-butyl ammonium iodide or tert-buty ammonium bromide, and in an appropriate solvent such as N,N,-dimethylformamide, N,N-dimethylacetamide, dimethylsulfoxide, tetrahydrofuran or acetonitrile, at temperatures between 0° C. and 150° C. by following an analogous procedure disclosed in the Journal of Medicinal Chemistry, 2002, 45(1), 219-232.

Alternatively, a compound of formula (I) can be prepared by metal catalysis. Examples of the metal catalyst to be used in such a reaction include a copper catalyst such as copper(I)iodide, copper(I)bromide, copper(I)chloride, copper(I)oxide, trifluromethanesulfonicacid copper(I)salt benzene complex, 2-thiophenecarboxylic acid copper(I)salt; and a nickel catalyst such as bis(cyclooctadiene)nickel(0) and nickel chloride; and a palladium catalyst such as palladium acetate and a ligand, for example 1,10-phenanthroline, 8-hydroquinoline, trans-1,2-cyclohexanediamine, trans-1,2-bis(methylamino)cyclohexane, N,N-dimethylethylenediamine, 2,2′-bipyridine, 2-aminoethanol, triphenyl phosphine, 1,2-bis(diphenylphosphino)ethane, 1,3-bis(diphenylphosphino)propane, XantPhos ((5-di-phenylphosphanyl-9,9-dimethyl-xanthen-4yl)diphenylphosphane), XPhos (2-dicyclohexylphosphino-2′,4′,6′-triisopropylbiphenyl), RuPhos (2-dicyclohexylphosphino-2′,6′-diisopropoxybiphenyl), BINAP (2,2′-bis(diphenylphosphino)-1,1′-binaphthalene), and 1,1′-bis(diphenylphosphino)ferrocene. Examples of the suitable base include but are not limited to alkalimetal carbonates, sodium or potassium tert-butoxide or diisopropylethylamine or pyridine or 1,8-diazabicyclo[5.4.0]undec-7-ene or 4-(dimethylamino)pyridine, in an appropriate solvent such as acetonitrile or dimethyl sulfoxide, N,N-dimethylformamide, N,N-dimethyl acetamide, N-methyl-2-pyrrolidone, at temperatures ranges from about 25° C. to about 120° C. by following an analogous procedure disclosed in the Chem. Rev. 2004, 248, 2337-2364.

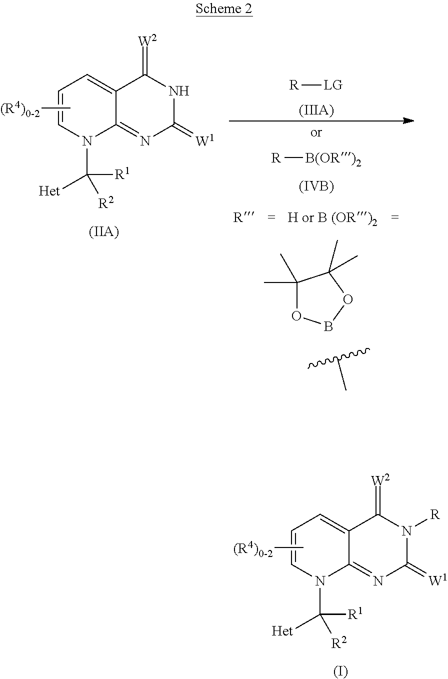

wherein, R, R¹, R², R⁴ and Het have the meanings as described above.

In other ways, a compound of formula (I), where R is aryl or heteroaryl, can be prepared from a compound of formula (4B) under Chan-Lam coupling conditions. The reaction conditions include but are not limited to, carrying out the reaction in the presence of a copper source such as copper(I) iodide or copper(II) acetate, optionally in the presence of a suitable base such as pyridine or potassium phosphate, in an inert solvent such as dichloromethane, dioxane or dimethyl sulfoxide, at temperatures ranging from about 25° C. to about 150° C., optionally under microwave conditions, preferentially under oxygen atmosphere by following an analogous procedure disclosed in the Tetrahedron Letters 2015, 56(33),4843-4847.

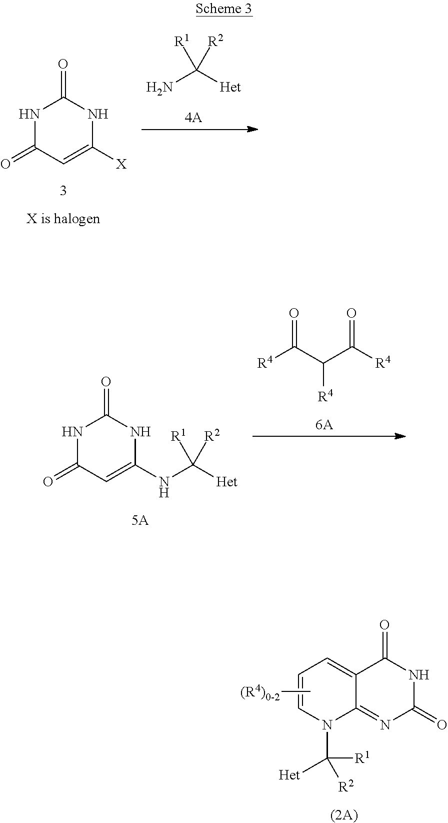

wherein, R¹, R², R⁴ and Het have the meanings as described above.

A compound of formula (3) can be converted to a compound of formula (5A) by reacting it with a compound of formula (4A) in the presence of an inert solvent such as ethanol, n-butanol, tetrahydrofuran, dimethylsulfoxide or a mixture thereof, and a suitable base such as triethylamine, diisopropyl amine 1,8-diazabicyclo[5.4.0]undec-7-ene, N-methyl pyrrolidine or pyridine. The reaction can be carried out at a temperature ranging from about 50° C. to about 180° C., optionally under microwave conditions by following an analogous procedure disclosed in the prior art for example WO2006020070, WO2007021450.

The compound of formula (5A) can undergo a cyclocondensation reaction when treated with a 1,3-diketone compound of formula (6A), optionally in the presence of a suitable base, to obtain a bicyclic compound of formula (2A). Examples of the suitable base include but are not limited to piperidine, morpholine, N-methylpiperazine, diethyl amine, triethylamine and the like. Examples of the suitable solvent include but are not limited to methanol, ethanol, isopropanol, ethyleneglycol and the like. The reaction can be carried out at temperatures ranging between 50° C. and 150° C. When one of the electrophilic centers in the compound of formula (6A) are protected (e.g. aldehyde masked as ketal) the condensation can be effected in the presence of a suitable solvent and in the presence of a suitable acid such as acetic acid, sulfonic acid, sulfuric acid, hydrochloric acid. Example of the suitable solvent include but are not limited to methanol, ethanol, isopropanol, ethyleneglycol and the like.

The reaction can be carried out at temperatures ranging from about 50° C. to about 150° C.

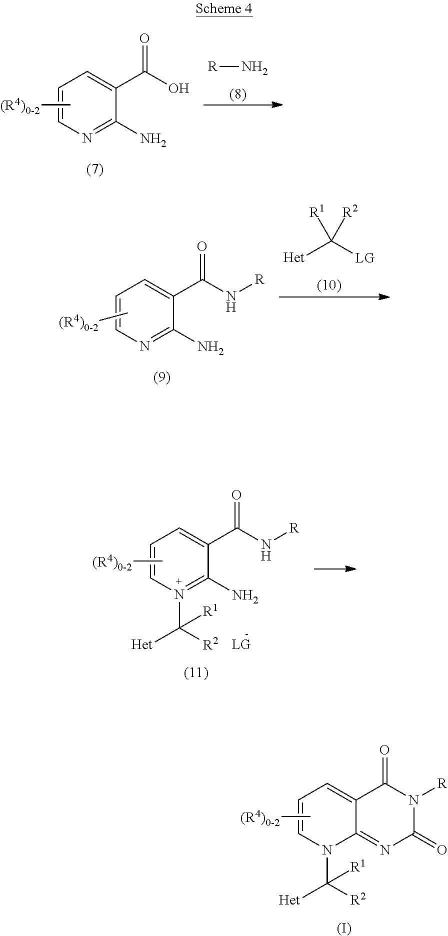

wherein, X is halogen; R, R¹, R², R⁴ and Het have the meanings as described above.

A compound of formula (I) can be prepared from a compound of formula (11) by reacting it with a carbonylating reagent such as CDI, diethylcarbonate, phosgene, triphosgene or ethylchloroformate in the presence of a suitable solvent such as tetrahydrofuran and a suitable base such as organic or inorganic base which is selected from but not limited to triethylamine or potassium carbonate, at a temperature ranging from about 0° C. to about 150° C.

The compound of formula (11) can be prepared from a compound of formula (9) via N-alkylation by reacting it with a compound of formula (10) wherein LG is a halogen or tosylate or nosylate, or mesylate, preferably a halogen. The reaction is performed optionally in the presence of a suitable base, and optionally in the presence of a suitable catalyst such as sodium iodide or tert-butyl ammonium iodide or tert-buty ammonium bromide, and in an appropriate solvent such as N,N,-dimethylformamide, N,N-dimethylacetamide, dimethylsulfoxide, acetone, tetrahydrofuran or acetonitrile, at temperatures ranging from about 0° C. to about 150° C.

Further the compound of formula (9) can be obtained by reacting a compound of formula (8) with a compound of formula (7) in the presence of a suitable reagent such as methane sulfonyl chloride, oxalyl chloride, thionyl chloride or a coupling reagent such as EDC.HCl/HOBt, HATU, T3P, in the presence of a suitable base such as organic or inorganic base selected from but not limited to triethylamine, N,N-diisopropylethylamine, or potassium carbonate, in a suitable solvent such as N,N-dimethylformamide, at temperatures ranging from about 0° C. to about 50° C.

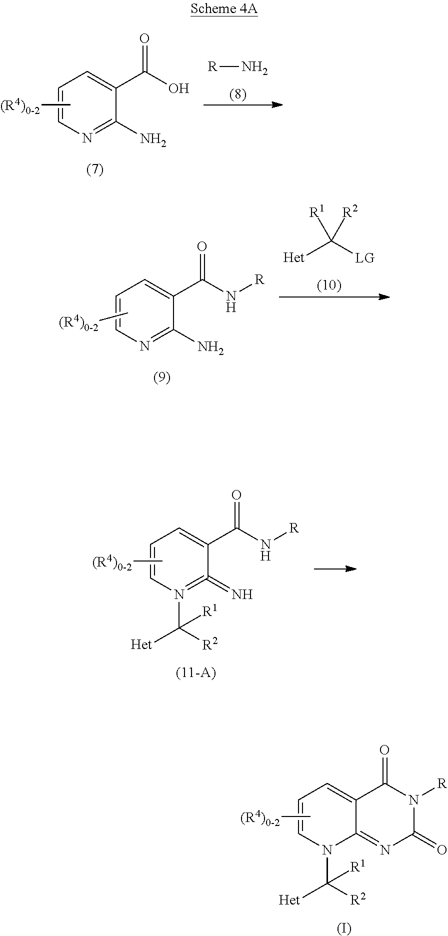

wherein, X is halogen; R, R¹, R², R⁴, LG and Het have the meanings as described above.

A compound of formula (I) can be prepared from a compound of formula (11-A) by reacting with a carbonylating reagent such as CDI, diethylcarbonate, phosgene, triphosgene or ethylchloroformate in the presence of a suitable solvent such as tetrahydrofuran and a suitable base such as organic or inorganic base which is selected from but not limited to triethylamine or potassium carbonate, at temperatures ranging from about 0° C. to about 150° C.

The compound of formula (11-A) can be prepared from a compound of formula (9) via N-alkylation by reacting it with a compound of formula (10) wherein LG is a halogen or tosylate or nosylate, or mesylate, preferably a halogen. The reaction is performed optionally in the presence of a suitable base, and optionally in the presence of a suitable catalyst such as sodium iodide or tert-butyl ammonium iodide or tert-buty ammonium bromide, and in an appropriate solvent such as N,N,-dimethylformamide, N,N-dimethylacetamide, dimethylsulfoxide, acetone, tetrahydrofuran or acetonitrile, at temperatures ranging from about 0° C. to about 150° C.

Further the compound of formula (9) can be obtained by reacting a compound of formula (8) with a compound of formula (7) in the presence of a suitable reagent such as methane sulfonyl chloride, oxalyl chloride, thionyl chloride or a coupling reagent such as EDC.HCl/HOBt, HATU, T3P, in the presence of a suitable base such as an organic or inorganic base selected from but not limited to triethylamine, N,N-diisopropylethylamine, or potassium carbonate, in a suitable solvent such as N,N-dimethylformamide, at temperatures ranging from about 0° C. to about 50° C.

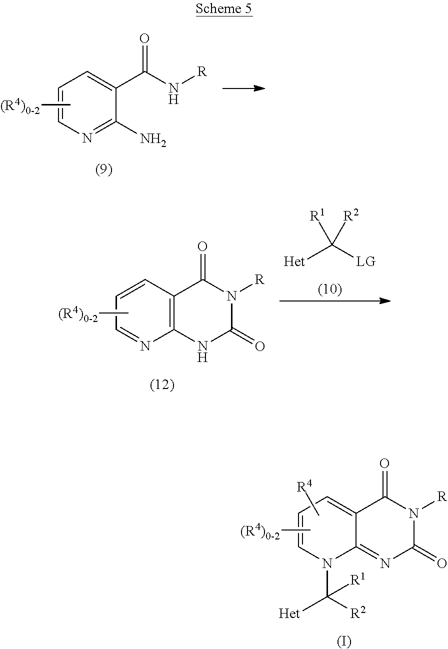

wherein, R, R¹, R², R⁴, LG and Het have the meanings as described above.

A compound of formula (I) can be prepared from a compound of formula (12) via N-alkylation by reacting it with a compound of formula (10). LG is preferably a halogen (preferably iodide, bromide or chloride) or tosylate or nosylate, or mesylate, whereas the reaction is performed optionally in the presence of a suitable base, and optionally in the presence of a suitable catalyst such as sodium iodide or tert-butyl ammonium iodide or tert-buty ammonium bromide, and in an appropriate solvent such as N,N,-dimethylformamide, N,N-dimethylacetamide, dimethylsulfoxide, tetrahydrofuran, acetonitrile or acetone, at temperatures ranging from about 0° C. to about 150° C. by following an analogous procedure disclosed in the Chem. Res. Toxicol. 12(1), 1999, 46-52 and FR2983200.

The compound of formula (12) can be prepared from a compound of formula (9) by reacting it with a carbonylating reagent such as CDI, diethylcarbonate, phosgene, triphosgene or ethylchloroformate in the presence of a suitable solvent such as tetrahydrofuran and in the presence of a suitable base such as organic or inorganic base which is selected from but not limited to triethylamine or potassium carbonate, at temperatures ranging from about 0° C. to about 150° C.

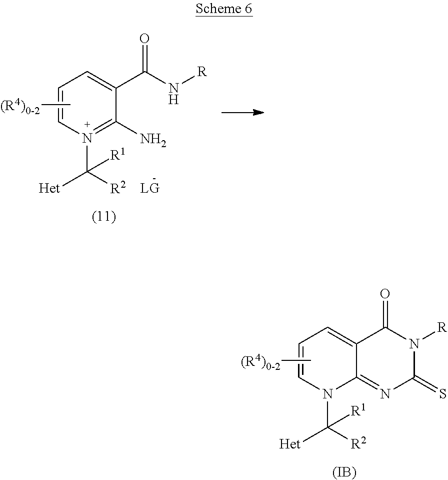

wherein, R, R¹, R², R⁴, LG and Het have the meanings as described above.

A compound of formula (IB) can be prepared from a compound of formula (11) by reacting it with a thiocarbonylating reagent such as thioCDI (1,1′-thiocarbonyldiimidazole) or thiophenylchloroformate, in the presence of a suitable solvent such as tetrahydrofuran, dichloromethane, 1,2-dichloroethane or acetonitrile and a suitable base such as an organic or inorganic base which is selected from but not limited to triethylamine, pyridine or potassium carbonate, at temperatures ranging from about 0° C. to about 150° C.

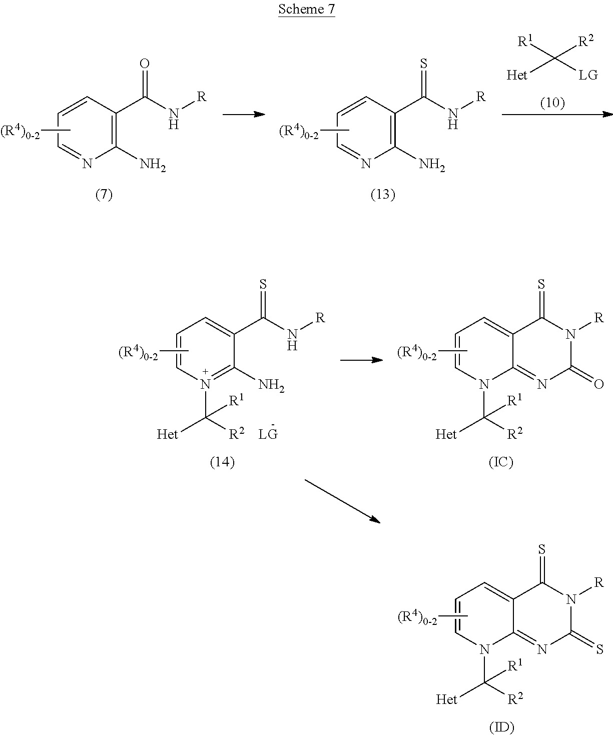

wherein, R, R¹, R², R⁴, LG and Het have the meanings as described above.

A compound of formula (13) can be prepared from a compound of formula (7) by treating it with Lawesson's reagent in the presence of a suitable solvent such as toluene, chlorobenzene, dioxane or tetrahydrofuran at temperatures ranging from about 50° C. to about 150° C. Alternatively the same transformation can be carried out by using phosphorous pentasulfide (P₂S₅), in the presence of a suitable solvent such as toluene, xylene, dioxane or tetrahydrofuran, optionally in the presence of a suitable base such as pyridine or diethylisopropylamine, at temperatures ranging from about 50° C. to about 150° C.

The compound of formula (14) can be prepared from a compound of formula (13) via N-alkylation by reacting it with a compound of formula (10). LG is preferably a halogen (preferably iodide, bromide or chloride) or tosylate or nosylate, or mesylate, whereas the reaction is performed optionally in the presence of a suitable base, and optionally in the presence of a suitable catalyst such as sodium iodide or tert-butyl ammonium iodide or tert-buty ammonium bromide, and in an appropriate solvent such as N,N,-dimethylformamide, N,N-dimethylacetamide, dimethylsulfoxide, tetrahydrofuran, acetonitrile or acetone, at temperatures ranging from about 0° C. to about 150° C.

The compound of formula (IC) can be prepared from a compound of formula (14) by reacting it with a carbonylating reagent such as CDI, diethylcarbonate, phosgene, triphosgene, ethylchloroformate or phenylchloroformate in the presence of a suitable solvent such as tetrahydrofuran, dichloromethane, 1,2-dichloroethane or acetonitrile and in the presence of a suitable base such as an organic or inorganic base which is selected from but not limited to triethylamine, pyridine or potassium carbonate, at temperatures ranging from about 0° C. to about 150° C.

The compound of formula (ID) can be prepared under the same conditions as mentioned for the compound of formula (IC), by using a thiocarbonylating reagent such as thioCDI or thiophenylchloroformate instead of a carbonylating agent.

In one embodiment, the present invention provides a method for the synthesis of a compound of formula (I), comprising the steps selected from a) to e):
a) reacting a compound of formula (III) with a compound of formula (VI), optionally in the presence of a suitable catalyst, a suitable solvent and a suitable base, to obtain a compound of formula (II) or (IIB), which on further reacting with a carbonylating or a thiocarbonylating reagent in the presence of a suitable solvent and a suitable base provided a compound of formula (I),

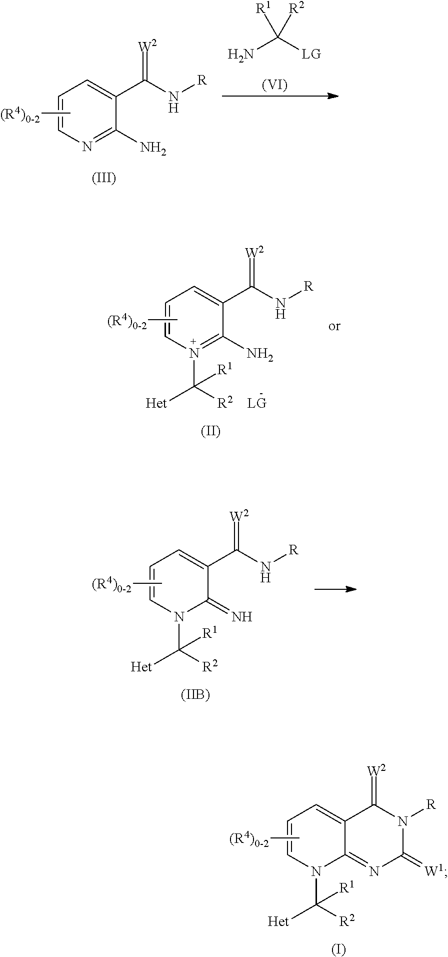

or
b) reacting a compound of formula (III) with a carbonylating or a thiocarbonylating reagent in the presence of a suitable solvent and a suitable base, to obtain a compound of formula (VIII), which on further reacting with a compound of formula (VI), optionally in the presence of a suitable catalyst, a suitable solvent and a suitable base, provided the compound of formula (I),

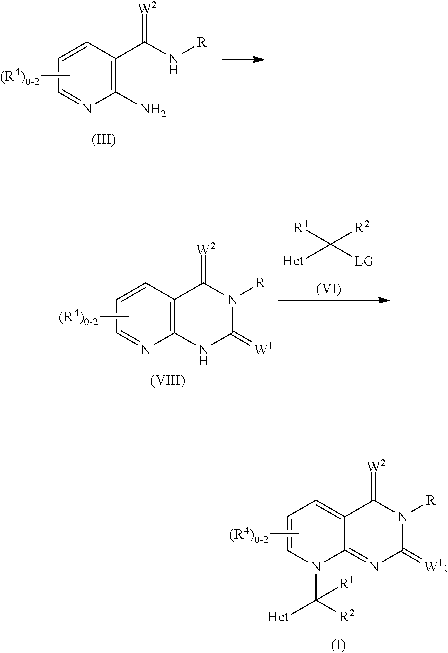

c) further, compound of formula (III) is obtained by reacting a compound of formula (IV) with a compound of formula (V) in the presence of a suitable reagent, a suitable solvent and a suitable base to obtain the compound of formula (III),

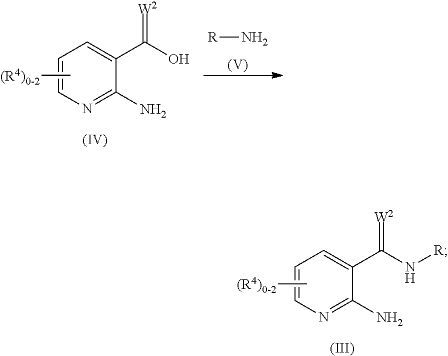

alternatively,
d) compound of formula (I) is obtained by reacting a compound of formula (IIA) with a compound of formula (IIIA) or (IVB), optionally in the presence of a suitable catalyst, a suitable solvent and a suitable base,

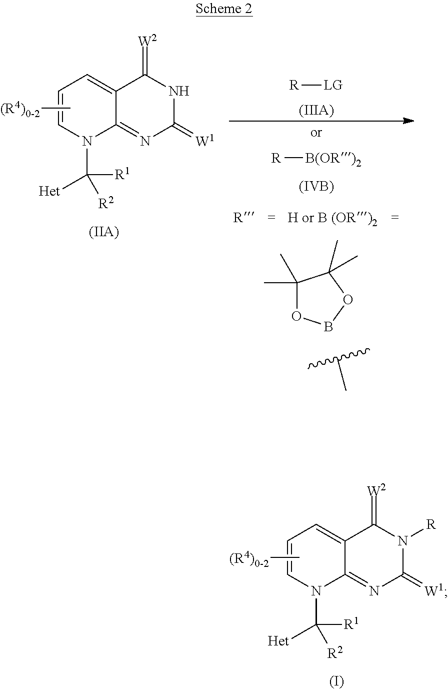

e) reacting a compound of formula (IIIB) with a compound of formula (IVA) to obtain a compound of formula (VA) in the presence of a suitable catalyst, a suitable solvent and a suitable base. Conducting a cyclocondensation reaction of the compound of formula (VA) and a compound of formula (VIA), optionally in the presence of a base to obtain a compound of formula (IIA),

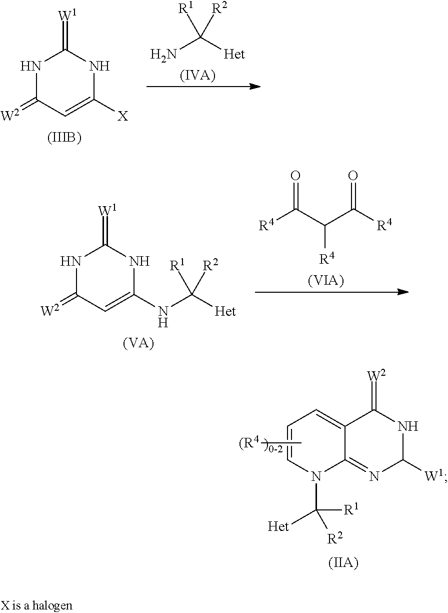

wherein, LG is a halogen or tosylate or nosylate, or mesylate; LG is preferably a halogen (preferably iodide, bromide or chloride) W¹, W², R¹, R², R⁴, R and Het are as defined above.

An embodiment of the present invention provides compounds of formula (II) and (IIA),

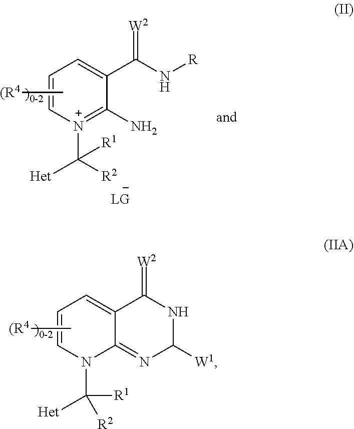

wherein, LG is a halogen (preferably iodide, bromide or chloride) or tosylate or nosylate, or mesylate; Het, R⁴, R¹, R², R, W¹ and W² are as defined above.

Yet another embodiment the present invention provides, is a compound of formula (IIB),

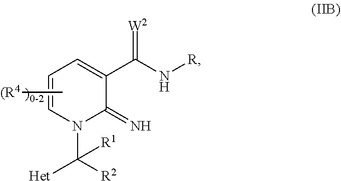

wherein, R=phenyl and C₃-C₁₀-heterocyclyl; wherein each group may optionally be substituted with one or more R^{x}; LG is a halogen (preferably iodide, bromide or chloride) or tosylate or nosylate, or mesylate; Het, R⁴, R¹, R², R, W¹ and W² are as defined above.

In one embodiment the present invention provides the compounds of formula (I) as mentioned in Tables 1 to 7 which can be prepared according to the methods described above. The examples which follow are intended to illustrate the invention and show preferred compounds of formula (I), in the form of a compound of formula (I-1); wherein formula (I-1) may optionally be substituted by one or more R^{x}.

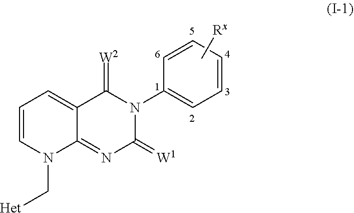

Table D describes the variables W¹, W² and R^{x} of the compounds of formula (I-1).

TABLE D

| Comp. | | | |
|---|---|---|---|
| No. | W¹ | W² | Rx |
| D.1 | O | O | 2-F |
| D.2 | O | O | 3-F |
| D.3 | O | O | 4-F |
| D.4 | O | O | 2-Cl |
| D.5 | O | O | 3-Cl |
| D.6 | O | O | 4-Cl |
| D.7 | O | O | 2-Br |
| D.8 | O | O | 3-Br |
| D.9 | O | O | 4-Br |
| D.10 | O | O | 4-I |
| D.11 | O | O | 2-CH₃ |
| D.12 | O | O | 3-CH₃ |
| D.13 | O | O | 4-CH₃ |
| D.14 | O | O | 2-OCH₃ |
| D.15 | O | O | 3-OCH₃ |
| D.16 | O | O | 4-OCH₃ |
| D.17 | O | O | 2-CHF₂ |
| D.18 | O | O | 3-CHF₂ |
| D.19 | O | O | 4-CHF₂ |
| D.20 | O | O | 2-CF₃ |
| D.21 | O | O | 3-CF₃ |
| D.22 | O | O | 4-CF₃ |
| D.23 | O | O | 2-OCHF₂ |
| D.24 | O | O | 2-OCHF₂ |
| D.25 | O | O | 4-OCHF₂ |
| D.26 | O | O | 2-OCF₃ |
| D.27 | O | O | 3-OCF₃ |
| D.28 | O | O | 4-OCF₃ |
| D.29 | O | O | 2-OCH₂CF₃ |
| D.30 | O | O | 3-OCH₂CF₃ |
| D.31 | O | O | 4-OCH₂CF₃ |
| D.32 | O | O | 2-SCF₃ |
| D.33 | O | O | 3-SCF₃ |
| D.34 | O | O | 4-SCF₃ |
| D.35 | O | O | 2-CN |
| D.36 | O | O | 3-CN |
| D.37 | O | O | 4-CN |
| D.38 | O | O | 3-SO₂CH₃ |
| D.39 | O | O | 4-SO₂CH₃ |
| D.40 | O | O | 2-CyPr |
| D.41 | O | O | 3-CyPr |
| D.42 | O | O | 4-CyPr |
| D.43 | O | O | 2-N(Me)₂ |
| D.44 | O | O | 3-N(Me)₂ |
| D.45 | O | O | 4-N(Me)₂ |
| D.46 | O | O | 2-NO₂ |
| D.47 | O | O | 3-NO₂ |
| D.48 | O | O | 4-NO₂ |
| D.49 | O | O | 2-SMe |
| D.50 | O | O | 3-SMe |
| D.51 | O | O | 4-SMe |
| D.52 | O | O | 3-OCH₂(C≡CH) |
| D.53 | O | O | 3-OCH₂(CH═CH₂) |
| D.54 | O | O | 4-OCyPr |
| D.55 | O | O | 4-(C≡C(Me)) |
| D.56 | O | O | 3-C≡C(CyPr) |
| D.57 | O | O | 3-NH(COMe) |
| D.58 | O | O | 4-NH(COCyPr) |
| D.59 | O | O | 3-NHMe |
| D.60 | O | O | 4-NH(CyPr) |
| D.61 | O | O | 3-CONHMe |
| D.62 | O | O | 4-CON(Me)2 |
| D.63 | O | O | 3-CONH(CyPr) |
| D.64 | O | O | 4-CONMe(CyPr) |
| D.65 | O | O | 3-S(O)Me(═NH) |
| D.66 | O | O | 3-S(O)Me(═NCyPr) |
| D.67 | O | O | 3-S(O)Me(═NMe) |
| D.68 | O | O | 4-S(O)Et(═NH) |
| D.69 | O | O | 4-S(O)Et(═NMe) |
| D.70 | O | O | 4-S(O)Et(═NCyPr) |
| D.71 | O | O | 3-N═S(O)Me₂ |
| D.72 | O | O | 3-N═S(O)MeCyPr |
| D.73 | O | O | 4-N═S(O)Me₂ |
| D.74 | O | O | 4-N═S(O)MeCyPr |
| D.75 | O | O | 2,3-di-F |
| D.76 | O | O | 2,4-di-F |
| D.77 | O | O | 2,5-di-F |
| D.78 | O | O | 2,6-di-F |
| D.79 | O | O | 3,4-di-F |
| D.80 | O | O | 3,5-di-F |
| D.81 | O | O | 2,3-di-Cl |
| D.82 | O | O | 2,4-di-Cl |
| D.83 | O | O | 2,5-di-Cl |
| D.84 | O | O | 2,6-di-Cl |
| D.85 | O | O | 3,4-di-Cl |
| D.86 | O | O | 3,5-di-Cl |
| D.87 | O | O | 2-F, 3-Cl |
| D.88 | O | O | 2-F, 4-Cl |
| D.89 | O | O | 2-F, 5-Cl |
| D.90 | O | O | 3-F, 4-Cl |
| D.91 | O | O | 3-F, 5-Cl |
| D.92 | O | O | 2-Cl, 3-F |
| D.93 | O | O | 2-Cl, 4-F |
| D.94 | O | O | 2-Cl, 5-F |
| D.95 | O | O | 3-Cl, 4-F |
| D.96 | O | O | 2-F, 4-Br |
| D.97 | O | O | 2-F, 5-Br |
| D.98 | O | O | 3-F, 4-Br |
| D.99 | O | O | 2-Cl, 4-Br |
| D.100 | O | O | 2-Cl, 5-Br |
| D.101 | O | O | 3-Cl, 4-Br |
| D.102 | O | O | 2-Br, 4-F |
| D.103 | O | O | 3-Br, 4-F |
| D.104 | O | O | 2-Br, 5-Cl |
| D.105 | O | O | 3-Br, 4-Cl |
| D.106 | O | O | 2-F, 3-CH₃ |
| D.107 | O | O | 2-F, 4-CH₃ |
| D.108 | O | O | 2-F, 5-CH₃ |
| D.109 | O | O | 2-F, 6-CH₃ |
| D.110 | O | O | 2-Cl, 3-CH₃ |
| D.111 | O | O | 2-CH₃, 4-Cl |
| D.112 | O | O | 2-F, 5-CH₃ |
| D.113 | O | O | 2-F, 6-CH₃ |
| D.114 | O | O | 2-Br, 5-CH₃ |
| D.115 | O | O | 2-CH₃, 3-Br |
| D.116 | O | O | 3-CH₃, 5-Br |
| D.117 | O | O | 2-CH₃, 4-Br |
| D.118 | O | O | 3-CN, 4-F |
| D.119 | O | O | 3-CN, 4-Cl |
| D.120 | O | O | 2-F, 5-CN |
| D.121 | O | O | 2-Cl, 5-CN |
| D.122 | O | O | 2-F, 4-CN |
| D.123 | O | O | 2-Cl, 4-CN |
| D.124 | O | O | 3-F, 4-CN |
| D.125 | O | O | 3-Cl, 4-CN |
| D.126 | O | O | 3-Cl, 5-CN |
| D.127 | O | O | 2-Br, 5-CN |
| D.128 | O | O | 2-CN, 3-Br |
| D.129 | O | O | 3-CN, 5-Br |
| D.130 | O | O | 2-CN, 4-Br |
| D.131 | O | O | 3-Br, 4-CN |
| D.132 | O | O | 2-Br, 4-CN |
| D.133 | O | O | 2-F, 3-CF₃ |
| D.134 | O | O | 2-F, 4-CF₃ |
| D.135 | O | O | 2-F, S-CF₃ |
| D.136 | O | O | 3-F, 4-CF₃ |
| D.137 | O | O | 3-F, S-CF₃ |
| D.138 | O | O | 4-F, 3-CF₃ |
| D.139 | O | O | 2-Cl, 3-CF₃ |
| D.140 | O | O | 2-Cl, 4-CF₃ |
| D.141 | O | O | 3-CF₃, 5-Br |
| D.142 | O | O | 3-CF₃, 4-Br |
| D.143 | O | O | 3-CF₃, 5-Me |
| D.144 | O | O | 2-Me, 4-CF₃ |
| D.145 | O | O | 2-Me, 5-CF₃ |
| D.146 | O | O | 3-CF₃, 4-Me |
| D.147 | O | O | 3-CF₃, 5-CN |
| D.148 | O | O | 3-CN, 4-CF₃ |
| D.149 | O | O | 2-OCHF₂, 4-Cl |
| D.150 | O | O | 2-OCHF₂, 4-F |
| D.151 | O | O | 2-Cl, 4-OCF₃ |
| D.152 | O | O | 2-Cl, S-CF₃ |
| D.153 | O | O | 3-Cl, S-CF₃ |
| D.154 | O | O | 4-Cl, 3-CF₃ |
| D.155 | O | O | 2-F, 4-SCH₃ |
| D.156 | O | O | 2-Cl, 4-SCH₃ |
| D.157 | O | O | 2-F, 4-SO₂CH₃ |
| D.158 | O | O | 2-Cl, 4-SO₂CH₃ |
| D.159 | O | O | 3-F, 4-SO₂CH₃ |
| D.160 | O | O | 3-Cl, 4-SO₂CH₃ |
| D.161 | O | O | 3-SO₂CH₃, 4-F |
| D.162 | O | O | 3-SO₂CH₃, 4-Cl |
| D.163 | O | O | 2-F, 5-SO₂CH₃ |
| D.164 | O | O | 2-Cl, 5-SO₂CH₃ |
| D.165 | O | O | 2-OCH₃, 4-Cl |
| D.166 | O | O | 2-F, 3-OCH₃ |
| D.167 | O | O | 2-Cl, 4-OCH₃ |
| D.168 | O | O | 3-OCH₃, 4-Br |
| D.169 | O | O | 3-Br, 5-OCH₃ |
| D.170 | O | O | 2,3,4-tri-F |
| D.171 | O | O | 2,3,5-tri-F |
| D.172 | O | O | 2,3,6-tri-F |
| D.173 | O | O | 3,4,5-tri-F |
| D.174 | O | O | 2,4,6-tri-F |
| D.175 | O | O | 2-Cl, 4,5-di-F |
| D.176 | O | O | 3,5-di-Cl, 4-F |
| D.177 | O | O | 3,4-di-F, S-CF₃ |
| D.178 | O | O | 3,5-di-Cl, 4-Me |
| D.179 | O | O | 3,5-di-F, 4-Me |
| D.180 | O | O | 3,5-di-Me, 4-Cl |
| D.181 | O | O | 3,5-di-Me, 4-F |
| D.182 | O | O | 3,5-di-Cl, 4-OMe |
| D.183 | O | O | 3,5-di-F, 4-OMe |
| D.184 | O | O | 2-O—CF₂—O-3 |
| D.185 | O | O | 3-O—CF₂—O-4 |
| D.186 | O | O | 2-Cl, 4-O—CF₂—O-5 |
| D.187 | O | O | 2-CH₂—CH₂—CH₂-3 |
| D.188 | O | O | 3-CH₂—CH₂—CH₂-4 |
| D.189 | O | O | 2-CH₂—CH₂—CH₂-3, 4-F |

* CyPr = cyclopropyl

Each of Tables 1-7, which follows the Table D above, consist of 189 compounds of the formula (I-1) in which W¹, W² and R^{x} have the values given in each row in Table D, and Het is defined in the relevant Tables 1-7.

Thus compound 1.1 corresponds to a compound of formula (I-1) where W¹, W² and R^{x} are as defined in row 1 of Table D and where Het is as defined in Table 1; compound 1.2 corresponds to a compound of formula (I-1) where W¹, W² and R^{x} are as defined in row 2 of Table D and where Het is as defined in Table 1; and so on.

Table 1: This table discloses 189 compounds 1.1 to 1.189 of the formula, wherein Het is

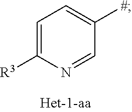

R³ is Cl; W¹, W² and R^{x} are as defined in Table D. For example compound No. 1.1 has the following structure:

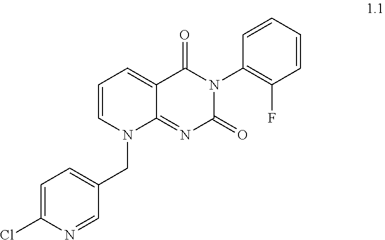 [SMILES: O=c1nc2n(Cc3ccc(Cl)nc3)cccc-2c(=O)n1-c1ccccc1F]

Table 2: This table discloses 189 compounds 2.1 to 2.189 of the formula, wherein Het is

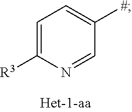

R³ is CF₃; W¹, W² and R^{x} are as defined in Table D.

Table 3: This table discloses 189 compounds 3.1 to 3.189 of the formula, wherein Het is

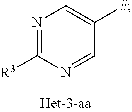

R³ is C₁; W¹, W² and R^{x} are as defined in Table D.

Table 4: This table discloses 189 compounds 4.1 to 4.189 of the formula, wherein Het is

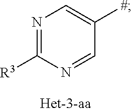

R³ is H; W¹, W^{Z} and R^{x} are as defined in Table D.

Table 5: This table discloses 189 compounds 5.1 to 5.189 of the formula, wherein Het is

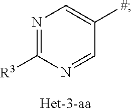

R³ is CF₃; W¹, W² and R^{x} are as defined in Table D.

Table 6: This table discloses 189 compounds 6.1 to 6.189 of the formula, wherein Het is

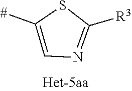

R³ is Cl; W¹, W² and R^{x} are as defined in Table D.

Table 7: This table discloses 189 compounds 7.1 to 7.189 of the formula, wherein Het is

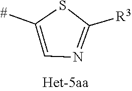

R³ is CF₃; W¹, W² and R^{x} are as defined in Table D.

Depending on the nature of the substituents, the compounds of formula (I), and intermediates thereof, may be present either in pure form or as mixtures of different possible isomeric forms such as stereoisomers. The various stereoisomers include enantiomers, diastereomers, chiral isomers, atropisomers, conformers, rotamers, tautomers, optical isomers, polymorphs, and geometric isomers.

Depending on the nature of the substituents, the compounds of formula (I) and intermediates thereof as mentioned in the general schemes, can also exist in one or more geometric isomer forms depending on the relative position (syn/anti or cis/trans) of the substituents. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by a person ordinary skilled in the art.

In another embodiment, the present invention provides a process for the preparation of the compound of formula (I) or of salts thereof.

The compounds of formula (I) including their stereoisomers, salts, and N-oxides, and their precursors in the synthesis process, can be prepared by the methods described above. If individual compounds cannot be prepared directly via the above-described routes, they can be prepared by derivatization of other compounds (I) or of the respective precursor or by customary modifications of the synthetic routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in a customary manner, e.g. by mixing with water, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds of formula (I) cannot be obtained by the routes described above, they can be prepared by derivatization of other compounds of formula (I). However, if the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant, or in the harmful fungus to be controlled.

In one embodiment, the present invention provides a composition for controlling or preventing invertebrate pests. The composition comprises a biologically effective amount of the compound of formula (I), agriculturally acceptable salts, stereo-isomers, metal complexes, polymorphs, or N-oxides thereof and at least one additional component selected from the group consisting of surfactants and auxiliaries.

In another embodiment, compounds of formula (I) of the present invention are also read as including salts thereof. Exemplary salts include, but are not limited to: hydrochloride, hydrobromide, hydroiodide, acetate, trifluoroacetate, and trifluoromethane sulfonate.

In yet another embodiment, the composition additionally comprises at least one additional biologically active and compatible compound selected from fungicides, insecticides, nematicides, acaricides, biopesticides, herbicides, plant growth regulators, antibiotics, fertilizers or nutrients.

In yet another embodiment, the present invention provides a compound of formula (I) or its N-oxides or salts having been processed into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further composition types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, “Formulation Technology”, Wiley VCH, Weinheim, 2001; or Knowles, “New Developments in Crop Protection Product Formulation”, Agrow Reports DS243, T and F Informa, London, 2005.

The formulation adjuvants that are suitable for the preparation of the compositions according to the invention are known per se. Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers or binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; dimethyl sulfoxide; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: “Emulsifiers and Detergents”, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-subsititued fatty acid amides are fatty acid glucamides or fatty acid alkanolamides. Examples of esters are fatty acid esters, glycerol esters or monoglycerides. Examples of sugarbased surfactants are sorbitans, ethoxylated sorbitans, sucrose and glucose esters or alkylpolyglucosides. Examples of polymeric surfactants are homeor copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide.

Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, TandF Informa UK, 2006, chapter 5.

Suitable thickeners are polysaccharides (e.g. xanthan gum, carboxymethylcellulose), anorganic clays (organically modified or unmodified), polycarboxylates, and silicates. Suitable bactericides are bronopol and isothiazolinone derivatives such as alkylisothiazolinones and benzisothiazolinones. Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and watersoluble dyes. Examples are inorganic colorants (e.g. iron oxide, titan oxide, iron hexacyanoferrate) and organic colorants (e.g. alizarin-, azoand phthalocyanine colorants). Suitable tackifiers or binders are polyvinylpyrrolidone, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound (I) or an N-oxide or salt thereof and 5-15 wt % wetting agent (e.g. alcohol alkoxylates) are dissolved in water and/or in a water-soluble solvent (e.g. alcohols) up to 100 wt %. The active substance dissolves upon dilution with water.

### ii) Dispersible Concentrates (DC)

5-25 wt % of a compound (I) or an N-oxide or salt thereof and 1-10 wt % dispersant (e. g. polyvinylpyrrolidone) are dissolved in up to 100 wt % organic solvent (e.g. cyclohexanone). Dilution with water gives a dispersion.

iii) Emulsifiable Concentrates (EC)

15-70 wt % of a compound I or an N-oxide or salt thereof and 5-10 wt % emulsifiers (e.g. calcium dodecylbenzenesulfonate and castor oil ethoxylate) are dissolved in up to 100 wt % waterinsoluble organic solvent (e.g. aromatic hydrocarbon). Dilution with water gives an emulsion.

### iv) Emulsions (EW, EO, ES)

5-40 wt % of a compound (I) or an N-oxide or salt thereof and 1-10 wt % emulsifiers (e.g. calcium dodecylbenzenesulfonate and castor oil ethoxylate) are dissolved in 20-40 wt % water- insoluble organic solvent (e.g. aromatic hydrocarbon). This mixture is introduced into up to 100 wt % water by means of an emulsifying machine and made into a homogeneous emulsion. Dilution with water gives an emulsion.

### v) Suspensions (SC, OD, FS)

In an agitated ball mill, 20-60 wt % of a compound (I) or an N-oxide or salt thereof are comminuted with the addition of 2-10 wt % dispersants and wetting agents (e.g. sodium lignosulfonate and alcohol ethoxylate), 0,1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound (I) or an N-oxide or salt thereof are ground finely with the addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound (I) or an N-oxide or salt thereof are ground in a rotor- stator mill with the addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gels (GW, GF)

In an agitated ball mill, 5-25 wt % of a compound (I) or an N-oxide or salt thereof are comminuted with the addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance,

### ix) Microemulsions (ME)

5-20 wt % of a compound (I) or an N-oxide or salt thereof are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

### x) Microcapsules (CS)

An oil phase comprising 5-50 wt % of a compound I or an N-oxide or salt thereof, 0-40 wt % water insoluble organic solvent (e.g. aromatic hydrocarbon), 2-15 wt % acrylic monomers (e.g. methylmethacrylate, methacrylic acid and a dior triacrylate) are dispersed into an aqueous solution of a protective colloid (e.g. polyvinyl alcohol). Radical polymerization initiated by a radical initiator results in the formation of poly(methyl acrylate) microcapsules. Alternatively, an oil phase comprising 5-50 wt % of a compound (I) according to the present invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition,

### xi) Dustable Powders (DP, DS)

1-10 wt % of a compound I or an N-oxide or salt thereof are ground finely and mixed intimately with up to 100 wt % solid carrier, e.g. finely divided kaolin.

xii) Granules (GR, FG)

0.5-30 wt % of a compound I or an N-oxide or salt thereof is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I or an N-oxide or salt thereof are dissolved in up to 100 wt % organic solvent, e.g. aromatic hydrocarbon.

The composition types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

In one another embodiment, the present invention provides agrochemical compositions containing a compound of formula (I), which comprises the active substance between 0.01 and 95% by weight, preferably between 0.1 and 90%, and more preferably between 1 and 70%, in particular between 10 and 60% by weight of active substance. The active substances are employed in a purity of 90% to 100%, preferably from 95% to 100% (according to NMR spectrum).

Water-soluble concentrates (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of the treatment of plant propagation materials, particularly seeds. The compositions in question give, after two-to-tenfold dilution, active substance concentrations of 0.01 to 60% by weight, preferably from 0.1 to 40% by weight, in the ready-to-use preparations. Application can be carried out before or during sowing.

Methods for applying compounds of formula (I) and compositions thereof, respectively, on to plant propagation material, especially seeds, include dressing, coating, pelleting, dusting, soaking and in-furrow application methods of the propagation material. Preferably, compounds of formula (I) or compositions thereof, respectively, are applied onto the plant propagation material in a way such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.02 to 0.9 kg per ha, in particular from 0.05 to 0.75 kg per ha.

For the treatment of plant propagation materials such as seeds, e.g. by dusting, coating or drenching seed, amounts of active substance ranging from 0.1 to 1000 g, preferably from 1 to 200 g, more preferably from 5 to 150 g and most preferably from 10 to 100 g, per 100 kilogram of plant propagation material (preferably seed) are generally required.

When used for the protection of materials or stored products, the amount of active substance applied depends on the kind of application area and on the desired effect. Amounts customarily applied in the protection of materials are ranging from 0.001 g to 2 kg, preferably from 0.005 g to 1 kg, of active substance per cubic meter of treated material.

Various types of oils, wetters, adjuvants, fertilizer, or micronutrients, and other pesticides (e.g. herbicides, insecticides, fungicides, growth regulators, safeners, antibiotics, fertilizers, nutrients and biostimulants) may be added to the active substances or the compositions comprising them as premix or, if appropriate, may be added not until immediately prior to use (tank mix). These agents can be admixed with the compositions according to the present invention in a weight ratio of 1:100 to 100:1, preferably 1:10 to 10:1.

The user can apply the composition according to the present invention usually from a predosage device, a knapsack sprayer, a spray tank, a spray plane, or an irrigation system. Usually, the agrochemical composition is made up with water, buffer, and/or further auxiliaries to the desired application concentration, and the ready-to-use spray liquor or the agrochemical composition according to the present invention is obtained in this way. Usually, 20 to 6000 liters, preferably 35 to 1000 liters, more preferably 50 to 500 liters, of the ready-to-use spray liquor are applied per hectare of the agricultural useful area.

According to one embodiment, individual components of the composition according to the present invention such as parts of a kit or parts of a binary or ternary mixture may be mixed by the user himself in a spray tank, and further auxiliaries may be used in addition, if appropriate.

The compounds and compositions of the present invention are in this way agronomically useful for protecting field crops from phytophagenic invertebrate pests, and also, nonagronomically, for protecting other horticultural crops and plants from phytophagenic invertebrate pests. This utility includes protecting crops and other plants (i.e. both agronomically and nonagronomically) that contain genetic material introduced by genetic engineering (i.e. transgenic) or modified by mutagenesis to provide advantageous traits.

The compounds of the present invention are characterized by favorable metabolic and/or soil residual patterns and exhibit activity controlling a spectrum of agronomic and nonagronomic invertebrate pests. The compounds of the present invention are preventively and/or curatively valuable active ingredients in the field of pest control, even at low rates of application, which can be used against insecticide resistant pests such as insects and mites, and are well tolerated by warm-blooded species, fish and plants.

In the context of the present invention “invertebrate pest control” means inhibition of invertebrate pest development (including mortality) that causes significant reduction in feeding or other injury or damage caused by the pest; related expressions are defined analogously. As referred to in the present invention, the term “invertebrate pest” includes arthropods, gastropods and nematodes of economic importance as pests. The term “arthropod” includes insects, mites, spiders, scorpions, centipedes, millipedes, pill bugs and symphylans.

The term “gastropod” includes snails, slugs and other stylommatophora. The term “nematode” includes all helminths, such as: roundworms, heartworms, and phytophagous nematodes (Nematoda), flukes (Tematoda), acanthocephala, and tapeworms (Cestoda). Those skilled in the art will recognize that not all compounds are equally effective against all pests.

The compounds of the present invention display activity against economically important agronomic, forest, greenhouse, nursery, ornamentals, turfgrass, food and fiber, public and animal health, domestic and commercial structure, household, and stored product pests. These include larvae of the order Lepidoptera, such as armyworms, cutworms, loopers, and heliothines in the family Noctuidae (e.g., fall armyworm (Spodoptera fugiperda J. E. Smith), beet armyworm (Spodoptera exigua Hubner), black cutworm (Agrotis ipsilon Hufnagel), cabbage looper (Trichoplusia ni Hubner), tobacco budworm (Heliothis virescens Fabricius); borers, casebearers, webworms, coneworms, cabbageworms and skeletonizers from the family Pyralidae (e.g., European corn borer (Ostrinia nubilalis Hubner)), navel orangeworm (Amyelois transitella Walker), corn root webworm (Crambus caliginosellus Clemens), sod webworm (Herpetogramma licarsisalis Walker)); leafroHers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck)); and many other economically important lepidoptera (e.g., diamondback moth (Plutella xylostella Linnaeus), pink bollworm (Pectinophora gossypiella Saunders), gypsy moth (Lymantria dispar Linnaeus)); nymphs and adults of the order Blattodea mcluding cockroaches from the families Blattellidae and Blattidae (e.g., oriental cockroach (Blatta orientalis Linnaeus), Asian cockroach (Blatella asahinai Mizukubo), German cockroach (Blattella gemnanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)); foliar feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scaribaeidae (e.g., Japanese beetle (Popillia japonica Newman) and European chafer (Rhizotrogus majalis Razoumowsky)); carpet beetles from the family Dermestidae; wireworms from the family Elateridae; bark beetles from the family Scolytidae and flour beetles from the family Tenebrionidae. In addition it includes: adults and larvae of the order Dermaptera including earwigs from the family Forficulidae (e.g., European earwig (Forficula auricularia Linnaeus), black earwig (Chelisoches mono Fabricius)); adults and nymphs of the orders Hemiptera and Homoptera such as, plant bugs from the family Miridae, cicadas from the family Cicadidae, leafhoppers (e.g. Empoasca spp.) from the family Cicadellidae, planthoppers from the families Fulgoroidae and Delphacidae, treehoppers from the family Membracidae, psyllids from the family Psyllidae, whitefiies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, cinch bugs (e.g., Blissus spp.) and other seed bugs from the family Lygaeidae, spittlebugs from the family Cercopidae squash bugs from the family Coreidae, and red bugs and cotton stainers from the family Pyrrhocoridae. Also included are adults and larvae of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)), flat mites in the family Tenuipalpidae (e.g., citrus flat mite (Brevipalpus lewisi McGregor)), rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae (e.g., deer tick (Ixodes scapularis Say), Australian paralysis tick (Ixodes holocyclus Neumann), American dog tick (Dermacentor variabilis Say), lone star tick (Amb/yomma americanum Linnaeus) and scab and itch mites in the families Psoroptidae, Pyemotidae, and Sarcoptidae; adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), house cricket (Acheta domesticus Linnaeus), mole crickets (Gryllotalpa spp.)); adults and immatures of the order Diptera including leafminers, midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F.femoralis Stein), stable flies (e.g., Stomoxys calcitrans Linnaeus), face flies, horn flies, blow flies (e.g., Chiysomya spp., Phonnia spp.), and other muscoid fly pests, horse flies (e.g., Tabanus spp.), botflies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; adults and immatures of the order Thysanoptera including onion thrips (Thrips tabaci Lindeman) and other foliar feeding thrips; insect pests of the order Hymenoptera including ants (e.g., red carpenter ant (Camponotus ferrugineus Fabricius), black carpenter ant (Camponotus pennsylvanicus De Geer), Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus FBrster), odorous house ant (Tapinoma sessile Say)), bees (including carpenter bees), hornets, yellow jackets and wasps; insect pests of the order Isoptera including the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder) and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus humanus Linnaeus), chicken body louse (Menacanthus stramineus Nitszch), dog biting louse (Trichodectes cams De Geer), fluff louse (Goniocotes gallinae De Geer), sheep body louse (Bovicola ovis Schrank), short-nosed cattle louse (Haematopinus eurystemus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephatides canis Curtis), hen flea (Ceratophyllus gallinae Schrank), sticktight flea (Echidnophaga gallinacea Westwood), human flea (Pulex irritans Linnaeus) and other fleas afflicting mammals and birds.

Additional arthropod pests covered include: spiders in the order Araneae such as the brown recluse spider (Loxosceles reclusa Gertsch and Mulaik) and the black widow spider (Latrodectus mactans Fabricius), and centipedes in the order Scutigeromorpha such as the house centipede (Scutigera coleoptrata Linnaeus). Activity also includes members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala peifoliata in horses, Fasciola hepatica Linnaeus in ruminants, etc.).

The compounds of the present invention show activity against pests in the order of Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenee (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoveipa armigera H6bner (American bollworm), Helicoverpa zea Boddie (corn earworm), Heliothis virescens Fabricius (tobacco budworm), Herpetogramma licarsisalis Walker (sod webworm), Lobesia botrana Denis and Schiffeicnvller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hvbner (cabbage looper) and Tula absoluta Meyrick (tomato leafminer)).

Compounds of the present invention also have commercially significant activity on members from the order Homoptera including: Acyrthisiplionpisum Harris (pea aphid), Aphis craccivora Koch (cowpea aphid), Aphisfabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (tarnip aphid), Metopolophium dirrhodum Walker (cereal aphid), Macrosipum euphorbiae Thomas (pqtato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii, Boyer de Fonscolombe (black citrus aphid), and Toxoptera citiicida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows and Perring (silverleaf whitefly), Dialeurodes citri Ashmead (citrus whitefly) and Trialeurodes vaporariorum Westwood (greenhouse whitefly); Empoascafabae Harris, (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stal (rice leafhopper), Nilaparvata lugens Stal (brown planthopper), Peregrinus maidis Ashmead (corn planthopper), Sogatella furcifera Horvath (white-backed planthopper), Sogatodes orizicola Muir (rice delphacid), Typhlocyba pomaria McAfee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

These compounds also have activity on members from the order of Hemiptera including: Acrostemum hilare Say (green stink bug), Anasa tristis De Geer (squash bug), Blissus leucopterus leucopterus Say (chinch bug), Corythuca gossypii Fabricius (cotton lace bug), Cyrtopeltis modesta Distant (tomato bug), Dysdercus suturellus Herrich-S chaffer (cotton stainer), Euchistus servus Say (brown stink bug), Euchistus variolrius Palisot deBeauvois (one-spotted stink bug), Graptdsthetus spp. (complex of seed bugs), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltusfasciatus DaEas (large milkweed bug), Pseudatomoscelis seriatus Reuter (cotton fleahopper).

Other insects which are controlled by compounds of formula (I) of the present invention include: Thysanoptera (e.g., Frankliniella occidentalis Pergande (western flower thrip), Scirthothnps citri Moulton (citrus thrip), Sericothrips variabilis Beach (soybean thrip), and Thrips tabaci Lindeman (onion thrip); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

In one embodiment of the present invention, the present invention provides the compound of formula (I) is useful for controlling insects preferably selected form sucking or piercing insects such as insects from the genera Thysanoptera, Diptera and Hemiptera, in particular the following species:
Thysanoptera: Frankliniella fusca, Frankliniella occidentalis, Frankliniella tritici, Scirtothrips citri, Thrips oryzae, Thrips palmi and Thrips tabaci,
Diptera: Aedes aegypti, Aedes albopictus, Aedes vexans, Anastrepha ludens, Anopheles maculipennis, Anopheles crucians, Anopheles albimanus, Anopheles gambiae, Anophelesfreeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa; Hemiptera, in particular aphids: Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mail, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii.

The compounds of formula (I), their N-oxides, their isomers, their polymorphs and their salts are also suitable for efficiently combating the following pests: Insects from the order of the lepidopterans (Lepidoptera), for example Agrotis ipsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Chilo infuscatellus, Choristoneura fumiferana, Choristoneura occidentalis, Cirphis unipuncta, Cydia pomonella, Dendrolimus pini, Diaphania nitidalis, Diatraea grandiosella, Earias insulana, Earias vittella, Elasmopalpus lignosellus, Eupoecilia ambiguella, Evetria bouliana, Feltia subterranea, Galleria mellonella, Grapholita funebrana, Grapholita molesta, Helicoverpa armigera, Helicoverpa virescens, Helicoverpa zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Leucinodes orbonalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scirpophaga incertulas, Scrobipalpula absoluta, Sitotroga cerealella, Sparganothis pilleriana, Spodoptera frugiperda, Spodoptera littoralis, Spodoptera litura, Spodoptera exigua, Thaumatopoea pityocampa, Tortrix viridana, Trichoplusia ni and Zeiraphera canadensis; and Beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica undecimpunctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria; flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa; termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus; cockroaches (Blattaria Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis; ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile; crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina; Araneida, e.g. Latrodectus mactans, and Loxosceles reclusa; fleas (Siphonaptera), e.g. Ctenocephalides felis, Ctenocephalides canis, Xenopsylla cheopis, Pulex irritans, Tunga penetrans, and Nosopsyllus fasciatus, silverfish, firebrat (Thysanura), e.g. Lepisma saccharina and Thermobia domestica, centipedes (Chilopoda), e.g. Scutigera oleoptrata, millipedes (Diplopoda), e.g. Narceus spp., Earwigs (Dermaptera), e.g. forficula auricularia, lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon allinae, Menacanthus stramineus and Solenopotes capillatus. Collembola (springtails), e.g. Onychiurus ssp.

The compounds of formula (I) of the present invention are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species: false rootknot nematodes, Nacobbus species: Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of formula (I) and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis.

In one embodiment of the present invention, the present invention provides the compounds of formula (I) as useful for controlling insects selected form sucking or piercing insects such as insects from the genera Thysanoptera, Diptera and Hemiptera and in particular the following species:
Thysanoptera: Frankliniellafusca, Frankliniella occidentalis, Frankliniella tritici, Scirtothrips citri, Thrips oryzae, Thrips palmi and Thrips tabaci,
Diptera: Aedes aegypti, Aedes albopictus, Aedes vexans, Anastrepha ludens, Anopheles maculipennis, Anopheles crucians, Anopheles albimanus, Anopheles gambiae, Anophelesfreeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;
Hemiptera, in particular aphids: Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribisnigri, Nilaparvata lugens, Pemphigus bursarius, Perkinsiella saccharicida, Phorodon humuli, Psylla mail, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii.

In one preferred embodiment, the present invention of the compounds of formula (I) are especially useful for controlling for example western flower thrips (Frankliniella occidentalis), potato leafhopper (Empoasca fabae), rice brown planthopper (Nilaparvata lugens), rice green leafhopper (Nephotettix virescens), cotton/melon aphid (Aphis gossypii), green peach aphid (Myzus persicae), tobacco/sweetpotato whitefly (Bemisia tabaci) and silverleaf whitefly (Bemisia argentifolii).

In one embodiment, the present invention provides a composition comprising a biologically effective amount of the compound of formula (I) and at least one additional biological active compatible compound selected from fungicides, insecticides, nematicides, acaricides, biopesticides, herbicides, plant growth regulators, antibiotics, fertilizers, nutrients and biostimulants. The compounds used in the composition and in combination with the compound of formula (I) are also termed as active compatible compounds.

The known and reported fungicides, insecticides, nematicides, acaricides, biopesticides, herbicides, plant growth regulators, antibiotics and nutrients can be combined with at least one compound of the formula (I) of the present disclosure. For example, fungicides, insecticides, nematicides, acaricides, biopesticides, herbicides, plant growth regulators, antibiotics, fertilizers, nutrients and biostimulants disclosed and reported in WO2016156129 and/or WO2017153200 can be combined with at least one compound of formula (I) of the present disclosure.

The fungicides, insecticides, nematicides, acaricides, biopesticides, herbicides, plant growth regulators, antibiotics, fertilizers and nutrients reported in WO2016156129 and or WO2017153200 are incorporated herein by way of reference as non-limiting examples to be combined with at least one compound of the formula (I) of the present disclosure.

Particularly, the compounds of the present invention can be mixed with at least one additional biological active compatible compound (mixing partner) which includes but is not limited to insecticides, fungicides, nematocides, bactericides, acaricides, growth regulators such as rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agricultural utility.

Examples of such biologically active compounds or agents/mixing partners with which compounds of formula (I) of the present invention can be combined/formulated are disclosed in the WO2019072906A1 (page 27 to 37).

In one embodiment, the biological agents for mixing with compounds of formula (I) of the present invention include Bacillus thuringiensis, Bacillus thuringiensis delta endotoxin as well as naturally occurring and genetically modified viral insecticides including members of the family Baculoviridae as well as entomophagous fungi.

In certain instances, combinations with other invertebrate pest control compounds or agents having a similar spectrum of control but a different mode of action will be particularly advantageous for resistance management. Thus, compositions of the present invention can further comprise a biologically effective amount of at least one additional invertebrate pest control compound or agent having a similar spectrum of control but a different mode of action. Contacting a plant genetically modified to express a plant protection compound (e.g., protein) or the locus of the plant with a biologically effective amount of a compound of the invention can also provide a broader spectrum of plant protection and be advantageous for resistance management.

In one embodiment of the present invention, the biologically effective amount of the compound of formula (I) in the compositions ranges from 0.1% to 99% by weight with respect to the total weight of the composition, preferably from 5% to 50% by weight with respect to the total weight of the composition.

The present invention furthermore provides a method of combating invertebrate pests, said method comprising contacting the invertebrate pests, their habitat, breeding ground, food supply, plant, seed, soil, area, material or environment in which the invertebrate pests are growing or may grow, or the materials, plants, seeds, soils, surfaces or spaces to be protected from pest attack or infestation with a biologically effective amount of the compound of formula (I) or agriculturally acceptable salts, stereo-isomers, metal complexes, polymorphs, or N-oxides thereof and/or compositions or combinations thereof.

Invertebrate pests are controlled and the protection of agronomic, horticultural and specialty crops, animal and human health is achieved by applying one or more of the compounds of the present invention, in an effective amount, to the environment of the pests including the agronomic and/or nonagronomic locus of infestation, to the area to be protected, or directly on the pests to be controlled. Thus, the present invention further comprises a method for the control of foliar- and soil-inhabiting invertebrates and protection of agronomic and/or nonagronomic crops, comprising contacting the invertebrates or their environment with abiologically effective amount of one or more of the compounds of the present invention, or with a composition comprising at least one such compound or a composition comprising at least one such compound and an effective amount of at least one additional biologically active compound or agent. A preferred method of contact is by spraying. Alternatively, a granular composition comprising a compound of the present invention can be applied to the plant foliage or the soil. Compounds of the present invention are effective in delivery through plant uptake by contacting the plant with a composition comprising a compound of the present invention applied as a soil drench of a liquid formulation, a granular formulation to the soil, a nursery box treatment or a dip of transplants. Other methods of contact include application of a compound or a composition of the present invention by direct and residual sprays, aerial sprays, seed coats, microencapsulations, systemic uptake, baits, eartags, boluses, foggers, fumigants, aerosols, dusts and many others.

The compounds of the present invention can be incorporated into baits that are consumed by the invertebrates or within devices such as traps and the like. Granules or baits comprising between 0.01-5% active ingredient, 0.05-10% moisture retaining agent(s) and 40-99% vegetable flour are effective in controlling soil insects at very low application rates, particularly at doses of active ingredient that are lethal by ingestion rather than by direct contact. The compounds of the present invention can be applied in their pure state, but most often application will be of a formulation comprising one or more compounds with suitable carriers, diluents, and surfactants and possibly in combination with a food depending on the contemplated end use. A preferred method of application involves spraying a water dispersion or refined oil solution of the compounds. Combinations with spray oils, spray oil concentrations, spreader stickers, adjuvants, other solvents, and synergists such as piperonyl butpxide often enhance compound efficacy.

The rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredient per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The animal pest, i.e. the arthropods, the plant, soil or water in which the plant is growing can be contacted with compounds of formula (I), their N-oxides and salts or composition(s) containing them by any application method known in the art. As used herein, “contacting” includes both direct contact (applying the compounds/compositions directly on the animal pest or plant typically to the foliage, stem or roots of the plant) and indirect contact (applying the compounds/compositions to the locus of the animal pest or plant).

The compounds of the present invention or the pesticidal compositions comprising them may be used to protect growing plants and crops from attack or infestation by animal pests, especially insects, and mites by contacting the plant/crop with a pesticidally effective amount of at least one compound of the present invention. The term “crop” refers both to growing and harvested crops. The compounds of the invention are also suitable for the use in combating or controlling animal pests. Therefore, the invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the invention.

In one embodiment, the present invention provides a method for protecting crops from an attack or infestation by invertebrate pests, which comprises contacting the crop with a biologically effective amount of the compound or the composition of the present invention, isomer, polymorph, N-oxide or salt thereof.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

In one embodiment, the present invention provides a method for the protection of seeds from soil insects and of the seedling's roots and shoots from soil and foliar insects comprising contacting the seeds before sowing and/or after pre-germination with the compound or the composition of the present invention, N-oxide or salt thereof.

Furthermore, the present invention provides a method for treating or protecting animals against an infestation or infection by parasites which comprises orally, topically or parenterally administering or applying to the animals a biologically effective amount of compounds of formula (I) or compositions of the present invention, isomer, polymorph, N-oxide or veterinary acceptable salt thereof.

For use in treating crop plants, the rate of application (applying effective dosages) of the compound of the present invention may be in the range of 1 g a.i. to 2000 g a.i. per hectare in agricultural or horticultural crops, preferably from 10 g to 500 g per hectare, more preferably from 50 g to 200 g per hectare.

The compounds and the compositions of the present invention are particularly useful in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

Particularly, the compound or the composition of the present invention are useful in protecting agricultural crops such as cereals, corn, sorghum, bajra, rice, soybean, oil seeds and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, ornamentals, brassica vegetables, fruiting vegetables, leafy vegetables, brassica leafy vegetables, and other vegetables.

The compounds of the present invention are effective through both contact (via soil, glass, wall, bed net, carpet, plant parts or animal parts), and ingestion (bait or plant part).

The compounds of the present invention may also be applied against non-crop invertebrate pests, such as ants, termites, wasps, flies, mosquitos, crickets, or cockroaches. For the use against said non-crop pests, compounds of the present invention are preferably used in a bait composition.

As used herein, the term “non-crop insect pest” refers to pests, which are particularly relevant for non-crop targets, such as ants, termites, wasps, flies, ticks, mosquitos, crickets, or cockroaches.

The bait can be a liquid, a solid or a semisolid preparation (e.g. a gel). Solid baits can be formed into various shapes and forms suitable to the respective application e.g. granules, blocks, sticks, disks.

Liquid baits can be filled into various devices to ensure proper application, e.g. open containers, spray devices, droplet sources, or evaporation sources. Gels can be based on aqueous or oily matrices and can be formulated to particular use in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish or blood meal, insect parts, egg yolk), from fats and oils of animal and/or plant origin, or mono-, oligo or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For the use in bait compositions, the typical content of active ingredient is from 0.001 weight % to 15 weight %, desirably from 0.001 weight % to 5 weight % of the active compound.

Formulations of compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in so far that no propellants are used. For the use in spray compositions, the content of active ingredient is from 0.001 to 80 weights %, preferably from 0.01 to 50 weight % and most preferably from 0.01 to 15 weight %.

The compounds of the present invention and their respective compositions can also be used in mosquito and fumigating coils, smoke cartridges, vaporizer plates or long-term vaporizers as well as in moth papers, moth pads or other heat-independent vaporizer systems.

The methods to control infectious diseases transmitted by insects (e.g. malaria, dengue and yellow fever, lymphatic filariasis, and leishmaniasis) with compounds of formula (I) and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2hydroxymethylcyclohexyl) acetic acid lactone, 2-ethyl-1 ,3-hexandiol, indalone, Methylneodecanamide (MNDA), a pyrethroid not used for insect control such as {(+/−)-3-allyl-2-methyl4-oxocyclopent-2-(+)-enyl-(+)-trans-chrysantemate (Esbiothrin), a repellent derived from or identical with plant extracts like limonene, eugenol, (+)-Eucamalol (1), (−)-1-epi- eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as vinyl acetate and vinyl versatate), acrylic and methacrylic esters of alcohols, such as butyl acrylate, 2-ethylhexylacrylate, and methyl acrylate, monoand di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of the present invention and their compositions can be used for protecting wooden materials such as trees, board fences, sleepers, etc. and buildings such as houses, outhouses, factories, but also construction materials, furniture, leathers, fibers, vinyl articles, electrie wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets and heat insulating material such as styrene foams, etc.

In case of an application against ants doing harm to crops, wooden dwellings or human beings, a compound of the present invention is applied to the crops or the surrounding soil, or is directly applied to the nest of ants or the like.

A preferred method of application in the field of crop protection is the application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

### Digital Technologies

The compounds of the invention can be used in combination with models e.g. embedded in computer programs for site specific crop management, satellite farming, precision farming or precision agriculture. Such models support the site specific management of agricultural sites with data from various sources such as soils, weather, crops (e.g. type, growth stage, plant health), weeds (e.g. type, growth stage), diseases, pests, nutrients, water, moisture, biomass, satellite data, yield etc. with the purpose to optimize profitability, sustainability and protection of the environment. In particular, such models can help to optimize agronomical decisions, control the precision of pesticide applications and record the work performed.

As an example, the compounds of the invention can be applied to a crop plant according to an appropriate dose regime if a model models the development of a pest and calculates that a threshold has been reached for which it is recommendable to apply the compound of the invention to the crop plant.

Commercially available systems which include agronomic models are e.g. FieldScriptsTM from The Climate Corporation, XarvioTM from BASF, AGLogicTM from John Deere, etc.

The compounds of the invention can also be used in combination with smart spraying equipment such as e.g. spot spraying or precision spraying equipment attached to or housed within a farm vehicle such as a tractor, robot, helicopter, airplane, unmanned aerial vehicle (UAV) such as a drone, etc. Such an equipment usually includes input sensors (such as e.g. a camera) and a processing unit configured to analyze the input data and configured to provide a decision based on the analysis of the input data to apply the compound of the invention to the crop plants (respectively the weeds) in a specific and precise manner. The use of such smart spraying equipment usually also requires positions systems (e.g. GPS receivers) to localize recorded data and to guide or to control farm vehicles; geographic information systems (GIS) to represent the information on intelligible maps, and appropriate farm vehicles to perform the required farm action such as the spraying.

In an example, pests can be detected from imagery acquired by a camera. The pests can be identified and/or classified based on that imagery. Such identification and/classification can make use of image processing algorithms. Such image processing algorithms can utilize machine learning algorithms, such as trained neutral networks, decision trees and utilize artificial intelligence algorithms. In this manner, the compounds described herein can be applied only where needed.

### Seed Treatment

The present invention further provides a seed comprising the compounds of the present invention, particularly in an amount ranging from about 0.0001% to about 1% by weight of the seed before treatment.

The compounds of the present invention are also suitable for the treatment of seeds to protect the seed from insect pests, in particular from soil-living insect and mite pests and the resulting plant's roots and shoots against soil pests and foliar insects.

The compounds of the present invention are particularly useful for the protection of the seed from soil pests and the resulting plant's roots (white grub, termites, wireworms) and shoots against soil pests and foliar insects. The protection of the resulting plant's roots and shoots is preferred. More preferred is the protection of resulting plant's shoots from piercing and sucking insects, wherein the protection from aphids, jassids, thrips and white flies is most preferred.

The present invention therefore comprises a method for the protection of seeds from insects, in particular from soil insects and of the seedling roots and shoots from insects, in particular from soil and foliar insects, said method comprising contacting the seeds before sowing and/or after pregermination with a compound of the present invention thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound of formula (I). The seeds can be coated with seed coating compositions containing the compounds of the present invention. For example, seed coating compositions reported in EP3165092, EP3158864, WO2016198644, WO2016039623, WO2015192923, CA2940002, US2006150489, US2004237395, WO2011028115, EP2229808, WO2007067042, EP1795071, EP1273219, WO200178507, EP1247436, NL1012918 and CA2083415.

The term “coated with and/or containing” generally signifies that the active ingredient is for the most part on the surface of the propagation product at the time of application, although a greater or lesser part of the ingredient may penetrate into the propagation product, depending on the method of application. When the propagation product is (re)planted, it may absorb the active ingredient along with moisture.

Suitable seed is seeds of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the compounds of the present invention may be used for treating seeds from plants, which tolerate the action of herbicides or fungicides or insecticides owing to breeding, including genetic engineering methods.

For example, the compounds of the present invention can be employed in the treatment of seeds from plants, which are resistant to herbicides from the group consisting of the sulfonylureas, iimidazolinones, glufosinate-ammonium or glyphosate-isopropylammonium and analogous active substances (see for example, EP242236, EP242246) (WO92/00377) (EP257993, U.S. Pat. No. 5,013,659) or in transgenic crop plants, for example cotton, with the capability of producing Bacillus thuringiensis toxins (Bt toxins) which make the plants resistant to certain pests (EP142924, EP193259).

Furthermore, the compound of the present invention can be used for the treatment of seeds from plants, which have modified characteristics in comparison with existing plants, which can be generated for example by traditional breeding methods and/or the generation of mutants, or by recombinant procedures). For example, a number of cases have been described of recombinant modifications of crop plants for the purpose of modifying the starch synthesized in the plants (e.g. WO92/11376, WO92/14827, WO91/19806) or of transgenic crop plants having a modified fatty acid composition (WO91/13972).

The seed treatment application of the compounds of the present invention are carried out by spraying or by dusting the seeds before sowing of the plants and before emergence of the plants.

Compositions which are especially useful for seed treatment are e.g.:
A. Soluble concentrates (SL, LS)
B. Emulsions (EW, EO, ES)
C. Suspensions (SC, OD, FS)
D. Water-dispersible granules and water-soluble granules (WG, SG)
E. Water-dispersible powders and water-soluble powders (WP, SP, WS)
F. Gel-Formulations (GF)
G. Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a one embodiment a FS formulation is used for seed treatment. Typically, a FS formulation may comprise 1-800 g/l of active ingredient, 1-200 g/l surfactant, 0 to 200 g/l antifreezing agent, 0 to 400 g/l of binder, 0 to 200 g/l of a pigment and up to 1 liter of a solvent, preferably water.

Especially FS formulations of compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed treatment formulations may additionally comprise binders and optionally colorants.

Binders can be added to improve the adhesion of the active materials on the seeds after treatment. Suitable binders are homo and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo and copolymers, polyethyleneamines, polyethyleneamides and polyethylenepyrimidines, polysaccharides like celluloses, tylose and starch, polyolefin homo and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 1 12, C.I. Solvent Red 1, pigment blue 15:4, pigment blue 15:3, pigment blue 15:2, pigment blue 15:1, pigment blue 80, pigment yellow 1, pigment yellow 13, pigment red 1:12, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

An example of a gelling agent is carrageen (Satiagel®).

In the treatment of seed, the application rates of the compounds of the present invention are generally from 0.1 g to 5 kg per 100 kg of seed, preferably from 1 g to 1 kg per 100 kg of seed, more preferably from 1 g to 200 g per 100 kg of seed and in particular from 5 g to 150 g per 100 kg of seed and 10 g to 100 g per 100 kg of seed. The present invention therefore also provides to seeds comprising a compound of formula (I), or an agriculturally useful salt of I, as defined herein. The amount of the compound of formula (I) or the agriculturally useful salt thereof will in general vary from 0.1 g to 5 kg per 100 kg of seed, preferably from 1 g to 1 kg per 100 kg of seed, in particular from 1 g to 200 g per 100 kg of seed and in particular from 5 g to 150 g per 100 kg of seeds and 10 g to 100 g per 100 kg of seed.

### Animal Health

The present invention also provides an agricultural and/or veterinary composition comprising at least one compound of formula (I).

In one embodiment, the present invention provides use of the compound of formula (I), agriculturally acceptable salts, stereo-isomers, metal complexes, polymorphs, or N-oxides, composition or combination thereof, for combating invertebrate pests in agricultural crops and/or horticultural crops or parasites on animals.

The compounds of formula (I), their N-oxides and/or veterinarily acceptable salts thereof are in particular also suitable for being used for combating parasites in and on animals.

The present invention also provides a compositions containing a parasiticidally effective amount of at least one compound of formula (I), N-oxide or veterinarily acceptable salt thereof and an acceptable carrier, for combating parasites in and on animals.

The present invention also provides a method for treating, controlling, preventing and protecting animals against infestation and infection by parasites, which comprises orally, topically, or parenteral administering or applying to the animals a parasiticidally effective amount of a compound of the present invention or a composition comprising it.

The present invention also provides a process for the preparation of a composition for treating, controlling, preventing or protecting animals against infestation or infection by parasites which comprises a parasiticidally effective amount of a compound of the present invention or a composition comprising it.

Activity of compounds against agricultural pests does not suggest their suitability for controlling of endo and ectoparasites in and on animals which requires, for example, low, non-emetic dosages in the case of oral application, metabolic compatibility with the animal, low toxicity, and a safe handling. Surprisingly it has now been found that compounds of the present invention are suitable for combating endo and ectoparasites in and on animals.

Compounds of the present invention and compositions comprising them are preferably used for controlling and preventing infestations and infections in animals including warm-blooded animals (including fish). They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as freshand salt-water fish such as trout, carp and eels.

Compounds of the present invention and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention and compositions comprising them are suitable for systemic and/or non-systemic control of ecto and/or endoparasites. They can be active against all or some stages of development.

The compounds of the present invention are especially useful for combating ectoparasites.

The compounds of the present invention are especially useful for combating parasites of the following orders and species, respectively: fleas (Siphonaptera), e.g. Ctenocephalides felis, Ctenocephalides cams, Xenopsylla cheopis, Pulex irritans, Tunga penetrans, and Nosopsyllus fasciatus, cockroaches (Blattaria Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis, flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pi pi ens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis, lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus. ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae, Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp, Bugs (Heteropterida): Cimex lectularius, Cimex hemipterus, Reduvius senilis, Triatoma spp., Rhodnius ssp., Panstrongylus ssp. and Arilus critatus, Anoplurida, e.g. Haematopinus spp., Linognathus spp., Pediculus spp., Phtirus spp., and Solenopotes spp, Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.

### Roundworms Nematoda:

Wipeworms and Trichinosis (Trichosyringida), e.g. Trichinellidae (Trichinella spp.), (Trichuridae,) Trichuris spp., Capillaria spp, Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale, Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi, Camallanida, e.g. Dracunculus medinensis (guinea worm) Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp, Thorny headed worms (Acanthocephala), e.g. Acanthocephalus spp., Macracanthorhynchus hirudinaceus and Oncicola spp, Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria a lata, Paragonimus spp., and Nanocyetes spp, Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I) and compositions containing them are particularly useful for the control of pests from the orders Diptera, Siphonaptera and Ixodida.

In one embodiment, the present invention provides use of the compounds of formula (I) and compositions containing them for combating mosquitoes.

In one embodiment, the present invention provides use of the compounds of formula (I) and compositions containing them for combating flies.

In one embodiment, the present invention provides use of the compounds of formula (I) and compositions containing them for combating fleas.

The use of the compounds of the present invention and compositions containing them for combating ticks is still another embodiment of the present invention.

The compounds of the present invention are also especially useful for combating endoparasites (roundworms nematoda, thorny headed worms and planarians).

In one embodiment, the administration of the compounds of the present invention can be carried out both prophylactically and therapeutically.

In another embodiment, administration of the compounds of the present invention is carried out directly or in the form of suitable preparations, orally, topically/dermally or parenterally.

For oral administration to warm-blooded animals, compounds of the present invention may be formulated as animal feeds, animal feed premixes, animal feed concentrates, pills, solutions, pastes, suspensions, drenches, gels, tablets, boluses and capsules. In addition, the compounds of the present invention may be administered to the animals in their drinking water. For oral administration, the dosage form chosen should provide the animal with 0.01 mg/kg to 100 mg/kg of animal body weight per day of the compound of the present invention, preferably with 0.5 mg/kg to 100 mg/kg of animal body weight per day.

Alternatively, the compounds of the present invention may be administered to animals parenterally, for example, by intraruminal, intramuscular, intravenous or subcutaneous injection. The compounds of the present invention may be dispersed or dissolved in a physiologically acceptable carrier for subcutaneous injection. Alternatively, the compounds of the present invention may be formulated into an implant for subcutaneous administration or the compound of the present invention may be transdermally administered to animals. For parenteral administration, the dosage form chosen should provide the animal with 0.01 mg/kg to 100 mg/kg of animal body weight per day of the compound of the present invention.

The compounds of the present invention may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compound of the present invention. In addition, the compounds of the present invention may be formulated as ear tags for animals, particularly quadrupeds such as cattle and sheep.

Suitable preparations are: Solutions such as oral solutions, concentrates for oral administration after dilution, solutions for use on the skin or in body cavities, pouring-on formulations and gels; emulsions and suspensions for oral or dermal administration; semi-solid preparations; formulations in which the active compound is processed in an ointment base or in an oil-inwater or water-in-oil emulsion base; solid preparations such as powders, premixes or concentrates, granules, pellets, tablets, boluses, capsules; aerosols and inhalants, and active compound-containing shaped articles.

The compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the used concentration. Oral solutions and concentrates are prepared according to the state of the art and as described above for injection solutions, sterile procedures not being necessary.

Solutions for use on the skin are trickled on, spread on, rubbed in, sprinkled or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency result. The thickeners employed are the thickeners given above.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are for example, water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, dimethylformamide, dimethylacetamide, n-alkylpyrrolidones such as methylpyrrolidone, n-butylpyrrolidone or noctylpyrrolidone, N-methylpyrrolidone, 2-pyrrolidone, 2,2-dimethyl-4-oxy-methylene-1 ,3-dioxolane or glycerol formal.

Suitable colorants are for example, all colorants permitted for a use on animals which can be dissolved or suspended.

Suitable absorption-promoting substances are for example, dimethyl sulfoxide, spreading oils such as isopropyl myristate, dipropylene glycol pelargonate, silicone oils and copolymers thereof with polyethers, fatty acid esters, triglycerides or fatty alcohols.

Suitable antioxidants are for example, sulfites or metabisulfites such as potassium metabisulfite, ascorbic acid, butylhydroxytoluene, butylhydroxyanisole or tocopherol.

Suitable light stabilizers are for example, novantisolic acid. Suitable adhesives are, for example, cellulose derivatives, starch derivatives, polyacrylates or natural polymers such as alginates, gelatin.

Emulsions can be administered orally, dermally or as injections. Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

### Suitable Hydrophobic Phases (Oils) are:

Liquid paraffins, silicone oils, natural vegetable oils such as sesame oil, almond oil, castor oil, synthetic triglycerides such as caprylic/capric biglyceride, triglyceride mixture with vegetable fatty acids of the chain length C₁-C₁₂ or other specially selected natural fatty acids, partial glyceride mixtures of saturated or unsaturated fatty acids possibly also containing hydroxyl groups, monoand diglycerides of the Cs-do fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof. Suitable hydrophilic phases are: water, alcohols such as propylene glycol, glycerol, sorbitol and mixtures thereof.

Suitable emulsifiers are for example, non-ionic surfactants, e.g. polyethoxylated castor oil, polyethoxylated sorbitan monooleate, sorbitan monostearate, glycerol monostearate, polyoxyethyl stearate, alkylphenol polyglycol ether; ampholytic surfactants such as di-sodium N-lauryl-p-iminodipropionate or lecithin.

Suitable anionic surfactants are for example, sodium lauryl sulfate, fatty alcohol ether sulfates, mono/dialkyl polyglycol ether orthophosphoric acid ester monoethanolamine salt; suitable cation-active surfactants are cetyltrimethylammonium chloride.

Suitable further auxiliaries are for example, substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of death, retardation, prevention and removal, destruction, or otherwise diminishing the occurrence and activity of the target organism. The parasiticidally effective amount can vary for the various compounds/compositions used in the present invention. A parasiticidally effective amount of the compositions will also vary according to the prevailing conditions such as desired parasiticidal effect and duration, target species, mode of application, and the like. The compositions which can be used in the present invention generally comprise from about 0.001 to 95% of the compound of the present invention.

Generally, it is favorable to apply the compounds of the present invention in total amounts of 0.5 mg/kg to 100 mg/kg per day, preferably 1 mg/kg to 50 mg/kg per day. Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight. Preparations diluted before use contain the compounds acting against ectoparasites in concentrations of 0.5 to 90 percent by weight, preferably of 1 to 50 percent by weight. Furthermore, the preparations comprise the compounds of the present invention against endoparasites in concentrations of 10 ppm to 2 percent by weight, preferably of 0.05 to 0.9 percent by weight, very particularly preferably of 0.005 to 0.25 percent by weight.

In a one embodiment, the compositions comprising the compounds of the present invention are applied dermally/topically.

In another embodiment, the topical application is conducted in the form of compound-containing shaped articles such as collars, medallions, ear tags, bands for fixing at body parts, and adhesive strips and foils.

Generally, it is favorable to apply solid formulations which release compounds of the present invention in total amounts of 10 mg/kg to 300 mg/kg, preferably 20 mg/kg to 200 mg/kg, most preferably 25 mg/kg to 100 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of the present invention. A detailed list of plastics and elastomers as well as preparation procedures for the shaped articles is given e.g. in WO 2003086075.

### Positive Crop Response:

The compounds of the present invention not only control insect and mite pests effectively but also show positive crop response such as plant growth enhancement effects like enhanced root growth, enhanced tolerance to drought, high salt, high temperature, chill, frost or light radiation, improved flowering, enhanced nutrient utilization (such as improved nitrogen assimilation), enhanced quality of plant products, higher number of productive tillers, enhanced resistance to fungi, insects, pests and the like, which results in higher yields.

### CHEMISTRY EXAMPLES

The following examples set forth the manner and process of making compounds of the present invention without being a limitation thereof and include the best mode contemplated by the inventors for carrying out the invention.

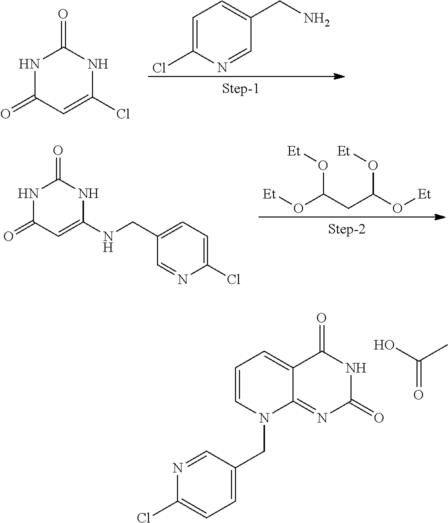

### Step-1: 6-(((6-chloropyridin-3-yl)methyl)amino)pyrimidine-2,4(1H,3H)-dione

To a stirred suspension of 6-chloropyrimidine-2,4(1H,3H)-dione (1.0 g, 6.82 mmol) and (6-chloropyridin-3-yl)methanamine (1.17 g, 8.19 mmol) in n-butanol (10 mL), N,N-diisopropylethylamine (2.4 mL, 13.65 mmol) was added at 25° C. The reaction mixture was stirred at 150° C. for 1.5 h under microwave conditions. The resulting solid was filtered, washed with ethyl acetate (3×20 mL) and dried under reduced pressure to obtain 6-(((6-chloropyridin-3-yl)methyl)amino)pyrimidine-2,4(1H,3H)-dione (1.5 g, 5.9 mmol, 87% yield). ¹H-NMR (400 MHz, DMSO-d6) δ 10.20 (d, J=20.2 Hz, 2H), 8.37 (d, J=1.8 Hz, 1H), 7.78 (dd, J=8.3, 2.4 Hz, 1H), 7.51 (d, J=8.3 Hz, 1H), 6.67 (t, J=6.0 Hz, 1H), 4.40 (d, J=1.5 Hz, 1H), 4.31 (d, J=5.8 Hz, 2H) ESI MS (m/z) 252.95 (MH)+.

### Step-2: 8-((6-chloropyridin-3-yl)methyl)pyrido[2,3-d]pyrimidine-2,4(3H,8H)-dione acetate

To a stirred suspension of 6-(((6-chloropyridin-3-yl)methyl)amino)pyrimidine-2,4(1H,3H)-dione (7.5 g, 29.7 mmol) in acetic acid (100 mL), 1,1,3,3-tetraethoxypropane (7.85 g, 35.6 mmol) was added, and the resulting reaction mixture was stirred at 80° C. for 4 h. After the completion of the reaction, the reaction mixture was cooled to 25° C. and concentrated under reduced pressure to get a crude product which was washed with ethyl acetate (3×30 mL) and dried under reduced pressure to obtain 8-((6-chloropyridin-3-yl)methyl)pyrido[2,3-d]pyrimidine-2,4(3H,8H)-dione acetate (7.6 g, 21.8 mmol, 73% yield). ¹H-NMR (400 MHz, DMSO-d6) δ 11.94 (s, 1H), 10.93 (s, 1H), 8.65 (dd, J=6.6, 1.7 Hz, 1H), 8.51 (d, J=2.1 Hz, 1H), 8.41 (dd, J=7.2, 1.7 Hz, 1H), 7.88 (dd, J=8.3, 2.4 Hz, 1H), 7.52 (dd, J=8.3, 0.6 Hz, 1H), 6.96 (dd, J=7.3, 6.7 Hz, 1H), 5.60 (d, J=17.7 Hz, 2H), 1.91-1.86 (m, 3H); ESI MS (m/z) 288.9 (MH)+.

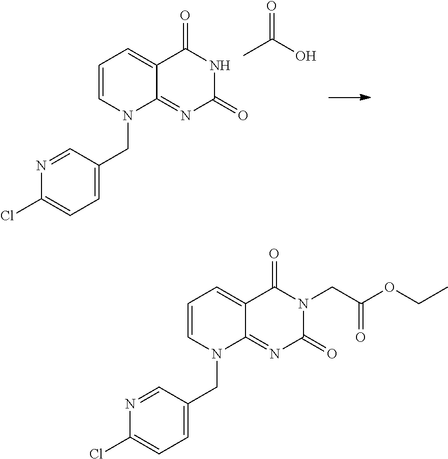

To a stirred mixture of 8-((6-chloropyridin-3-yl)methyl)pyrido[2,3-d]pyrimidine-2,4(3H,8H)-dione acetate (Step 2 of example-1) (300 mg, 1.04 mmol) and ethyl 2-bromoacetate (0.148 mL, 1.25 mmol) in N,N-dimethylformamide (4 mL), potassium carbonate (1.0 g, 7.27 mmol) was added. The reaction mixture was stirred at 25° C. for 24 h. After the completion of the reaction, the reaction mixture was diluted with dichloromethane (5 mL) and filtered. The filtrate was purified by column chromatography on silica gel to obtain ethyl 2-(8-((6-chloropyridin-3-yl)methyl)-2,4-dioxo-4,8-dihydropyrido[2,3-d]pyrimidin-3(2H)-yl)acetate (61 mg, 0.163 mmol, 15.66% yield). ¹H-NMR (400 MHz, DMSO-d6) δ 8.77 (dd, J=6.4, 1.7 Hz, 1H), 8.54-8.51 (m, 2H), 7.90 (dd, J=8.3, 2.4 Hz, 1H), 7.52 (d, J=8.3 Hz, 1H), 7.08 (t, J=7.0 Hz, 1H), 5.63 (s, 2H), 4.57 (s, 2H), 4.14-4.08 (m, 2H), 1.22-1.16 (m, 3H); ESI MS (m/z) 374.9 (MH)+.

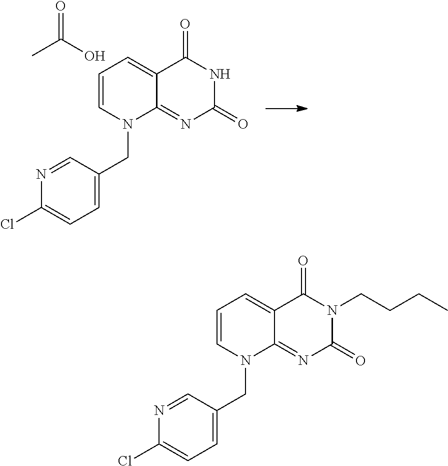

To a stirred suspension of 8-((6-chloropyridin-3-yl)methyl)pyrido[2,3-d]pyrimidine-2,4(3H,8H)-dione acetate (Step 2 of example-1) (300 mg, 1.04 mmol) in N,N-dimethylformamide (4 mL), sodium hydride (83 mg, 2.08 mmol) was added at 0° C. The resulting mixture was stirred at 50° C. for 0.5 h followed by the addition of 1-bromobutane (214 mg, 1.56 mmol) at 50° C. Stirring was continued for 6 h at the same temperature. After the completion of the reaction, the reaction mixture was cooled to 25° C. and few drops of water were added to quench the excess of sodium hydride.

The resulting reaction mixture was diluted with dichloromethane (5 mL) and centrifuged. The obtained liquid was concentrated under reduced pressure to obtain a crude product which was purified by column chromatography on silica gel to obtain 3-butyl-8-((6-chloropyridin-3-yl)methyl)pyrido[2,3-d]pyrimidine-2,4(3H,8H)-dione (57 mg, 0.165 mmol, 15.91% yield). ¹H-NMR (400 MHz, DMSO-d6) δ 8.68 (dd, J=6.6, 1.7 Hz, 1H), 8.52 (d, J=2.2 Hz, 1H), 8.46 (dd, J=7.5, 1.8 Hz, 1H), 7.89 (dd, J=8.3, 2.4 Hz, 1H), 7.52 (d, J=8.1 Hz, 1H), 7.01 (t, J=7.0 Hz, 1H), 5.60 (s, 2H), 3.82 (t, J=7.3 Hz, 2H), 1.53-1.46 (m, 2H), 1.27 (td, J=14.9, 7.6 Hz, 2H), 0.88 (t, J=7.3 Hz, 3H); ESI MS (m/z) 344.90 (MH)+.

### Example-4: Synthesis of 8-((6-chloropyridin-3-yl)methyl)-3-phenylpyrido[2,3-d]pyrimidine-2,4(3H,8H)-dione (Compound 53)

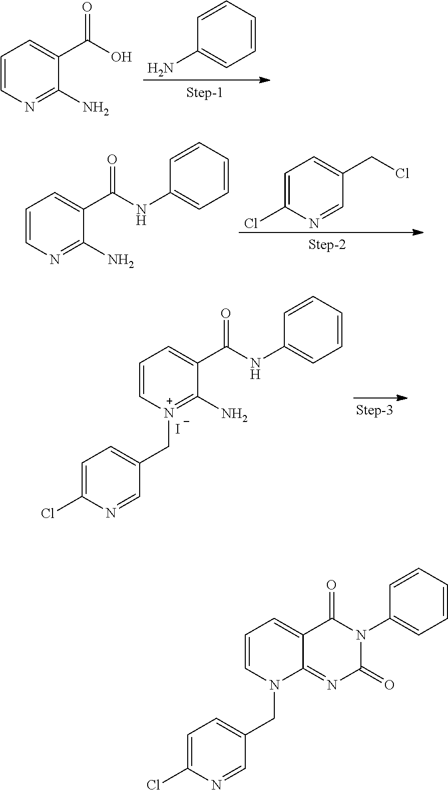

### Step-1: 2-Amino-N-phenylnicotinamide

To a stirred solution of 2-aminonicotinic acid (0.5 g, 3.62 mmol) in N,N-dimethyl formamide (5 mL), EDC.HCl (1.04 g, 5.43 mmol), HOBt (0.83 g, 5.43 mmol), N,N-diisopropylethylamine (1.90 ml, 10.86 mmol) and aniline (0.494 mL, 5.43 mmol) were sequentially added at 25° C. and the resulting mixture was stirred at 25° C. for 16 h. After the completion of the reaction, the reaction mixture was poured into water (100 mL). The aqueous layer was extracted with ethyl acetate (2×200 mL) and the combined organic layers were washed with a saturated aqueous brine solution (50 mL), dried over anhydrous sodium sulphate and concentrated under reduced pressure to obtain a crude product which was purified by column chromatography on silica gel to obtain the desired product as solid (0.672 g, 3.15 mmol, 87% yield) ¹H-NMR (400 MHz, DMSO-d6) δ 10.12 (s, 1H), 8.12 (dd, J=4.8, 1.8 Hz, 1H), 8.02 (dd, J=7.7, 1.8 Hz, 1H), 7.69-7.66 (m, 2H), 7.36-7.31 (m, 2H), 7.11-7.07 (m, 1H), 6.94 (s, 2H), 6.65 (dd, J=7.7, 4.8 Hz, 1H); ESI MS (m/z) 213.95 (MH)+.

Step-2: 2-amino-1-((6-chloropyridin-3-yl)methyl)-3-(phenylcarbamoyl)pyridin-1-ium iodide: To a stirred solution of 2-amino-N-phenylnicotinamide (0.44 g, 2.04 mmol) in N,N-dimethyl formamide (5 mL), 2-chloro-5-(chloromethyl)pyridine (0.40 g, 2.45 mmol) and sodium iodide (0.46 g, 3.07 mmol) were added at 25° C. The reaction mixture was stirred at 90° C. for 16 h. After completion of the reaction, the reaction mixture was cooled to 25° C. and concentrated under reduced pressure to obtain a crude product which was purified by column chromatography on silica gel to obtain the desired product as a solid (0.532 g, 1.570 mmol, 77% yield) ¹H-NMR (400 MHz, DMSO-d6) δ 10.84 (s, 1H), 9.00 (s, 2H), 8.55 (dd, J=7.6, 1.2 Hz, 1H), 8.48-8.41 (m, 2H), 7.78 (dd, J=8.3, 2.7 Hz, 1H), 7.70 (d, J=7.6 Hz, 2H), 7.60 (d, J=8.3 Hz, 1H), 7.40-7.36 (m, 2H), 7.18-7.14 (m, 2H), 5.70 (s, 2H); ESI MS (m/z) 338.85 (MH-I)+.

### Step-3: 8-((6-chloropyridin-3-yl)methyl)-3-phenylpyrido[2,3-d]pyrimidine-2,4(3H,8H)-dione (Compound 53)

To a stirred solution of 2-amino-1-((6-chloropyridin-3-yl)methyl)-3-(phenylcarbamoyl)pyridin-1-ium iodide (512 mg, 1.51 mmol) in tetrahydrofruan (6 mL) at 25° C., triethylamine (0.42 mL, 3.02 mmol) was added at 25° C. followed by the addition of CDI (490 mg, 3.02 mmol). The reaction mixture was stirred at 100° C. for 4-5 h. After the completion of the reaction, the reaction mixture was cooled to 25° C. and poured into water (20 mL). The aqueous layer was extracted with ethyl acetate (2×80 mL), the combined organic layers were washed with a saturated brine solution (2×20 mL), dried over anhydrous sodium sulphate, filtered and evaporated under reduced pressure to obtain a the crude solid which was purified by flash chromatography using a mixture of dichloromethane in methanol as an eluent to get the desired product as a solid (150 mg, 0.411 mmol, 27.2% yield). ¹H-NMR (400 MHz, DMSO-d6) δ 8.74 (dd, J=6.6, 1.7 Hz, 1H), 8.56 (d, J=2.2 Hz, 1H), 8.49 (dd, J=7.3, 1.7 Hz, 1H), 7.94 (dd, J=8.3, 2.4 Hz, 1H), 7.56-7.54 (m, 1H), 7.44 (td, J=6.7, 1.5 Hz, 2H), 7.38-7.34 (m, 1H), 7.19-7.17 (m, 2H), 7.05 (dd, J=7.2, 6.5 Hz, 1H), 5.65 (s, 2H); ESI MS (m/z) 364.95 (MH)+

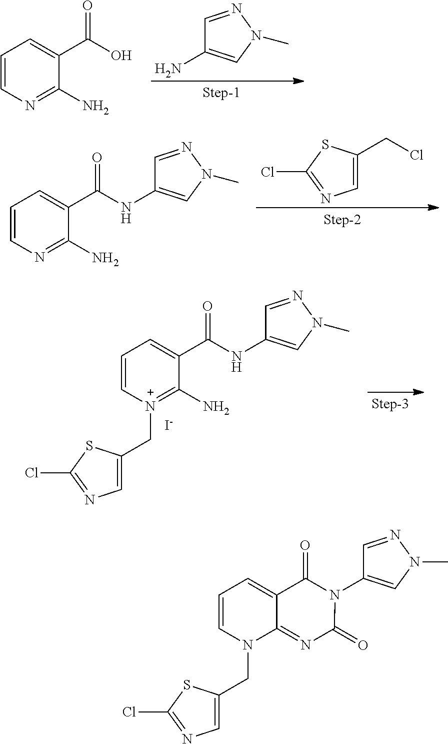

### Step-1: 2-Amino-N-(1-methyl-1H-pyrazol-4-yl)nicotinamide

The title compound was prepared from 2-aminonicotinic acid (1.0 g, 7.24 mmol) and 1-methyl-1H-pyrazol-4-amine (2.0 g, 20.63 mmol) by following the reaction conditions as described in step-1 of example-4 (2.68 g, 12.34 mmol, 90% yield). ¹H-NMR (400 MHz, DMSO-d6) δ 10.30 (s, 1H), 8.10 (dd, J=4.9, 1.8 Hz, 1H), 8.00-7.97 (m, 2H), 7.52 (d, J=0.6 Hz, 1H), 7.05 (s, 2H), 6.63 (dd, J=7.8, 4.7 Hz, 1H), 3.81 (s, 3H); ESI MS (m/z) 218.00 (MH)+.

Step-2: 2-amino-1-((2-chlorothiazol-5-yl)methyl)-3-((1-methyl-1H-pyrazol-4-yl)carbamoyl)pyridin-1-ium iodide:

The title compound was prepared from 2-amino-N-(1-methyl-1H-pyrazol-4-yl)nicotinamide (0.80 g, 3.68 mmol) and 2-chloro-5-(chloromethyl)thiazole (0.68 g, 4.05 mmol) following the reaction conditions as described in step-2 of example-4 (1.10 g, 2.307 mmol, 62% yield). ¹H-NMR (400 MHz, DMSO-d6) δ 11.00 (s, 1H), 9.26 (s, 2H), 8.49-8.45 (m, 2H), 8.00 (s, 1H), 7.88 (s, 1H), 7.60 (d, J=0.6 Hz, 1H), 7.15 (t, J=7.0 Hz, 1H), 5.75 (s, 2H), 3.82 (s, 3H); ESI MS (m/z) 348.95 (MH-I)+.

### Step-3: 8-((2-chlorothiazol-5-yl)methyl)-3-(1-methyl-1H-pyrazol-4-yl)pyrido[2,3-d]pyrimidine-2,4(3H,8H)-dione (Compound 89)

The title compound was prepared from 2-amino-1-((2-chlorothiazol-4-yl)methyl)-3-((1-methyl-1H-pyrazol-5-yl)carbamoyl)pyridin-1-ium iodide (0.50 g, 1.05 mmol) following the reaction conditions as described in step-3 of example-4 (0.150 g, 0.40 mmol, 38% yield). ¹H-NMR (400 MHz, DMSO-d6) δ 8.73 (dd, J=6.6, 1.7 Hz, 1H), 8.49 (dd, J=7.3, 1.7 Hz, 1H), 7.88 (s, 1H), 7.84 (d, J=0.5 Hz, 1H), 7.46 (d, J=0.6 Hz, 1H), 7.06 (dd, J=7.5, 6.5 Hz, 1H), 5.70 (s, 2H), 3.86 (s, 3H); ESI MS (m/z) 374.85 (MH)+.

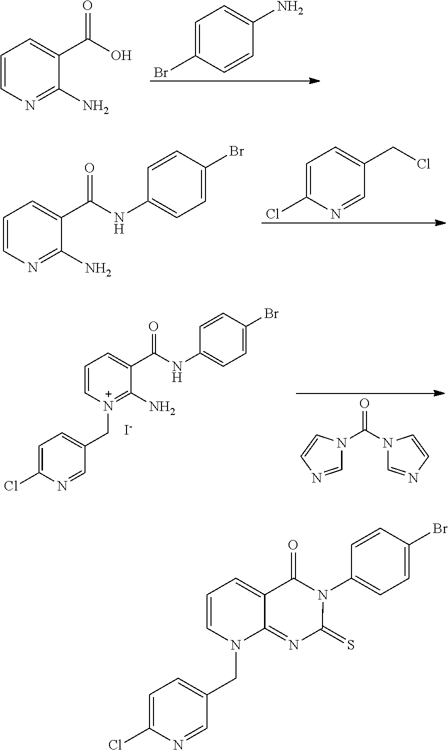

### Step-1: 2-Amino-N-(4-bromophenyl)nicotinamide

To a stirred solution of 2-aminonicotinic acid (1.3 g, 9.41 mmol) in N,N-dimethylformamide (12 mL) at 25° C., EDC.HCl (2.71 g, 14.12 mmol), HOBt (2.162 g, 14.12 mmol), N,N-diisopropylethylamine (4.93 mL, 28.2 mmol) and 4-bromoaniline (1.78 g, 10.35 mmol) were added sequentially. The resulting reaction mixture was stirred at 25° C. for 16 h. After the completion of the reaction, the reaction mixture was poured into water (250 mL) and the aqueous layer was extracted with ethyl acetate (2×200 mL). The combined organic layers were separated, washed with a brine solution (2×150 mL), dried over anhydrous sodium sulphate and concentrated under reduced pressure to obtain a crude compound which was purified by washing with a mixture of dichloromethane and pentane to obtain 2-amino-N-(4-bromophenyl)nicotinamide (2.50 g, 8.56 mmol, 91% yield) as solid. ESI MS (m/z) 293.90 (MH)+

### Step-2: 2-Amino-3-((4-bromophenyl)carbamoyl)-1-((6-chloropyridin-3-yl)methyl)pyridin-1-ium iodide

To a stirred solution of 2-amino-N-(4-bromophenyl)nicotinamide (1 g, 3.42 mmol) in N,N-dimethylformamide (10 mL), 2-chloro-5-(chloromethyl)pyridine (0.61 g, 3.77 mmol) and sodium iodide (0.77 g, 5.13 mmol) were added at 25° C. The resulting reaction mixture was stirred at 90° C. for 16 h. After the completion of the reaction, the reaction mixture was concentrated under reduced pressure to obtain a crude compound which was purified by column chromatography on silica gel to obtain 2-amino-3-((4-bromophenyl)carbamoyl)-1-((6-chloropyridin-3-yl)methyl)pyridin-1-ium iodide (1.47 g, 2.69 mmol, 79% yield) as a solid. ¹H-NMR (400 MHz, DMSO-d6) δ 10.92 (s, 1H), 8.95 (s, 2H), 8.54-8.52 (m, 1H), 8.45-8.41 (m, 2H), 7.76 (dd, J=8.4, 2.6 Hz, 1H), 7.68 (dt, J=9.3, 2.4 Hz, 2H), 7.61-7.56 (m, 3H), 7.17 (t, J=7.0 Hz, 1H), 5.63 (s, 2H); ESI MS (m/z) 418.45 (MH)+

### Step-3: 3-(4-Bromophenyl)-8-((6-chloropyridin-3-yl)methyl)-2-thioxo-2,8-dihydropyrido[2,3-d]pyrimidin-4(3H)-one

To a stirred solution of 2-amino-3-((4-bromophenyl)carbamoyl)-1-((6-chloropyridin-3-yl)methyl)pyridin-1-ium iodide (0.65 g, 1.19 mmol) in tetrahydrofuran (6 mL), triethylamine (0.331 ml, 2.372 mmol) and di(1H-imidazol-1-yl)methanethione (0.53 g, 2.37 mmol) were added at 25° C.

The reaction mixture was stirred at 100° C. for 5 h. After the completion of the reaction, the reaction mixture was poured into water (200 mL) and extracted with ethyl acetate (2×200 mL). The combined organic layers were washed with 1N HCl (100 mL), brine solution (2×100 mL), dried over anhydrous sodium sulphate, filtered and concentrated under reduced pressure to obtain a crude compound which was purified by column chromatography on silica gel to obtain the desired product 3-(4-bromophenyl)-8-((6-chloropyridin-3-yl)methyl)-2-thioxo-2,8-dihydropyrido[2,3-d]pyrimidin-4(3H)-one (0.150 g, 0.326 mmol, 27.5% yield) as a solid. ¹H-NMR (400 MHz, DMSO-d6) δ 8.96 (dd, J=6.4, 1.5 Hz, 1H), 8.67 (d, J=2.1 Hz, 1H), 8.56 (dd, J=7.5, 1.7 Hz, 1H), 8.06 (dd, J=8.4, 2.6 Hz, 1H), 7.62 (dd, J=6.6, 2.0 Hz, 2H), 7.56 (d, J=8.3 Hz, 1H), 7.24 (dd, J=7.5, 6.6 Hz, 1H), 7.11 (dd, J=6.6, 2.0 Hz, 2H), 5.71 (s, 2H) ESI MS (m/z) 460.85 (MH)+

The below compounds in Table-A were obtained using appropriate starting materials and following analogues procedures as described in the example 1 to 6.

TABLE A

| Compd | | |
|---|---|---|
| No | Compound Name | Analytical data |
| 1 | 3-allyl-8-((6-chloropyridin- | ¹H-NMR (400 MHz, DMSO-d6) δ 8.70 (dd, J = 6.6, 1.7 |
| | 3-yl)methyl)pyrido[2,3- | Hz, 1H), 8.52 (d, J = 2.4 Hz, 1H), 8.47 (dd, J = 7.3, 1.5 Hz, |
| | d]pyrimidine-2,4(3H,8H)- | 1H), 7.89 (dd, J = 8.6, 2.4 Hz, 1H), 7.52 (d, J = 8.6 Hz, |
| | dione | 1H), 7.03 (t, J = 6.9 Hz, 1H), 5.82 (qd, J = 11.0, 5.6 Hz, |
| | | 1H), 5.63 (d, J = 17.4 Hz, 2H), 5.07 (dd, J = 6.7, 1.5 Hz, |
| | | 1H), 5.04 (s, 1H), 4.44 (d, J = 5.2 Hz, 2H); ESI MS (m/z) |
| | | 329.6 (MH)+. |
| 2 | 8-((6-chloropyridin-3- | ¹H-NMR (400 MHz, DMSO-d6) δ 8.69 (dd, J = 6.6, 1.7 |
| | yl)methyl)-3- | Hz, 1H), 8.52 (d, J = 2.1 Hz, 1H), 8.46 (dd, J = 7.3, 1.5 Hz, |
| | ethylpyrido[2,3- | 1H), 7.89 (dd, J = 8.3, 2.4 Hz, 1H), 7.52 (d, J = 8.3 Hz, |
| | d]pyrimidine-2,4(3H,8H)- | 1H), 7.01 (t, J = 6.9 Hz, 1H), 5.62 (d, J = 17.7 Hz, 2H), |
| | dione | 3.86 (q, J = 6.9 Hz, 2H), 1.08 (t, J = 7.0 Hz, 3H); ESI MS |
| | | (m/z) 317.5(MH)+. |
| 3 | 8-((6-chloropyridin-3- | ¹H-NMR (400 MHz, DMSO-d6) δ 8.69 (dd, J = 6.6, 1.7 |
| | yl)methyl)-3- | Hz, 1H), 8.52 (d, J = 2.4 Hz, 1H), 8.47 (dd, J = 7.3, 1.8 Hz, |
| | methylpyrido[2,3- | 1H), 7.88 (dd, J = 8.3, 2.4 Hz, 1H), 7.51 (d, J = 8.3 Hz, |
| | d]pyrimidine-2,4(3H,8H)- | 1H), 7.02 (dd, J = 7.3, 6.4 Hz, 1H), 5.61 (s, 2H), 3.19 (s, |
| | dione | 3H); ESI MS (m/z) 303.0 (MH)+. |
| 4 | 8-((6-chloropyridin-3- | ¹H-NMR (400 MHz, DMSO-d6) δ 8.69 (dd, J = 6.5, 1.8 |
| | yl)methyl)-3- | Hz, 1H), 8.53 (d, J = 2.2 Hz, 1H), 8.47 (dd, J = 7.3, 2.0 Hz, |
| | (cyclopropylmethyl)pyrido[2,3- | 1H), 7.89 (dd, J = 8.2, 2.6 Hz, 1H), 7.52 (d, J = 8.3 Hz, |
| | d]pyrimidine- | 1H), 7.02 (dd, J = 7.3, 6.6 Hz, 1H), 5.60 (s, 2H), 3.72 (d, J = |
| | 2,4(3H,8H)-dione | 7.1 Hz, 2H), 1.19-1.11 (m, 1H), 0.40-0.29 (m, 4H). |
| 5 | 8-((6-chloropyridin-3- | ¹H-NMR (400 MHz, DMSO-d6) δ 8.69 (dd, J = 6.6, 1.7 |
| | yl)methyl)-3-(2- | Hz, 1H), 8.53 (d, J = 2.1 Hz, 1H), 8.47 (dd, J = 7.5, 1.7 Hz, |
| | methylallyl)pyrido[2,3- | 1H), 7.90 (dd, J = 8.4, 2.6 Hz, 1H), 7.52 (dd, J = 8.3, 0.6 |
| | d]pyrimidine-2,4(3H,8H)- | Hz, 1H), 7.02 (dd, J = 7.3, 6.4 Hz, 1H), 5.62 (s, 2H), 4.69 |
| | dione | (t, J = 1.4 Hz, 1H), 4.49 (d, J = 0.6 Hz, 1H), 4.35 (s, 2H), |
| | | 1.69 (s, 3H); ESI MS (m/z) 343.0 (MH)+. |
| 6 | 8-((6-chloropyridin-3- | ¹H-NMR (400 MHz, DMSO-d6) δ 8.73 (dd, J = 6.6, 1.7 |
| | yl)methyl)-3-((2- | Hz, 1H), 8.52 (dd, J = 7.6, 1.8 Hz, 2H), 7.87 (dd, J = 8.3, |
| | chlorothiazol-5- | 2.4 Hz, 1H), 7.63 (s, 1H), 7.51 (d, J = 8.3 Hz, 1H), 7.06 |
| | yl)methyl)pyrido[2,3- | (dd, J = 7.3, 6.4 Hz, 1H), 5.63 (d, J = 14.7 Hz, 2H), 5.11 |
| | d]pyrimidine-2,4(3H,8H)- | (d, J = 14.7 Hz, 2H); ESI MS (m/z) 419.75 (MH)+. |
| | dione | |
| 7 | 3,8-bis((6-chloropyridin-3- | ¹H-NMR (400 MHz, DMSO-d6) δ 8.71 (dd, J = 6.6, 1.7 |
| | yl)methyl)pyrido[2,3- | Hz, 1H), 8.52-8.49 (m, 2H), 8.38 (d, J = 2.4 Hz, 1H), 7.89 |
| | d]pyrimidine-2,4(3H,8H)- | (dd, J = 8.3, 2.8 Hz, 1H), 7.77 (dd, J = 8.3, 2.8 Hz, 1H), |
| | dione | 7.51 (d, J = 8.3 Hz, 1H), 7.43 (d, J = 7.9 Hz, 1H), 7.05 (dd, |
| | | J = 7.3, 6.7 Hz, 1H), 5.62 (s, 2H), 5.04 (s, 2H); ESI MS |
| | | (m/z) 413.95(MH)+. |
| 8 | ethyl 2-(8-((6- | ¹H-NMR (400 MHz, DMSO-d6) δ 8.77 (dd, J = 6.4, 1.7 |
| | chloropyridin-3-yl)methyl)- | Hz, 1H), 8.54-8.51 (m, 2H), 7.90 (dd, J = 8.3, 2.4 Hz, 1H), |
| | 2,4-dioxo-4,8- | 7.52 (d, J = 8.3 Hz, 1H), 7.08 (t, J = 7.0 Hz, 1H), 5.63 (s, |
| | dihydropyrido[2,3- | 2H), 4.57 (s, 2H), 4.14-4.08 (m, 2H), 1.22-1.16 (m, 3H); |
| | d]pyrimidin-3(2H)- | ESI MS (m/z) 374.9 (MH)+. |
| | yl)acetate | |
| 9 | 3-(3-chloropropyl)-8-((6- | ¹H-NMR (400 MHz, DMSO-d6) δ 8.70-8.66 (m, 1H), 8.52 |
| | chloropyridin-3- | (d, J = 2.0 Hz, 1H), 8.46 (dd, J = 7.5, 1.8 Hz, 1H), 7.89 (dd, |
| | yl)methyl)pyrido[2,3- | J = 8.3, 2.7 Hz, 1H), 7.53-7.47 (m, 1H), 7.02 (dd, J = 7.3, |
| | d]pyrimidine-2,4(3H,8H)- | 6.6 Hz, 1H), 5.63 (d, J = 18.3 Hz, 2H), 4.01-3.91 (m, 2H), |
| | dione | 3.64 (t, J = 6.6 Hz, 2H), 2.02-1.95 (m, 2H); ESI MS (m/z) |
| | | 364.85 (MH)+. |
| 10 | 8-((6-chloropyridin-3- | ¹H-NMR (400 MHz, DMSO-d6) δ 11.94 (s, 1H), 10.93 (s, |
| | yl)methyl)pyrido[2,3- | 1H), 8.65 (dd, J = 6.6, 1.7 Hz, 1H), 8.51 (d, J = 2.1 Hz, |
| | d]pyrimidine-2,4(3H,8H)- | 1H), 8.41 (dd, J = 7.2, 1.7 Hz, 1H), 7.88 (dd, J = 8.3, 2.4 |
| | dione acetate | Hz, 1H), 7.52 (dd, J = 8.3, 0.6 Hz, 1H), 6.96 (dd, J = 7.3, |
| | | 6.7 Hz, 1H), 5.60 (d, J = 17.7 Hz, 2H), 1.91-1.86 (m, 3H); |
| | | ESI MS (m/z) 288.9 (MH)+. |
| 11 | 8-((6-chloropyridin-3- | ¹H-NMR (400 MHz, DMSO-d6) δ 8.69 (dd, J = 6.6, 1.7 |
| | yl)methyl)-3-(3- | Hz, 1H), 8.52 (d, J = 2.0 Hz, 1H), 8.47 (dd, J = 7.3, 1.7 Hz, |
| | fluoropropyl)pyrido[2,3- | 1H), 7.89 (dd, J = 8.3, 2.7 Hz, 1H), 7.52 (dd, J = 8.2, 0.6 |
| | d]pyrimidine-2,4(3H,8H)- | Hz, 1H), 7.02 (dd, J = 7.3, 6.6 Hz, 1H), 5.61 (s, 2H), 4.52 |
| | dione | (t, J = 6.0 Hz, 1H), 4.40 (t, J = 5.9 Hz, 1H), 3.94 (t, J = 7.1 |
| | | Hz, 2H), 1.98-1.85 (m, 2H); ESI MS (m/z) 348.55 (MH)+. |
| 12 | 4-(8-((6-chloropyridin-3- | ¹H-NMR (400 MHz, DMSO-d6) δ 8.69 (dd, J = 6.5, 1.8 |
| | yl)methyl)-2,4-dioxo-4,8- | Hz, 1H), 8.52-8.46 (m, 2H), 7.89 (dd, J = 8.3, 2.4 Hz, 1H), |
| | dihydropyrido[2,3- | 7.52 (dd, J = 8.3, 0.5 Hz, 1H), 7.02 (dd, J = 7.3, 6.6 Hz, |
| | d]pyrimidin-3(2H)- | 1H), 5.61 (s, 2H), 3.92 (t, J = 6.8 Hz, 2H), 2.72 (s, OH), |
| | yl)butanenitrile | 2.52-2.50 (m, 2H), 1.87-1.80 (m, 2H); ESI MS (m/z) 355.9 |
| | | (MH)+ |
| 13 | 3-butyl-8-((6- | ¹H-NMR (400 MHz, DMSO-d6) δ 8.68 (dd, J = 6.6, 1.7 |
| | chloropyridin-3- | Hz, 1H), 8.52 (d, J = 2.2 Hz, 1H), 8.46 (dd, J = 7.5, 1.8 Hz, |
| | yl)methyl)pyrido[2,3- | 1H), 7.89 (dd, J = 8.3, 2.4 Hz, 1H), 7.52 (d, J = 8.1 Hz, |
| | d]pyrimidine-2,4(3H,8H)- | 1H), 7.01 (t, J = 7.0 Hz, 1H), 5.60 (s, 2H), 3.82 (t, J = 7.3 |
| | dione | Hz, 2H), 1.53-1.46 (m, 2H), 1.27 (td, J = 14.9, 7.6 Hz, 2H), |
| | | 0.88 (t, J = 7.3 Hz, 3H); ESI MS (m/z) 344.90 (MH)+. |
| 14 | 8-((6-chloropyridin-3- | ¹H-NMR (400 MHz, DMSO-d6) δ 8.68 (dd, J = 6.6, 1.7 |
| | yl)methyl)-3- | Hz, 1H), 8.52 (d, J = 2.0 Hz, 1H), 8.46 (dd, J = 7.3, 1.7 Hz, |
| | propylpyrido[2,3- | 1H), 7.89 (dd, J = 8.3, 2.7 Hz, 1H), 7.52 (dd, J = 8.3, 0.7 |
| | d]pyrimidine-2,4(3H,8H)- | Hz, 1H), 7.01 (dd, J = 7.3, 6.4 Hz, 1H), 5.60 (s, 2H), 3.80- |
| | dione | 3.76 (m, 2H), 1.56-1.48 (m, 2H), 0.84 (t, J = 7.5 Hz, 3H); |
| | | ESI MS (m/z) 330.50 (MH)+. |
| 15 | 8-((6-chloropyridin-3- | ¹H-NMR (400 MHz, DMSO-d6) δ 8.73 (dd, J = 6.5, 1.8 |
| | yl)methyl)-3-(prop-2-yn-1- | Hz, 1H), 8.53-8.50 (m, 2H), 7.89 (dd, J = 8.3, 2.4 Hz, 1H), |
| | yl)pyrido[2,3-d]pyrimidine- | 7.52 (dd, J = 8.3, 0.5 Hz, 1H), 7.06 (dd, J = 7.3, 6.6 Hz, |
| | 2,4(3H,8H)-dione | 1H), 5.62 (s, 2H), 4.56 (d, J = 2.4 Hz, 2H), 3.02 (t, J = 2.4 |
| | | Hz, 1H); ESI MS (m/z) 349.74 (M + Na)+. |
| 16 | 8-((6-chloropyridin-3- | ¹H-NMR (400 MHz, DMSO-d6) δ 8.65 (dd, J = 6.6, 1.7 |
| | yl)methyl)-3- | Hz, 1H), 8.52 (d, J = 2.0 Hz, 1H), 8.43 (dd, J = 7.3, 1.7 Hz, |
| | isopropylpyrido[2,3- | 1H), 7.89 (dd, J = 8.3, 2.4 Hz, 1H), 7.52 (d, J = 8.3 Hz, |
| | d]pyrimidine-2,4(3H,8H)- | 1H), 6.98 (dd, J = 7.3, 6.4 Hz, 1H), 5.57 (s, 2H), 5.11-5.04 |
| | dione | (m, 1H), 1.36 (d, J = 6.8 Hz, 6H); ESI MS (m/z) 354.20 |
| | | (M + Na)+. |
| 17 | 2-(8-((6-chloropyridin-3- | ¹H-NMR (400 MHz, DMSO-d6) δ 8.70 (dd, J = 6.6, 1.7 |
| | yl)methyl)-2,4-dioxo-4,8- | Hz, 1H), 8.52-8.47 (m, 2H), 7.89 (dd, J = 8.2, 2.6 Hz, 1H), |
| | dihydropyrido[2,3- | 7.52 (d, J = 8.3 Hz, 1H), 7.03 (dd, J = 7.3, 6.6 Hz, 1H), |
| | d]pyrimidin-3(2H)- | 5.61 (s, 2H), 4.50 (dtd, J = 26.9, 8.0, 2.6 Hz, 1H), 3.88 (t, |
| | yl)acetonitrile | J = 7.2 Hz, 2H), 2.23 (q, J = 7.4 Hz, 2H); ESI MS (m/z) |
| | | 400.90 (M+ Na)+. |
| 18 | 8-((6-chloropyridin-3- | ¹H-NMR (400 MHz, DMSO-d6) δ 8.68 (dd, J = 6.6, 1.7 |
| | yl)methyl)-3-(pent-4-en-1- | Hz, 1H), 8.52 (d, J = 2.2 Hz, 1H), 8.46 (dd, J = 7.3, 2.0 Hz, |
| | yl)pyrido[2,3-d]pyrimidine- | 1H), 7.89 (dd, J = 8.2, 2.6 Hz, 1H), 7.53-7.51 (m, 1H), 7.01 |
| | 2,4(3H,8H)-dione | (dd, J = 7.3, 6.6 Hz, 1H), 5.86-5.76 (m, 1H), 5.60 (s, 2H), |
| | | 5.06-5.01 (m, 1H), 4.94 (dd, J = 10.3, 2.2 Hz, 1H), 3.83 (t, |
| | | J = 7.5 Hz, 2H), 2.06-2.00 (m, 2H), 1.64-1.57 (m, 2H); ESI |
| | | MS (m/z) 457.10 (MH)+. |
| 19 | 8-((6-chloropyridin-3- | ¹H-NMR (400 MHz, DMSO-d6) δ 8.67 (dd, J = 6.5, 1.8 |
| | yl)methyl)-3- | Hz, 1H), 8.52 (d, J = 2.0 Hz, 1H), 8.46 (dd, J = 7.3, 1.7 Hz, |
| | (cyclobutylmethyl)pyrido[2,3- | 1H), 7.89 (dd, J = 8.2, 2.6 Hz, 1H), 7.52 (d, J = 8.3 Hz, |
| | d]pyrimidine- | 1H), 7.00 (dd, J = 7.3, 6.6 Hz, 1H), 5.59 (s, 2H), 3.90 (d, J = |
| | 2,4(3H,8H)-dione | 7.1 Hz, 2H), 2.66-2.60 (m, 1H), 1.90-1.85 (m, 2H), 1.80- |
| | | 1.72 (m, 4H); ESI MS (m/z) 357.00 (MH)+. |
| 20 | 3-(but-3-en-1-yl)-8-((6- | ¹H-NMR (400 MHz, DMSO-d6) δ 8.69 (dd, J = 6.5, 1.8 |
| | chloropyridin-3- | Hz, 1H), 8.52 (d, J = 2.0 Hz, 1H), 8.46 (dd, J = 7.5, 1.8 Hz, |
| | yl)methyl)pyrido[2,3- | 1H), 7.89 (dd, J = 8.3, 2.7 Hz, 1H), 7.52 (d, J = 8.3 Hz, |
| | d]pyrimidine-2,4(3H,8H)- | 1H), 7.01 (dd, J = 7.3, 6.6 Hz, 1H), 5.82-5.73 (m, 1H), 5.60 |
| | dione | (s, 2H), 5.03-4.94 (m, 2H), 3.90 (t, J = 7.3 Hz, 2H), 2.29 |
| | | (q, J = 7.3 Hz, 2H); ESI MS (m/z) 343.00 (MH)+. |
| 21 | 8-((6-chloropyridin-3- | ¹H-NMR (400 MHz, DMSO-d6) δ 8.69 (dd, J = 6.6, 1.7 |
| | yl)methyl)-3-(4- | Hz, 1H), 8.51 (d, J = 2.1 Hz, 1H), 8.47 (dd, J = 7.5, 1.7 Hz, |
| | methylpent-3-en-1- | 1H), 7.88 (dd, J = 8.4, 2.6 Hz, 1H), 7.52 (d, J = 8.3 Hz, |
| | yl)pyrido[2,3-d]pyrimidine- | 1H), 7.02 (dd, J = 7.3, 6.7 Hz, 1H), 5.60 (s, 2H), 5.13-5.09 |
| | 2,4(3H,8H)-dione | (m, 1H), 3.79 (t, J = 7.6 Hz, 2H), 2.19 (q, J = 7.4 Hz, 2H), |
| | | 1.61 (s, 3H), 1.53 (s, 3H); ESI MS (m/z) 371.15 (MH)+. |
| 22 | 8-((6-chloropyridin-3- | ¹H-NMR (400 MHz, DMSO-d6) δ 8.69 (dd, J = 6.4, 1.8 |
| | yl)methyl)-3-(4,4,4- | Hz, 1H), 8.52 (d, J = 2.1 Hz, 1H), 8.47 (dd, J = 7.3, 1.8 Hz, |
| | trifluorobutyl)pyrido[2,3- | 1H), 7.89 (dd, J = 8.3, 2.8 Hz, 1H), 7.52 (dd, J = 8.3, 0.6 |
| | d]pyrimidine-2,4(3H,8H)- | Hz, 1H), 7.02 (dd, J = 7.3, 6.4 Hz, 1H), 5.61 (s, 2H), 3.91 |
| | dione | (t, J = 7.2 Hz, 2H), 2.35-2.23 (m, 2H), 1.80-1.72 (m, 2H); |
| | | ESI MS (m/z) 439.45 (MH + ACN)+. |
| 23 | 8-((6-chloropyridin-3- | ¹H-NMR (400 MHz, DMSO-d6) δ 8.72 (dd, J = 6.4, 1.7 |
| | yl)methyl)-3-(2- | Hz, 1H), 8.53 (d, J = 2.0 Hz, 1H), 8.49 (dd, J = 7.5, 1.8 Hz, |
| | fluoroethyl)pyrido[2,3- | 1H), 7.89 (dd, J = 8.3, 2.4 Hz, 1H), 7.52 (d, J = 8.3 Hz, |
| | d]pyrimidine-2,4(3H,8H)- | 1H), 7.06-7.02 (m, 1H), 5.61 (s, 2H), 4.57 (dt, J = 47.4, 5.4 |
| | dione | Hz, 2H), 4.17 (dt, J = 23.1, 5.4 Hz, 2H); ESI MS (m/z) |
| | | 375.45 (M + ACN)+. |
| 24 | 8-((6-chloropyridin-3- | ¹H-NMR (400 MHz, DMSO-d6) δ 8.70 (dd, J = 6.4, 1.7 |
| | yl)methyl)-3-(5,6,6- | Hz, 1H), 8.54 (d, J = 2.0 Hz, 1H), 8.48 (dd, J = 7.3, 1.7 Hz, |
| | trifluorohex-5-en-1- | 1H), 7.91 (dd, J = 8.3, 2.4 Hz, 1H), 7.55-7.53 (m, 1H), 7.03 |
| | yl)pyrido[2,3-d]pyrimidine- | (dd, J = 7.3, 6.6 Hz, 1H), 5.62 (s, 2H), 3.86 (t, J = 7.0 Hz, |
| | 2,4(3H,8H)-dione | 2H), 2.41-2.34 (m, 2H), 1.61-1.49 (m, 4H); ESI MS (m/z) |
| | | 465.45 (M + ACN)+. |
| 25 | 8-((6-chloropyridin-3- | ¹H-NMR (400 MHz, DMSO-d6) δ 8.70 (dd, J = 6.6, 1.7 |
| | yl)methyl)-3-(2- | Hz, 1H), 8.52 (d, J = 2.2 Hz, 1H), 8.47 (dd, J = 7.3, 1.7 Hz, |
| | ethoxyethyl)pyrido[2,3- | 1H), 7.89 (dd, J = 8.3, 2.4 Hz, 1H), 7.52 (d, J = 8.3 Hz, |
| | d]pyrimidine-2,4(3H,8H)- | 1H), 7.02 (dd, J = 7.3, 6.6 Hz, 1H), 5.60 (s, 2H), 4.01 (t, J = |
| | dione | 6.6 Hz, 2H), 3.49 (t, J = 6.6 Hz, 2H), 3.42 (q, J = 7.0 Hz, |
| | | 2H), 1.04 (t, J = 7.0 Hz, 3H); ESI MS (m/z) 361.00 (MH)+. |
| 26 | 8-((6-chloropyridin-3- | ¹H-NMR (400 MHz, DMSO-d6): δ 8.72 (dd, J = 6.4, 1.8 |
| | yl)methyl)-3-((2,2- | Hz, 1H), 8.53 (d, J = 2.1 Hz, 1H), 8.50 (dd, J = 7.5, 1.7 Hz, |
| | difluorocyclo- | 1H), 7.90 (dd, J = 8.3, 2.4 Hz, 1H), 7.52 (d, J = 8.3 Hz, |
| | propyl)methyl)pyrido[2,3- | 1H), 7.05 (dd, J = 7.3, 6.4 Hz, 1H), 5.61 (t, J = 14.8 Hz, |
| | d]pyrimidine- | 2H), 4.04 (dd, J = 13.6, 6.0 Hz, 1H), 3.93 (dd, J = 13.4, 8.6 |
| | 2,4(3H,8H)-dione | Hz, 1H), 2.13-2.01 (m, 1H), 1.59-1.50 (m, 1H), 1.37-1.29 |
| | | (m, 1H); ESI MS (m/z) 378.90 (MH)+. |
| 27 | 8-((6-chloropyridin-3- | ¹H-NMR (400 MHz, DMSO-d6) δ 8.70 (dd, J = 6.6, 1.7 |
| | yl)methyl)-3-(4,4- | Hz, 1H), 8.52-8.47 (m, 2H), 7.89 (dd, J = 8.2, 2.6 Hz, 1H), |
| | difluorobut-3-en-1- | 7.52 (d, J = 8.3 Hz, 1H), 7.03 (dd, J = 7.3, 6.6 Hz, 1H), |
| | yl)pyrido[2,3-d]pyrimidine- | 5.61 (s, 2H), 4.50 (dtd, J = 26.9, 8.0, 2.6 Hz, 1H), 3.88 (t, |
| | 2,4(3H,8H)-dione | J = 7.2 Hz, 2H), 2.23 (q, J = 7.4 Hz, 2H); ESI MS (m/z) |
| | | 400.90 (M + Na)+ |
| 28 | 8-((6-chloropyridin-3- | ¹H-NMR (400 MHz, DMSO-d6) δ 8.76 (dd, J = 6.6, 1.7 |
| | yl)methyl)-3-(2,2,2- | Hz, 1H), 8.54 (dd, J = 7.5, 1.8 Hz, 2H), 7.90 (dd, J = 8.3, |
| | trifluoroethyl)pyrido[2,3- | 2.7 Hz, 1H), 7.53 (d, J = 8.3 Hz, 1H), 7.08 (t, J = 6.8 Hz, |
| | d]pyrimidine-2,4(3H,8H)- | 1H), 5.60 (d, J = 19.3 Hz, 2H), 4.67 (q, J = 9.3 Hz, 2H); |
| | dione | ESI MS (m/z) 370.85 (MH)+. |
| 29 | 8-((6-chloropyridin-3- | ¹H-NMR (400 MHz, DMSO-d6) δ 8.75 (dd, J = 6.5, 1.8 |
| | yl)methyl)-3-(2,2- | Hz, 1H), 8.53-8.51 (m, 2H), 7.90 (dd, J = 8.3, 2.4 Hz, 1H), |
| | difluoroethyl)pyrido[2,3- | 7.52 (dd, J = 8.3, 0.7 Hz, 1H), 7.07 (dd, J = 7.3, 6.6 Hz, |
| | d]pyrimidine-2,4(3H,8H)- | 1H), 6.19 (tt, J = 56.5, 4.4 Hz, 1H), 5.75 (s, OH), 5.62 (s, |
| | dione | 2H), 4.26 (td, J = 14.3, 4.4 Hz, 2H); ESI MS (m/z) 352.90 |
| | | (MH)+. |
| 30 | 8-((6-chloropyridin-3- | ¹H-NMR (400 MHz, DMSO-d6) δ 8.86 (dd, J = 6.6, 1.7 |
| | yl)methyl)-2,4-dioxo-4,8- | Hz, 1H), 8.62 (dd, J = 7.6, 1.7 Hz, 1H), 8.49 (d, J = 2.4 Hz, |
| | dihydropyrido[2,3- | 1H), 7.88 (dd, J = 8.3, 2.7 Hz, 1H), 7.54 (d, J = 8.1 Hz, |
| | d]pyrimidine-3(2H)- | 1H), 7.20 (dd, J = 7.6, 6.6 Hz, 1H), 5.66 (s, 2H); ESI MS |
| | carbonitrile | (m/z) 313.95 (MH)+. |
| 31 | 8-((6-chloropyridin-3- | ¹H-NMR (400 MHz, DMSO-d6) δ 8.77 (dd, J = 6.6, 1.7 |
| | yl)methyl)-3-(2,2,3,3,3- | Hz, 1H), 8.54 (dd, J = 8.9, 1.6 Hz, 2H), 7.90 (dd, J = 8.3, |
| | pentafluoropropyl)pyrido[2,3- | 2.4 Hz, 1H), 7.53 (d, J = 8.3 Hz, 1H), 7.09 (t, J = 7.0 Hz, |
| | d]pyrimidine- | 1H), 5.63 (s, 2H), 4.70 (t, J = 15.4 Hz, 2H); ESI MS (m/z) |
| | 2,4(3H,8H)-dione | 421.00 (MH)+. |
| 32 | 8-((6-chloropyridin-3- | ¹H-NMR (400 MHz, DMSO-d6) δ 8.71 (dd, J = 6.5, 1.8 |
| | yl)methyl)-3- | Hz, 1H), 8.52 (d, J = 2.0 Hz, 1H), 8.49 (dd, J = 7.3, 1.7 Hz, |
| | (methoxymethyl)pyrido[2,3- | 1H), 7.90 (dd, J = 8.3, 2.7 Hz, 1H), 7.52 (dd, J = 8.3, 0.5 |
| | d]pyrimidine-2,4(3H,8H)- | Hz, 1H), 7.03 (dd, J = 7.3, 6.6 Hz, 1H), 5.61 (s, 2H), 5.22 |
| | dione | (s, 2H), 3.26 (d, J = 2.9 Hz, 3H); ESI MS (m/z) 333.00 |
| | | (MH)+. |
| 33 | 8-((2-chlorothiazol-5- | ¹H-NMR (400 MHz, DMSO-d6) δ 11.03 (s, 1H), 8.66 (dd, |
| | yl)methyl)pyrido[2,3- | J = 6.4, 1.7 Hz, 1H), 8.41 (dd, J = 7.3, 1.7 Hz, 1H), 7.86 (s, |
| | d]pyrimidine-2,4(3H,8H)- | 1H), 6.98 (t, J = 7.0 Hz, 1H), 5.64 (s, 2H); ESI MS (m/z) |
| | dione | 294.90 (MH)+. |
| 34 | 8-((2-chlorothiazol-5- | ¹H-NMR (400 MHz, CHLOROFORM-d) δ 8.53 (dd, J = |
| | yl)methyl)-3- | 7.3, 1.7 Hz, 1H), 7.96 (dd, J = 6.6, 1.7 Hz, 1H), 7.67 (s, |
| | ethylpyrido[2,3- | 1H), 6.83 (dd, J = 7.3, 6.6 Hz, 1H), 5.63 (d, J = 0.5 Hz, |
| | d]pyrimidine-2,4(3H,8H)- | 2H), 4.12 (q, J = 7.1 Hz, 2H), 1.29 (t, J = 7.1 Hz, 3H); ESI |
| | dione | MS (m/z) 322.65 (MH)+. |
| 35 | 8-((2-chlorothiazol-5- | ¹H-NMR (400 MHz, DMSO-d6) δ 8.71 (dd, J = 6.5, 1.8 |
| | yl)methyl)-3- | Hz, 1H), 8.47 (dd, J = 7.3, 1.7 Hz, 1H), 7.86 (s, 1H), 7.04 |
| | methylpyrido[2,3- | (dd, J = 7.5, 6.5 Hz, 1H), 5.68 (s, 2H), 3.24 (d, J = 6.6 Hz, |
| | d]pyrimidine-2,4(3H,8H)- | 3H); ESI MS (m/z) 308.90 (MH)+. |
| | dione | |
| 36 | 2-(8-((2-chlorothiazol-5- | ¹H-NMR (400 MHz, DMSO-d6) δ 8.79 (dd, J = 6.6, 1.7 |
| | yl)methyl)-2,4-dioxo-4,8- | Hz, 1H), 8.56 (dd, J = 7.6, 1.7 Hz, 1H), 7.87 (s, 1H), 7.12 |
| | dihydropyrido[2,3- | (dd, J = 7.3, 6.6 Hz, 1H), 5.71 (s, 2H), 4.87 (s, 2H); ESI |
| | d]pyrimidin-3(2H)- | MS (m/z) 333.90 (MH)+. |
| | yl)acetonitrile | |
| 37 | 8-((2-chlorothiazol-5- | ¹H-NMR (400 MHz, DMSO-d6) δ 8.66 (dd, J = 6.6, 1.7 |
| | yl)methyl)-3- | Hz, 1H), 8.43 (dd, J = 7.5, 1.8 Hz, 1H), 7.86 (s, 1H), 7.00 |
| | isopropylpyrido[2,3- | (dd, J = 7.3, 6.4 Hz, 1H), 5.64 (s, 2H), 5.14-5.07 (m, 1H), |
| | d]pyrimidine-2,4(3H,8H)- | 1.39 (d, J = 7.1 Hz, 6H); ESI MS (m/z) 336.90 (MH)+. |
| | dione | |
| 38 | 8-((2-chlorothiazol-5- | ¹H-NMR (400 MHz, DMSO-d6) δ 8.87 (dd, J = 6.6, 1.7 |
| | yl)methyl)-2,4-dioxo-4,8- | Hz, 1H), 8.62 (dd, J = 7.3, 1.7 Hz, 1H), 7.87 (s, 1H), 7.21 |
| | dihydropyrido[2,3- | (dd, J = 7.5, 6.5 Hz, 1H), 5.74 (s, 2H); ESI MS (m/z) |
| | d]pyrimidine-3(2H)- | 319.80 (MH)+. |
| | carbonitrile | |
| 39 | 8-((2-chlorothiazol-5- | ¹H-NMR (400 MHz, DMSO-d6) δ 8.73 (dd, J = 6.6, 1.7 |
| | yl)methyl)-3-(2- | Hz, 1H), 8.49 (dd, J = 7.3, 1.7 Hz, 1H), 7.87 (s, 1H), 7.06 |
| | fluoroethyl)pyrido[2,3- | (dd, J = 7.3, 6.6 Hz, 1H), 5.69 (s, 2H), 4.61 (dt, J = 47.4, |
| | d]pyrimidine-2,4(3H,8H)- | 5.3 Hz, 2H), 4.21 (dt, J = 23.2, 5.4 Hz, 2H); ESI MS (m/z) |
| | dione | 340.90 (MH)+. |
| 40 | 8-((2-chlorothiazol-5- | ¹H-NMR (400 MHz, DMSO-d6) δ 8.77 (dd, J = 6.4, 1.7 |
| | yl)methyl)-3-(2,2,2- | Hz, 1H), 8.54 (dd, J = 7.3, 1.7 Hz, 1H), 7.89 (d, J = 10.0 |
| | trifluoroethyl)pyrido[2,3- | Hz, 1H), 7.10 (dd, J = 7.4, 6.5 Hz, 1H), 5.70 (s, 2H), 4.71 |
| | d]pyrimidine-2,4(3H,8H)- | (q, J = 9.2 Hz, 2H); ESI MS (m/z) 376.80 (MH)+. |
| | dione | |
| 41 | 8-((2-chlorothiazol-5- | ¹H-NMR (400 MHz, DMSO-d6) δ 8.76 (dd, J = 6.6, 1.7 |
| | yl)methyl)-3-(2,2- | Hz, 1H), 8.52 (dd, J = 7.6, 1.7 Hz, 1H), 7.87 (s, 1H), 7.09 |
| | difluoroethyl)pyrido[2,3- | (dd, J = 7.3, 6.6 Hz, 1H), 6.38-6.08 (m, 1H), 5.70 (s, 2H), |
| | d]pyrimidine-2,4(3H,8H)- | 4.30 (td, J = 14.4, 4.3 Hz, 2H); ESI MS (m/z) 358.90 |
| | dione | (MH)+. |
| 42 | 8-((2-chlorothiazol-5- | ¹H-NMR (400 MHz, DMSO-d6) δ 8.78 (dd, J = 6.6, 1.7 |
| | yl)methyl)-3-(2,2,3,3,3- | Hz, 1H), 8.55 (dd, J = 7.5, 1.8 Hz, 1H), 7.88 (s, 1H), 7.11 |
| | pentafluoropropyl)pyrido[2,3- | (dd, J = 7.5, 6.5 Hz, 1H), 5.71 (s, 2H), 4.74 (t, J = 15.4 Hz, |
| | d]pyrimidine- | 2H); ESI MS (m/z) 466.90 (M + K)+. |
| | 2,4(3H,8H)-dione | |
| 43 | 3-allyl-8-((2-chlorothiazol- | ¹H-NMR (400 MHz, DMSO-d6) δ 8.71 (dd, J = 6.5, 1.6 |
| | 5-yl)methyl)pyrido[2,3- | Hz, 1H), 8.47 (dd, J = 7.5, 1.6 Hz, 1H), 7.87 (s, 1H), 7.04 |
| | d]pyrimidine-2,4(3H,8H)- | (dd, J = 7.1, 6.6 Hz, 1H), 5.90-5.80 (m, 1H), 5.68 (s, 2H), |
| | dione | 5.09-5.05 (m, 2H), 4.47 (d, J = 5.1 Hz, 2H); ESI MS (m/z) |
| | | 334.90 (MH)+. |
| 44 | 8-((2-chlorothiazol-5- | ¹H-NMR (400 MHz, DMSO-d6) δ 8.74 (dd, J = 6.5, 1.8 |
| | yl)methyl)-3-(prop-2-yn-1- | Hz, 1H), 8.51 (dd, J = 7.6, 1.7 Hz, 1H), 7.87 (s, 1H), 7.08 |
| | yl)pyrido[2,3-d]pyrimidine- | (dd, J = 7.3, 6.6 Hz, 1H), 5.69 (s, 2H), 4.60 (d, J = 2.4 Hz, |
| | 2,4(3H,8H)-dione | 2H), 3.05 (t, J = 2.4 Hz, 1H); ESI MS (m/z) 332.85 (MH)+. |
| 45 | 3-acetyl-8-((2- | ¹H-NMR (400 MHz, DMSO-d6) δ 8.79 (dd, J = 6.4, 1.7 |
| | chlorothiazol-5- | Hz, 1H), 8.53 (dd, J = 7.6, 1.7 Hz, 1H), 7.87 (d, J = 6.8 Hz, |
| | yl)methyl)pyrido[2,3- | 1H), 7.12 (dd, J = 7.3, 6.6 Hz, 1H), 5.71 (s, 2H), 2.50 (s, |
| | d]pyrimidine-2,4(3H,8H)- | 3H); ESI MS (m/z) 336.75 (MH)+. |
| | dione | |
| 46 | 8-((2-chlorothiazol-5- | ¹H-NMR (400 MHz, DMSO-d6) δ 8.70 (dd, J = 6.6, 1.7 |
| | yl)methyl)-3-(4,4,4- | Hz, 1H), 8.47 (dd, J = 7.5, 1.8 Hz, 1H), 7.86 (s, 1H), 7.04 |
| | trifluorobutyl)pyrido[2,3- | (dd, J = 7.5, 6.5 Hz, 1H), 5.68 (s, 2H), 3.94 (t, J = 7.2 Hz, |
| | d]pyrimidine-2,4(3H,8H)- | 2H), 2.34-2.24 (m, 2H), 1.82-1.75 (m, 2H); ESI MS (m/z) |
| | dione | 404.90 (MH)+. |
| 47 | 8-((2-chlorothiazol-5- | ¹H-NMR (400 MHz, DMSO-d6) δ 8.70 (dd, J = 6.6, 1.7 |
| | yl)methyl)-3- | Hz, 1H), 8.46 (dd, J = 7.3, 1.7 Hz, 1H), 7.92-7.99 (OH), |
| | (cyclopropylmethyl)pyrido[2,3- | 7.86 (s, 1H), 7.03 (dd, J = 7.3, 6.6 Hz, 1H), 5.85-5.75 (m, |
| | d]pyrimidine- | 1H), 5.67 (s, 2H), 5.03-4.95 (m, 2H), 3.94 (t, J = 7.3 Hz, |
| | 2,4(3H,8H)-dione | 2H), 2.32 (dd, J = 14.5, 6.7 Hz, 2H); ESI MS (m/z) 348.95 |
| | | (MH)+. |
| 48 | 8-((2-chlorothiazol-5- | ¹H-NMR (400 MHz, DMSO-d6) δ 8.78 (dd, J = 6.4, 1.7 |
| | yl)methyl)-3- | Hz, 1H), 8.52 (dd, J = 7.3, 1.7 Hz, 1H), 7.88 (s, 1H), 7.11 |
| | (cyclopropanecarbonyl)pyrido[2,3- | (dd, J = 7.5, 6.5 Hz, 1H), 5.71 (s, 2H), 2.23-2.17 (m, 1H), |
| | d]pyrimidine- | 1.17-1.08 (m, 4H); ESI MS (m/z) 362.90 (MH)+. |
| | 2,4(3H,8H)-dione | |
| 49 | 3-acetyl-8-((6- | ¹H-NMR (400 MHz, DMSO-d6) δ 8.78 (dd, J = 6.4, 1.7 |
| | chloropyridin-3- | Hz, 1H), 8.53 (dd, J = 7.3, 2.0 Hz, 2H), 7.90 (dd, J = 8.3, |
| | yl)methyl)pyrido[2,3- | 2.4 Hz, 1H), 7.53 (dd, J = 8.3, 0.7 Hz, 1H), 7.10 (dd, J = |
| | d]pyrimidine-2,4(3H,8H)- | 7.5, 6.5 Hz, 1H), 5.63 (s, 2H), 2.46 (s, 3H); ESI MS (m/z) |
| | dione | 330.85 (MH)+. |
| 50 | 8-((2-chlorothiazol-5- | ¹H-NMR (400 MHz, DMSO-d6) δ 8.73 (dd, J = 6.5, 1.6 |
| | yl)methyl)-3-(4,4- | Hz, 1H), 8.50 (dd, J = 7.3, 1.7 Hz, 1H), 7.88 (s, 1H), 7.06 |
| | difluorobut-3-en-1- | (t, J = 7.0 Hz, 1H), 5.70 (s, 2H), 4.58-4.47 (m, 1H), 3.95 |
| | yl)pyrido[2,3-d]pyrimidine- | (t, J = 7.1 Hz, 2H), 2.28 (q, J = 7.4 Hz, 2H); ESI MS (m/z) |
| | 2,4(3H,8H)-dione | 384.90 (MH)+. |
| 51 | 8-((6-chloropyridin-3- | ¹H-NMR (400 MHz, DMSO-d6) δ 8.77 (dd, J = 6.6, 1.7 |
| | yl)methyl)-3- | Hz, 1H), 8.52 (dd, J = 7.2, 1.8 Hz, 2H), 7.91 (dd, J = 8.3, |
| | (cyclopropanecarbonyl)pyrido[2,3- | 2.4 Hz, 1H), 7.53 (d, J = 8.3 Hz, 1H), 7.09 (t, J = 7.0 Hz, |
| | d]pyrimidine- | 1H), 5.64 (s, 2H), 2.18-2.14 (m, 1H), 1.12-1.07 (m, 4H); |
| | 2,4(3H,8H)-dione | ESI MS (m/z) 356.90 (MH)+. |
| 52 | 3-(3-chloropropyl)-8-((2- | ¹H-NMR (400 MHz, DMSO-d6) δ 8.71 (d, J = 5.1 Hz, 1H), |
| | chlorothiazol-5- | 8.48-8.46 (m, 1H), 7.86 (s, 1H), 7.04 (t, J = 7.0 Hz, 1H), |
| | yl)methyl)pyrido[2,3- | 5.68 (s, 2H), 3.99 (t, J = 7.0 Hz, 2H), 3.66 (t, J = 6.6 Hz, |
| | d]pyrimidine-2,4(3H,8H)- | 2H), 2.05-1.98 (m, 2H); ESI MS (m/z) 370.70 (MH)+. |
| | dione | |
| 53 | 8-((6-chloropyridin-3- | ¹H-NMR (400 MHz, DMSO-d6) δ 8.74 (dd, J = 6.6, 1.7 |
| | yl)methyl)-3- | Hz, 1H), 8.56 (d, J = 2.2 Hz, 1H), 8.49 (dd, J = 7.3, 1.7 Hz, |
| | phenylpyrido[2,3- | 1H), 7.94 (dd, J = 8.3, 2.4 Hz, 1H), 7.56-7.54 (m, 1H), 7.44 |
| | d]pyrimidine-2,4(3H,8H)- | (td, J = 6.7, 1.5 Hz, 2H), 7.38-7.34 (m, 1H), 7.19-7.17 (m, |
| | dione | 2H), 7.05 (dd, J = 7.2, 6.5 Hz, 1H), 5.65 (s, 2H); ESI MS |
| | | (m/z) 364.95 (MH)+ |
| 54 | 8-((6-chloropyridin-3- | ¹H-NMR (400 MHz, DMSO-d6) δ 8.68 (dd, J = 6.6, 1.7 |
| | yl)methyl)-3- | Hz, 1H), 8.52 (d, J = 1.8 Hz, 1H), 8.45 (dd, J = 7.3, 1.5 Hz, |
| | isopentylpyrido[2,3- | 1H), 7.89 (dd, J = 8.4, 2.6 Hz, 1H), 7.52 (dd, J = 8.3, 0.6 |
| | d]pyrimidine-2,4(3H,8H)- | Hz, 1H), 7.01 (dd, J = 7.3, 6.4 Hz, 1H), 5.59 (s, 2H), 3.86- |
| | dione | 3.82 (m, 2H), 1.57-1.51 (m, 1H), 1.42-1.36 (m, 2H), 0.89 |
| | | (d, J = 6.4 Hz, 6H); ESI MS (m/z) 359.00 (MH)+. |
| 55 | 8-((6-chloropyridin-3- | ¹H-NMR (400 MHz, DMSO-d6) δ 8.72 (dd, J = 6.6, 1.7 |
| | yl)methyl)-3-(3- | Hz, 1H), 8.53-8.48 (m, 2H), 7.89 (dd, J = 8.4, 2.6 Hz, 1H), |
| | cyclopropylprop-2-yn-1- | 7.52 (dd, J = 8.3, 0.6 Hz, 1H), 7.05 (dd, J = 7.3, 6.4 Hz, |
| | yl)pyrido[2,3-d]pyrimidine- | 1H), 5.61 (s, 2H), 4.51 (d, J = 2.1 Hz, 2H), 1.26-1.18 (m, |
| | 2,4(3H,8H)-dione | 1H), 0.72-0.63 (m, 2H), 0.56-0.48 (m, 2H); ESI MS (m/z) |
| | | 367.15 (MH)+. |
| 56 | 8-((6-chloropyridin-3- | ¹H-NMR (400 MHz, DMSO-d6) δ 8.78 (dd, J = 6.4, 1.8 |
| | yl)methyl)-3-(4- | Hz, 1H), 8.55 (d, J = 2.1 Hz, 1H), 8.50 (dd, J = 7.3, 1.8 Hz, |
| | methoxypyridin-2- | 1H), 8.35 (d, J = 5.8 Hz, 1H), 7.93 (dd, J = 8.4, 2.6 Hz, |
| | yl)pyrido[2,3-d]pyrimidine- | 1H), 7.55 (d, J = 8.3 Hz, 1H), 7.09-7.01 (m, 3H), 6.95 (d, |
| | 2,4(3H,8H)-dione | J = 2.1 Hz, 1H), 5.64 (d, J = 9.5 Hz, 2H), 3.84 (d, J = 4.0 |
| | | Hz, 3H); ESI MS (m/z) 396.10 (MH)+ |
| 57 | 8-((6-chloropyridin-3- | ¹H-NMR (400 MHz, DMSO-d6) δ 8.74 (dd, J = 6.6, 1.7 |
| | yl)methyl)-3-(3- | Hz, 1H), 8.55 (d, J = 1.8 Hz, 1H), 8.48 (dd, J = 7.3, 1.8 Hz, |
| | methoxyphenyl)pyrido[2,3- | 1H), 7.94 (dd, J = 8.3, 2.4 Hz, 1H), 7.55 (dd, J = 8.3, 0.6 |
| | d]pyrimidine-2,4(3H,8H)- | Hz, 1H), 7.34 (t, J = 7.9 Hz, 1H), 7.06-7.00 (m, 2H), 6.94 |
| | dione | (ddd, J = 8.5, 2.5, 0.8 Hz, 1H), 6.78-6.74 (m, 2H), 5.64 (s, |
| | | 2H), 3.74 (s, 3H); ESI MS (m/z) 394.95 (MH)+ |
| 58 | 8-((6-chloropyridin-3- | ¹H-NMR (400 MHz, DMSO-d6) δ 8.75 (dd, J = 6.4, 1.5 |
| | yl)methyl)-3-(2- | Hz, 1H), 8.57 (d, J = 2.1 Hz, 1H), 8.47 (dd, J = 7.3, 1.5 Hz, |
| | methoxyphenyl)pyrido[2,3- | 1H), 7.95 (dd, J = 8.4, 2.3 Hz, 1H), 7.56 (d, J = 8.3 Hz, |
| | d]pyrimidine-2,4(3H,8H)- | 1H), 7.38-7.34 (m, 1H), 7.12-7.10 (m, 2H), 7.04 (t, J = 7.0 |
| | dione | Hz, 1H), 6.99 (t, J = 7.5 Hz, 1H), 5.70 (d, J = 14.4 Hz, 1H), |
| | | 5.56 (d, J = 14.7 Hz, 1H), 3.67 (s, 3H) ESI MS (m/z) |
| | | 394.90 (MH)+ |
| 59 | 8-((6-chloropyridin-3- | ¹H-NMR (400 MHz, DMSO-d6) δ 8.73 (dd, J = 6.6, 1.7 |
| | yl)methyl)-3-(4- | Hz, 1H), 8.55 (d, J = 2.1 Hz, 1H), 8.47 (dd, J = 7.3, 1.8 Hz, |
| | methoxyphenyl)pyrido[2,3- | 1H), 7.94 (dd, J = 8.3, 2.4 Hz, 1H), 7.55 (d, J = 8.3 Hz, |
| | d]pyrimidine-2,4(3H,8H)- | 1H), 7.09-7.02 (m, 3H), 6.97 (dd, J = 6.9, 2.3 Hz, 2H), 5.64 |
| | dione | (s, 2H), 3.78 (s, 3H) ESI MS (m/z) 394.90 (MH)+ |
| 60 | 3-(tert-butyl)-8-((6- | ¹H-NMR (400 MHz, DMSO-d6) δ 8.53 (td, J = 6.8, 1.8 Hz, |
| | chloropyridin-3- | 2H), 8.32 (dd, J = 7.2, 1.8 Hz, 1H), 7.89 (dd, J = 8.3, 2.4 |
| | yl)methyl)pyrido[2,3- | Hz, 1H), 7.52 (dd, J = 8.2, 0.6 Hz, 1H), 6.89 (dd, J = 7.2, |
| | d]pyrimidine-2,4(3H,8H)- | 6.5 Hz, 1H), 5.51 (s, 2H), 1.58 (s, 9H) ESI MS (m/z) |
| | dione | 344.95 (MH)+ |
| 61 | 8-((6-chloropyridin-3- | ¹H-NMR (400 MHz, CHLOROFORM-D) δ 8.52-8.47 (m, |
| | yl)methyl)-3-(3- | 2H), 7.97 (dd, J = 8.3, 2.4 Hz, 1H), 7.86 (dd, J = 6.6, 1.7 |
| | methylbutan-2- | Hz, 1H), 7.39 (d, J = 8.3 Hz, 1H), 6.79 (dd, J = 7.1, 6.6 Hz, |
| | yl)pyrido[2,3-d]pyrimidine- | 1H), 5.62 (s, 2H), 4.72 (td, J = 6.8, 3.7 Hz, 1H), 2.70-2.64 |
| | 2,4(3H,8H)-dione | (m, 1H), 1.51 (d, J = 6.8 Hz, 3H), 1.05 (d, J = 6.8 Hz, 3H), |
| | | 0.81 (d, J = 6.6 Hz, 3H); ESI MS (m/z) 359.00 (MH)+. |
| 62 | 8-((2-chlorothiazol-5- | ¹H-NMR (400 MHz, DMSO-d6) δ 8.75 (dd, J = 6.6, 1.7 |
| | yl)methyl)-3- | Hz, 1H), 8.49 (dd, J = 7.5, 1.8 Hz, 1H), 7.90 (s, 1H), 7.48- |
| | phenylpyrido[2,3- | 7.43 (m, 2H), 7.40-7.36 (m, 1H), 7.23-7.21 (m, 2H), 7.06 |
| | d]pyrimidine-2,4(3H,8H)- | (dd, J = 7.3, 6.6 Hz, 1H), 5.72 (s, 2H); ESI MS (m/z) |
| | dione | 370.90 (MH)+ |
| 63 | 3-(sec-butyl)-8-((6- | ¹H-NMR (400 MHz, DMSO-d6) δ 8.65 (dd, J = 6.6, 1.7 |
| | chloropyridin-3- | Hz, 1H), 8.52 (d, J = 2.2 Hz, 1H), 8.43 (dd, J = 7.3, 1.7 Hz, |
| | yl)methyl)pyrido[2,3- | 1H), 7.90 (dd, J = 8.3, 2.7 Hz, 1H), 7.52 (dd, J = 8.3, 0.5 |
| | d]pyrimidine-2,4(3H,8H)- | Hz, 1H), 6.98 (dd, J = 7.3, 6.6 Hz, 1H), 5.57 (s, 2H), 4.87- |
| | dione | 4.79 (m, 1H), 2.06-1.94 (m, 1H), 1.77-1.66 (m, 1H), 1.33 |
| | | (d, J = 6.8 Hz, 3H), 0.73 (t, J = 7.5 Hz, 3H); ESI MS (m/z) |
| | | 345.05 (MH)+. |
| 64 | 8-((6-chloropyridin-3- | ¹H-NMR (400 MHz, DMSO-d6) δ 8.63 (dd, J = 6.6, 1.7 |
| | yl)methyl)-3- | Hz, 1H), 8.51 (dd, J = 2.6, 0.6 Hz, 1H), 8.41 (dd, J = 7.3, |
| | cyclopropylpyrido[2,3- | 1.7 Hz, 1H), 7.88 (dd, J = 8.3, 2.7 Hz, 1H), 7.51 (dd, J = |
| | d]pyrimidine-2,4(3H,8H)- | 8.2, 0.6 Hz, 1H), 6.96 (dd, J = 7.3, 6.6 Hz, 1H), 5.57 (s, |
| | dione | 2H), 2.61-2.55 (m, 1H), 0.95 (td, J = 7.4, 5.9 Hz, 2H), 0.64- |
| | | 0.60 (m, 2H); ESI MS (m/z) 329.10 (MH)+. |
| 65 | 8-((2-chlorothiazol-5- | ¹H-NMR (400 MHz, DMSO-d6) δ 8.66 (dd, J = 6.6, 1.7 |
| | yl)methyl)-3-(3- | Hz, 1H), 8.44 (dd, J = 7.3, 1.7 Hz, 1H), 7.86 (s, 1H), 7.00 |
| | methylbutan-2- | (dd, J = 7.5, 6.5 Hz, 1H), 5.64 (d, J = 14.7 Hz, 2H), 4.58- |
| | yl)pyrido[2,3-d]pyrimidine- | 4.51 (m, 1H), 2.57-2.50 (m, 1H), 1.37 (d, J = 6.8 Hz, 3H), |
| | 2,4(3H,8H)-dione | 0.95 (d, J = 6.6 Hz, 3H), 0.66 (d, J = 6.6 Hz, 3H); ESI MS |
| | | (m/z) 365.00 (MH)+. |
| 66 | 3-(sec-butyl)-8-((2- | ¹H-NMR (400 MHz, DMSO-d6) δ 8.66 (dd, J = 6.6, 1.7 |
| | chlorothiazol-5- | Hz, 1H), 8.43 (dd, J = 7.3, 1.7 Hz, 1H), 7.86 (s, 1H), 7.00 |
| | yl)methyl)pyrido[2,3- | (dd, J = 7.3, 6.6 Hz, 1H), 5.65 (s, 2H), 4.91-4.82 (m, 1H), |
| | d]pyrimidine-2,4(3H,8H)- | 2.09-1.98 (m, 1H), 1.79-1.68 (m, 1H), 1.38-1.32 (m, 3H), |
| | dione | 0.75 (t, J = 7.5 Hz, 3H); ESI MS (m/z) 351.00 (MH)+. |
| 67 | 8-((2-chlorothiazol-5- | ¹H-NMR (400 MHz, DMSO-d6) δ 8.64 (dd, J = 6.4, 1.7 |
| | yl)methyl)-3- | Hz, 1H), 8.41 (dd, J = 7.5, 1.8 Hz, 1H), 7.85 (s, 1H), 6.98 |
| | cyclopropylpyrido[2,3- | (dd, J = 7.3, 6.6 Hz, 1H), 5.64 (s, 2H), 2.66-2.59 (m, 1H), |
| | d]pyrimidine-2,4(3H,8H)- | 1.00-0.93 (m, 2H), 0.68-0.64 (m, 2H); ESI MS (m/z) |
| | dione | 334.90 (MH)+. |
| 68 | 1-(8-((6-chloropyridin-3- | ¹H-NMR (400 MHz, DMSO-d6) δ 8.72 (dd, J = 6.6, 1.7 |
| | yl)methyl)-2,4-dioxo-4,8- | Hz, 1H), 8.51-8.48 (m, 2H), 7.89 (dd, J = 8.3, 2.4 Hz, 1H), |
| | dihydropyrido[2,3- | 7.53 (dd, J = 8.3, 0.6 Hz, 1H), 7.05 (dd, J = 7.3, 6.7 Hz, |
| | d]pyrimidin-3(2H)- | 1H), 5.60 (s, 2H), 1.79 (dd, J = 9.0, 6.3 Hz, 2H), 1.35 (dd, |
| | yl)cyclopropane-1- | J = 8.7, 6.3 Hz, 2H); ESI MS (m/z) 353.95 (MH)+. |
| | carbonitrile | |
| 69 | 1-(8-((2-chloropyrimidin-5- | ¹H-NMR (400 MHz, DMSO-d6) δ 8.87 (s, 2H), 8.73 (dd, |
| | yl)methyl)-2,4-dioxo-4,8- | J = 6.6, 1.7 Hz, 1H), 8.49 (dd, J = 7.3, 1.7 Hz, 1H), 7.05 |
| | dihydropyrido[2,3- | (dd, J = 7.3, 6.6 Hz, 1H), 5.59 (s, 2H), 1.80 (dd, J = 8.9, |
| | d]pyrimidin-3(2H)- | 6.2 Hz, 2H), 1.34 (dd, J = 8.9, 6.2 Hz, 2H); ESI MS (m/z) |
| | yl)cyclopropane-1- | 354.90 (MH)+. |
| | carbonitrile | |
| 70 | 1-(8-((2-chlorothiazol-5- | ¹H-NMR (400 MHz, DMSO-d6) δ 8.73 (dd, J = 6.5, 1.8 |
| | yl)methyl)-2,4-dioxo-4,8- | Hz, 1H), 8.49 (dd, J = 7.5, 1.8 Hz, 1H), 7.86 (s, 1H), 7.07 |
| | dihydropyrido[2,3- | (dd, J = 7.5, 6.5 Hz, 1H), 5.67 (s, 2H), 1.82 (dd, J = 8.9, |
| | d]pyrimidin-3(2H)- | 6.2 Hz, 2H), 1.39 (dd, J = 8.9, 6.2 Hz, 2H); ESI MS (m/z) |
| | yl)cyclopropane-1- | 359.90 (MH)+. |
| | carbonitrile | |
| 71 | 8-((2-chlorothiazol-5- | ¹H-NMR (400 MHz, DMSO-^{d}6) δ 8.74 (dd, J = 6.6, 1.7 |
| | yl)methyl)-3-(4- | Hz, 1H), 8.47 (dd, J = 7.3, 1.7 Hz, 1H), 7.89 (s, 1H), 7.14- |
| | methoxyphenyl)pyrido[2,3- | 7.10 (m, 2H), 7.05 (dd, J = 7.3, 6.6 Hz, 1H), 7.01-6.97 (m, |
| | d]pyrimidine-2,4(3H,8H)- | 2H), 5.71 (s, 2H), 3.79 (s, 3H); ESI MS (m/z) 401.00 |
| | dione | (MH)+ |
| 72 | 8-((2-chlorothiazol-5- | ¹H-NMR (400 MHz, DMSO-d6) δ 8.75 (dd, J = 6.6, 1.7 |
| | yl)methyl)-3-(3- | Hz, 1H), 8.48 (dd, J = 7.5, 1.8 Hz, 1H), 7.89 (s, 1H), 7.36 |
| | methoxyphenyl)pyrido[2,3- | (t, J = 8.1 Hz, 1H), 7.06 (dd, J = 7.3, 6.4 Hz, 1H), 6.96 |
| | d]pyrimidine-2,4(3H,8H)- | (ddd, J = 8.4, 2.6, 0.9 Hz, 1H), 6.82-6.78 (m, 2H), 5.73 (d, |
| | dione | J = 10.8 Hz, 2H), 3.75 (s, 3H); ESI MS 400.85 (MH)+ |
| 73 | 8-((2-chlorothiazol-5- | ¹H-NMR (400 MHz, DMSO-d6) δ 8.75 (dd, J = 6.5, 1.8 |
| | yl)methyl)-3-(2- | Hz, 1H), 8.48 (dd, J = 7.3, 1.7 Hz, 1H), 7.90 (s, 1H), 7.40- |
| | methoxyphenyl)pyrido[2,3- | 7.35 (m, 1H), 7.17-7.12 (m, 2H), 7.06 (dd, J = 7.3, 6.4 Hz, |
| | d]pyrimidine-2,4(3H,8H)- | 1H), 7.01 (td, J = 7.5, 1.2 Hz, 1H), 5.77-5.73 (m, 1H), 5.68 |
| | dione | (d, J = 14.4 Hz, 1H), 3.69 (s, 3H); ESI MS (m/z) 401.00 |
| | | (MH)+ |
| 74 | 8-((6-chloropyridin-3- | ¹H-NMR (400 MHz, DMSO-d6) δ 8.65 (dd, J = 6.4, 1.5 |
| | yl)methyl)-3-(4- | Hz, 1H), 8.52 (d, J = 2.1 Hz, 1H), 8.43 (dd, J = 7.3, 1.5 Hz, |
| | methylpentan-2- | 1H), 7.90 (dd, J = 8.3, 2.4 Hz, 1H), 7.53 (d, J = 8.3 Hz, |
| | yl)pyrido[2,3-d]pyrimidine- | 1H), 6.98 (t, J = 7.0 Hz, 1H), 5.57 (s, 2H), 5.02 (dd, J = |
| | 2,4(3H,8H)-dione | 15.4, 6.6 Hz, 1H), 2.10-2.03 (m, 1H), 1.46-1.39 (m, 1H), |
| | | 1.33-1.22 (m, 4H), 0.81 (dd, J = 9.9, 6.6 Hz, 6H) ESI MS |
| | | (m/z) 373.20 (MH)+ |
| 75 | 8-((6-chloropyridin-3- | ¹H-NMR (400 MHz, DMSO-d6) δ 8.66-8.36 (m, 3H), |
| | yl)methyl)-3-(pentan-2- | 7.90-7.77 (m, 1H), 7.52 (d, J = 7.9 Hz, 1H), 6.98 (t, J = 6.4 |
| | yl)pyrido[2,3-d]pyrimidine- | Hz, 1H), 5.66 (d, J = 68.8 Hz, 2H), 4.94 (d, J = 6.7 Hz, |
| | 2,4(3H,8H)-dione | 1H), 2.03-2.00 (m, 1H), 1.62 (d, J = 5.2 Hz, 1H), 1.33 (d, |
| | | J = 6.4 Hz, 3H), 1.21-0.97 (m, 2H), 0.96-0.65 (m, 3H) ESI |
| | | MS (m/z) 359.15 (MH)+ |
| 76 | 8-((6-chloropyridin-3- | 1H-NMR (400 MHz, DMSO-d6) δ 8.67 (dd, J = 6.6, 1.7 |
| | yl)methyl)-3-(1- | Hz, 1H), 8.53 (d, J = 2.0 Hz, 1H), 8.44 (dd, J = 7.3, 1.7 Hz, |
| | methoxypropan-2- | 1H), 7.90 (dd, J = 8.2, 2.6 Hz, 1H), 7.53 (dd, J = 8.3, 0.7 |
| | yl)pyrido[2,3-d]pyrimidine- | Hz, 1H), 7.00 (dd, J = 7.3, 6.7 Hz, 1H), 5.57 (s, 2H), 5.17- |
| | 2,4(3H,8H)-dione | 5.08 (m, 1H), 3.90 (dd, J = 9.5, 8.1 Hz, 1H), 3.54-3.49 (m, |
| | | 1H), 3.16 (d, J = 11.2 Hz, 3H), 1.33-1.28 (m, 3H) ESI MS |
| | | (m/z) 361.05 (MH)+ |
| 77 | 8-((6-chloropyridin-3- | 1H-NMR (400 MHz, DMSO-d6) δ 8.67 (dd, J = 6.6, 1.7 |
| | yl)methyl)-3-(1- | Hz, 1H), 8.53 (d, J = 2.0 Hz, 1H), 8.44 (dd, J = 7.2, 1.6 Hz, |
| | methoxybutan-2- | 1H), 7.91 (dd, J = 8.3, 2.4 Hz, 1H), 7.53 (dd, J = 8.3, 0.7 |
| | yl)pyrido[2,3-d]pyrimidine- | Hz, 1H), 7.00 (dd, J = 7.3, 6.6 Hz, 1H), 5.59 (d, J = 9.8 Hz, |
| | 2,4(3H,8H)-dione | 2H), 5.02-4.94 (m, 1H), 3.90 (dd, J = 9.8, 8.1 Hz, 1H), 3.53 |
| | | (dd, J = 9.8, 5.6 Hz, 1H), 3.17 (d, J = 6.8 Hz, 3H), 1.99- |
| | | 1.91 (m, 1H), 1.70-1.63 (m, 1H), 0.75 (t, J = 7.6 Hz, 3H) |
| | | ESI MS (m/z) 375.25 (MH)+ |
| 78 | 8-((2-chlorothiazol-5- | ¹H-NMR (400 MHz, DMSO-d6) δ 8.66 (dd, J = 6.6, 1.7 |
| | yl)methyl)-3-(4- | Hz, 1H), 8.44 (dd, J = 7.6, 1.7 Hz, 1H), 7.86 (s, 1H), 7.00 |
| | methylpentan-2- | (dd, J = 7.3, 6.6 Hz, 1H), 5.65 (s, 2H), 5.08-5.02 (m, 1H), |
| | yl)pyrido[2,3-d]pyrimidine- | 2.13-2.06 (m, 1H), 1.49-1.42 (m, 1H), 1.34 (dd, J = 11.7, |
| | 2,4(3H,8H)-dione | 6.8 Hz, 4H), 0.82 (dd, J = 8.6, 6.6 Hz, 6H) ESI MS (m/z) |
| | | 379.00 (MH)+ |
| 79 | 3-(4-chlorophenyl)-8-((6- | ¹H-NMR (400 MHz, DMSO-d6) δ 8.75 (dd, J = 6.6, 1.7 |
| | chloropyridin-3- | Hz, 1H), 8.55 (d, J = 2.0 Hz, 1H), 8.49 (dd, J = 7.5, 1.8 Hz, |
| | yl)methyl)pyrido[2,3- | 1H), 7.94 (dd, J = 8.2, 2.6 Hz, 1H), 7.55 (dd, J = 8.2, 0.6 |
| | d]pyrimidine-2,4(3H,8H)- | Hz, 1H), 7.52-7.48 (m, 2H), 7.26-7.22 (m, 2H), 7.06 (dd, J = |
| | dione | 7.3, 6.6 Hz, 1H), 5.65 (s, 2H); ESI MS (m/z) 440.20 |
| | | (M + K)+ |
| 80 | 3-(4-chlorophenyl)-8-((2- | ¹H-NMR (400 MHz, DMSO-d6) δ 8.76 (dd, J = 6.6, 1.7 |
| | chlorothiazol-5- | Hz, 1H), 8.50 (dd, J = 7.3, 1.8 Hz, 1H), 7.89 (s, 1H), 7.53- |
| | yl)methyl)pyrido[2,3- | 7.50 (m, 2H), 7.28 (t, J = 5.7 Hz, 2H), 7.07 (t, J = 7.0 Hz, |
| | d]pyrimidine-2,4(3H,8H)- | 1H), 5.72 (s, 2H); ESI MS (m/z) 446.00 (M + K)+ |
| | dione | |
| 81 | 8-((6-chloropyridin-3- | ¹H-NMR (400 MHz, DMSO-d6) δ 8.72 (dd, J = 6.5, 1.8 |
| | yl)methyl)-3-(1-methyl-1H- | Hz, 1H), 8.54 (d, J = 2.0 Hz, 1H), 8.49 (dd, J = 7.3, 1.7 Hz, |
| | pyrazol-4-yl)pyrido[2,3- | 1H), 7.91 (dd, J = 8.3, 2.7 Hz, 1H), 7.79 (s, 1H), 7.53 (dd, |
| | d]pyrimidine-2,4(3H,8H)- | J = 8.3, 0.5 Hz, 1H), 7.42 (s, 1H), 7.04 (dd, J = 7.3, 6.6 Hz, |
| | dione | 1H), 5.63 (s, 2H), 3.84 (s, 3H); ESI MS (m/z) 368.95 |
| | | (MH)+ |
| 82 | 3-(4-chlorophenyl)-8-((2- | ¹H-NMR (400 MHz, DMSO-d6) δ 8.92 (s, 2H), 8.78-8.74 |
| | chloropyrimidin-5- | (m, 1H), 8.50-8.48 (m, 1H), 7.52-7.48 (m, 2H), 7.25-7.21 |
| | yl)methyl)pyrido[2,3- | (m, 2H), 7.06 (dd, J = 7.3, 6.6 Hz, 1H), 5.64 (s, 2H); ESI |
| | d]pyrimidine-2,4(3H,8H)- | MS (m/z) 441.05 (M + K)+ |
| | dione | |
| 83 | 8-((2-chloropyrimidin-5- | ¹H-NMR (400 MHz, DMSO-d6) δ 8.89 (s, 2H), 8.70 (dd, |
| | yl)methyl)-3- | J = 6.6, 1.7 Hz, 1H), 8.45 (dd, J = 7.3, 1.7 Hz, 1H), 7.01 |
| | ethylpyrido[2,3- | (dd, J = 7.3, 6.6 Hz, 1H), 5.58 (s, 2H), 3.86 (q, J = 7.0 Hz, |
| | d]pyrimidine-2,4(3H,8H)- | 2H), 1.08 (t, J = 7.0 Hz, 3H); ESI MS (m/z) 317.95 (MH)+. |
| | dione | |
| 84 | 8-((2-chloropyrimidin-5- | ¹H-NMR (400 MHz, DMSO-d6) δ 8.89 (s, 2H), 8.66 (dd, |
| | yl)methyl)-3- | J = 6.6, 1.7 Hz, 1H), 8.42 (dd, J = 7.5, 1.8 Hz, 1H), 6.98 |
| | isopropylpyrido[2,3- | (dd, J = 7.3, 6.4 Hz, 1H), 5.56 (s, 2H), 5.10-5.03 (m, 1H), |
| | d]pyrimidine-2,4(3H,8H)- | 1.36 (d, J = 6.8 Hz, 6H); ESI MS (m/z) 331.95 (MH)+. |
| | dione | |
| 85 | 8-((6-chloropyridin-3- | ¹H-NMR (400 MHz, DMSO-d6) δ 8.53 (td, J = 6.8, 1.9 Hz, |
| | yl)methyl)-3-(tert- | 2H), 8.32 (dd, J = 7.3, 1.8 Hz, 1H), 7.89 (dd, J = 8.3, 2.8 |
| | pentyl)pyrido[2,3- | Hz, 1H), 7.52 (dd, J = 8.3, 0.6 Hz, 1H), 6.89 (dd, J = 7.3, |
| | d]pyrimidine-2,4(3H,8H)- | 6.7 Hz, 1H), 5.52 (s, 2H), 2.07 (q, J = 7.4 Hz, 2H), 1.55 (s, |
| | dione | 6H), 0.70 (t, J = 7.5 Hz, 3H) ESI MS (m/z) 358.95 (MH)+ |
| 86 | 8-((2-chlorothiazol-5- | ¹H-NMR (400 MHz, DMSO-d6) δ 8.66 (dd, J = 6.6, 1.7 |
| | yl)methyl)-3-(pentan-2- | Hz, 1H), 8.43 (dd, J = 7.3, 1.8 Hz, 1H), 7.86 (s, 1H), 7.00 |
| | yl)pyrido[2,3-d]pyrimidine- | (dd, J = 7.3, 6.4 Hz, 1H), 5.65 (s, 2H), 5.00-4.95 (m, 1H), |
| | 2,4(3H,8H)-dione | 2.10-2.01 (m, 1H), 1.70-1.61 (m, 1H), 1.37 (d, J = 6.7 Hz, |
| | | 3H), 1.22-1.10 (m, 2H), 0.83 (t, J = 7.3 Hz, 3H) ESI MS |
| | | (m/z) 364.90 (MH)+ |
| 87 | 8-((6-chloropyridin-3- | ¹H-NMR (400 MHz, DMSO-d6) δ 8.78 (dd, J = 6.4, 1.7 |
| | yl)methyl)-3-(3- | Hz, 1H), 8.55 (d, J = 2.2 Hz, 1H), 8.51 (dd, J = 7.5, 1.8 Hz, |
| | (trifluoromethyl)phenyl)pyrido[2,3- | 1H), 7.95 (dd, J = 8.3, 2.4 Hz, 1H), 7.75 (d, J = 7.8 Hz, |
| | d]pyrimidine- | 1H), 7.69 (t, J = 7.8 Hz, 1H), 7.65 (s, 1H), 7.57-7.54 (m, |
| | 2,4(3H,8H)-dione | 2H), 7.08 (dd, J = 7.3, 6.6 Hz, 2H), 5.67 (s, 2H); ESI MS |
| | | (m/z) 432.85 (MH)+ |
| 88 | 8-((2-chlorothiazol-5- | ¹H-NMR (400 MHz, DMSO-d6) δ 8.78 (dd, J = 6.6, 1.7 |
| | yl)methyl)-3-(3- | Hz, 1H), 8.51 (dd, J = 7.3, 1.7 Hz, 1H), 7.90 (s, 1H), 7.78- |
| | (trifluoromethyl)phenyl)pyrido[2,3- | 7.69 (m, 3H), 7.59 (d, J = 7.8 Hz, 1H), 7.09 (dd, J = 7.3, |
| | d]pyrimidine- | 6.4 Hz, 1H), 5.74 (s, 2H); ESI MS (m/z) 438.95 (MH)+ |
| | 2,4(3H,8H)-dione | |
| 89 | 8-((2-chlorothiazol-5- | ¹H-NMR (400 MHz, DMSO-d6) δ 8.73 (dd, J = 6.6, 1.7 |
| | yl)methyl)-3-(1-methyl-1H- | Hz, 1H), 8.49 (dd, J = 7.3, 1.7 Hz, 1H), 7.88 (s, 1H), 7.84 |
| | pyrazol-4-yl)pyrido[2,3- | (d, J = 0.5 Hz, 1H), 7.46 (d, J = 0.6 Hz, 1H), 7.06 (dd, J = |
| | d]pyrimidine-2,4(3H,8H)- | 7.5, 6.5 Hz, 1H), 5.70 (s, 2H), 3.86 (s, 3H); ESI MS (m/z) |
| | dione | 374.85 (MH)+ |
| 90 | 8-((2-chlorothiazol-5- | ¹H-NMR (400 MHz, DMSO-d6) δ 8.68 (dd, J = 6.6, 1.7 |
| | yl)methyl)-3-(1- | Hz, 1H), 8.44 (dd, J = 7.3, 1.5 Hz, 1H), 7.86 (s, 1H), 7.02 |
| | methoxypropan-2- | (dd, J = 7.3, 6.7 Hz, 1H), 5.65 (s, 2H), 5.19-5.11 (m, 1H), |
| | yl)pyrido[2,3-d]pyrimidine- | 3.93 (dd, J = 9.8, 7.9 Hz, 1H), 3.55 (dd, J = 9.8, 6.1 Hz, |
| | 2,4(3H,8H)-dione | 1H), 3.19 (s, 3H), 1.33-1.28 (m, 3H) ESI MS (m/z) 366.45 |
| | | (MH)+ |
| 91 | 8-((2-chlorothiazol-5- | ¹H-NMR (400 MHz, DMSO-d6) δ 8.71-8.67 (m, 1H), 8.45 |
| | yl)methyl)-3-(1- | (dd, J = 7.3, 1.5 Hz, 1H), 7.87 (s, 1H), 7.02 (dd, J = 7.3, |
| | methoxybutan-2- | 6.4 Hz, 2H), 5.69-5.62 (m, 2H), 5.05-4.97 (m, 1H), 3.93 |
| | yl)pyrido[2,3-d]pyrimidine- | (dd, J = 9.9, 8.1 Hz, 1H), 3.58-3.53 (m, 1H), 3.17 (d, J = |
| | 2,4(3H,8H)-dione | 15.3 Hz, 3H), 2.04-1.90 (m, 1H), 1.75-1.64 (m, 1H), 0.77 |
| | | (t, J = 7.3 Hz, 3H) ESI MS (m/z) 380.45 (MH)+ |
| 92 | 8-((6-chloropyridin-3- | ¹H-NMR (400 MHz, DMSO-d6) δ 8.62 (dd, J = 6.5, 1.8 |
| | yl)methyl)-3-(1- | Hz, 1H), 8.51 (d, J = 2.0 Hz, 1H), 8.40 (dd, J = 7.3, 1.7 Hz, |
| | methylcyclopropyl)pyrido[2,3- | 1H), 7.88 (dd, J = 8.3, 2.4 Hz, 1H), 7.52 (dd, J = 8.2, 0.6 |
| | d]pyrimidine- | Hz, 1H), 6.95 (dd, J = 7.3, 6.6 Hz, 1H), 5.56 (s, 2H), 1.31 |
| | 2,4(3H,8H)-dione | (s, 3H), 0.83 (s, 2H), 0.73 (s, 2H); ESI MS (m/z) 342.45 |
| | | (MH)+. |
| 93 | 8-((6-chloropyridin-3- | ¹H-NMR (400 MHz, DMSO-d6) δ 8.70 (dd, J = 6.6, 1.7 |
| | yl)methyl)-3-(2- | Hz, 1H), 8.52 (d, J = 2.0 Hz, 1H), 8.47 (dd, J = 7.3, 1.7 Hz, |
| | methoxyethyl)pyrido[2,3- | 1H), 7.89 (dd, J = 8.3, 2.4 Hz, 1H), 7.52 (dd, J = 8.3, 0.5 |
| | d]pyrimidine-2,4(3H,8H)- | Hz, 1H), 7.02 (dd, J = 7.5, 6.5 Hz, 1H), 5.60 (s, 2H), 4.02 |
| | dione | (t, J = 6.4 Hz, 2H), 3.47 (t, J = 6.2 Hz, 2H), 3.22 (s, 3H); |
| | | ESI MS (m/z) 346.45 (MH)+. |
| 94 | 3-(3-chlorophenyl)-8-((6- | ¹H-NMR (400 MHz, DMSO-d6) δ 8.77 (dd, J = 6.4, 1.8 |
| | chloropyridin-3- | Hz, 1H), 8.55 (d, J = 2.1 Hz, 1H), 8.50 (dd, J = 7.3, 1.8 Hz, |
| | yl)methyl)pyrido[2,3- | 1H), 7.94 (dd, J = 8.4, 2.6 Hz, 1H), 7.56-7.52 (m, 1H), |
| | d]pyrimidine-2,4(3H,8H)- | 7.50-7.43 (m, 2H), 7.35 (t, J = 1.5 Hz, 1H), 7.23-7.18 (m, |
| | dione | 1H), 7.06 (dd, J = 7.3, 6.4 Hz, 1H), 5.65 (s, 2H); ESI MS |
| | | (m/z) 398.85 (MH)+ |
| 95 | 3-(8-((6-chloropyridin-3- | ¹H-NMR (400 MHz, DMSO-d6) δ 8.79 (dd, J = 6.6, 1.7 |
| | yl)methyl)-2,4-dioxo-4,8- | Hz, 1H), 8.56-8.51 (m, 2H), 7.95 (dd, J = 8.3, 2.8 Hz, 1H), |
| | dihydropyrido[2,3- | 7.86 (dt, J = 7.5, 1.5 Hz, 1H), 7.77 (q, J = 1.2 Hz, 1H), |
| | d]pyrimidin-3(2H)- | 7.69-7.65 (m, 1H), 7.62-7.59 (m, 1H), 7.57-7.54 (m, 1H), |
| | yl)benzonitrile | 7.08 (dd, J = 7.3, 6.7 Hz, 1H), 5.66 (s, 2H); ESI MS (m/z) |
| | | 389.90 (MH)+ |
| 96 | 3-(3-chlorophenyl)-8-((2- | ¹H-NMR (400 MHz, DMSO-d6) δ 8.92 (s, 2H), 8.78 (dd, |
| | chloropyrimidin-5- | J = 6.4, 1.8 Hz, 1H), 8.50 (dd, J = 7.3, 1.5 Hz, 1H), 7.50- |
| | yl)methyl)pyrido[2,3- | 7.43 (m, 2H), 7.34-7.33 (m, 1H), 7.19 (dt, J = 7.0, 1.8 Hz, |
| | d]pyrimidine-2,4(3H,8H)- | 1H), 7.07 (dd, J = 7.3, 6.7 Hz, 1H), 5.65 (s, 2H); ESI MS |
| | dione | (m/z) 399.85 (MH)+ |
| 97 | 3-(8-((2-chloropyrimidin-5- | ¹H-NMR (400 MHz, DMSO-d6) δ 8.94 (d, J = 6.7 Hz, 2H), |
| | yl)methyl)-2,4-dioxo-4,8- | 8.80 (dd, J = 6.6, 1.7 Hz, 1H), 8.52 (dd, J = 7.5, 1.7 Hz, |
| | dihydropyrido[2,3- | 1H), 7.87-7.85 (m, 1H), 7.76 (q, J = 1.2 Hz, 1H), 7.69-7.65 |
| | d]pyrimidin-3(2H)- | (m, 1H), 7.61-7.58 (m, 1H), 7.09 (dd, J = 7.5, 6.6 Hz, 1H), |
| | yl)benzonitrile | 5.66 (s, 2H); ESI MS (m/z) 390.90 (MH)+ |
| 98 | 8-((6-chloropyridin-3- | ¹H-NMR (400 MHz, DMSO-d6) δ 8.78-8.70 (m, 1H), |
| | yl)methyl)-3-(1,1,1- | 8.58-8.50 (m, 2H), 7.91 (dd, J = 8.4, 2.6 Hz, 1H), 7.54 (t, |
| | trifluoropropan-2- | J = 8.1 Hz, 1H), 7.07 (s, 1H), 5.75-5.50 (m, 3H), 1.61 (d, J = |
| | yl)pyrido[2,3-d]pyrimidine- | 6.7 Hz, 3H) ESI MS (m/z) 384.90 (MH)+ |
| | 2,4(3H,8H)-dione | |
| 99 | 8-((2-chloropyrimidin-5- | ¹H-NMR (400 MHz, DMSO-d6) δ 8.88 (s, 2H), 8.64 (dd, |
| | yl)methyl)-3-(1- | J = 6.6, 1.7 Hz, 1H), 8.39 (dd, J = 7.3, 1.7 Hz, 1H), 6.95 |
| | methylcyclopropyl)pyrido[2,3- | (dd, J = 7.3, 6.6 Hz, 1H), 5.54 (s, 2H), 1.31 (s, 3H), 0.83 |
| | d]pyrimidine- | (s, 2H), 0.73 (s, 2H); ESI MS (m/z) 343.50 (MH)+. |
| | 2,4(3H,8H)-dione | |
| 100 | 8-((2-chloropyrimidin-5- | ¹H-NMR (400 MHz, DMSO-d6) δ 8.89 (s, 2H), 8.71 (dd, |
| | yl)methyl)-3-(2- | J = 6.5, 1.6 Hz, 1H), 8.46 (dd, J = 7.5, 1.8 Hz, 1H), 7.02 |
| | methoxyethyl)pyrido[2,3- | (dd, J = 7.3, 6.6 Hz, 1H), 5.59 (s, 2H), 4.02 (t, J = 6.2 Hz, |
| | d]pyrimidine-2,4(3H,8H)- | 2H), 3.47 (t, J = 6.2 Hz, 2H), 3.21 (s, 3H); ESI MS (m/z) |
| | dione | 347.45 (MH)+. |
| 101 | 8-((6-chloropyridin-3- | ¹H-NMR (400 MHz, DMSO-d6) δ 8.79 (dd, J = 6.6, 1.7 |
| | yl)methyl)-3-(3,5- | Hz, 1H), 8.55-8.50 (m, 2H), 7.94 (dd, J = 8.3, 2.4 Hz, 1H), |
| | dichlorophenyl)pyrido[2,3- | 7.65 (t, J = 2.0 Hz, 1H), 7.56 (d, J = 8.2 Hz, 1H), 7.40 (d, |
| | d]pyrimidine-2,4(3H,8H)- | J = 2.0 Hz, 2H), 7.08 (dd, J = 7.3, 6.6 Hz, 1H), 5.66 (s, |
| | dione | 2H); ESI MS (m/z) 434.45 (MH)+ |
| 102 | 8-((6-chloropyridin-3- | ¹H-NMR (400 MHz, DMSO-d6) δ 8.77 (dd, J = 6.6, 1.7 |
| | yl)methyl)-3-(4- | Hz, 1H), 8.56 (d, J = 2.2 Hz, 1H), 8.52 (dd, J = 7.5, 1.8 Hz, |
| | (trifluoromethyl)phenyl)pyrido[2,3- | 1H), 7.95 (dd, J = 8.3, 2.7 Hz, 1H), 7.82 (d, J = 8.3 Hz, |
| | d]pyrimidine- | 2H), 7.55 (dd, J = 8.3, 0.5 Hz, 1H), 7.47 (d, J = 8.1 Hz, |
| | 2,4(3H,8H)-dione | 2H), 7.08 (dd, J = 7.3, 6.6 Hz, 1H), 5.66 (s, 2H); ESI MS |
| | | (m/z) 432.50 (MH)+ |
| 103 | 4-(8-((6-chloropyridin-3- | ¹H-NMR (400 MHz, DMSO-d6) δ 8.78 (dd, J = 6.6, 1.7 |
| | yl)methyl)-2,4-dioxo-4,8- | Hz, 1H), 8.55 (d, J = 2.2 Hz, 1H), 8.51 (dd, J = 7.3, 1.7 Hz, |
| | dihydropyrido[2,3- | 1H), 7.96-7.91 (m, 3H), 7.55 (d, J = 8.3 Hz, 1H), 7.46 (dt, |
| | d]pyrimidin-3(2H)- | J = 8.8, 2.0 Hz, 2H), 7.08 (dd, J = 7.3, 6.6 Hz, 1H), 5.66 (s, |
| | yl)benzonitrile | 2H); ESI MS (m/z) 389.50 (MH)+ |
| 104 | 8-((6-chloropyridin-3- | ¹H-NMR (400 MHz, DMSO-d6) δ 8.78 (dd, J = 6.6, 1.7 |
| | yl)methyl)-3-(3,4- | Hz, 1H), 8.58-8.48 (m, 2H), 7.94 (dd, J = 8.3, 2.4 Hz, 1H), |
| | dichlorophenyl)pyrido[2,3- | 7.77-7.67 (m, 1H), 7.60 (d, J = 2.4 Hz, 1H), 7.57-7.53 (m, |
| | d]pyrimidine-2,4(3H,8H)- | 1H), 7.31-7.25 (m, 1H), 7.09-7.04 (m, 1H), 5.64 (d, J = |
| | dione | 14.7 Hz, 2H); ESI MS (m/z) 432.80 (MH)+ |
| 105 | 8-((6-chloropyridin-3- | ¹H-NMR (400 MHz, DMSO-d6) δ 8.75 (dd, J = 6.6, 1.7 |
| | yl)methyl)-3-(4- | Hz, 1H), 8.55 (d, J = 2.1 Hz, 1H), 8.49 (dd, J = 7.3, 1.8 Hz, |
| | fluorophenyl)pyrido[2,3- | 1H), 7.94 (dd, J = 8.3, 2.4 Hz, 1H), 7.56-7.54 (m, 1H), |
| | d]pyrimidine-2,4(3H,8H)- | 7.29-7.22 (m, 4H), 7.05 (dd, J = 7.3, 6.7 Hz, 1H), 5.65 (s, |
| | dione | 2H); ESI MS (m/z) 382.85 (MH)+ |
| 106 | 3-(4-bromophenyl)-8-((6- | ¹H-NMR (400 MHz, DMSO-d6) δ 8.76 (dd, J = 6.6, 1.7 |
| | chloropyridin-3- | Hz, 1H), 8.55 (d, J = 2.1 Hz, 1H), 8.50 (dd, J = 7.3, 1.5 Hz, |
| | yl)methyl)pyrido[2,3- | 1H), 7.94 (dd, J = 8.4, 2.6 Hz, 1H), 7.65-7.61 (m, 2H), 7.55 |
| | d]pyrimidine-2,4(3H,8H)- | (dd, J = 8.3, 0.6 Hz, 1H), 7.19-7.16 (m, 2H), 7.06 (dd, J = |
| | dione | 7.3, 6.7 Hz, 1H), 5.65 (s, 2H); ESI MS (m/z) 444.65 |
| | | (MH)+ |
| 107 | 3-(3,5- | ¹H-NMR (400 MHz, DMSO-d6) δ 8.82 (dd, J = 6.4, 1.8 |
| | bis(trifluoromethyl)phenyl)- | Hz, 1H), 8.55-8.52 (m, 2H), 8.16 (s, 1H), 8.09 (s, 2H), 7.95 |
| | 8-((6-chloropyridin-3- | (dd, J = 8.4, 2.6 Hz, 1H), 7.56 (dd, J = 8.3, 0.6 Hz, 1H), |
| | yl)methyl)pyrido[2,3- | 7.11 (dd, J = 7.3, 6.4 Hz, 1H), 5.68 (s, 2H); ESI MS (m/z) |
| | d]pyrimidine-2,4(3H,8H)- | 500.95 (MH)+ |
| | dione | |
| 108 | 3-(3-bromo-5- | ¹H-NMR (400 MHz, DMSO-d6) δ 8.79 (dd, J = 6.5, 1.8 |
| | chlorophenyl)-8-((6- | Hz, 1H), 8.55-8.50 (m, 2H), 7.94 (dd, J = 8.3, 2.4 Hz, 1H), |
| | chloropyridin-3- | 7.76 (t, J = 1.8 Hz, 1H), 7.56 (d, J = 8.3 Hz, 1H), 7.52 (t, J = |
| | yl)methyl)pyrido[2,3- | 1.7 Hz, 1H), 7.44 (t, J = 1.7 Hz, 1H), 7.08 (dd, J = 7.3, |
| | d]pyrimidine-2,4(3H,8H)- | 6.4 Hz, 1H), 5.66 (s, 2H); ESI MS (m/z) 478.35 (MH)+ |
| | dione | |
| 109 | 3-(3-chloro-5- | ¹H-NMR (400 MHz, DMSO-d6) δ 8.82 (dd, J = 6.6, 1.7 |
| | (trifluoromethyl)phenyl)-8- | Hz, 1H), 8.57-8.53 (m, 2H), 7.98-7.93 (m, 2H), 7.78 (t, J = |
| | ((6-chloropyridin-3- | 1.6 Hz, 1H), 7.73-7.72 (m, 1H), 7.59-7.57 (m, 1H), 7.11 |
| | yl)methyl)pyrido[2,3- | (dd, J = 7.3, 6.6 Hz, 1H), 5.69 (s, 2H); ESI MS (m/z) |
| | d]pyrimidine-2,4(3H,8H)- | 466.45 (MH)+ |
| | dione | |
| 110 | 8-((6-chloropyridin-3- | ¹H-NMR (400 MHz, DMSO-d6) δ 8.72 (dd, J = 6.6, 1.7 |
| | yl)methyl)-3-(1-ethyl-1H- | Hz, 1H), 8.54 (d, J = 2.0 Hz, 1H), 8.49 (dd, J = 7.5, 1.8 Hz, |
| | pyrazol-4-yl)pyrido[2,3- | 1H), 7.91 (dd, J = 8.2, 2.6 Hz, 1H), 7.84 (d, J = 0.7 Hz, |
| | d]pyrimidine-2,4(3H,8H)- | 1H), 7.53 (dd, J = 8.2, 0.6 Hz, 1H), 7.44 (d, J = 0.7 Hz, |
| | dione | 1H), 7.04 (dd, J = 7.3, 6.6 Hz, 1H), 5.63 (s, 2H), 4.13 (q, J = |
| | | 7.3 Hz, 2H), 1.38 (t, J = 7.2 Hz, 3H); ESI MS (m/z) |
| | | 382.90 (MH)+ |
| 111 | 8-((6-chloropyridin-3- | ¹H-NMR (400 MHz, DMSO-d6) δ 8.74 (dd, J = 6.6, 1.7 |
| | yl)methyl)-3-(3- | Hz, 1H), 8.55 (d, J = 2.2 Hz, 1H), 8.48 (dd, J = 7.3, 1.7 Hz, |
| | ethoxyphenyl)pyrido[2,3- | 1H), 7.94 (dd, J = 8.3, 2.4 Hz, 1H), 7.55 (d, J = 8.1 Hz, |
| | d]pyrimidine-2,4(3H,8H)- | 1H), 7.32 (t, J = 7.9 Hz, 1H), 7.04 (t, J = 7.0 Hz, 1H), 6.92 |
| | dione | (dd, J = 8.3, 1.7 Hz, 1H), 6.75-6.72 (m, 2H), 5.64 (s, 2H), |
| | | 4.00 (q, J = 6.9 Hz, 2H), 1.31 (t, J = 7.0 Hz, 3H); ESI MS |
| | | (m/z) 408.95 (MH)+ |
| 112 | 8-((6-chloropyridin-3- | ¹H-NMR (400 MHz, DMSO-^{d}6) δ 8.77 (dd, J = 6.6, 1.7 |
| | yl)methyl)-3-(1-methyl-1H- | Hz, 1H), 8.55-8.52 (m, 2H), 7.94 (dd, J = 8.3, 2.4 Hz, 1H), |
| | pyrazol-5-yl)pyrido[2,3- | 7.55 (dd, J = 8.3, 0.5 Hz, 1H), 7.46 (d, J = 2.0 Hz, 1H), |
| | d]pyrimidine-2,4(3H,8H)- | 7.09 (dd, J = 7.3, 6.6 Hz, 1H), 6.16 (d, J = 2.0 Hz, 1H), |
| | dione | 5.66 (dd, J = 24.5, 14.4 Hz, 2H), 3.54 (s, 3H); ESI MS |
| | | (m/z) 368.95 (MH)+ |
| 113 | 8-((6-chloropyridin-3- | ¹H-NMR (400 MHz, DMSO-^{d}6) δ 8.78 (dd, J = 6.6, 1.7 |
| | yl)methyl)-3-(2- | Hz, 1H), 8.56 (d, J = 2.4 Hz, 1H), 8.53 (dd, J = 7.3, 1.7 Hz, |
| | fluorophenyl)pyrido[2,3- | 1H), 7.94 (dd, J = 8.3, 2.4 Hz, 1H), 7.55 (d, J = 8.3 Hz, |
| | d]pyrimidine-2,4(3H,8H)- | 1H), 7.47-7.42 (m, 1H), 7.36-7.31 (m, 2H), 7.29-7.26 (m, |
| | dione | 1H), 7.09 (t, J = 7.0 Hz, 1H), 5.74-5.69 (m, 1H), 5.60 (d, J = |
| | | 14.4 Hz, 1H); ESI MS (m/z) 382.45 (MH)+ |
| 114 | 8-((6-chloropyridin-3- | ¹H-NMR (400 MHz, DMSO-^{d}6) δ 8.76 (dd, J = 6.5, 1.8 |
| | yl)methyl)-3-(3- | Hz, 1H), 8.55 (d, J = 2.0 Hz, 1H), 8.50 (dd, J = 7.3, 1.7 Hz, |
| | fluorophenyl)pyrido[2,3- | 1H), 7.94 (dd, J = 8.2, 2.6 Hz, 1H), 7.55 (dd, J = 8.2, 0.6 |
| | d]pyrimidine-2,4(3H,8H)- | Hz, 1H), 7.48 (td, J = 8.1, 6.5 Hz, 1H), 7.25-7.20 (m, 1H), |
| | dione | 7.13 (dt, J = 9.9, 2.1 Hz, 1H), 7.08-7.04 (m, 2H), 5.65 (s, |
| | | 2H); ESI MS (m/z) 382.55 (MH)+ |
| 115 | 8-((6-chloropyridin-3- | ¹H-NMR (400 MHz, DMSO-^{d}6) δ 8.77 (dd, J = 6.5, 1.8 |
| | yl)methyl)-3-(3,4- | Hz, 1H), 8.55 (d, J = 2.0 Hz, 1H), 8.50 (dd, J = 7.5, 1.8 Hz, |
| | difluorophenyl)pyrido[2,3- | 1H), 7.94 (dd, J = 8.3, 2.4 Hz, 1H), 7.55 (dd, J = 8.3, 0.5 |
| | d]pyrimidine-2,4(3H,8H)- | Hz, 1H), 7.52-7.47 (m, 1H), 7.41 (ddd, J = 11.4, 7.5, 2.4 |
| | dione | Hz, 1H), 7.12-7.05 (m, 2H), 5.65 (s, 2H); ESI MS (m/z) |
| | | 400.45 (MH)+ |
| 116 | 8-((6-chloropyridin-3- | ¹H-NMR (400 MHz, DMSO-d6) δ 8.79 (dd, J = 6.4, 1.7 |
| | yl)methyl)-3-(3,5- | Hz, 1H), 8.55 (d, J = 2.2 Hz, 1H), 8.51 (dd, J = 7.3, 1.7 Hz, |
| | difluorophenyl)pyrido[2,3- | 1H), 7.94 (dd, J = 8.2, 2.6 Hz, 1H), 7.56 (d, J = 8.1 Hz, |
| | d]pyrimidine-2,4(3H,8H)- | 1H), 7.32-7.27 (m, 1H), 7.10-7.06 (m, 3H), 5.65 (s, 2H); |
| | dione | ESI MS (m/z) 400.55 (MH)+ |
| 117 | 8-((2-chloropyrimidin-5- | ¹H-NMR (400 MHz, DMSO-d6) δ 8.92 (s, 2H), 8.76 (dd, |
| | yl)methyl)-3-(4- | J = 6.5, 1.8 Hz, 1H), 8.49 (dd, J = 7.5, 1.8 Hz, 1H), 7.29- |
| | fluorophenyl)pyrido[2,3- | 7.21 (m, 4H), 7.06 (dd, J = 7.3, 6.6 Hz, 1H), 5.64 (s, 2H); |
| | d]pyrimidine-2,4(3H,8H)- | ESI MS (m/z) 383.90 (MH)+ |
| | dione | |
| 118 | 8-((2-chloropyrimidin-5- | ¹H-NMR (400 MHz, DMSO-d6) δ 8.92 (s, 2H), 8.77 (dd, |
| | yl)methyl)-3-(3- | J = 6.6, 1.7 Hz, 1H), 8.50 (dd, J = 7.3, 1.7 Hz, 1H), 7.48 |
| | fluorophenyl)pyrido[2,3- | (dd, J = 14.7, 8.1 Hz, 1H), 7.23 (td, J = 8.3, 2.5 Hz, 1H), |
| | d]pyrimidine-2,4(3H,8H)- | 7.12 (dt, J = 9.8, 2.2 Hz, 1H), 7.07 (t, J = 7.0 Hz, 2H), 5.64 |
| | dione | (s, 2H); ESI MS (m/z) 384.00 (MH)+ |
| 119 | 8-((6-chloropyridin-3- | ¹H-NMR (400 MHz, DMSO-d6) δ 8.80 (dd, J = 6.4, 1.7 |
| | yl)methyl)-3-(2,4- | Hz, 1H), 8.56-8.53 (m, 2H), 7.95 (dd, J = 8.3, 2.4 Hz, 1H), |
| | dichlorophenyl)pyrido[2,3- | 7.79 (d, J = 2.2 Hz, 1H), 7.57-7.52 (m, 2H), 7.43 (d, J = |
| | d]pyrimidine-2,4(3H,8H)- | 8.6 Hz, 1H), 7.09 (t, J = 7.0 Hz, 1H), 5.78-5.74 (m, 1H), |
| | dione | 5.56 (d, J = 14.4 Hz, 1H); ESI MS (m/z) 432.90 (MH)+ |
| 120 | 8-((2-chloropyrimidin-5- | ¹H-NMR (400 MHz, DMSO-d6) δ 8.92 (s, 2H), 8.78 (dd, |
| | yl)methyl)-3-(3,4- | J = 6.6, 1.7 Hz, 1H), 8.50 (dd, J = 7.3, 1.7 Hz, 1H), 7.51 |
| | difluorophenyl)pyrido[2,3- | (dd, J = 19.6, 8.8 Hz, 1H), 7.42-7.37 (m, 1H), 7.10-7.05 |
| | d]pyrimidine-2,4(3H,8H)- | (m, 2H), 5.64 (s, 2H); ESI MS (m/z) 402.00 (MH)+ |
| | dione | |
| 121 | 8-((6-chloropyridin-3- | ¹H-NMR (400 MHz, DMSO-d6) δ 8.74 (dd, J = 6.6, 1.7 |
| | yl)methyl)-3-(m- | Hz, 1H), 8.55 (d, J = 2.2 Hz, 1H), 8.48 (dd, J = 7.5, 1.8 Hz, |
| | tolyl)pyrido[2,3- | 1H), 7.94 (dd, J = 8.3, 2.4 Hz, 1H), 7.55 (d, J = 8.1 Hz, |
| | d]pyrimidine-2,4(3H,8H)- | 1H), 7.31 (t, J = 7.6 Hz, 1H), 7.17 (d, J = 7.6 Hz, 1H), 7.04 |
| | dione | (dd, J = 7.1, 6.6 Hz, 1H), 6.99-6.96 (m, 2H), 5.65 (s, 2H), |
| | | 2.30 (d, J = 12.0 Hz, 3H); ESI MS (m/z) 378.95 (MH)+. |
| 122 | 8-((6-chloropyridin-3- | ¹H-NMR (400 MHz, DMSO-d6) δ 8.74 (dd, J = 6.4, 1.7 |
| | yl)methyl)-3-(4- | Hz, 1H), 8.55 (d, J = 2.0 Hz, 1H), 8.50-8.46 (m, 1H), 7.94 |
| | (methylthio)phenyl)pyrido[2,3- | (dd, J = 8.3, 2.4 Hz, 1H), 7.56-7.54 (m, 1H), 7.32-7.29 (m, |
| | d]pyrimidine- | 2H), 7.12 (dd, J = 6.6, 2.2 Hz, 2H), 7.04 (dd, J = 7.3, 6.6 |
| | 2,4(3H,8H)-dione | Hz, 1H), 5.64 (s, 2H), 2.50 (s, 3H); ESI MS (m/z) 410.90 |
| | | (MH)+. |
| 123 | 8-((6-chloropyridin-3- | ¹H-NMR (400 MHz, DMSO-^{d}6) δ 8.77 (dd, J = 6.6, 1.7 |
| | yl)methyl)-3-(3- | Hz, 1H), 8.55 (d, J = 2.2 Hz, 1H), 8.50 (dd, J = 7.3, 1.7 Hz, |
| | (trifluoromethoxy)phenyl)p | 1H), 7.94 (dd, J = 8.3, 2.4 Hz, 1H), 7.60-7.54 (m, 2H), |
| | yrido[2,3-d]pyrimidine- | 7.40-7.37 (m, 1H), 7.30-7.26 (m, 2H), 7.07 (dd, J = 7.3, |
| | 2,4(3H,8H)-dione | 6.4 Hz, 1H), 5.66 (s, 2H); ESI MS (m/z) 449.00 (MH)+. |
| 124 | 8-((6-chloropyridin-3- | ¹H-NMR (400 MHz, DMSO-^{d}6) δ 8.76 (dd, J = 6.5, 1.8 |
| | yl)methyl)-3-(3- | Hz, 1H), 8.55 (d, J = 2.0 Hz, 1H), 8.50 (dd, J = 7.3, 1.7 Hz, |
| | (difluoromethoxy)phenyl)pyrido[2,3- | 1H), 7.94 (dd, J = 8.3, 2.4 Hz, 1H), 7.55 (dd, J = 8.3, 0.5 |
| | d]pyrimidine- | Hz, 1H), 7.49 (t, J = 8.1 Hz, 1H), 7.44-7.04 (m, 5H), 5.65 |
| | 2,4(3H,8H)-dione | (s, 2H); ESI MS (m/z) 430.95 (MH)+. |
| 125 | 8-((6-chloropyridin-3- | ¹H-NMR (400 MHz, DMSO-^{d}6) δ 8.73 (dd, J = 6.6, 1.7 |
| | yl)methyl)-3-(p- | Hz, 1H), 8.55 (d, J = 2.2 Hz, 1H), 8.47 (dd, J = 7.3, 1.7 Hz, |
| | tolyl)pyrido[2,3- | 1H), 7.94 (dd, J = 8.3, 2.4 Hz, 1H), 7.55 (dd, J = 8.3, 0.5 |
| | d]pyrimidine-2,4(3H,8H)- | Hz, 1H), 7.23 (d, J = 8.1 Hz, 2H), 7.04 (dd, J = 7.7, 6.2 Hz, |
| | dione | 3H), 5.64 (s, 2H), 2.34-2.32 (m, 3H); ESI MS (m/z) 379.20 |
| | | (MH)+ |
| 126 | 8-((6-chloropyridin-3- | ¹H-NMR (400 MHz, DMSO-^{d}6) δ 8.76 (dd, J = 6.5, 1.8 |
| | yl)methyl)-3-(4- | Hz, 1H), 8.55 (d, J = 2.2 Hz, 1H), 8.50 (dd, J = 7.3, 1.7 Hz, |
| | (trifluoromethoxy)phenyl)pyrido[2,3- | 1H), 7.94 (dd, J = 8.3, 2.4 Hz, 1H), 7.56-7.51 (m, 1H), |
| | d]pyrimidine- | 7.48-7.43 (m, 2H), 7.37-7.31 (m, 2H), 7.08-7.03 (m, 1H), |
| | 2,4(3H,8H)-dione | 5.66 (s, 2H); ESI MS (m/z) 449.00 (MH)+ |
| 127 | 8-((6-chloropyridin-3- | ¹H-NMR (400 MHz, DMSO-^{d}6) δ 8.75 (dd, J = 6.5, 1.8 |
| | yl)methyl)-3-(4- | Hz, 1H), 8.55 (d, J = 2.2 Hz, 1H), 8.49 (dd, J = 7.3, 1.7 Hz, |
| | (difluoromethoxy)phenyl)pyrido[2,3- | 1H), 7.94 (dd, J = 8.3, 2.7 Hz, 1H), 7.55 (dd, J = 8.3, 0.5 |
| | d]pyrimidine- | Hz, 1H), 7.48-7.11 (m, 5H), 7.05 (dd, J = 7.3, 6.6 Hz, 1H), |
| | 2,4(3H,8H)-dione | 5.65 (s, 2H); ESI MS (m/z) 431.00 (MH)+ |
| 128 | 8-((2-chloropyrimidin-5- | ¹H-NMR (400 MHz, DMSO-^{d}6) δ 8.92 (s, 2H), 8.75 (dd, J = |
| | yl)methyl)-3-(m- | 6.6, 1.7 Hz, 1H), 8.47 (dd, J = 7.5, 1.7 Hz, 1H), 7.31 (t, |
| | tolyl)pyrido[2,3- | J = 7.8 Hz, 1H), 7.17 (d, J = 7.6 Hz, 1H), 7.04 (dd, J = 7.2, |
| | d]pyrimidine-2,4(3H,8H)- | 6.6 Hz, 1H), 6.98-6.95 (m, 2H), 5.64 (s, 2H), 2.32 (s, 3H); |
| | dione | ESI MS (m/z) 379.95 (MH)+. |
| 129 | 8-((2-chloropyrimidin-5- | ¹H-NMR (400 MHz, DMSO-^{d}6) δ 8.91 (d, J = 7.1 Hz, 2H), |
| | yl)methyl)-3-(4- | 8.75 (dd, J = 6.6, 1.7 Hz, 1H), 8.48 (dd, J = 7.3, 1.7 Hz, |
| | (methylthio)phenyl)pyrido[2,3- | 1H), 7.31 (dt, J = 9.0, 2.3 Hz, 2H), 7.11 (dt, J = 9.0, 2.3 Hz, |
| | d]pyrimidine- | 2H), 7.05 (dd, J = 7.3, 6.4 Hz, 1H), 5.63 (s, 2H), 2.50 (s, |
| | 2,4(3H,8H)-dione | 3H); ESI MS (m/z) 411.90 (MH)+. |
| 130 | 8-((2-chloropyrimidin-5- | ¹H-NMR (400 MHz, DMSO-d6) δ 8.92 (s, 2H), 8.78 (dd, |
| | yl)methyl)-3-(3- | J = 6.5, 1.8 Hz, 1H), 8.50 (dd, J = 7.3, 1.7 Hz, 1H), 7.58 (t, |
| | (trifluoromethoxy)phenyl)pyrido[2,3- | J = 8.2 Hz, 1H), 7.39 (dt, J = 8.4, 1.2 Hz, 1H), 7.29-7.25 |
| | d]pyrimidine- | (m, 2H), 7.07 (dd, J = 7.3, 6.6 Hz, 1H), 5.65 (s, 2H); ESI |
| | 2,4(3H,8H)-dione | MS (m/z) 450.10 (MH)+. |
| 131 | 8-((2-chloropyrimidin-5- | ¹H-NMR (400 MHz, DMSO-d6) δ 8.91 (d, J = 6.8 Hz, 2H), |
| | yl)methyl)-3-(3- | 8.77 (dd, J = 6.5, 1.8 Hz, 1H), 8.49 (dd, J = 7.6, 1.7 Hz, |
| | (difluoromethoxy)phenyl)pyrido[2,3- | 1H), 7.49 (t, J = 8.1 Hz, 1H), 7.44-7.05 (m, 5H), 5.65 (s, |
| | d]pyrimidine- | 2H); ESI MS (m/z) 431.40 (MH)+. |
| | 2,4(3H,8H)-dione | |
| 132 | 8-((6-chloropyridin-3- | ¹H-NMR (400 MHz, DMSO-d6) δ 8.64 (dd, J = 6.6, 1.7 |
| | yl)methyl)-3- | Hz, 1H), 8.52 (d, J = 1.8 Hz, 1H), 8.42 (dd, J = 7.3, 1.8 Hz, |
| | cyclohexylpyrido[2,3- | 1H), 7.88 (dd, J = 8.3, 2.8 Hz, 1H), 7.52 (dd, J = 8.3, 0.6 |
| | d]pyrimidine-2,4(3H,8H)- | Hz, 1H), 6.98 (dd, J = 7.3, 6.4 Hz, 1H), 5.57 (s, 2H), 4.67 |
| | dione | (tt, J = 12.0, 3.6 Hz, 1H), 2.41-2.31 (m, 2H), 1.76 (d, J = |
| | | 13.1 Hz, 2H), 1.61 (d, J = 12.2 Hz, 1H), 1.48 (d, J = 10.4 |
| | | Hz, 2H), 1.31-1.22 (m, 2H), 1.18-1.07 (m, 1H) ESI MS |
| | | (m/z) 370.50 (MH)+ |
| 133 | 8-((2-chlorothiazol-5- | ¹H-NMR (400 MHz, DMSO-d6) δ 8.66 (dd, J = 6.4, 1.8 |
| | yl)methyl)-3- | Hz, 1H), 8.42 (dd, J = 7.3, 1.8 Hz, 1H), 7.85 (s, 1H), 7.00 |
| | cyclohexylpyrido[2,3- | (dd, J = 7.3, 6.4 Hz, 1H), 5.64 (s, 2H), 4.70 (tt, J = 12.0, |
| | d]pyrimidine-2,4(3H,8H)- | 3.6 Hz, 1H), 2.44-2.32 (m, 2H), 1.78 (d, J = 12.8 Hz, 2H), |
| | dione | 1.62 (d, J = 12.5 Hz, 1H), 1.52 (d, J = 10.4 Hz, 2H), 1.33- |
| | | 1.22 (m, 2H), 1.18-1.09 (m, 1H) ESI MS (m/z) 376.50 |
| | | (MH)+ |
| 134 | 2-chloro-5-(8-((6- | ¹H-NMR (400 MHz, DMSO-d6) δ 8.81 (dd, J = 6.6, 1.7 |
| | chloropyridin-3-yl)methyl)- | Hz, 1H), 8.55-8.52 (m, 2H), 7.94 (dd, J = 8.9, 2.4 Hz, 2H), |
| | 2,4-dioxo-4,8- | 7.85 (d, J = 8.6 Hz, 1H), 7.66 (dd, J = 8.9, 2.4 Hz, 1H), |
| | dihydropyrido[2,3- | 7.56 (d, J = 8.3 Hz, 1H), 7.10 (dd, J = 7.2, 6.6 Hz, 1H), |
| | d]pyrimidin-3(2H)- | 5.66 (s, 2H); ESI MS (m/z) 464.50 (M + K)+ |
| | yl)benzonitrile | |
| 135 | 3-(4-chloro-2- | ¹H-NMR (400 MHz, DMSO-d6) δ 8.80 (dd, J = 6.6, 1.7 |
| | fluorophenyl)-8-((6- | Hz, 1H), 8.56-8.53 (m, 2H), 7.94 (dd, J = 8.2, 2.6 Hz, 1H), |
| | chloropyridin-3- | 7.62-7.59 (m, 1H), 7.55 (d, J = 8.3 Hz, 1H), 7.43-7.37 (m, |
| | yl)methyl)pyrido[2,3- | 2H), 7.10 (dd, J = 7.1, 6.6 Hz, 1H), 5.74-5.70 (m, 1H), 5.60 |
| | d]pyrimidine-2,4(3H,8H)- | (d, J = 14.4 Hz, 1H); ESI MS (m/z) 457.40 (M + K)+ |
| | dione | |
| 136 | 8-((6-chloropyridin-3- | ¹H-NMR (400 MHz, DMSO-d6) δ 8.66 (dd, J = 6.5, 1.8 |
| | yl)methyl)-3- | Hz, 1H), 8.52 (d, J = 2.0 Hz, 1H), 8.44 (dd, J = 7.3, 1.7 Hz, |
| | cyclopentylpyrido[2,3- | 1H), 7.89 (dd, J = 8.3, 2.7 Hz, 1H), 7.52 (d, J = 8.3 Hz, |
| | d]pyrimidine-2,4(3H,8H)- | 1H), 6.99 (dd, J = 7.3, 6.6 Hz, 1H), 5.58 (s, 2H), 5.24 (t, J = |
| | dione | 8.7 Hz, 1H), 2.06-2.01 (m, 2H), 1.88-1.86 (m, 2H), 1.70 |
| | | (s, 2H), 1.53 (t, J = 5.1 Hz, 2H) ESI MS (m/z) 356.50 |
| | | (MH)+ |
| 137 | 8-((6-chloropyridin-3- | ¹H-NMR (400 MHz, DMSO-d6) δ 8.66-8.63 (m, 1H), 8.52 |
| | yl)methyl)-3-(4- | (t, J = 2.2 Hz, 1H), 8.44-8.40 (m, 1H), 7.89 (td, J = 5.6, 2.8 |
| | methylcyclohexyl)pyrido[2,3- | Hz, 1H), 7.52 (d, J = 8.3 Hz, 1H), 7.00-6.96 (m, 1H), 5.57 |
| | d]pyrimidine- | (s, 2H), 4.65 (tt, J = 12.1, 3.9 Hz, 1H), 2.63-2.54 (m, 1H), |
| | 2,4(3H,8H)-dione | 2.40 (td, J = 12.5, 3.2 Hz, 1H), 1.73-1.70 (m, 1H), 1.54- |
| | | 1.45 (m, 3H), 1.26-1.22 (m, 1H), 1.03-0.93 (m, 3H), 0.88 |
| | | (d, J = 6.6 Hz, 2H)ESI MS (m/z) 384.55 (MH)+ |
| 138 | 3-(4-chloro-3- | ¹H-NMR (400 MHz, DMSO-d6) δ 8.78 (dd, J = 6.4, 1.7 |
| | fluorophenyl)-8-((6- | Hz, 1H), 8.55 (d, J = 2.2 Hz, 1H), 8.51 (dd, J = 7.6, 1.7 Hz, |
| | chloropyridin-3- | 1H), 7.94 (dd, J = 8.3, 2.4 Hz, 1H), 7.67 (t, J = 8.3 Hz, 1H), |
| | yl)methyl)pyrido[2,3- | 7.55 (d, J = 8.6 Hz, 1H), 7.40 (dd, J = 10.3, 2.2 Hz, 1H), |
| | d]pyrimidine-2,4(3H,8H)- | 7.15-7.12 (m, 1H), 7.07 (t, J = 7.0 Hz, 1H), 5.65 (s, 2H); |
| | dione | ESI MS (m/z) 416.90 (MH)+ |
| 139 | 8-((6-chloropyridin-3- | ¹H-NMR (400 MHz, DMSO-d6) δ 8.81 (dd, J = 6.6, 1.7 |
| | yl)methyl)-3-(2,4- | Hz, 1H), 8.58-8.54 (m, 2H), 7.96 (dd, J = 8.3, 2.4 Hz, 1H), |
| | difluorophenyl)pyrido[2,3- | 7.57 (d, J = 8.6 Hz, 1H), 7.47-7.40 (m, 2H), 7.22-7.17 (m, |
| | d]pyrimidine-2,4(3H,8H)- | 1H), 7.11 (dd, J = 7.3, 6.4 Hz, 1H), 5.76-5.71 (m, 1H), 5.62 |
| | dione | (d, J = 14.4 Hz, 1H); ESI MS (m/z) 400.45 (MH)+ |
| 140 | 8-((2-chloropyrimidin-5- | ¹H-NMR (400 MHz, DMSO-d6) δ 8.90 (d, J = 13.8 Hz, |
| | yl)methyl)-3-(3,5- | 2H), 8.80 (dd, J = 6.6, 1.7 Hz, 1H), 8.51 (dd, J = 7.3, 1.8 |
| | difluorophenyl)pyrido[2,3- | Hz, 1H), 7.30 (tt, J = 9.5, 2.3 Hz, 1H), 7.10-7.04 (m, 3H), |
| | d]pyrimidine-2,4(3H,8H)- | 5.65 (s, 2H); ESI MS (m/z) 401.40 (MH)+ |
| | dione | |
| 141 | 8-((2-chlorothiazol-5- | ¹H-NMR (400 MHz, DMSO-d6) δ 8.79 (dd, J = 6.6, 1.7 |
| | yl)methyl)-3-(3,5- | Hz, 1H), 8.52 (dd, J = 7.3, 1.8 Hz, 1H), 7.90 (s, 1H), 7.32 |
| | difluorophenyl)pyrido[2,3- | (tt, J = 9.4, 2.4 Hz, 1H), 7.14-7.08 (m, 3H), 5.73 (s, 2H); |
| | d]pyrimidine-2,4(3H,8H)- | ESI MS (m/z) 406.35 (MH)+ |
| | dione | |
| 142 | 8-((2-chlorothiazol-5- | ¹H-NMR (400 MHz, DMSO-d6) δ 8.74 (dd, J = 6.6, 1.7 |
| | yl)methyl)-3-(p- | Hz, 1H), 8.48 (dd, J = 7.3, 1.7 Hz, 1H), 7.89 (s, 1H), 7.25 |
| | tolyl)pyrido[2,3- | (d, J = 8.1 Hz, 2H), 7.09-7.04 (m, 3H), 5.71 (s, 2H), 2.35 |
| | d]pyrimidine-2,4(3H,8H)- | (s, 3H); ESI MS (m/z) 384.45 (MH)+ |
| | dione | |
| 143 | 8-((2-chlorothiazol-5- | ¹H-NMR (400 MHz, DMSO-d6) δ 8.77 (dd, J = 6.6, 1.7 |
| | yl)methyl)-3-(3- | Hz, 1H), 8.50 (dd, J = 7.5, 1.7 Hz, 1H), 7.90 (s, 1H), 7.50 |
| | fluorophenyl)pyrido[2,3- | (td, J = 8.1, 6.6 Hz, 1H), 7.27-7.16 (m, 2H), 7.11-7.06 (m, |
| | d]pyrimidine-2,4(3H,8H)- | 2H), 5.74 (d, J = 7.0 Hz, 2H); ESI MS (m/z) 388.40 (MH)+ |
| | dione | |
| 144 | 8-((2-chloropyrimidin-5- | ¹H-NMR (400 MHz, DMSO-d6) δ 8.92 (s, 2H), 8.74 (dd, |
| | yl)methyl)-3-(p- | J = 6.6, 1.7 Hz, 1H), 8.47 (dd, J = 7.3, 1.7 Hz, 1H), 7.23 |
| | tolyl)pyrido[2,3- | (d, J = 7.8 Hz, 2H), 7.06-7.02 (m, 3H), 5.69 (d, J = 45.7 |
| | d]pyrimidine-2,4(3H,8H)- | Hz, 2H), 2.33 (d, J = 8.8 Hz, 3H); ESI MS (m/z) 379.95 |
| | dione | (MH)+ |
| 145 | 3-(4-chlorophenyl)-8-((6- | ¹H-NMR (400 MHz, DMSO-d6) δ 8.87 (d, J = 1.5 Hz, 1H), |
| | (trifluoromethyl)pyridin-3- | 8.78 (dd, J = 6.6, 1.7 Hz, 1H), 8.52 (dd, J = 7.3, 1.7 Hz, |
| | yl)methyl)pyrido[2,3- | 1H), 8.11-8.09 (m, 1H), 7.93 (d, J = 8.1 Hz, 1H), 7.50 (dt, |
| | d]pyrimidine-2,4(3H,8H)- | J = 9.3, 2.6 Hz, 2H), 7.23 (dt, J = 9.3, 2.6 Hz, 2H), 7.08 |
| | dione | (dd, J = 7.3, 6.6 Hz, 1H), 5.76 (s, 2H); ESI MS (m/z) |
| | | 432.95 (MH)+ |
| 146 | 2-chloro-5-(8-((2- | ¹H-NMR (400 MHz, DMSO-d6) δ 8.92 (s, 2H), 8.81 (dd, |
| | chloropyrimidin-5- | J = 6.6, 1.7 Hz, 1H), 8.53 (dd, J = 7.3, 1.7 Hz, 1H), 7.94 |
| | yl)methyl)-2,4-dioxo-4,8- | (d, J = 2.4 Hz, 1H), 7.85 (d, J = 8.6 Hz, 1H), 7.64 (dd, J = |
| | dihydropyrido[2,3- | 8.6, 2.4 Hz, 1H), 7.10 (dd, J = 7.1, 6.6 Hz, 1H), 5.66 (s, |
| | d]pyrimidin-3(2H)- | 2H); ESI MS (m/z) 465.45 (M + K)+ |
| | yl)benzonitrile | |
| 147 | 3-(4-chloro-2- | ¹H-NMR (400 MHz, DMSO-d6) δ 8.92 (s, 2H), 8.80 (dd, |
| | fluorophenyl)-8-((2- | J = 6.4, 1.7 Hz, 1H), 8.53 (dd, J = 7.3, 1.7 Hz, 1H), 7.61 |
| | chloropyrimidin-5- | (dd, J = 10.3, 1.5 Hz, 1H), 7.42-7.37 (m, 2H), 7.10 (dd, J = |
| | yl)methyl)pyrido[2,3- | 7.5, 6.5 Hz, 1H), 5.74-5.68 (m, 1H), 5.60 (d, J = 14.7 Hz, |
| | d]pyrimidine-2,4(3H,8H)- | 1H); ESI MS (m/z) 458.35 (M + K)+ |
| | dione | |
| 148 | 8-((2-chloropyrimidin-5- | ¹H-NMR (400 MHz, DMSO-d6) δ 8.88 (s, 2H), 8.66 (dd, |
| | yl)methyl)-3- | J = 6.4, 1.8 Hz, 1H), 8.41 (dd, J = 7.3, 1.8 Hz, 1H), 6.98 (t, |
| | cyclohexylpyrido[2,3- | J = 6.9 Hz, 1H), 5.55 (s, 2H), 4.70-4.64 (m, 1H), 2.38 (td, |
| | d]pyrimidine-2,4(3H,8H)- | J = 12.4, 3.2 Hz, 2H), 1.76 (d, J = 13.1 Hz, 2H), 1.61 (d, J = |
| | dione | 12.2 Hz, 1H), 1.48 (d, J = 11.0 Hz, 2H), 1.32-1.22 (m, |
| | | 2H), 1.17-1.06 (m, 1H)ESI MS (m/z) 372.00 (MH)+ |
| 149 | 8-((2-chlorothiazol-5- | ¹H-NMR (400 MHz, DMSO-d6) δ 8.75 (dd, J = 6.6, 1.7 |
| | yl)methyl)-3-(4- | Hz, 1H), 8.49 (dd, J = 7.3, 1.7 Hz, 1H), 7.89 (s, 1H), 7.33 |
| | (methylthio)phenyl)pyrido[2,3- | (dt, J = 9.0, 2.3 Hz, 2H), 7.16 (dt, J = 9.0, 2.3 Hz, 2H), 7.06 |
| | d]pyrimidine- | (dd, J = 7.3, 6.6 Hz, 1H), 5.72 (s, 2H), 2.51 (s, 3H); ESI |
| | 2,4(3H,8H)-dione | MS (m/z) 416.40 (MH)+. |
| 150 | 8-((2-chlorothiazol-5- | ¹H-NMR (400 MHz, DMSO-d6) δ 8.77 (dd, J = 6.6, 1.7 |
| | yl)methyl)-3-(3- | Hz, 1H), 8.50 (dd, J = 7.3, 1.8 Hz, 1H), 7.90 (s, 1H), 7.60 |
| | (trifluoromethoxy)phenyl)pyrido[2,3- | (t, J = 8.1 Hz, 1H), 7.41 (dt, J = 8.4, 1.1 Hz, 1H), 7.35 (s, |
| | d]pyrimidine- | 1H), 7.31 (dq, J = 7.9, 0.9 Hz, 1H), 7.09 (t, J = 6.9 Hz, 1H), |
| | 2,4(3H,8H)-dione | 5.74 (d, J = 6.4 Hz, 2H); ESI MS (m/z) 454.40 (MH)+. |
| 151 | 8-((2-chlorothiazol-5- | ¹H-NMR (400 MHz, DMSO-d6) δ 8.76 (dd, J = 6.5, 1.8 |
| | yl)methyl)-3-(3- | Hz, 1H), 8.50 (dd, J = 7.3, 1.7 Hz, 1H), 7.90 (s, 1H), 7.51 |
| | (difluoromethoxy)phenyl)pyrido[2,3- | (t, J = 8.2 Hz, 1H), 7.46-7.06 (m, 5H); ESI MS (m/z) |
| | d]pyrimidine- | 436.40 (MH)+. |
| | 2,4(3H,8H)-dione | |
| 152 | 8-((6-chloropyridin-3- | ¹H-NMR (400 MHz, DMSO-d6) δ 8.78 (dd, J = 6.6, 1.7 |
| | yl)methyl)-3-(4- | Hz, 1H), 8.55 (d, J = 2.2 Hz, 1H), 8.52 (dd, J = 7.5, 1.8 Hz, |
| | (methylsulfonyl)phenyl)pyrido[2,3- | 1H), 8.00 (dt, J = 8.9, 2.1 Hz, 2H), 7.94 (dd, J = 8.3, 2.4 |
| | d]pyrimidine- | Hz, 1H), 7.55 (d, J = 8.3 Hz, 1H), 7.52 (dt, J = 8.8, 2.1 Hz, |
| | 2,4(3H,8H)-dione | 2H), 7.08 (dd, J = 7.3, 6.6 Hz, 1H), 5.67 (s, 2H), 3.29 (s, |
| | | 3H); ESI MS (m/z) 442.85 (MH)+. |
| 153 | 8-((2-chlorothiazol-5- | ¹H-NMR (400 MHz, DMSO-d6) δ 8.76 (dd, J = 6.6, 1.7 |
| | yl)methyl)-3-(4- | Hz, 1H), 8.49 (dd, J = 7.3, 1.8 Hz, 1H), 7.90 (s, 1H), 7.28 |
| | fluorophenyl)pyrido[2,3- | (d, J = 7.0 Hz, 4H), 7.07 (dd, J = 7.3, 6.7 Hz, 1H), 5.73 (d, |
| | d]pyrimidine-2,4(3H,8H)- | J = 9.5 Hz, 2H); ESI MS (m/z) 388.40 (MH)+ |
| | dione | |
| 154 | 3-(3-chloro-4- | ¹H-NMR (400 MHz, DMSO-d6) δ 8.91 (d, J = 6.8 Hz, 2H), |
| | fluorophenyl)-8-((2- | 8.78 (dd, J = 6.5, 1.8 Hz, 1H), 8.50 (dd, J = 7.5, 1.8 Hz, |
| | chloropyrimidin-5- | 1H), 7.54-7.47 (m, 2H), 7.28-7.24 (m, 1H), 7.07 (dd, J = |
| | yl)methyl)pyrido[2,3- | 7.3, 6.6 Hz, 1H), 5.65 (s, 2H); ESI MS (m/z) 458.90 |
| | d]pyrimidine-2,4(3H,8H)- | (M + K)+ |
| | dione | |
| 155 | 8-((6-chloropyridin-3- | ¹H-NMR (400 MHz, DMSO-d6) δ 8.71 (dd, J = 6.4, 1.8 |
| | yl)methyl)-3-(4- | Hz, 1H), 8.55 (d, J = 2.1 Hz, 1H), 8.46 (dd, J = 7.3, 1.8 Hz, |
| | (dimethylamino)phenyl)pyrido[2,3- | 1H), 7.93 (dd, J = 8.3, 2.4 Hz, 1H), 7.55 (d, J = 8.3 Hz, |
| | d]pyrimidine- | 1H), 7.02 (t, J = 6.9 Hz, 1H), 6.95-6.92 (m, 2H), 6.73 (d, J = |
| | 2,4(3H,8H)-dione | 8.9 Hz, 2H), 5.63 (s, 2H), 2.91 (s, 6H); ESI MS (m/z) |
| | | 408.00 (MH)+ |
| 156 | 3-(4-fluorophenyl)-8-((6- | ¹H-NMR (400 MHz, DMSO-d6) δ 8.87 (d, J = 2.0 Hz, 1H), |
| | (trifluoromethyl)pyridin-3- | 8.78 (dd, J = 6.6, 1.7 Hz, 1H), 8.51 (dd, J = 7.5, 1.8 Hz, |
| | yl)methyl)pyrido[2,3- | 1H), 8.11-8.09 (m, 1H), 7.93 (d, J = 7.6 Hz, 1H), 7.29-7.21 |
| | d]pyrimidine-2,4(3H,8H)- | (m, 4H), 7.08 (dd, J = 7.3, 6.6 Hz, 1H), 5.75 (d, J = 6.8 Hz, |
| | dione | 2H); ESI MS (m/z) 417.00 (MH)+ |
| 157 | 3-(3-chlorophenyl)-8-((6- | ¹H-NMR (400 MHz, DMSO-d6) δ 8.87 (s, 1H), 8.79 (dd, |
| | (trifluoromethyl)pyridin-3- | J = 6.4, 1.8 Hz, 1H), 8.52 (dd, J = 7.3, 1.8 Hz, 1H), 8.11 |
| | yl)methyl)pyrido[2,3- | (d, J = 8.3 Hz, 1H), 7.94 (d, J = 7.9 Hz, 1H), 7.49-7.43 (m, |
| | d]pyrimidine-2,4(3H,8H)- | 2H), 7.35 (t, J = 1.7 Hz, 1H), 7.19 (dt, J = 7.1, 1.9 Hz, 1H), |
| | dione | 7.09 (t, J = 6.9 Hz, 1H), 5.77 (s, 2H); ESI MS (m/z) 432.95 |
| | | (MH)+ |
| 158 | 8-((2-chloropyrimidin-5- | ¹H-NMR (400 MHz, DMSO-d6) δ 8.92 (s, 2H), 8.79 (dd, |
| | yl)methyl)-3-(3,4- | J = 6.6, 1.7 Hz, 1H), 8.51 (dd, J = 7.3, 1.8 Hz, 1H), 7.72 |
| | dichlorophenyl)pyrido[2,3- | (d, J = 8.6 Hz, 1H), 7.58 (d, J = 2.1 Hz, 1H), 7.25 (dd, J = |
| | d]pyrimidine-2,4(3H,8H)- | 8.6, 2.4 Hz, 1H), 7.08 (dd, J = 7.3, 6.7 Hz, 1H), 5.65 (s, |
| | dione | 2H); ESI MS (m/z) 435.30 (MH)+ |
| 159 | 3-(4-bromophenyl)-8-((2- | ¹H-NMR (400 MHz, DMSO-d6) δ 8.92 (s, 2H), 8.77 (dd, |
| | chloropyrimidin-5- | J = 6.4, 1.8 Hz, 1H), 8.49 (dd, J = 7.3, 1.8 Hz, 1H), 7.65- |
| | yl)methyl)pyrido[2,3- | 7.61 (m, 2H), 7.18-7.15 (m, 2H), 7.06 (dd, J = 7.3, 6.7 Hz, |
| | d]pyrimidine-2,4(3H,8H)- | 1H), 5.64 (s, 2H); ESI MS (m/z) 445.35 (MH)+ |
| | dione | |
| 160 | 8-((2-chloropyrimidin-5- | ¹H-NMR (400 MHz, DMSO-d6) δ 8.92 (s, 2H), 8.76 (dd, |
| | yl)methyl)-3-(4- | J = 6.6, 1.7 Hz, 1H), 8.49 (dd, J = 7.3, 1.5 Hz, 1H), 7.48- |
| | (difluoromethoxy)phenyl)pyrido[2,3- | 7.11 (m, 5H), 7.06 (t, J = 7.0 Hz, 1H), 5.64 (s, 2H); ESI |
| | d]pyrimidine- | MS (m/z) 431.40 (MH)+ |
| | 2,4(3H,8H)-dione | |
| 161 | 8-((6-chloropyridin-3- | ¹H-NMR (400 MHz, DMSO-d6) δ 8.66 (dd, J = 6.6, 1.7 |
| | yl)methyl)-3-(tetrahydro- | Hz, 1H), 8.52 (d, J = 2.0 Hz, 1H), 8.45 (dd, J = 7.3, 1.7 Hz, |
| | 2H-pyran-4-yl)pyrido[2,3- | 1H), 7.89 (dd, J = 8.3, 2.4 Hz, 1H), 7.52 (d, J = 8.3 Hz, |
| | d]pyrimidine-2,4(3H,8H)- | 1H), 7.00 (t, J = 7.0 Hz, 1H), 5.58 (s, 2H), 4.94-4.90 (m, |
| | dione | 1H), 3.91 (dd, J = 11.1, 4.3 Hz, 2H), 3.35 (d, J = 12.0 Hz, |
| | | 2H), 2.66-2.60 (m, 2H), 1.40 (dd, J = 12.0, 3.2 Hz, 2H) ESI |
| | | MS (m/z) 372.95 (MH)+ |
| 162 | 3-(2-fluorophenyl)-8-((6- | ¹H-NMR (400 MHz, DMSO-d6) δ 8.89 (s, 1H), 8.81 (dd, |
| | (trifluoromethyl)pyridin-3- | J = 6.5, 1.8 Hz, 1H), 8.55 (dd, J = 7.5, 1.7 Hz, 1H), 8.11 |
| | yl)methyl)pyrido[2,3- | (d, J = 8.3 Hz, 1H), 7.94 (d, J = 7.9 Hz, 1H), 7.46-7.42 (m, |
| | d]pyrimidine-2,4(3H,8H)- | 1H), 7.36-7.31 (m, 2H), 7.29-7.25 (m, 1H), 7.11 (dd, J = |
| | dione | 7.3, 6.7 Hz, 1H), 5.82 (d, J = 15.0 Hz, 1H), 5.72 (d, J = |
| | | 15.0 Hz, 1H); ESI MS (m/z) 416.95 (MH)+ |
| 163 | 8-((2-chlorothiazol-5- | ¹H-NMR (400 MHz, DMSO-d6) δ 8.67 (dd, J = 6.6, 1.7 |
| | yl)methyl)-3-(tetrahydro- | Hz, 1H), 8.45 (dd, J = 7.6, 1.7 Hz, 1H), 7.86 (s, 1H), 7.01 |
| | 2H-pyran-4-yl)pyrido[2,3- | (t, J = 6.8 Hz, 1H), 5.65 (s, 2H), 4.99-4.92 (m, 1H), 3.93 |
| | d]pyrimidine-2,4(3H,8H)- | (dd, J = 11.0, 4.2 Hz, 2H), 3.37 (d, J = 11.0 Hz, 2H), 2.71- |
| | dione | 2.62 (m, 2H), 1.44 (dd, J = 11.9, 2.6 Hz, 2H) ESI MS (m/z) |
| | | 378.90 (MH)+ |
| 164 | 3-(3-chloro-4- | ¹H-NMR (400 MHz, DMSO-d6) δ 8.78 (dd, J = 6.6, 1.7 |
| | fluorophenyl)-8-((6- | Hz, 1H), 8.55 (d, J = 2.0 Hz, 1H), 8.50 (dd, J = 7.3, 1.7 Hz, |
| | chloropyridin-3- | 1H), 7.94 (dd, J = 8.3, 2.7 Hz, 1H), 7.56-7.53 (m, 2H), 7.49 |
| | yl)methyl)pyrido[2,3- | (t, J = 9.0 Hz, 1H), 7.27 (qd, J = 4.4, 2.4 Hz, 1H), 7.07 (dd, |
| | d]pyrimidine-2,4(3H,8H)- | J = 7.3, 6.6 Hz, 1H), 5.65 (s, 2H); ESI MS (m/z) 458.05 |
| | dione | (M + K)+ |
| 165 | 3-(3-chloro-4- | ¹H-NMR (400 MHz, DMSO-d6) δ 8.77 (dd, J = 6.4, 1.7 |
| | fluorophenyl)-8-((2- | Hz, 1H), 8.51 (dd, J = 7.3, 1.7 Hz, 1H), 7.90 (s, 1H), 7.59 |
| | chlorothiazol-5- | (dd, J = 6.8, 2.4 Hz, 1H), 7.51 (t, J = 9.0 Hz, 1H), 7.31 (qd, |
| | yl)methyl)pyrido[2,3- | J = 4.4, 2.6 Hz, 1H), 7.09 (dd, J = 7.3, 6.6 Hz, 1H), 5.73 (s, |
| | d]pyrimidine-2,4(3H,8H)- | 2H); ESI MS (m/z) 464.05 (M + K)+ |
| | dione | |
| 166 | 2-chloro-5-(8-((2- | ¹H-NMR (400 MHz, DMSO-d6) δ 8.80 (dd, J = 6.5, 1.6 |
| | chlorothiazol-5-yl)methyl)- | Hz, 1H), 8.53 (dd, J = 7.3, 1.7 Hz, 1H), 8.00 (d, J = 2.4 Hz, |
| | 2,4-dioxo-4,8- | 1H), 7.90 (s, 1H), 7.87 (d, J = 8.8 Hz, 1H), 7.70 (dd, J = |
| | dihydropyrido[2,3- | 8.6, 2.4 Hz, 1H), 7.11 (dd, J = 7.3, 6.6 Hz, 1H), 5.74 (s, |
| | d]pyrimidin-3(2H)- | 2H); ESI MS (m/z) 431.90 (MH)+ |
| | yl)benzonitrile | |
| 167 | 3-(4-chloro-3- | ¹H-NMR (400 MHz, DMSO-d6) δ 8.76 (d, J = 5.4 Hz, 1H), |
| | methoxyphenyl)-8-((2- | 8.50 (d, J = 5.9 Hz, 1H), 7.90 (s, 1H), 7.49 (d, J = 8.2 Hz, |
| | chlorothiazol-5- | 1H), 7.09-7.06 (m, 2H), 6.84 (dd, J = 8.1, 2.0 Hz, 1H), 5.72 |
| | yl)methyl)pyrido[2,3- | (s, 2H), 3.81 (s, 3H); ESI MS (m/z) 477.85 (M + K)+ |
| | d]pyrimidine-2,4(3H,8H)- | |
| | dione | |
| 168 | 3-(2-chloro-4- | ¹H-NMR (400 MHz, DMSO-d6) δ 8.80 (dd, J = 6.6, 1.7 |
| | fluorophenyl)-8-((6- | Hz, 1H), 8.57-8.53 (m, 2H), 7.95 (dd, J = 8.3, 2.4 Hz, 1H), |
| | chloropyridin-3- | 7.61 (dd, J = 8.6, 2.7 Hz, 1H), 7.56 (dd, J = 8.3, 0.5 Hz, |
| | yl)methyl)pyrido[2,3- | 1H), 7.45 (dd, J = 8.9, 5.7 Hz, 1H), 7.32 (td, J = 8.6, 2.8 |
| | d]pyrimidine-2,4(3H,8H)- | Hz, 1H), 7.09 (dd, J = 7.5, 6.5 Hz, 1H), 5.76 (d, J = 14.4 |
| | dione | Hz, 1H), 5.56 (d, J = 14.4 Hz, 1H); ESI MS (m/z) 457.40 |
| | | (M + K)+ |
| 169 | 3-(3-chloro-2- | ¹H-NMR (400 MHz, DMSO-d6) δ 8.80 (dd, J = 6.6, 1.7 |
| | fluorophenyl)-8-((6- | Hz, 1H), 8.56-8.54 (m, 2H), 7.95 (dd, J = 8.3, 2.4 Hz, 1H), |
| | chloropyridin-3- | 7.67-7.63 (m, 1H), 7.56-7.54 (m, 1H), 7.39-7.30 (m, 2H), |
| | yl)methyl)pyrido[2,3- | 7.11 (dd, J = 7.3, 6.6 Hz, 1H), 5.73 (d, J = 14.2 Hz, 1H), |
| | d]pyrimidine-2,4(3H,8H)- | 5.60 (d, J = 14.7 Hz, 1H); ESI MS (m/z) 457.55 (M + K)+ |
| | dione | |
| 170 | 3-(4-chloro-3- | ¹H-NMR (400 MHz, DMSO-d6) δ 8.78-8.76 (m, 1H), 8.55 |
| | methoxyphenyl)-8-((6- | (d, J = 2.4 Hz, 1H), 8.50 (dd, J = 7.3, 1.7 Hz, 1H), 7.94 (dd, |
| | chloropyridin-3- | J = 8.3, 2.7 Hz, 1H), 7.56 (d, J = 7.8 Hz, 1H), 7.47 (d, J = |
| | yl)methyl)pyrido[2,3- | 8.6 Hz, 1H), 7.08-7.04 (m, 2H), 6.81 (dd, J = 8.4, 2.1 Hz, |
| | d]pyrimidine-2,4(3H,8H)- | 1H), 5.65 (s, 2H), 3.79 (s, 3H); ESI MS (m/z) 428.40 |
| | dione | (MH)+ |
| 171 | 3-(4-chloro-3- | ¹H-NMR (400 MHz, DMSO-d6) δ 8.92 (s, 2H), 8.77 (dd, |
| | methoxyphenyl)-8-((2- | J = 6.6, 1.7 Hz, 1H), 8.50 (dd, J = 7.5, 1.8 Hz, 1H), 7.48 |
| | chloropyrimidin-5- | (d, J = 8.3 Hz, 1H), 7.08-7.03 (m, 2H), 6.80 (dd, J = 8.3, |
| | yl)methyl)pyrido[2,3- | 2.2 Hz, 1H), 5.64 (s, 2H), 3.79 (s, 3H); ESI MS (m/z) |
| | d]pyrimidine-2,4(3H,8H)- | 472.55 (M + K)+ |
| | dione | |
| 172 | 3-(3- | ¹H-NMR (400 MHz, DMSO-d6) δ 8.87 (d, J = 1.8 Hz, 1H), |
| | (trifluoromethyl)phenyl)-8- | 8.80 (dd, J = 6.4, 1.8 Hz, 1H), 8.53 (dd, J = 7.3, 1.8 Hz, |
| | ((6- | 1H), 8.11 (dd, J = 8.1, 1.7 Hz, 1H), 7.94 (d, J = 8.3 Hz, |
| | (trifluoromethyl)pyridin-3- | 1H), 7.76-7.65 (m, 3H), 7.54 (d, J = 7.9 Hz, 1H), 7.10 (t, J = |
| | yl)methyl)pyrido[2,3- | 6.9 Hz, 1H), 5.76 (d, J = 13.8 Hz, 2H); ESI MS (m/z) |
| | d]pyrimidine-2,4(3H,8H)- | 466.95 (MH)+ |
| | dione | |
| 173 | 8-((6-chloropyridin-3- | ¹H-NMR (400 MHz, DMSO-d6) δ 8.80 (dd, J = 6.4, 1.8 |
| | yl)methyl)-3-(2,3- | Hz, 1H), 8.56-8.54 (m, 2H), 7.95 (dd, J = 8.4, 2.6 Hz, 1H), |
| | difluorophenyl)pyrido[2,3- | 7.56-7.47 (m, 2H), 7.33-7.27 (m, 1H), 7.23-7.19 (m, 1H), |
| | d]pyrimidine-2,4(3H,8H)- | 7.12-7.07 (m, 1H), 5.74-5.59 (m, 2H); ESI MS (m/z) |
| | dione | 400.90 (MH)+ |
| 174 | 8-((6-chloropyridin-3- | ¹H-NMR (400 MHz, DMSO-d6) δ 8.82 (dd, J = 6.6, 1.7 |
| | yl)methyl)-3-(2,6- | Hz, 1H), 8.58 (dd, J = 7.5, 1.7 Hz, 2H), 7.94 (dd, J = 8.4, |
| | difluorophenyl)pyrido[2,3- | 2.6 Hz, 1H), 7.58-7.50 (m, 2H), 7.27 (t, J = 8.1 Hz, 2H), |
| | d]pyrimidine-2,4(3H,8H)- | 7.13 (dd, J = 7.3, 6.7 Hz, 1H), 5.66 (s, 2H); ESI MS (m/z) |
| | dione | 400.90 (MH)+ |
| 175 | 3-benzyl-8-((6- | ¹H-NMR (400 MHz, DMSO-d6) δ 8.70 (dd, J = 6.5, 1.8 |
| | chloropyridin-3- | Hz, 1H), 8.53 (d, J = 2.2 Hz, 1H), 8.49 (dd, J = 7.5, 1.8 Hz, |
| | yl)methyl)pyrido[2,3- | 1H), 7.90 (dd, J = 8.3, 2.7 Hz, 1H), 7.51 (d, J = 7.6 Hz, |
| | d]pyrimidine-2,4(3H,8H)- | 1H), 7.29-7.19 (m, 5H), 7.04 (dd, J = 7.3, 6.4 Hz, 1H), 5.61 |
| | dione | (s, 2H), 5.03 (s, 2H) ESI MS (m/z) 378.95 (MH)+ |
| 176 | 8-((2-chloropyrimidin-5- | ¹H-NMR (400 MHz, DMSO-d6) δ 8.88 (s, 2H), 8.67 (d, J = |
| | yl)methyl)-3- | 4.9 Hz, 1H), 8.43 (d, J = 7.3 Hz, 1H), 6.99 (t, J = 7.0 Hz, |
| | cyclopentylpyrido[2,3- | 1H), 5.56 (s, 2H), 5.24 (t, J = 8.6 Hz, 1H), 2.07-1.99 (m, |
| | d]pyrimidine-2,4(3H,8H)- | 2H), 1.87-1.83 (m, 2H), 1.68 (d, J = 9.2 Hz, 2H), 1.54-1.51 |
| | dione | (m, 2H) ESI MS (m/z) 357.45 (MH)+ |
| 177 | 8-((2-chlorothiazol-5- | ¹H-NMR (400 MHz, DMSO-d6) δ 8.67 (dd, J = 6.6, 1.7 |
| | yl)methyl)-3- | Hz, 1H), 8.44 (dd, J = 7.5, 1.7 Hz, 1H), 7.86 (s, 1H), 7.01 |
| | cyclopentylpyrido[2,3- | (dd, J = 7.3, 6.7 Hz, 1H), 5.65 (s, 2H), 5.27 (t, J = 8.7 Hz, |
| | d]pyrimidine-2,4(3H,8H)- | 1H), 2.08-2.01 (m, 2H), 1.89-1.86 (m, 2H), 1.76-1.68 (m, |
| | dione | 2H), 1.58-1.52 (m, 2H) ESI MS (m/z) 362.45(MH)+ |
| 178 | 8-((6-chloropyridin-3- | ¹H-NMR (400 MHz, DMSO-d6) δ 8.73 (dd, J = 6.5, 1.8 |
| | yl)methyl)-3-(thiophen-3- | Hz, 1H), 8.54 (d, J = 2.0 Hz, 1H), 8.49 (dd, J = 7.3, 2.0 Hz, |
| | yl)pyrido[2,3-d]pyrimidine- | 1H), 7.92 (dd, J = 8.3, 2.4 Hz, 1H), 7.55-7.50 (m, 2H), 7.41 |
| | 2,4(3H,8H)-dione | (q, J = 1.5 Hz, 1H), 7.05 (dd, J = 7.3, 6.6 Hz, 1H), 6.96 (dd, |
| | | J = 5.1, 1.5 Hz, 1H), 5.64 (s, 2H); ESI MS (m/z) 370.50 |
| | | (MH)+ |
| 179 | 8-((2-chloropyrimidin-5- | ¹H-NMR (400 MHz, DMSO-d6) δ 8.91 (s, 2H), 8.75 (dd, |
| | yl)methyl)-3-(thiophen-3- | J = 6.6, 1.7 Hz, 1H), 8.48 (dd, J = 7.6, 1.7 Hz, 1H), 7.53 |
| | yl)pyrido[2,3-d]pyrimidine- | (dd, J = 5.1, 3.2 Hz, 1H), 7.40 (q, J = 1.5 Hz, 1H), 7.05 (t, |
| | 2,4(3H,8H)-dione | J = 7.0 Hz, 1H), 6.96 (dd, J = 5.1, 1.2 Hz, 1H), 5.63 (s, |
| | | 2H); ESI MS (m/z) 371.50 (MH)+ |
| 180 | 8-((2-chlorothiazol-5- | ¹H-NMR (400 MHz, DMSO-d6) δ 8.74 (dd, J = 6.4, 1.7 |
| | yl)methyl)-3-(thiophen-3- | Hz, 1H), 8.49 (dd, J = 7.3, 1.7 Hz, 1H), 7.89 (s, 1H), 7.55 |
| | yl)pyrido[2,3-d]pyrimidine- | (dd, J = 5.1, 3.2 Hz, 1H), 7.46 (q, J = 1.5 Hz, 1H), 7.06 (dd, |
| | 2,4(3H,8H)-dione | J = 7.3, 6.4 Hz, 1H), 7.00 (dd, J = 5.0, 1.3 Hz, 1H), 5.71 (s, |
| | | 2H); ESI MS (m/z) 376.80 (MH)+ |
| 181 | 3-ethyl-8-((6- | ¹H-NMR (400 MHz, DMSO-d6) δ 8.84 (s, 1H), 8.71 (dd, |
| | (trifluoromethyl)pyridin-3- | J = 6.5, 1.8 Hz, 1H), 8.49 (dd, J = 7.5, 1.8 Hz, 1H), 8.03 |
| | yl)methyl)pyrido[2,3- | (d, J = 8.1 Hz, 1H), 7.90 (d, J = 8.1 Hz, 1H), 7.03 (dd, J = |
| | d]pyrimidine-2,4(3H,8H)- | 7.3, 6.6 Hz, 1H), 5.71 (s, 2H), 3.86 (q, J = 7.0 Hz, 2H), |
| | dione | 1.08 (t, J = 7.0 Hz, 3H) ESI MS (m/z) 351.00 (MH)+ |
| 182 | 8-((2-chlorothiazol-5- | ¹H-NMR (400 MHz, DMSO-d6) δ 8.78 (dd, J = 6.6, 1.7 |
| | yl)methyl)-3-(3,4- | Hz, 1H), 8.51 (dd, J = 7.5, 1.7 Hz, 1H), 7.90 (s, 1H), 7.74 |
| | dichlorophenyl)pyrido[2,3- | (d, J = 8.6 Hz, 1H), 7.65 (d, J = 2.4 Hz, 1H), 7.30 (dd, J = |
| | d]pyrimidine-2,4(3H,8H)- | 8.6, 2.4 Hz, 1H), 7.09 (dd, J = 7.3, 6.7 Hz, 1H), 5.73 (s, |
| | dione | 2H) ESI MS (m/z) 440.85 (MH)+ |
| 183 | 8-((2-chlorothiazol-5- | ¹H-NMR (400 MHz, DMSO-d6) δ 8.76 (dd, J = 6.6, 1.7 |
| | yl)methyl)-3-(4- | Hz, 1H), 8.49 (dd, J = 7.3, 1.7 Hz, 1H), 7.90 (s, 1H), 7.49- |
| | (difluoromethoxy)phenyl)pyrido[2,3- | 7.12 (m, 5H), 7.07 (dd, J = 7.3, 6.4 Hz, 1H), 5.73 (d, J = |
| | d]pyrimidine- | 8.3 Hz, 2H) ESI MS (m/z) 436.90 (MH)+ |
| | 2,4(3H,8H)-dione | |
| 184 | 8-((2-chloropyrimidin-5- | ¹H-NMR (400 MHz, DMSO-d6) δ 8.89 (d, J = 2.4 Hz, 2H), |
| | yl)methyl)-3-(4- | 8.66 (qd, J = 3.3, 1.8 Hz, 1H), 8.43-8.40 (m, 1H), 7.00- |
| | methylcyclohexyl)pyrido[2,3- | 6.96 (m, 1H), 5.55 (s, 2H), 4.65 (tt, J = 12.1, 3.8 Hz, 1H), |
| | d]pyrimidine- | 2.63-2.54 (m, 1H), 2.40 (td, J = 12.7, 3.4 Hz, 1H), 1.72 (d, |
| | 2,4(3H,8H)-dione | J = 11.2 Hz, 1H), 1.53 (q, J = 3.3 Hz, 2H), 1.46 (d, J = 9.5 |
| | | Hz, 1H), 1.35-1.22 (m, 2H), 1.02 (d, J = 7.3 Hz, 2H), 0.97- |
| | | 0.84 (m, 2H) ESI MS (m/z) 385.60 (MH)+ |
| 185 | 8-((2-chlorothiazol-5- | ¹H-NMR (400 MHz, DMSO-d6) δ 8.66 (dd, J = 6.6, 2.0 |
| | yl)methyl)-3-(4- | Hz, 1H), 8.44-8.41 (m, 1H), 7.85 (s, 1H), 7.00 (td, J = 6.9, |
| | methylcyclohexyl)pyrido[2,3- | 2.6 Hz, 1H), 5.64 (s, 2H), 4.68 (s, 1H), 2.66-2.59 (m, 1H), |
| | d]pyrimidine- | 2.32 (d, J = 2.0 Hz, 1H), 1.74 (d, J = 14.2 Hz, 1H), 1.57- |
| | 2,4(3H,8H)-dione | 1.48 (m, 3H), 1.26 (d, J = 29.8 Hz, 2H), 1.04 (d, J = 7.1 |
| | | Hz, 2H), 0.89 (d, J = 6.6 Hz, 2H) ESI MS (m/z) 390.60 |
| | | (MH)+ |
| 186 | 8-((6-chloropyridin-3- | ¹H-NMR (400 MHz, DMSO-d6) δ 8.76 (dd, J = 6.4, 1.8 |
| | yl)methyl)-3-(3- | Hz, 1H), 8.55 (d, J = 2.1 Hz, 1H), 8.50 (dd, J = 7.3, 1.8 Hz, |
| | (difluoromethyl)phenyl)pyrido[2,3- | 1H), 7.95 (dd, J = 8.4, 2.6 Hz, 1H), 7.62-7.54 (m, 3H), 7.43 |
| | d]pyrimidine- | (s, 1H), 7.39 (d, J = 6.4 Hz, 1H), 7.21-6.93 (m, 2H), 5.66 |
| | 2,4(3H,8H)-dione | (s, 2H); ESI MS (m/z) 414.95 (MH)+ |
| 187 | 8-((2-chlorothiazol-5- | ¹H-NMR (400 MHz, DMSO-d6) δ 8.77 (dd, J = 6.6, 1.7 |
| | yl)methyl)-3-(3- | Hz, 1H), 8.50 (dd, J = 7.3, 1.7 Hz, 1H), 7.90 (s, 1H), 7.61 |
| | (difluoromethyl)phenyl)pyrido[2,3- | (d, J = 6.8 Hz, 2H), 7.47 (s, 1H), 7.43 (d, J = 6.6 Hz, 1H), |
| | d]pyrimidine- | 7.22-6.94 (m, 2H), 5.74 (d, J = 4.6 Hz, 2H); ESI MS (m/z) |
| | 2,4(3H,8H)-dione | 420.85 (M + Na)+. |
| 188 | 3-(4-chlorophenyl)-8-((6- | ¹H-NMR (400 MHz, DMSO-d6) δ 8.96 (dd, J = 6.4, 1.8 |
| | chloropyridin-3-yl)methyl)- | Hz, 1H), 8.67 (d, J = 2.1 Hz, 1H), 8.56 (dd, J = 7.5, 1.7 Hz, |
| | 2-thioxo-2,8- | 1H), 8.06 (dd, J = 8.3, 2.4 Hz, 1H), 7.56 (d, J = 8.3 Hz, |
| | dihydropyrido[2,3- | 1H), 7.50-7.47 (m, 2H), 7.24 (dd, J = 7.5, 6.6 Hz, 1H), |
| | d]pyrimidin-4(3H)-one | 7.19-7.16 (m, 2H), 5.72 (s, 2H) ESI MS (m/z) 414.85 |
| | | (MH)+ |
| 189 | 8-((6-chloropyridin-3- | ¹H-NMR (400 MHz, DMSO-d6) δ 8.95 (dd, J = 6.4, 1.7 |
| | yl)methyl)-3-(4- | Hz, 1H), 8.67 (d, J = 2.0 Hz, 1H), 8.55 (dd, J = 7.6, 1.7 Hz, |
| | fluorophenyl)-2-thioxo-2,8- | 1H), 8.06 (dd, J = 8.3, 2.4 Hz, 1H), 7.56 (d, J = 8.3 Hz, |
| | dihydropyrido[2,3- | 1H), 7.27-7.22 (m, 3H), 7.19-7.15 (m, 2H), 5.71 (s, 2H) |
| | d]pyrimidin-4(3H)-one | ESI MS (m/z) 398.85 (MH)+ |
| 190 | 8-((6-chloropyridin-3- | ¹H-NMR (400 MHz, DMSO-d6) δ 8.98 (dd, J = 6.6, 1.7 |
| | yl)methyl)-2-thioxo-3-(3- | Hz, 1H), 8.67 (d, J = 2.2 Hz, 1H), 8.57 (dd, J = 7.5, 1.6 Hz, |
| | (trifluoromethyl)phenyl)- | 1H), 8.07 (dd, J = 8.3, 2.4 Hz, 1H), 7.73-7.65 (m, 2H), 7.57 |
| | 2,8-dihydropyrido[2,3- | (d, J = 8.3 Hz, 2H), 7.48 (d, J = 7.6 Hz, 1H), 7.27 (dd, J = |
| | d]pyrimidin-4(3H)-one | 7.3, 6.6 Hz, 1H), 5.73 (dd, J = 20.3, 14.2 Hz, 2H) ESI MS |
| | | (m/z) 448.95 (MH)+ |
| 191 | 3-(4-bromophenyl)-8-((6- | ¹H-NMR (400 MHz, DMSO-d6) δ 8.96 (dd, J = 6.4, 1.5 |
| | chloropyridin-3-yl)methyl)- | Hz, 1H), 8.67 (d, J = 2.1 Hz, 1H), 8.56 (dd, J = 7.5, 1.7 Hz, |
| | 2-thioxo-2,8- | 1H), 8.06 (dd, J = 8.4, 2.6 Hz, 1H), 7.62 (dd, J = 6.6, 2.0 |
| | dihydropyrido[2,3- | Hz, 2H), 7.56 (d, J = 8.3 Hz, 1H), 7.24 (dd, J = 7.5, 6.6 Hz, |
| | d]pyrimidin-4(3H)-one | 1H), 7.11 (dd, J = 6.6, 2.0 Hz, 2H), 5.71 (s, 2H) ESI MS |
| | | (m/z) 460.85 (MH)+ |
| 192 | 3-(4-chlorophenyl)-8- | ¹H-NMR (400 MHz, DMSO-d6) δ 8.61 (dd, J = 6.4, 1.5 |
| | ((tetrahydrofuran-3- | Hz, 1H), 8.48 (dd, J = 7.5, 1.7 Hz, 1H), 7.52-7.49 (m, 2H), |
| | yl)methyl)pyrido[2,3- | 7.26-7.23 (m, 2H), 7.02 (dd, J = 7.3, 6.4 Hz, 1H), 4.45- |
| | d]pyrimidine-2,4(3H,8H)- | 4.36 (m, 2H), 3.87-3.81 (m, 1H), 3.71-3.61 (m, 2H), 3.53 |
| | dione | (dd, J = 8.9, 5.5 Hz, 1H), 3.00-2.91 (m, 1H), 1.96-1.87 (m, |
| | | 1H), 1.72-1.63 (m, 1H); ESI MS (m/z) 357.45 (MH)+ |
| 193 | 3-(4-chloro-2- | ¹H-NMR (400 MHz, DMSO-d6) δ 8.81 (dd, J = 6.5, 1.8 |
| | fluorophenyl)-8-((2- | Hz, 1H), 8.55 (dd, J = 7.3, 1.7 Hz, 1H), 7.90 (s, 1H), 7.69- |
| | chlorothiazol-5- | 7.65 (m, 1H), 7.44-7.40 (m, 1H), 7.34 (td, J = 8.0, 1.1 Hz, |
| | yl)methyl)pyrido[2,3- | 1H), 7.13 (dd, J = 7.3, 6.6 Hz, 1H), 5.75 (dd, J = 26.2, 14.7 |
| | d]pyrimidine-2,4(3H,8H)- | Hz, 2H); ESI MS (m/z) 422.30 (MH)+ |
| | dione | |
| 194 | 8-((6-chloropyridin-3- | ¹H-NMR (400 MHz, DMSO-d6) δ 8.73 (dd, J = 6.5, 1.8 |
| | yl)methyl)-3-(4-methoxy-3- | Hz, 1H), 8.53-8.50 (m, 2H), 7.89 (dd, J = 8.3, 2.4 Hz, 1H), |
| | (trifluoromethoxy)phenyl)pyrido[2,3- | 7.52 (dd, J = 8.3, 0.5 Hz, 1H), 7.06 (dd, J = 7.3, 6.6 Hz, |
| | d]pyrimidine- | 1H), 5.62 (s, 2H), 4.56 (d, J = 2.4 Hz, 2H), 3.02 (t, J = 2.4 |
| | 2,4(3H,8H)-dione | Hz, 1H); ESI MS (m/z) 349.74 (M + Na)+ |
| 195 | 3-(3-chloro-4- | ¹H-NMR (400 MHz, DMSO-d6) δ 8.79 (dd, J = 6.6, 1.7 |
| | (trifluoromethoxy)phenyl)- | Hz, 1H), 8.55 (d, J = 2.2 Hz, 1H), 8.52 (dd, J = 7.5, 1.8 Hz, |
| | 8-((6-chloropyridin-3- | 1H), 7.94 (dd, J = 8.2, 2.6 Hz, 1H), 7.68-7.65 (m, 2H), 7.56 |
| | yl)methyl)pyrido[2,3- | (d, J = 8.1 Hz, 1H), 7.38 (dd, J = 8.7, 2.3 Hz, 1H), 7.08 (t, |
| | d]pyrimidine-2,4(3H,8H)- | J = 7.0 Hz, 1H), 5.66 (s, 2H) ESI MS (m/z) 482.85 (MH)+ |
| | dione | |
| 196 | 8-((6-chloropyridin-3- | ¹H-NMR (400 MHz, DMSO-d6) δ 8.75 (dd, J = 6.6, 1.7 |
| | yl)methyl)-3-(o- | Hz, 1H), 8.57 (d, J = 2.0 Hz, 1H), 8.51 (dd, J = 7.3, 2.0 Hz, |
| | tolyl)pyrido[2,3- | 1H), 7.96 (dd, J = 8.3, 2.4 Hz, 1H), 7.56 (dd, J = 8.2, 0.6 |
| | d]pyrimidine-2,4(3H,8H)- | Hz, 1H), 7.32-7.22 (m, 3H), 7.10-7.04 (m, 2H), 5.66 (dd, J = |
| | dione | 57.8, 14.3 Hz, 2H), 1.99 (s, 3H),ESI MS (m/z) 378.55 |
| | | (MH)+ |
| 197 | 3-(2-chlorophenyl)-8-((6- | ¹H-NMR (400 MHz, DMSO-d6) δ 8.79 (dd, J = 6.4, 1.7 |
| | chloropyridin-3- | Hz, 1H), 8.57-8.52 (m, 2H), 7.95 (dd, J = 8.3, 2.7 Hz, 1H), |
| | yl)methyl)pyrido[2,3- | 7.61-7.55 (m, 2H), 7.43 (dt, J = 9.7, 3.7 Hz, 2H), 7.39-7.35 |
| | d]pyrimidine-2,4(3H,8H)- | (m, 1H), 7.08 (dd, J = 7.3, 6.6 Hz, 1H), 5.76 (d, J = 14.4 |
| | dione | Hz, 1H), 5.56 (d, J = 14.4 Hz, 1H),ESI MS (m/z) 399.40 |
| | | (MH)+ |
| 198 | 8-((2-chloropyrimidin-5- | ¹H-NMR (400 MHz, DMSO-d6) δ 8.92 (s, 2H), 8.78 (dd, |
| | yl)methyl)-3-(2,2- | J = 6.6, 1.7 Hz, 1H), 8.50 (dd, J = 7.5, 1.7 Hz, 1H), 7.48 |
| | difluorobenzo[d][1,3]dioxol- | (d, J = 8.6 Hz, 1H), 7.36 (d, J = 1.8 Hz, 1H), 7.09-7.05 (m, |
| | 5-yl)pyrido[2,3- | 2H), 5.65 (s, 2H); ESI MS (m/z) 445.40 (MH)+ |
| | d]pyrimidine-2,4(3H,8H)- | |
| | dione | |
| 199 | 8-((6-chloropyridin-3- | ¹H-NMR (400 MHz, DMSO-d6) δ 8.77 (dd, J = 6.6, 1.6 |
| | yl)methyl)-3-(2,2- | Hz, 1H), 8.55 (d, J = 2.1 Hz, 1H), 8.51 (dd, J = 7.3, 1.8 Hz, |
| | difluorobenzo[d][1,3]dioxol- | 1H), 7.94 (dd, J = 8.3, 2.4 Hz, 1H), 7.56 (d, J = 8.3 Hz, |
| | 5-yl)pyrido[2,3- | 1H), 7.47 (d, J = 8.6 Hz, 1H), 7.38 (d, J = 1.8 Hz, 1H), |
| | d]pyrimidine-2,4(3H,8H)- | 7.09-7.05 (m, 2H), 5.65 (s, 2H); ESI MS (m/z) 444.45 |
| | dione | (MH)+ |
| 200 | 8-((2-chloropyrimidin-5- | ¹H-NMR (400 MHz, DMSO-d6) δ 8.93 (s, 2H), 8.79 (dd, |
| | yl)methyl)-3-(2- | J = 6.6, 1.7 Hz, 1H), 8.52 (dd, J = 7.3, 1.7 Hz, 1H), 7.48- |
| | fluorophenyl)pyrido[2,3- | 7.42 (m, 1H), 7.36-7.25 (m, 3H), 7.09 (dd, J = 7.3, 6.6 Hz, |
| | d]pyrimidine-2,4(3H,8H)- | 1H), 5.69 (d, J = 14.7 Hz, 1H), 5.60 (d, J = 14.7 Hz, 1H) |
| | dione | ESI MS (m/z) 383.90 (MH)+ |
| 201 | 8-((2-chloropyrimidin-5- | ¹H-NMR (400 MHz, DMSO-d6) δ 8.92 (s, 2H), 8.80 (dd, |
| | yl)methyl)-3-(3,5- | J = 6.6, 1.7 Hz, 1H), 8.51 (dd, J = 7.3, 1.7 Hz, 1H), 7.66 (t, |
| | dichlorophenyl)pyrido[2,3- | J = 1.8 Hz, 1H), 7.39 (d, J = 2.0 Hz, 2H), 7.09 (dd, J = 7.3, |
| | d]pyrimidine-2,4(3H,8H)- | 6.6 Hz, 1H), 5.65 (s, 2H) ESI MS (m/z) 433.80 (MH)+ |
| | dione | |
| 202 | 3-(3-bromophenyl)-8-((6- | ¹H-NMR (400 MHz, DMSO-d6) δ 8.76 (dd, J = 6.6, 1.7 |
| | chloropyridin-3- | Hz, 1H), 8.55 (d, J = 2.4 Hz, 1H), 8.50 (dd, J = 7.3, 1.5 Hz, |
| | yl)methyl)pyrido[2,3- | 1H), 7.94 (dd, J = 8.4, 2.6 Hz, 1H), 7.59-7.54 (m, 2H), 7.48 |
| | d]pyrimidine-2,4(3H,8H)- | (t, J = 1.8 Hz, 1H), 7.41 (t, J = 7.9 Hz, 1H), 7.25-7.23 (m, |
| | dione | 1H), 7.06 (t, J = 7.0 Hz, 1H), 5.65 (s, 2H); ESI MS (m/z) |
| | | 444.20 (MH)+ |
| 203 | 8-((2-chloropyrimidin-5- | ¹H-NMR (400 MHz, DMSO-d6) δ 8.92 (s, 2H), 8.77 (dd, |
| | yl)methyl)-3-(3- | J = 6.6, 1.7 Hz, 1H), 8.50 (dd, J = 7.5, 1.8 Hz, 1H), 7.60 (t, |
| | (difluoromethyl)phenyl)pyrido[2,3- | J = 7.5 Hz, 2H), 7.42 (s, 1H), 7.38 (dd, J = 6.6, 2.2 Hz, 1H), |
| | d]pyrimidine- | 7.21-6.93 (m, 2H), 5.65 (s, 2H); ESI MS (m/z) 415.45 |
| | 2,4(3H,8H)-dione | (MH)+ |
| 204 | 8-((6-chloropyridin-3- | ¹H-NMR (400 MHz, DMSO-d6) δ 8.96 (dd, J = 6.4, 1.7 |
| | yl)methyl)-3-(3- | Hz, 1H), 8.67 (d, J = 2.0 Hz, 1H), 8.56 (dd, J = 7.6, 1.7 Hz, |
| | (difluoromethyl)phenyl)-2- | 1H), 8.06 (dd, J = 8.3, 2.4 Hz, 1H), 7.60-7.53 (m, 3H), 7.36 |
| | thioxo-2,8- | (s, 1H), 7.33 (d, J = 7.6 Hz, 1H), 7.25 (dd, J = 7.5, 6.5 Hz, |
| | dihydropyrido[2,3- | 1H), 7.06 (t, J = 55.8 Hz, 1H), 5.73 (s, 2H); ESI MS (m/z) |
| | d]pyrimidin-4(3H)-one | 430.90 (MH)+ |
| 205 | 8-((2-chloropyrimidin-5- | ¹H-NMR (400 MHz, DMSO-d6) δ 8.93 (s, 2H), 8.81 (dd, |
| | yl)methyl)-3-(2,3- | J = 6.7, 1.8 Hz, 1H), 8.54 (dd, J = 7.3, 1.8 Hz, 1H), 7.54- |
| | difluorophenyl)pyrido[2,3- | 7.47 (m, 1H), 7.33-7.27 (m, 1H), 7.20 (dd, J = 8.1, 6.3 Hz, |
| | d]pyrimidine-2,4(3H,8H)- | 1H), 7.11 (dd, J = 7.5, 6.6 Hz, 1H), 5.74-5.68 (m, 1H), 5.61 |
| | dione | (d, J = 14.7 Hz, 1H) ESI MS (m/z) 401.95 (MH)+ |
| 206 | 8-((2-chloropyrimidin-5- | ¹H-NMR (400 MHz, DMSO-d6) δ 8.93 (s, 2H), 8.83 (dd, |
| | yl)methyl)-3-(2,6- | J = 6.4, 1.8 Hz, 1H), 8.57 (dd, J = 7.5, 1.7 Hz, 1H), 7.53 |
| | difluorophenyl)pyrido[2,3- | (d, J = 8.6 Hz, 1H), 7.27 (t, J = 8.1 Hz, 2H), 7.13 (dd, J = |
| | d]pyrimidine-2,4(3H,8H)- | 7.5, 6.6 Hz, 1H), 5.65 (s, 2H) ESI MS (m/z) 401.85 (MH)+ |
| | dione | |
| 207 | 8-((2-chloropyrimidin-5- | ¹H-NMR (400 MHz, DMSO-d6) δ 8.93 (s, 2H), 8.80 (dd, |
| | yl)methyl)-3-(2,4- | J = 6.6, 1.7 Hz, 1H), 8.53 (dd, J = 7.3, 1.7 Hz, 1H), 7.44- |
| | difluorophenyl)pyrido[2,3- | 7.38 (m, 2H), 7.21-7.16 (m, 1H), 7.09 (dd, J = 7.3, 6.6 Hz, |
| | d]pyrimidine-2,4(3H,8H)- | 1H), 5.69 (d, J = 14.7 Hz, 1H), 5.60 (d, J = 14.7 Hz, 1H) |
| | dione | ESI MS (m/z) 401.85 (MH)+ |
| 208 | 3-(4-chloro-3- | ¹H-NMR (400 MHz, DMSO-d6) δ 8.92 (s, 2H), 8.79 (dd, |
| | fluorophenyl)-8-((2- | J = 6.6, 1.7 Hz, 1H), 8.51 (dd, J = 7.3, 1.7 Hz, 1H), 7.67 (t, |
| | chloropyrimidin-5- | J = 8.4 Hz, 1H), 7.39 (dd, J = 10.0, 2.2 Hz, 1H), 7.13 (dt, J = |
| | yl)methyl)pyrido[2,3- | 8.4, 1.2 Hz, 1H), 7.08 (dd, J = 7.3, 6.6 Hz, 1H), 5.65 (s, |
| | d]pyrimidine-2,4(3H,8H)- | 2H) ESI MS (m/z) 417.85 (MH)+ |
| | dione | |
| 209 | 8-((6-chloropyridin-3- | ¹H-NMR (400 MHz, DMSO-d6) δ 8.96 (dd, J = 6.5, 1.6 |
| | yl)methyl)-2-thioxo-3-(4- | Hz, 1H), 8.67 (d, J = 2.2 Hz, 1H), 8.57 (dd, J = 7.6, 1.7 Hz, |
| | (trifluoromethoxy)phenyl)- | 1H), 8.06 (dd, J = 8.2, 2.6 Hz, 1H), 7.57 (d, J = 8.3 Hz, |
| | 2,8-dihydropyrido[2,3- | 1H), 7.43 (d, J = 8.3 Hz, 2H), 7.29-7.23 (m, 3H), 5.72 (s, |
| | d]pyrimidin-4(3H)-one | 2H) ESI MS (m/z) 464.70 (MH)+ |
| 210 | 3-(4-chlorophenyl)-8-((2- | ¹H-NMR (400 MHz, DMSO-d6) δ 9.04 (s, 2H), 8.97 (dd, |
| | chloropyrimidin-5- | J = 6.5, 1.6 Hz, 1H), 8.56 (dd, J = 7.5, 1.6 Hz, 1H), 7.50- |
| | yl)methyl)-2-thioxo-2,8- | 7.48 (m, 2H), 7.25 (t, J = 7.0 Hz, 1H), 7.17-7.15 (m, 2H), |
| | dihydropyrido[2,3- | 5.69 (s, 2H) ESI MS (m/z) 415.90 (MH)+ |
| | d]pyrimidin-4(3H)-one | |
| 211 | 8-((6-chloropyridin-3- | ¹H-NMR (400 MHz, DMSO-d6) δ 8.76 (dd, J = 6.6, 1.7 |
| | yl)methyl)-3-(3-(2,2,2- | Hz, 1H), 8.55 (d, J = 2.2 Hz, 1H), 8.49 (dd, J = 7.5, 1.6 Hz, |
| | trifluoroethoxy)phenyl)pyrido[2,3- | 1H), 7.94 (dd, J = 8.3, 2.4 Hz, 1H), 7.55 (d, J = 8.3 Hz, |
| | d]pyrimidine- | 1H), 7.39 (t, J = 8.1 Hz, 1H), 7.08-7.04 (m, 2H), 6.93 (d, J = |
| | 2,4(3H,8H)-dione | 2.4 Hz, 1H), 6.87 (d, J = 7.3 Hz, 1H), 5.65 (s, 2H), 4.74 |
| | | (q, J = 8.9 Hz, 2H) ESI MS (m/z) 462.95 (MH)+ |
| 212 | 3-(3-chloro-4- | ¹H-NMR (400 MHz, DMSO-d6) δ 8.79 (dd, J = 6.6, 1.7 |
| | (trifluoromethoxy)phenyl)- | Hz, 1H), 8.52 (dd, J = 7.3, 1.7 Hz, 1H), 7.90 (s, 1H), 7.72- |
| | 8-((2-chlorothiazol-5- | 7.67 (m, 2H), 7.42 (dd, J = 8.6, 2.4 Hz, 1H), 7.10 (t, J = 7.0 |
| | yl)methyl)pyrido[2,3- | Hz, 1H), 5.74 (s, 2H) ESI MS (m/z) 488.60 (MH)+ |
| | d]pyrimidine-2,4(3H,8H)- | |
| | dione | |
| 213 | 8-((2-chloropyrimidin-5- | ¹H-NMR (400 MHz, DMSO-d6) δ 8.93 (d, J = 6.7 Hz, 2H), |
| | yl)methyl)-3-(4-fluoro-3- | 8.79 (dd, J = 6.6, 1.7 Hz, 1H), 8.51 (dd, J = 7.3, 1.8 Hz, |
| | (trifluoromethyl)phenyl)pyrido[2,3- | 1H), 7.73-7.71 (m, 1H), 7.66-7.58 (m, 2H), 7.09 (dd, J = |
| | d]pyrimidine- | 7.3, 6.7 Hz, 1H), 5.66 (s, 2H) ESI MS (m/z) 451.35 (MH)+ |
| | 2,4(3H,8H)-dione | |
| 214 | 8-((2-chlorothiazol-5- | ¹H-NMR (400 MHz, DMSO-d6) δ 8.81 (dd, J = 6.4, 1.7 |
| | yl)methyl)-3-(2,3- | Hz, 1H), 8.55 (dd, J = 7.5, 1.8 Hz, 1H), 7.90 (s, 1H), 7.53 |
| | difluorophenyl)pyrido[2,3- | (dd, J = 16.3, 8.7 Hz, 1H), 7.35-7.24 (m, 2H), 7.12 (dd, J = |
| | d]pyrimidine-2,4(3H,8H)- | 7.5, 6.5 Hz, 1H), 5.75 (dd, J = 24.9, 14.7 Hz, 2H) ESI |
| | dione | MS (m/z) 406.85 (MH)+ |
| 215 | 8-((2-chlorothiazol-5- | ¹H-NMR (400 MHz, DMSO-d6) δ 8.83 (dd, J = 6.5, 1.8 |
| | yl)methyl)-3-(2,6- | Hz, 1H), 8.58 (dd, J = 7.3, 1.7 Hz, 1H), 7.90 (s, 1H), 7.60- |
| | difluorophenyl)pyrido[2,3- | 7.53 (m, 1H), 7.29 (t, J = 8.1 Hz, 2H), 7.15 (dd, J = 7.5, 6.5 |
| | d]pyrimidine-2,4(3H,8H)- | Hz, 1H), 5.75 (s, 2H) ESI MS (m/z) 406.85 (MH)+ |
| | dione | |
| 216 | 8-((2-chloropyrimidin-5- | ¹H-NMR (400 MHz, DMSO-d6) δ 8.92 (s, 2H), 8.75 (dd, |
| | yl)methyl)-3-(4-(2,2,2- | J = 6.6, 1.7 Hz, 1H), 8.48 (dd, J = 7.3, 1.5 Hz, 1H), 7.15- |
| | trifluoroethoxy)phenyl)pyrido[2,3- | 7.08 (m, 4H), 7.04 (dd, J = 7.3, 6.4 Hz, 1H), 5.63 (s, 2H), |
| | d]pyrimidine- | 4.80 (q, J = 8.9 Hz, 2H) ESI MS (m/z) 463.40 (MH)+ |
| | 2,4(3H,8H)-dione | |
| 217 | 3-(3-chloro-2- | ¹H-NMR (400 MHz, DMSO-d6) δ 8.81 (dd, J = 6.6, 1.7 |
| | fluorophenyl)-8-((2- | Hz, 1H), 8.55 (dd, J = 7.3, 1.7 Hz, 1H), 7.90 (s, 1H), 7.69- |
| | chlorothiazol-5- | 7.65 (m, 1H), 7.44-7.40 (m, 1H), 7.34 (dd, J = 9.0, 8.1 Hz, |
| | yl)methyl)pyrido[2,3- | 1H), 7.13 (dd, J = 7.3, 6.6 Hz, 1H), 5.78 (d, J = 14.4 Hz, |
| | d]pyrimidine-2,4(3H,8H)- | 1H), 5.71 (d, J = 14.4 Hz, 1H); ESI MS (m/z) 422.85 |
| | dione | (MH)+ |
| 218 | 3-(3-chloro-4- | ¹H-NMR (400 MHz, DMSO-d6) δ 8.98 (dd, J = 6.4, 1.7 |
| | fluorophenyl)-8-((6- | Hz, 1H), 8.67 (d, J = 2.2 Hz, 1H), 8.57 (dd, J = 7.3, 1.7 Hz, |
| | chloropyridin-3-yl)methyl)- | 1H), 8.06 (dd, J = 8.3, 2.4 Hz, 1H), 7.57 (d, J = 8.3 Hz, |
| | 2-thioxo-2,8- | 1H), 7.51-7.46 (m, 2H), 7.26 (dd, J = 7.5, 6.5 Hz, 1H), 7.20 |
| | dihydropyrido[2,3- | (qd, J = 4.4, 2.4 Hz, 1H), 5.72 (s, 2H); ESI MS (m/z) |
| | d]pyrimidin-4(3H)-one | 432.85 (MH)+ |
| 219 | 3-(4-chloro-3- | ¹H-NMR (400 MHz, DMSO-d6) δ 8.79 (dd, J = 6.4, 1.7 |
| | (trifluoromethyl)phenyl)-8- | Hz, 1H), 8.55 (d, J = 2.2 Hz, 1H), 8.52 (dd, J = 7.3, 1.7 Hz, |
| | ((6-chloropyridin-3- | 1H), 7.94 (dd, J = 8.3, 2.4 Hz, 1H), 7.83 (d, J = 8.8 Hz, |
| | yl)methyl)pyrido[2,3- | 2H), 7.60 (dd, J = 8.4, 2.3 Hz, 1H), 7.56 (d, J = 8.1 Hz, |
| | d]pyrimidine-2,4(3H,8H)- | 1H), 7.09 (t, J = 7.0 Hz, 1H), 5.66 (s, 2H) ESI MS (m/z) |
| | dione | 467.00 (MH)+ |
| 220 | 3-(4-chloro-3- | ¹H-NMR (400 MHz, DMSO-d6) δ 8.96 (dd, J = 6.4, 1.8 |
| | methoxyphenyl)-8-((6- | Hz, 1H), 8.67 (d, J = 2.1 Hz, 1H), 8.57 (dd, J = 7.5, 1.7 Hz, |
| | chloropyridin-3-yl)methyl)- | 1H), 8.06 (dd, J = 8.3, 2.4 Hz, 1H), 7.57 (d, J = 8.3 Hz, |
| | 2-thioxo-2,8- | 1H), 7.47 (d, J = 8.3 Hz, 1H), 7.25 (dd, J = 7.6, 6.4 Hz, |
| | dihydropyrido[2,3- | 1H), 7.00 (d, J = 2.1 Hz, 1H), 6.76 (dd, J = 8.3, 2.1 Hz, |
| | d]pyrimidin-4(3H)-one | 1H), 5.74-5.69 (m, 2H), 3.77 (s, 3H); ESI MS (m/z) 444.90 |
| | | (MH)+ |
| 221 | 8-((6-chloropyridin-3- | ¹H-NMR (400 MHz, DMSO-d6) δ 8.93 (dd, J = 6.4, 1.5 |
| | yl)methyl)-3-(thiophen-3- | Hz, 1H), 8.66 (d, J = 2.1 Hz, 1H), 8.54 (dd, J = 7.5, 1.7 Hz, |
| | yl)-2-thioxo-2,8- | 1H), 8.05 (dd, J = 8.3, 2.4 Hz, 1H), 7.56 (d, J = 8.3 Hz, |
| | dihydropyrido[2,3- | 1H), 7.52 (dd, J = 5.0, 3.2 Hz, 1H), 7.32 (q, J = 1.5 Hz, |
| | d]pyrimidin-4(3H)-one | 1H), 7.23 (dd, J = 7.5, 6.6 Hz, 1H), 6.90 (dd, J = 5.0, 1.4 |
| | | Hz, 1H), 5.70 (s, 2H); ESI MS (m/z) 386.80 (MH)+ |
| 222 | 8-((6-chloropyridin-3- | ¹H-NMR (400 MHz, DMSO-d6) δ 8.79 (dd, J = 6.6, 1.7 |
| | yl)methyl)-3-(4-fluoro-3- | Hz, 1H), 8.55 (d, J = 2.0 Hz, 1H), 8.51 (dd, J = 7.3, 1.7 Hz, |
| | (trifluoromethyl)phenyl)pyrido[2,3- | 1H), 7.94 (dd, J = 8.3, 2.7 Hz, 1H), 7.75-7.73 (m, 1H), |
| | d]pyrimidine- | 7.66-7.55 (m, 3H), 7.08 (dd, J = 7.3, 6.6 Hz, 1H), 5.66 (s, |
| | 2,4(3H,8H)-dione | 2H) ESI MS (m/z) 450.90 (MH)+ |
| 223 | 8-((6-chloropyridin-3- | ¹H-NMR (400 MHz, DMSO-d6) δ 8.74 (dd, J = 6.6, 1.7 |
| | yl)methyl)-3-(4-(2,2,2- | Hz, 1H), 8.55 (d, J = 2.2 Hz, 1H), 8.48 (dd, J = 7.3, 1.7 Hz, |
| | trifluoroethoxy)phenyl)pyrido[2,3- | 1H), 7.94 (dd, J = 8.3, 2.4 Hz, 1H), 7.55 (d, J = 8.1 Hz, |
| | d]pyrimidine- | 1H), 7.16-7.13 (m, 2H), 7.10-7.08 (m, 2H), 7.04 (t, J = 7.0 |
| | 2,4(3H,8H)-dione | Hz, 1H), 5.64 (s, 2H), 4.80 (q, J = 8.9 Hz, 2H) ESI MS |
| | | (m/z) 463.00 (MH)+ |
| 224 | 3-(4-chloro-3- | ¹H-NMR (400 MHz, DMSO-d6) δ 8.92 (s, 2H), 8.80 (dd, |
| | (trifluoromethyl)phenyl)-8- | J = 6.7, 1.8 Hz, 1H), 8.51 (dd, J = 7.3, 1.8 Hz, 1H), 7.84- |
| | ((2-chloropyrimidin-5- | 7.79 (m, 2H), 7.59 (dd, J = 8.6, 2.4 Hz, 1H), 7.09 (dd, J = |
| | yl)methyl)pyrido[2,3- | 7.3, 6.7 Hz, 1H), 5.66 (s, 2H) ESI MS (m/z) 467.95 (MH)+ |
| | d]pyrimidine-2,4(3H,8H)- | |
| | dione | |
| 225 | 3-(2-chloro-4- | ¹H-NMR (400 MHz, DMSO-d6) δ 8.93 (s, 2H), 8.81 (dd, |
| | fluorophenyl)-8-((2- | J = 6.5, 1.8 Hz, 1H), 8.53 (dd, J = 7.3, 1.7 Hz, 1H), 7.61 |
| | chloropyrimidin-5- | (dd, J = 8.6, 2.7 Hz, 1H), 7.43 (dd, J = 8.8, 5.6 Hz, 1H), |
| | yl)methyl)pyrido[2,3- | 7.32 (td, J = 8.5, 2.9 Hz, 1H), 7.09 (dd, J = 7.3, 6.6 Hz, |
| | d]pyrimidine-2,4(3H,8H)- | 1H), 5.74 (d, J = 14.7 Hz, 1H), 5.56 (d, J = 14.7 Hz, 1H); |
| | dione | ESI MS (m/z) 417.95 (MH)+ |
| 226 | 3-(3-chloro-2- | ¹H-NMR (400 MHz, DMSO-d6) δ 8.94 (d, J = 6.8 Hz, 2H), |
| | fluorophenyl)-8-((2- | 8.81 (dd, J = 6.6, 1.7 Hz, 1H), 8.54 (dd, J = 7.6, 1.7 Hz, |
| | chloropyrimidin-5- | 1H), 7.67-7.63 (m, 1H), 7.38-7.30 (m, 2H), 7.11 (t, J = 7.0 |
| | yl)methyl)pyrido[2,3- | Hz, 1H), 5.71 (d, J = 14.7 Hz, 1H), 5.61 (d, J = 14.4 Hz, |
| | d]pyrimidine-2,4(3H,8H)- | 1H); ESI MS (m/z) 417.90 (MH)+ |
| | dione | |
| 227 | 3-(3-chloro-4- | ¹H-NMR (400 MHz, DMSO-d6) δ 8.92 (s, 2H), 8.80 (dd, |
| | (trifluoromethoxy)phenyl)- | J = 6.6, 1.7 Hz, 1H), 8.52 (dd, J = 7.3, 1.7 Hz, 1H), 7.68- |
| | 8-((2-chloropyrimidin-5- | 7.65 (m, 2H), 7.37 (dd, J = 8.7, 2.3 Hz, 1H), 7.09 (t, J = 7.0 |
| | yl)methyl)pyrido[2,3- | Hz, 1H), 5.66 (s, 2H) ESI MS (m/z) 483.90 (MH)+ |
| | d]pyrimidine-2,4(3H,8H)- | |
| | dione | |
| 228 | 8-((6-chloropyridin-3- | ¹H-NMR (400 MHz, DMSO-d6) δ 8.98 (dd, J = 6.4, 1.5 |
| | yl)methyl)-3-(2,4- | Hz, 1H), 8.67 (d, J = 2.4 Hz, 1H), 8.59 (dd, J = 7.6, 1.5 Hz, |
| | difluorophenyl)-2-thioxo- | 1H), 8.06 (dd, J = 8.3, 2.4 Hz, 1H), 7.57 (d, J = 8.3 Hz, |
| | 2,8-dihydropyrido[2,3- | 1H), 7.41-7.33 (m, 2H), 7.28 (t, J = 6.9 Hz, 1H), 7.17 (dd, |
| | d]pyrimidin-4(3H)-one | J = 8.6, 1.8 Hz, 1H), 5.78 (d, J = 14.1 Hz, 1H), 5.66 (d, J = |
| | | 14.4 Hz, 1H); ESI MS (m/z) 416.90 (MH)+ |
| 229 | 8-((6-chloropyridin-3- | ¹H-NMR (400 MHz, DMSO-d6) δ 8.75 (dd, J = 6.4, 1.8 |
| | yl)methyl)-3-(3-fluoro-4- | Hz, 1H), 8.55 (d, J = 2.4 Hz, 1H), 8.48 (dd, J = 7.3, 1.8 Hz, |
| | methoxyphenyl)pyrido[2,3- | 1H), 7.94 (dd, J = 8.4, 2.6 Hz, 1H), 7.55 (d, J = 8.3 Hz, |
| | d]pyrimidine-2,4(3H,8H)- | 1H), 7.20 (t, J = 9.2 Hz, 1H), 7.13 (dd, J = 12.1, 2.3 Hz, |
| | dione | 1H), 7.05 (t, J = 6.9 Hz, 1H), 7.01-6.96 (m, 1H), 5.64 (s, |
| | | 2H), 3.87 (s, 3H); ESI MS (m/z) 412.90 (MH)+ |
| 230 | 3-(3-bromo-4- | ¹H-NMR (400 MHz, DMSO-d6) δ 8.75 (dd, J = 6.4, 1.8 |
| | methoxyphenyl)-8-((6- | Hz, 1H), 8.55 (d, J = 2.1 Hz, 1H), 8.48 (dd, J = 7.5, 1.7 Hz, |
| | chloropyridin-3- | 1H), 7.94 (dd, J = 8.4, 2.6 Hz, 1H), 7.55 (d, J = 8.3 Hz, |
| | yl)methyl)pyrido[2,3- | 1H), 7.44 (d, J = 2.1 Hz, 1H), 7.21-7.15 (m, 2H), 7.05 (t, J = |
| | d]pyrimidine-2,4(3H,8H)- | 6.9 Hz, 1H), 5.65 (s, 2H), 3.88 (s, 3H); ESI MS (m/z) |
| | dione | 474.80 (MH)+ |
| 231 | 3-(3-bromophenyl)-8-((2- | ¹H-NMR (400 MHz, DMSO-d6) δ 8.93 (d, J = 10.4 Hz, |
| | chloropyrimidin-5- | 2H), 8.77 (dd, J = 6.4, 1.5 Hz, 1H), 8.49 (dd, J = 7.5, 1.7 |
| | yl)methyl)pyrido[2,3- | Hz, 1H), 7.59-7.57 (m, 1H), 7.49-7.46 (m, 1H), 7.41 (t, J = |
| | d]pyrimidine-2,4(3H,8H)- | 7.9 Hz, 1H), 7.23 (d, J = 7.9 Hz, 1H), 7.07 (t, J = 6.9 Hz, |
| | dione | 1H), 5.74 (s, 1H), 5.63 (d, J = 14.4 Hz, 2H); ESI MS (m/z) |
| | | 445.85 (MH)+ |
| 232 | 3-(3-bromo-4- | ¹H-NMR (400 MHz, DMSO-d6) δ 8.78 (dd, J = 6.6, 1.7 |
| | fluorophenyl)-8-((6- | Hz, 1H), 8.55 (d, J = 2.0 Hz, 1H), 8.50 (dd, J = 7.6, 1.7 Hz, |
| | chloropyridin-3- | 1H), 7.94 (dd, J = 8.3, 2.4 Hz, 1H), 7.64 (dd, J = 6.4, 2.4 |
| | yl)methyl)pyrido[2,3- | Hz, 1H), 7.56-7.54 (m, 1H), 7.46 (t, J = 8.7 Hz, 1H), 7.30 |
| | d]pyrimidine-2,4(3H,8H)- | (qd, J = 4.4, 2.4 Hz, 1H), 7.07 (dd, J = 7.3, 6.6 Hz, 1H), |
| | dione | 5.65 (s, 2H); ESI MS (m/z) 462.75 (MH)+ |
| 233 | 3-(4-bromo-3- | ¹H-NMR (400 MHz, DMSO-d6) δ 8.78 (dd, J = 6.6, 1.7 |
| | fluorophenyl)-8-((6- | Hz, 1H), 8.55 (d, J = 2.0 Hz, 1H), 8.51 (dd, J = 7.5, 1.8 Hz, |
| | chloropyridin-3- | 1H), 7.94 (dd, J = 8.3, 2.4 Hz, 1H), 7.79 (t, J = 8.2 Hz, 1H), |
| | yl)methyl)pyrido[2,3- | 7.55 (dd, J = 8.3, 0.5 Hz, 1H), 7.36 (dd, J = 9.5, 2.2 Hz, |
| | d]pyrimidine-2,4(3H,8H)- | 1H), 7.09-7.06 (m, 2H), 5.65 (s, 2H); ESI MS (m/z) 462.80 |
| | dione | (MH)+ |
| 234 | 3-(3-bromo-4- | ¹H-NMR (400 MHz, DMSO-d6) δ 8.92 (s, 2H), 8.78 (dd, |
| | fluorophenyl)-8-((2- | J = 6.4, 1.7 Hz, 1H), 8.50 (dd, J = 7.3, 1.7 Hz, 1H), 7.63 |
| | chloropyrimidin-5- | (dd, J = 6.4, 2.4 Hz, 1H), 7.46 (t, J = 8.8 Hz, 1H), 7.29 (qd, |
| | yl)methyl)pyrido[2,3- | J = 4.4, 2.4 Hz, 1H), 7.07 (t, J = 7.0 Hz, 1H), 5.65 (s, 2H); |
| | d]pyrimidine-2,4(3H,8H)- | ESI MS (m/z) 463.80 (MH)+ |
| | dione | |
| 235 | 3-(4-bromo-3- | ¹H-NMR (400 MHz, DMSO-d6) δ 8.92 (s, 2H), 8.79 (dd, |
| | fluorophenyl)-8-((2- | J = 6.4, 1.7 Hz, 1H), 8.51 (dd, J = 7.3, 1.7 Hz, 1H), 7.79 (t, |
| | chloropyrimidin-5- | J = 8.2 Hz, 1H), 7.35 (dd, J = 9.5, 2.2 Hz, 1H), 7.10-7.05 |
| | yl)methyl)pyrido[2,3- | (m, 2H), 5.64 (s, 2H); ESI MS (m/z) 463.90 (MH)+ |
| | d]pyrimidine-2,4(3H,8H)- | |
| | dione | |
| 236 | 3-(2-chloro-4- | ¹H-NMR (400 MHz, DMSO-d6) δ 8.98 (dd, J = 6.6, 1.7 |
| | fluorophenyl)-8-((6- | Hz, 1H), 8.67 (d, J = 2.1 Hz, 1H), 8.60 (dd, J = 7.5, 1.7 Hz, |
| | chloropyridin-3-yl)methyl)- | 1H), 8.07 (dd, J = 8.4, 2.6 Hz, 1H), 7.57 (dd, J = 8.4, 2.3 |
| | 2-thioxo-2,8- | Hz, 2H), 7.38 (dd, J = 8.9, 5.8 Hz, 1H), 7.33-7.25 (m, 2H), |
| | dihydropyrido[2,3- | 5.82 (d, J = 14.1 Hz, 1H), 5.62 (d, J = 14.1 Hz, 1H) ESI |
| | d]pyrimidin-4(3H)-one | MS (m/z) 432.95 (MH)+ |
| 237 | 3-(2-chloro-4- | ¹H-NMR (400 MHz, DMSO-d6) δ 8.80 (dd, J = 6.6, 1.7 |
| | fluorophenyl)-8-((2- | Hz, 1H), 8.54 (dd, J = 7.5, 1.8 Hz, 1H), 7.90 (s, 1H), 7.63 |
| | chlorothiazol-5- | (dd, J = 8.7, 2.8 Hz, 1H), 7.50 (dd, J = 8.8, 5.9 Hz, 1H), |
| | yl)methyl)pyrido[2,3- | 7.34 (td, J = 8.5, 2.8 Hz, 1H), 7.11 (dd, J = 7.5, 6.5 Hz, |
| | d]pyrimidine-2,4(3H,8H)- | 1H), 5.79 (d, J = 14.4 Hz, 1H), 5.70 (d, J = 14.4 Hz, 1H); |
| | dione | ESI MS (m/z) 422.85 (MH)+ |
| 238 | 8-((6-chloropyridin-3- | ¹H-NMR (400 MHz, DMSO-d6) δ 8.73 (dd, J = 6.6, 1.7 |
| | yl)methyl)-3-(1-methyl-1H- | Hz, 1H), 8.54 (d, J = 2.1 Hz, 1H), 8.47 (dd, J = 7.5, 1.7 Hz, |
| | pyrazol-3-yl)pyrido[2,3- | 1H), 7.91 (dd, J = 8.3, 2.4 Hz, 1H), 7.71 (d, J = 2.1 Hz, |
| | d]pyrimidine-2,4(3H,8H)- | 1H), 7.53 (d, J = 8.3 Hz, 1H), 7.04 (dd, J = 7.2, 6.6 Hz, |
| | dione | 1H), 6.10 (d, J = 2.4 Hz, 1H), 5.63 (s, 2H), 3.82 (s, 3H) |
| | | ESI MS (m/z) 368.95 (MH)+ |
| 239 | 8-((6-chloropyridin-3- | ¹H-NMR (400 MHz, DMSO-d6) δ 8.67 (dd, J = 6.6, 1.7 |
| | yl)methyl)-3-(1- | Hz, 1H), 8.53 (d, J = 2.2 Hz, 1H), 8.42 (dd, J = 7.3, 1.7 Hz, |
| | phenylethyl)pyrido[2,3- | 1H), 7.90 (dd, J = 8.3, 2.4 Hz, 1H), 7.53 (d, J = 8.3 Hz, |
| | d]pyrimidine-2,4(3H,8H)- | 1H), 7.28-7.23 (m, 4H), 7.19-7.15 (m, 1H), 7.00 (dd, J = |
| | dione | 7.3, 6.6 Hz, 1H), 6.15 (q, J = 7.1 Hz, 1H), 5.58 (s, 2H), |
| | | 1.75 (d, J = 7.1 Hz, 3H) ESI MS (m/z) 392.95 (MH)+ |
| 240 | 8-((6-chloropyridin-3- | ¹H-NMR (400 MHz, DMSO-d6) δ 8.99 (dd, J = 6.6, 1.7 |
| | yl)methyl)-3-(3,4- | Hz, 1H), 8.66 (d, J = 2.2 Hz, 1H), 8.58 (dd, J = 7.3, 1.7 Hz, |
| | dichlorophenyl)-2-thioxo- | 1H), 8.06 (dd, J = 8.3, 2.4 Hz, 1H), 7.71 (d, J = 8.3 Hz, |
| | 2,8-dihydropyrido[2,3- | 1H), 7.57 (d, J = 8.1 Hz, 1H), 7.53 (d, J = 2.4 Hz, 1H), 7.27 |
| | d]pyrimidin-4(3H)-one | (dd, J = 7.5, 6.5 Hz, 1H), 7.20 (dd, J = 8.4, 2.3 Hz, 1H), |
| | | 5.73 (d, J = 10.8 Hz, 2H) ESI MS (m/z) 450.75 (MH)+ |
| 241 | 8-((6-chloropyridin-3- | ¹H-NMR (400 MHz, DMSO-d6) δ 8.88 (dd, J = 6.4, 1.5 |
| | yl)methyl)-3-(1- | Hz, 1H), 8.68 (d, J = 2.1 Hz, 1H), 8.41 (dd, J = 7.6, 1.5 Hz, |
| | phenylethyl)-2-thioxo-2,8- | 1H), 8.07 (dd, J = 8.3, 2.4 Hz, 1H), 7.57 (d, J = 8.3 Hz, |
| | dihydropyrido[2,3- | 1H), 7.46 (d, J = 7.0 Hz, 1H), 7.30-7.23 (m, 4H), 7.21-7.14 |
| | d]pyrimidin-4(3H)-one | (m, 2H), 5.75-5.72 (m, 1H), 5.65 (d, J = 14.1 Hz, 1H), 1.77 |
| | | (d, J = 7.0 Hz, 3H) ESI MS (m/z) 409.05 (MH)+ |
| 242 | 8-((2-chlorothiazol-5- | ¹H-NMR (400 MHz, DMSO-d6) δ 8.79 (dd, J = 6.6, 1.7 |
| | yl)methyl)-3-(4-fluoro-3- | Hz, 1H), 8.51 (dd, J = 7.5, 1.7 Hz, 1H), 7.90 (s, 1H), 7.79 |
| | (trifluoromethyl)phenyl)pyrido[2,3- | (dd, J = 6.6, 2.3 Hz, 1H), 7.70-7.60 (m, 2H), 7.10 (t, J = |
| | d]pyrimidine- | 6.9 Hz, 1H), 5.74 (s, 2H) ESI MS (m/z) 456.95 (MH)+ |
| | 2,4(3H,8H)-dione | |
| 243 | 8-((2-chlorothiazol-5- | ¹H-NMR (400 MHz, DMSO-d6) δ 8.74 (dd, J = 6.6, 1.7 |
| | yl)methyl)-3-(4-(2,2,2- | Hz, 1H), 8.48 (dd, J = 7.3, 1.5 Hz, 1H), 7.89 (s, 1H), 7.20- |
| | trifluoroethoxy)phenyl)pyrido[2,3- | 7.17 (m, 2H), 7.13-7.10 (m, 2H), 7.06 (dd, J = 7.3, 6.7 Hz, |
| | d]pyrimidine- | 1H), 5.72 (s, 2H), 4.81 (q, J = 8.9 Hz, 2H) ESI MS (m/z) |
| | 2,4(3H,8H)-dione | 468.90 (MH)+ |
| 244 | 8-((2-chloropyrimidin-5- | ¹H-NMR (400 MHz, DMSO-d6) δ 8.92 (s, 2H), 8.81 (dd, |
| | yl)methyl)-3-(3-fluoro-5- | J = 6.6, 1.7 Hz, 1H), 8.52 (dd, J = 7.3, 1.7 Hz, 1H), 7.75 |
| | (trifluoromethyl)phenyl)pyrido[2,3- | (d, J = 8.6 Hz, 1H), 7.56 (d, J = 7.6 Hz, 2H), 7.10 (dd, J = |
| | d]pyrimidine- | 7.3, 6.6 Hz, 1H), 5.66 (s, 2H) ESI MS (m/z) 451.90 (MH)+ |
| | 2,4(3H,8H)-dione | |
| 245 | 8-((2-chloropyrimidin-5- | ¹H-NMR (400 MHz, DMSO-d6) δ 8.92 (s, 2H), 8.74 (dd, |
| | yl)methyl)-3-(4- | J = 6.6, 1.7 Hz, 1H), 8.47 (dd, J = 7.5, 1.8 Hz, 1H), 7.12 |
| | cyclopropylphenyl)pyrido[2,3- | (d, J = 8.6 Hz, 2H), 7.05-7.01 (m, 3H), 5.63 (s, 2H), 1.95 |
| | d]pyrimidine- | (t, J = 8.2 Hz, 1H), 0.99-0.94 (m, 2H), 0.71-0.68 (m, 2H) |
| | 2,4(3H,8H)-dione | ESI MS (m/z) 406.00 (MH)+ |
| 246 | 8-((6-chloropyridin-3- | ¹H-NMR (400 MHz, DMSO-d6) δ 8.77 (dd, J = 6.6, 1.7 |
| | yl)methyl)-3-(3- | Hz, 1H), 8.55 (d, J = 2.2 Hz, 1H), 8.50 (dd, J = 7.3, 1.7 Hz, |
| | ((trifluoromethyl)thio)phenyl)pyrido[2,3- | 1H), 7.94 (dd, J = 8.3, 2.4 Hz, 1H), 7.73 (d, J = 8.1 Hz, |
| | d]pyrimidine- | 1H), 7.62 (t, J = 7.8 Hz, 2H), 7.57-7.54 (m, 1H), 7.48 (dq, |
| | 2,4(3H,8H)-dione | J = 7.8, 1.1 Hz, 1H), 7.07 (dd, J = 7.3, 6.6 Hz, 1H), 5.66 (s, |
| | | 2H),ESI MS (m/z) 464.90 (MH)+ |
| 247 | 8-((2-chlorothiazol-5- | ¹H-NMR (400 MHz, DMSO-d6) δ 8.77 (dd, J = 6.6, 1.7 |
| | yl)methyl)-3-(2,2- | Hz, 1H), 8.51 (dd, J = 7.3, 1.7 Hz, 1H), 7.90 (s, 1H), 7.50 |
| | difluorobenzo[d][1,3]dioxol- | (d, J = 8.6 Hz, 1H), 7.43 (d, J = 1.7 Hz, 1H), 7.13-7.07 (m, |
| | 5-yl)pyrido[2,3- | 2H), 5.73 (s, 2H); ESI MS (m/z) 450.85 (MH)+ |
| | d]pyrimidine-2,4(3H,8H)- | |
| | dione | |
| 248 | 3-(4-bromo-3- | ¹H-NMR (400 MHz, DMSO-d6) δ 8.77 (dd, J = 6.4, 1.7 |
| | fluorophenyl)-8-((2- | Hz, 1H), 8.50 (dd, J = 7.3, 1.7 Hz, 1H), 7.90 (s, 1H), 7.69 |
| | chlorothiazol-5- | (dd, J = 6.4, 2.4 Hz, 1H), 7.48 (t, J = 8.7 Hz, 1H), 7.37- |
| | yl)methyl)pyrido[2,3- | 7.32 (m, 1H), 7.09 (dd, J = 7.3, 6.6 Hz, 1H), 5.73 (s, 2H); |
| | d]pyrimidine-2,4(3H,8H)- | ESI MS (m/z) 466.90 (MH)+ |
| | dione | |
| 249 | 3-(3-bromophenyl)-8-((2- | ¹H-NMR (400 MHz, DMSO-d6) δ 8.77 (dd, J = 6.4, 1.7 |
| | chlorothiazol-5- | Hz, 1H), 8.50 (dd, J = 7.6, 1.7 Hz, 1H), 7.90 (s, 1H), 7.61- |
| | yl)methyl)pyrido[2,3- | 7.59 (m, 1H), 7.52 (t, J = 1.8 Hz, 1H), 7.43 (t, J = 7.9 Hz, |
| | d]pyrimidine-2,4(3H,8H)- | 1H), 7.29-7.26 (m, 1H), 7.08 (t, J = 7.0 Hz, 1H), 5.73 (s, |
| | dione | 2H); ESI MS (m/z) 450.80 (MH)+ |

* Compound names generated using Chemdraw Professional 19.1

As described herein, the compounds of formula (I) show insecticidal activities which are exerted with respect to numerous insects which attack on important agricultural crops. The compounds of the present invention were assessed for their activity as described in the following tests:

### BIOLOGY EXAMPLES

### Example A: Bemisia tabaci

The leaf dip method was used for testing, wherein the required quantity of the compound was weighed and dissolved in a tube containing a solvent solution. The tube was put on a vortex at 2000 rpm for 90 min for proper mixing and then the mixture was diluted with a 0.01% Triton-X solution to the desired test concentration. Brinjal leaves were dipped in the compound solution for 10 seconds, shade dried for 20 min and then placed, with the abaxial side of the leaf up, on 4 ml of a 1% agar-agar solution in respective container caps. Known numbers of freshly emerged whitefly adults were collected, using a modified aspirator, and released into a perforated container in which the cap containing the treated leaf was placed. The containers were kept in a plant growth chamber at a temperature of 25° C. and a relative humidity of 70%. Observations on dead, moribund and alive adults were recorded 72 h after the release. Percent mortality was calculated by combining dead and moribund adults and comparing the result to the one of the untreated control. The compounds 32 53 58 79 80 81 82 84 87 88 89 94 95 96 101 102 103 104 105 106 107 108 109 112 113 114 115 116 117 118 119 120 123 124 125 126 127 130 135 138 139 140 144 145 147 150 154 156 158 159 164 167 168 169 170 171 188 189 191 198 199 200 201 202 206 207 208 209 210 213 216 223 224 225 227 228 231 232 233 234 235 236 238 242 243 244 246 and 247 recorded≥70 percent mortality @ 300 PPM.

### Example B: Myzus persicae

The leaf dip method was used for testing, wherein the required quantity of the compound was weighed and dissolved in a tube containing a solvent solution. The tube was put on a vortex at 2000 rpm for 90 min for proper mixing, then the mixture was diluted with a 0.01% Triton-X solution to the desired test concentration. Capsicum leaves were dipped in the compound solution for 10 seconds, shade dried for 20 min and then placed, with the abaxial side of the leaf up, in single cells of a bio-assay tray containing 4 ml of a solidified 1% agar-agar solution. Known numbers of third instar nymphs, collected in petri plates, were released into the cell with the treated leaf and the cell was covered with a perforated lid for better aeration. The trays were kept in a plant growth chamber at a temperature of 25° C. and a relative humidity of 70%. Observations on dead, moribund and alive nymphs were recorded 72 h after the release. Percent mortality was calculated by combining dead and moribund nymphs and comparing the result with the one of the untreated control. The compounds 1 2 3 4 11 14 15 16 17 22 23 24 26 28 29 32 53 56 58 59 64 68 73 76 79 80 81 82 83 84 87 88 89 92 94 95 96 98 101 102 103 104 105 106 107 108 109 111 113 114 115 116 117 118 119 120 123 124 125 126 127 135 138 139 140 141 150 151 154 159 160 168 169 172 174 178 179 186 187 188 189 190 191 193 195 196 197 198 199 201 203 204 205 207 208 210 211 212 213 214 216 217 218 222 223 225 226 228 231 232 233 234 235 236 238 239 240 242 243 244 246 and 247 recorded ≥70 percent mortality @ 300 PPM.

### Example C: Nilaparvata lugens

The seedling dip method was used for testing, wherein the required quantity of the compound was weighed and dissolved in a tube containing solvent solution. The tube was put on a vortex at 2000 rpm for 90 min for proper mixing, then the mixture was diluted with a 0.01% Triton-X solution to the desired test concentration. Paddy seedlings were dipped in the compound solution for 10 seconds, shade dried for 20 min and then the seedlings were placed in glass test tubes with the roots kept in water. 15 third instar nymphs were released into each test tube and the tubes were kept in a plant growth chamber at a temperature of 25° C. and a relative humidity of 75%. Observations on dead, moribund and alive nymphs were recorded 72 h after the release. Percent mortality was calculated by combining dead and moribund nymphs and comparing the result to the one of the untreated control. The compounds 2 3 4 11 15 16 22 23 28 29 32 34 35 37 41 44 53 57 58 59 62 64 67 72 73 74 76 79 82 87 88 92 94 95 96 101 102 103 105 106 109 111 112 113 114 115 116 117 118 119 120 121 123 124 125 127 128 130 135 138 139 140 141 143 147 150 151 153 154 159 160 162 164 165 168 169 171 173 174 178 179 186 187 188 189 190 191 193 195 196 197 198 199 200 201 202 203 204 205 206 207 208 209 210 211 212 213 214 215 216 217 218 219 221 223 224 225 226 228 231 232 233 234 235 236 237 238 242 243 244 245 246 247 and 249 recorded ≥70 percent mortality @ 300 PPM.

Having described the invention with reference to certain preferred embodiments, other embodiments will become apparent to one skilled in the art from the consideration of the specification. It will be apparent to those skilled in the art that many modifications, both to materials and methods, may be practiced without departing from the scope of the invention.

1. A compound of formula (I) 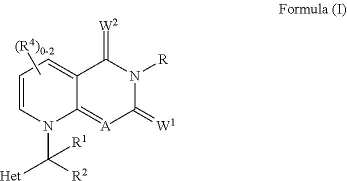
wherein,
R is selected from the group consisting of hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₁₀-alkyl-OR⁵, —C(═O)—R″, —C₁-C₁₀-alkyl-C(═O)—R″, —C₁-C₆-alkyl-NR′R″, —C(═O)—NR′R″, —C₁-C₁₀-alkyl-C(═O)—NR′R″, —C₂-C₆-alkenyl-C(═O)—N(R′R″), —C₁-C₁₀-alkyl-S(O)ₙR⁶, —S(O)ₙR⁶, —S(O)₂-phenyl, —S(O)₂-C₃-C₁₀-heterocyclyl, C₃-C₁₀-cycloalkyl, C₃-C₁₀-halocycloalkyl, C₃-C₁₀-cyanocycloalkyl, (halo)C₃-C₁₀-cycloalkyl-C₁-C₁₀-alkyl, C₃-C₁₀-cyanocycloalkyl-C₁-C₁₀-alkyl, C₄-C₁₀-cycloalkenyl, C₄-C₁₀-cycloalkynyl, bicyclic C₅-C₁₂-alkyl, bicyclic C₇-C₁₂-alkenyl, C₆-C₁₀-aryl, C₆-C₁₀-aryl-C₁-C₆-alkyl, C₃-C₁₀-heterocyclyl and C₃-C₁₀-heterocyclyl-C₁-C₆-alkyl; wherein each group may optionally be substituted with one or more R^{b};
R^{b} is selected from the group consisting of halogen, CN, C₁-C₆-alkyl, C₁-C₆-cyanoalkyl, C₁-C₆-haloalkyl, —OR⁵, —C(═O)—R″, —NR′R″, —S(O)ₙR″ and C₃-C₆-cycloalkyl;
or
R is selected from the group consisting of phenyl, naphthyl and C₃-C₁₀-heterocyclyl; wherein each group may optionally be substituted with one or more R^{x};
R^{x} is independently selected from the group consisting of halogen, hydrogen, cyano, NO₂, C₁-C₁-alkyl, C₁-C₁-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₆-alkyl-OR⁵, —C(═O)—R″, —NR′R″, —C₁-C₆-alkyl-NR′R″, —NR′C(═O)—R″, —C(═O)—NR′R″, —C₁-C₁₀-alkyl-C(═O)—NR′R″, —C₁-C₆-alkyl-S(O)ₙR⁶, —S(O)ₙR⁶, C₃-C₁-cycloalkyl, C₃-C₁₀-halocycloalkyl, C₃-C₁₀-cyanocycloalkyl, —SF₅, —SCN, —S(═O)₀₋₁(R⁸)(═NR⁷), —N═S(═O)₀₋₁(R⁹)(R¹⁰), (halo)C₃-C₁₀-cycloalkyl-C₁-C₆-alkyl, C₃-C₁₀-cycloalkyl-C₁-C₆-alkenyl, C₃-C₁₀-cycloalkyl-C₁-C₆-alkynyl, C₃-C₁₀-cyanocycloalkyl-C₁-C₆-alkyl, C₄-C₁₀-cycloalkenyl, bicyclic C₅-C₁₂-alkyl, bicyclic C₇-C₁₂-alkenyl, phenyl, benzyl, phenylethyl, C₃-C₆-heterocyclyl and C₃-C₆-heterocyclyl-C₁-C₆-alkyl; wherein each group may optionally be substituted with one or more R^{c};
R^{c} is selected from the group consisting of halogen, CN, C₁-C₆-alkyl and C₁-C₆-alkoxy;
A represents CR^{a} or N;
R^{a} is selected from the group consisting of hydrogen, cyano, C₁-C₄ alkyl, C₁-C₄ haloalkyl, C₁-C₄ cycloalkyl and —(C═O)—R″;
W¹ and W² are independently selected from O or S;
“n” is an integer ranging from 0 to 2;
R¹ and R² are independently selected from the group consisting of hydrogen, halogen, cyano, C₁-C₄ alkyl and C₁-C₄ haloalkyl;
Het is selected from the 5-6 membered heterocyclyl ring; which may optionally be substituted with one or more R³;
R³ is independently selected from the group consisting of hydrogen, halogen, cyano, C₁-C₆ alkyl and C₁-C₆ haloalkyl;
R⁴ is selected from the group consisting of hydrogen, halogen, cyano, C₁-C₆ alkyl, C₂-C₆ alkenyl, C₂-C₆ alkynyl, C₂-C₆ haloalkenyl, C₂-C₆ haloalkynyl, C₁-C₆ haloalkyl, C₁-C₆ alkoxy and C₁-C₆ haloalkoxy;
R⁵ is selected from the group consisting of hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₂-C₆ haloalkynyl-C₁-C₄-alkyl, C₃-C₁₀-cycloalkyl, C₃-C₁₀-halocycloalkyl and (halo)C₃-C₁₀-cycloalkyl-C₁-C₆-alkyl;
R⁶ is selected from the group consisting of hydrogen, —NR′R″, C₁-C₆ alkyl, C₁-C₆ haloalkyl, C₃-C₁₀-cycloalkyl, C₃-C₁₀-halocycloalkyl and (halo)C₃-C₁₀-cycloalkyl-C₁-C₆-alkyl;
R⁷ is selected from the group consisting of hydrogen, cyano, C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₆ alkylcarbonyl and C₁-C₄ haloalkylcarbonyl;
R⁸ is selected from the group consisting of C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₆ haloalkyl and C₃-C₆ halocycloalkyl;
R⁹ and R¹⁰ are independently selected from the group consisting of C₁-C₆ alkyl, C₂-C₆ alkenyl, C₂-C₆ alkynyl, C₃-C₆ cycloalkyl, (halo)C₃-C₆ cycloalkyl-C₁-C₄ alkyl, C₁-C₄ haloalkyl and C₃-C₆ halocycloalkyl; or
R⁹ and R¹⁰ together with the S atom to which they are attached may form a 4-to 6-membered heterocyclic ring, wherein the C atoms of the heterocyclic ring may be optionally replaced by C(═O) or C(═S); wherein said ring may optionally be substituted with one or more R^{c};
R′ is selected from the group consisting of hydrogen and C₁-C₆-alkyl; wherein said alkyl may optionally be substituted with one or more halogen or C₃-C₆-cycloalkyl;
R″ is selected from the group consisting of hydrogen, C₁-C₆-alkyl, C₁-C₆-haloalkyl, C₃-C₁₀-cycloalkyl, —N(R′)₂ and —OR′; wherein each group may optionally be substituted with one or more halogen; or
R″ represent phenyl; wherein said phenyl may optionally be substituted with the group selected from halogen, cyano, C₁-C₄ alkyl, C₁-C₄ alkoxy or C₁-C₄ haloalkyl;
or
R′ and R″ together with the N atom to which they are attached may form a 4-to 6-membered heterocyclic ring, wherein the C atoms of the heterocyclic ring may be optionally replaced by C(═O) or C(═S); wherein said ring may optionally be substituted with one or more R^{C};
and/or salts, stereo-isomers, metal complexes, polymorphs, or N-oxides thereof.

2. The compound of formula (I) according to claim 1, wherein R is selected from the group consisting of phenyl, naphthyl and C₃-C₁₀-heterocyclyl; wherein each group may optionally be substituted with one or more R^{x}; said R^{x} is independently selected from the group consisting of halogen, hydrogen, cyano, NO₂, C₁-C₁₀-alkyl, C₁-C₁₀-cyanoalkyl, C₂-C₁₀-alkenyl, C₂-C₁₀-alkynyl, C₁-C₁₀-haloalkyl, C₂-C₁₀-haloalkenyl, C₂-C₁₀-haloalkynyl, —OR⁵, —C₁-C₆-alkyl-OR⁵, —C(═O)—R″, —NR′R″, —C₁-C₆-alkyl-NR′R″, —NR′C(═O)—R″, —C(═O)—NR′R″, —C₁-C₁₀-alkyl-C(═O)—NR′R″, —C₁-C₆-alkyl-S(O)ₙR⁶, —S(O)ₙR⁶, C₃-C₁₀-cycloalkyl, C₃-C₁₀-halocycloalkyl, C₃-C₁₀-cyanocycloalkyl, —SF₅, —SCN, —S(═O)₀₋₁(R′)(═NR⁷), —N═S(═O)₀₋₁(R⁹)(R¹⁰), (halo)C₃-C₁₀-cycloalkyl-C₁-C₆-alkyl, C₃-C₁₀-cycloalkyl-C₁-C₆-alkenyl, C₃-C₁₀-cycloalkyl-C₁-C₆-alkynyl, C₃-C₁₀-cyanocycloalkyl-C₁-C₆-alkyl, C₄-C₁₀-cycloalkenyl, bicyclic C₅-C₁₂-alkyl, bicyclic C₇-C₁₂-alkenyl, phenyl, benzyl, phenylethyl, C₃-C₆-heterocyclyl and C₃-C₆-heterocyclyl-C₁-C₆-alkyl; wherein each group may optionally be substituted with one or more R^{c};
R^{c} is selected from the group consisting of halogen, CN, C₁-C₆-alkyl and C₁-C₆-alkoxy;
A represents CR^{a} or N;
R^{a} is selected from the group consisting of hydrogen, cyano, C₁-C₄ alkyl, C₁-C₄ haloalkyl, C₁-C₄ cycloalkyl and —(C═O)—R″;
W¹ and W² are independently selected from O or S;
“n” is an integer ranging from 0 to 2;
R¹ and R² are independently selected from the group consisting of hydrogen, halogen, cyano, C₁-C₄ alkyl and C₁-C₄ haloalkyl;
Het is selected from the 5-6 membered heterocyclyl ring; which may optionally be substituted with one or more R³;
R³ is independently selected from the group consisting of hydrogen, halogen, cyano, C₁-C₄ alkyl and C₁-C₄ haloalkyl;
R⁴ is selected from the group consisting of hydrogen, halogen, cyano, C₁-C₄ alkyl, C₂-C₄ alkenyl, C₂-C₄ alkynyl, C₂-C₄ haloalkenyl, C₂-C₄ haloalkynyl, C₁-C₄ haloalkyl, C₁-C₄ alkoxy and C₁-C₄ haloalkoxy;
R⁵is selected from the group consisting of hydrogen, C₁-C₄ alkyl, C₂-C₄ alkenyl, C₂-C₄ alkynyl, C₂-C₄ haloalkenyl, C₂-C₄ haloalkynyl, C₂-C₄ haloalkynyl-C₁-C₂-alkyl, C₁-C₄ haloalkyl, C₃-C₆-cycloalkyl, C₃-C₆-halocycloalkyl and (halo)C₃-C₆-cycloalkyl-C₁-C₄-alkyl;
R⁶ is selected from the group consisting of hydrogen, —NR′R″, C₁-C₄ alkyl, C₁-C₄ haloalkyl, C₃-C₁₀-cycloalkyl, C₃-C₁₀-halocycloalkyl and (halo)C₃-C₁₀-cycloalkyl-C₁-C₆-alkyl;
R⁷ is selected from the group consisting of hydrogen, cyano, C₁-C₄ alkyl, C₂-C₄ alkenyl, C₂-C₄ alkynyl, C₃-C₆ cycloalkyl, (halo)C₃-C₆ cycloalkyl-C₁-C₃ alkyl, C₁-C₃ alkylcarbonyl and C₁-C₃ haloalkylcarbonyl;
R⁸ is independently selected from the group consisting of C₁-C₄ alkyl, C₂-C₄ alkenyl, C₂-C₄ alkynyl, C₃-C₆ cycloalkyl, (halo)C₃-C₆ cycloalkyl-C₁-C₃ alkyl, C₁-C₄ haloalkyl and C₃-C₆ halocycloalkyl;
R⁹ and R¹⁰ are independently selected from the group consisting of C₁-C₄ alkyl, C₂-C₄ alkenyl, C₂-C₄ alkynyl, C₃-C₆ cycloalkyl, (halo)C₃-C₆ cycloalkyl-C₁-C₃ alkyl, C₁-C₄ haloalkyl and C₃-C₆ halocycloalkyl; or
R⁹ and R¹⁰ together with the S atom to which they are attached may form a 4-to 6-membered heterocyclic ring, wherein the C atoms of the heterocyclic ring may be optionally replaced by C(═O) or C(═S); wherein said ring may optionally be substituted with one or more R^{C};
R′ is selected from the group consisting of hydrogen and C₁-C₆-alkyl; wherein said alkyl may optionally be substituted with one or more halogen or C₃-C₆-cycloalkyl;
R″ is selected from the group consisting of hydrogen, C₁-C₆-alkyl, C₁-C₆-haloalkyl, C₃-C₁₀-cycloalkyl, —N(R′)₂ and —OR′; wherein each group may optionally be substituted with one or more halogen; or
R″ represents phenyl; wherein said phenyl may optionally be substituted with the group selected from halogen, cyano, C₁-C₄ alkyl, C₁-C₄ alkoxy or C₁-C₄ haloalkyl; wherein said alkyl may optionally be substituted with one or more halogen;
or
R′ and R″ together with the N atom to which they are attached may form a 4-to 6-membered heterocyclic ring, wherein the C atoms of the heterocyclic ring may be optionally replaced by C(═O) or C(═S); wherein said ring may optionally be substituted with one or more R^{c};
and/or salts, stereo-isomers, metal complexes, polymorphs, or N-oxides thereof.

3. The compound of formula (I) according to claim 1, wherein Het is selected from one of Het-1 to Het-6: 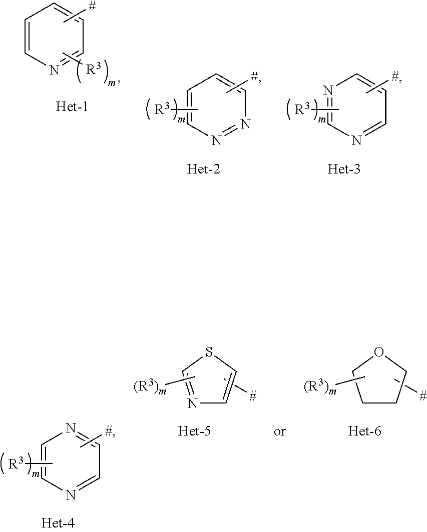
wherein,
#denote the attachment point to the remainder of the molecule;
m is 0, 1, or 2; and R³ is independently selected from the group consisting of hydrogen, halogen, cyano, C₁-C₄ alkyl and C₁-C₄ haloalkyl.

4. The compound of formula (I) according to claim 3, wherein Het is selected from one of Het-1-a to Het-6-b:

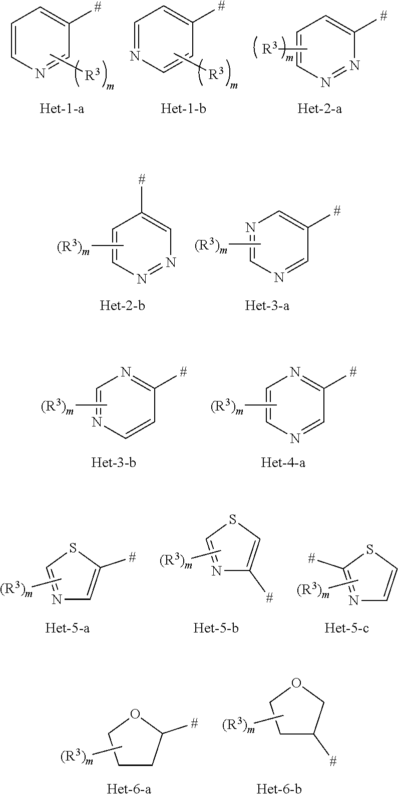

5. The compound of formula (I) according to claim 1, wherein W¹ and W² are O.

6. The compound of formula (I) according to claim 1, wherein A is nitrogen (N).

7. The compound of formula (I) according to claim 1, wherein:
R is selected from hydrogen, cyano, C₁-C₆-alkyl, C₁-C₆-cyanoalkyl, C₂-C₆-alkenyl, C₂-C₆-alkynyl, C₁-C₆-haloalkyl, —C(═O)—R″, —OR⁵, —C₁-C₆-alkyl-C(═O)—R″, —C₁-C₆-alkyl-OR⁵, —S(O)₂-phenyl, —S(O)₂-C₃-C₁₀-heterocyclyl, C₃-C₆-cycloalkyl, C₃-C₆-cyanocycloalkyl, (halo)C₃-C₆-cycloalkyl-C₁-C₃-alkyl, phenyl-C₁-C₃-alkyl or C₃-C₆-heterocyclyl-C₁-C₃-alkyl; wherein each group may optionally be substituted with one or more R^{b}; or
R is selected from phenyl, naphthyl, oxetanyl, azetidinyl, thietanyl, thietanyl 1-oxide, thietanyl 1,1-dioxide, tetrahydro-2H-pyran-4-yl, thienyl, furanyl, pyrrolyl, isoxazolyl, isothiazolyl, pyrazolyl, oxazolyl, thiazolyl, imidazolyl, I,2,4-oxadiazolyl, I,2,4-thiadiazolyl, I,2,4-triazolyl, I,2,3-triazolyl, I,3,4-oxadiazolyl, I,3,4-thiadiazolyl, I,3,4-triazolyl, tetrazolyl, pyridinyl, pyridazinyl, pyrimidinyl, pyrazinyl, indolyl, benzimidazolyl, indazolyl, benzofuranyl, benzothiazolyl, benzoxazolyl, 2,2-difluorobenzo[d][1,3]dioxol-5-yl, quinolinyl or isoquinolinyl; wherein each group may optionally be substituted with one or more R^{x}.

8. The compound of formula (I) according to claim 1, wherein: R is phenyl; wherein said phenyl may optionally be substituted with one or more R^{x}.

9. The compound of formula (I) according to claim 1, wherein R^{b} is independently selected from the group consisting of halogen, CN, C₁-C₆-alkyl, C₁-C₆-cyanoalkyl, C₁-C₆-haloalkyl, —OR⁵, —C(═O)—R″, —NR′R″, —S(O)ₙR″ and C₃-C₆-cycloalkyl;
R^{x} is independently selected from halogen, hydrogen, cyano, NO₂, C₁-C₆-alkyl, C₁-C₆-cyanoalkyl, C₂-C₆-alkenyl, C₂-C₆-alkynyl, C₁-C₆-haloalkyl, C₂-C₆-haloalkenyl, C₂-C₆-haloalkynyl, C₃-C₆-cycloalkyl, —OR⁵, —C(═O)—R″, —NR′R″, —C(═O)—NR′R″, —S(═O)₀₋₁ (R⁸)(═NR⁷), —N═S(═O)₀₋₁(R⁹)(R¹⁰) or —S(O)ₙR⁶; wherein each group of said R^{x} may optionally be substituted with one or more R^{c}.

10. The compound of formula (I) according to claim 1, wherein R¹ and R² are hydrogen.

11. The compound of formula (I) according to claim 1, wherein R³ is independently selected from halogen or C₁-C₄ haloalkyl.

12. A method for the preparation of the compound of formula (I) according to claim 1, wherein said method comprises the steps selected from a) to e):
a) reacting a compound of formula (III) with a compound of formula (VI), optionally in the presence of a suitable catalyst, a suitable solvent and a suitable base, to obtain a compound of formula (II) or (IIB), which on further reacting with a carbonylating or a thiocarbonylating reagent in the presence of a suitable solvent and a suitable base provided a compound of formula (I), 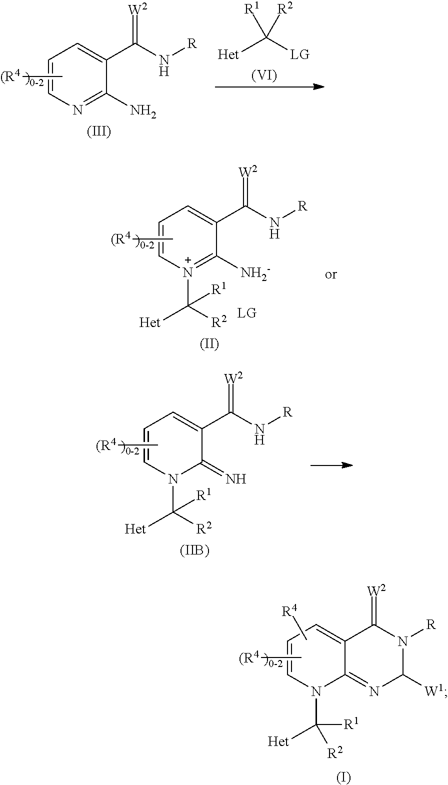
or
b) reacting a compound of formula (III) with a carbonylating or a thiocarbonylating reagent in the presence of a suitable solvent and a suitable base, to obtain a compound of formula (VIII), which on further reacting with a compound of formula (VI), optionally in the presence of a suitable catalyst, a suitable solvent and a suitable base, provided the compound of formula (I), 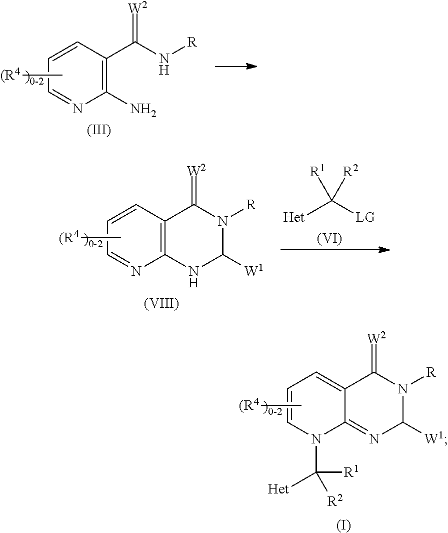
c) further, the compound of formula (III) is obtained by reacting a compound of formula (IV) with a compound of formula (V) in the presence of a suitable reagent, a suitable solvent and a suitable base to obtain the compound of formula (III) 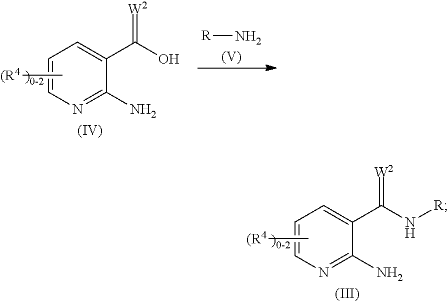
alternatively,
d) compound of formula (I) is obtained by reacting a compound of formula (IIA) with a compound of formula (IIIA) or (IVB), optionally in the presence of a suitable catalyst, a suitable solvent and a suitable base, 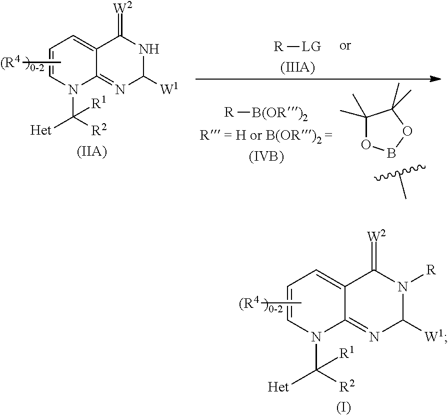
e) reacting a compound of formula (IIIB) with a compound of formula (IVA) to obtain a compound of formula (VA) in the presence of a suitable catalyst, a suitable solvent and a suitable base. Conducting a cyclocondensation reaction of the compound of formula (VA) and a compound of formula (VIA), optionally in the presence of a base, to obtain the compound of formula (IIA), 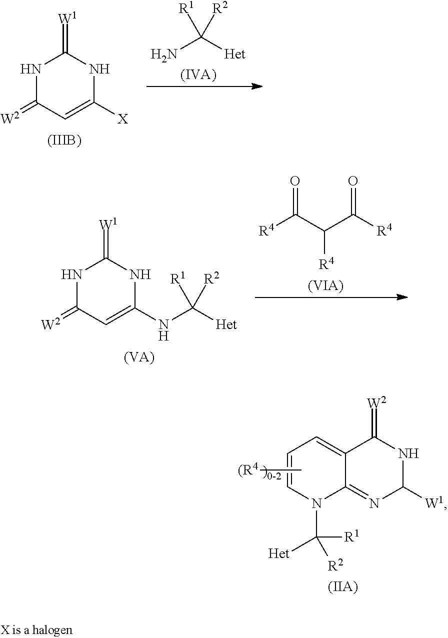
wherein, LG is a halogen or tosylate or nosylate, or mesylate.

13. A compound of formulae (II) and (IIA), 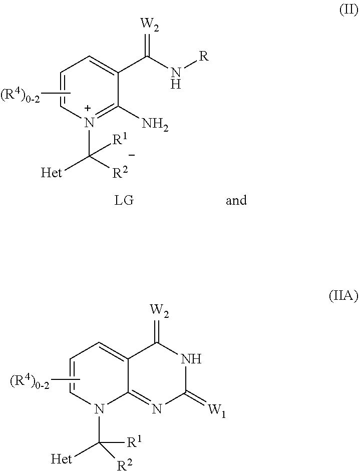
wherein, LG is a halogen or tosylate or nosylate, or mesylate; Het, R⁴, R¹, R², R, W¹ and W² are as defined in claim 1.

14. A compound of formula (IIB), 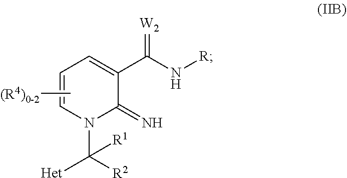
wherein, R═phenyl or C₃-C₁₀-heterocyclyl; wherein each group may optionally be substituted with one or more R^{x}; LG is a halogen or tosylate or nosylate, or mesylate; Het, R⁴, R¹, R², R, W¹ and W² are as defined in claim 1.

15. A composition comprising the compound of formula (I) or salts, metal complexes, stereo-isomers, polymorphs or N-oxides thereof according to claim 1 and at least one additional component selected from the group consisting of surfactants and auxiliaries.

16. The composition according to claim 15, wherein the said composition additionally comprises at least one biological active compatible compound selected from fungicides, insecticides, nematicides, acaricides, biopesticides, herbicides, plant growth regulators, antibiotics, fertilizers or nutrients.

17. The composition according to claim 15, wherein the said compound of formula (I) ranges from 0.1% to 99% by weight with respect to the total weight of the composition.

18. A combination comprising a biologically effective amount of the compound of formula (I) according to claim 1 and at least one additional biological active compatible compound selected from fungicides, insecticides, nematicides, acaricides, biopesticides, herbicides, plant growth regulators, antibiotics, fertilizers and nutrients.

19. A method for combating insects and mite pests comprising contacting the insects and mite pests, their habitat, breeding ground, food supply, plant, seed, soil, area, material or environment in which the insect and mite pests are growing or may grow, or the materials, plants, seeds, soils, surfaces or spaces to be protected from a pest attack or an infestation with a biologically effective amount of a compound of formula (I), salts, stereo-isomers, polymorphs, metal complexes, or N-oxides thereof according to claim 1.

20. (canceled)

21. (canceled)

22. A seed comprising a compound of formula (I) or salts, metal complexes, N-oxides, stereo-isomers, or polymorphs thereof according to claim 1, wherein the amount of the compound of formula (I) in the said seed is ranging from 0.0001% to 1% by weight.
